(19) United States
(12) Patent Application Publication
(10) Pub. No.: US 2016/0193154 A1
(43) Pub. Date: Jul. 7, 2016

(54) Pharmaceutical Composition for Oral Insulin Administration Comprising a Tablet Core and an Anionic Copolymer Coating

(71) Applicant: NOVO NORDISK A/S, Bagsværd (DK)

(72) Inventors: Lars Hovgaard, Farum (DK); Hanne Refsgaard, Bagsvaerd (DK); Thomas Boerglum Kjeldsen, Virum (DK); Peter Madsen, Bagsvaerd (DK)

(21) Appl. No.: 14/906,180

(22) PCT Filed: Jul. 11, 2014
(86) PCT No.: PCT/EP2014/064927

§ 371 Date: Jan. 19, 2016

(30) Foreign Application Priority Data
Jul. 24, 2013 (EP) 13177786.4
Apr. 10, 2014 (EP) 14164198.5

(51) Int. Cl. A61K 9/28; A61K 9/20; A61K 38/28

(52) U.S. Cl. CPC A61K 9/2846; A61K 38/28; A61K 9/2013; A61K 9/2886

(57) ABSTRACT

The present invention relates to a solid oral insulin composition comprising a salt of capric acid which enhances the bioavailability and/or the absorption of said insulin in combination with an anionic copolymer coating, which is resistant to dissolution at pH below 5.0 and dissolved at pH above 5.0.

### TECHNICAL FIELD

The present invention relates to a solid oral insulin composition consisting of a tablet core and an anionic copolymer coating, wherein said tablet core comprises a salt of capric acid.

### BACKGROUND

Many pathological states due to deficiencies in or complete failure of the production of certain macromolecules (e.g. proteins and peptides) are treated with an invasive and inconvenient parenteral administration of therapeutic macromolecules. One example hereof is the administration of insulin in the treatment of insulin dependent patients, who are in need of one or more daily doses of insulin. The oral route is desirable for administration due to its non-invasive nature and has a great potential to decrease the patient's discomfort related to drug administration and to increased drug compliance. However, several barriers exist; such as the enzymatic degradation in the gastrointestinal (GI) tract, drug efflux pumps, insufficient and variable absorption from the intestinal mucosa, as well as first pass metabolism in the liver. Thus until now no products for oral delivery of insulins are found to be marketed.

One example of such macromolecules is human insulin which is degraded by various digestive enzymes found in the stomach (pepsin), in the intestinal lumen (chymotrypsin, trypsin, elastase, carboxypeptidases, etc.) and in the mucosal surfaces of the GI tract (aminopeptidases, carboxypeptidases, enteropeptidases, dipeptidyl peptidases, endopeptidases, etc.).

The pH of the gastrointestinal tract varies from quite acidic pH 1-3 in the stomach through pH 5.5 in the duodenum to pH 7.5 in the ileum. Then entering the colon pH drops to pH 5 before again increasing to pH 7 in the rectum (Dan Med Bull. 1999 June; 46(3):183-96. Intraluminal pH of the human gastrointestinal tract. Fallingborg J.) Provision of a solid oral dosage form which would facilitate the administration of insulin is desirable. The advantages of solid oral dosage forms over other dosage forms include ease of manufacture and administration. There may also be advantages relating to convenience of administration increasing patient compliance. US2007/0026082 discloses oral multiparticulate pharmaceutical form comprising pellets having a size in the range from 50 to 2500 μm, which are composed of a) an inner matrix having a mucoadhesive effect and b) an outer film coating. The polymer having a mucoadhesive effect is chosen so that it exhibits a mucoadhesive effect of at least eta b=150 to 1000 mPas and a water uptake of from 10 to 750 percent in 15 min in a range of +/−0.5 pH units relative to the pH at which an outer coating starts to dissolve, and the active substance content of the matrix layer is a maximum of 40 percent by weight of the content of polymer having a mucoadhesive effect. Suitable polymers having a mucoadhesive effect are in particular a chitosan (chitosan and derivatives, chitosans), (meth)acrylate copolymers consisting of 20-45 percent by weight methyl methacrylate and 55 to 80 percent by weight methacrylic acid, celluloses, especially methyl celluloses such as Na carboxymethylcellulose (e.g. Blanose or Methocel).

US2006/018874 discloses tablets containing sodium caprate and IN105 insulin. CA 2187741, US 2207/0238707, WO2010/032140 and WO2011/084618 disclose a formulation comprising sodium caprate and a coating. WO2011/103920 discloses pharmaceutical compositions comprising a tablet core consisting of active pharmaceutical ingredient such as insulin, a penetration promoter, a bioavailability promoting agent, such as an enzyme inhibitor and a polymeric coating. The oral route of administration is rather complex and a need for establishment of an acceptable pharmaceutical composition suitable for the treatment of patients, with an effective bioavailability of insulins, is existent.

### SUMMARY

The present invention provides a pharmaceutical composition which is effective in providing therapeutically effective blood levels of insulins in a subject, when administered to said subject's gastrointestinal tract (e.g. by oral administration of a composition according to the present invention).

One embodiment of the present invention concerns a pharmaceutical composition consisting of a tablet core and an anionic copolymer coating, wherein said tablet core comprises a salt of a medium-chain fatty acid and a protease stabilised insulina protease stabilised insulin, wherein said protease stabilised insulin comprises a linker and a fatty acid or fatty diacid chain having 14-22 carbon atoms and/or one or more additional disulfide bridges relative to human insulin.

In one embodiment, said tablet core comprises a salt of capric acid.

In one embodiment, said anionic copolymer coating is a dispersion comprising between 25-35% such as 30% (meth)acrylate copolymer, wherein said (meth)acrylate copolymer consists of 10-30% (w/w) methyl methacrylate, 50-70% (w/w) methyl acrylate and 5-15% (w/w) methacrylic acid.

In one embodiment said anionic copolymer coating is at least partly in direct contact with an outer surface of a tablet core.

### BRIEF DESCRIPTION OF THE DRAWINGS

FIG. 1 shows the dissolution rate of compositions according to the present invention (tablet core+EUDRAGIT® FS30D coating as sold by Evonik Industries (in 2013)+no sub coat) and a composition wherein a standard sub coat is added between tablet core and anionic copolymer coating (tablet core+sub coat+EUDRAGIT® FS30D coating as sold by Evonik Industries (in 2013)).

FIG. 2A shows the PK profiles for this insulin in tablet cores with Opadry®II sub coat and a functional coat of EUDRAGIT® FS30D as sold by Evonik Industries (in 2013), squares show the PK profile for tablets tested at time 0 and circles show the PK profile for tablets tested after 12 or more weeks storage at 5° C.

FIG. 2B shows the PK profiles for this insulin in tablet cores coated with a functional coat of EUDRAGIT® FS30D as sold by Evonik Industries (in 2013) without an Opadry®II sub coat, squares show the PK profile for tablets tested at time 0 and circles show the PK profile for tablets tested after 12 or more weeks of storage at 5° C.

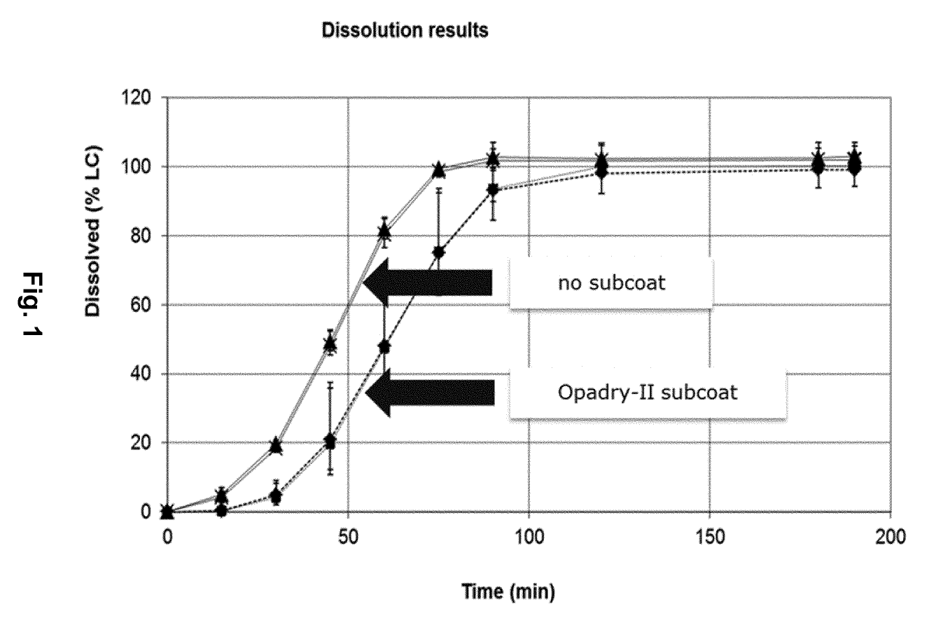

### DESCRIPTION

The present invention provides a pharmaceutical composition which is effective in providing therapeutically effective blood levels of insulin, such as protease stabilised insulin, in a subject, when administered to said subject's gastrointestinal (GI) tract (e.g. per os (oral administration) of a composition according to the present invention).

It was surprisingly found that a pharmaceutical composition according to the embodiments of the present invention are suitable for administration of protease stabilised insulins to the GI tract (e.g. per os (oral administration)). It was surprisingly found that the combination of oral bioavailability and pharmacokinetic/pharmacodynamic (PK/PD) profile for protease stabilised insulins comprised in the tablet core of the pharmaceutical compositions according to the embodiments results in an attractive overall profile for protease stabilised insulins for administering said protease stabilised insulins to the GI tract (e.g. per os (oral administration)). It has surprisingly been found that a pharmaceutical composition according to the embodiments of the present invention increase the bioavailability of administered protease stabilised insulin when administered to the GI tract (e.g. per os (oral administration)).

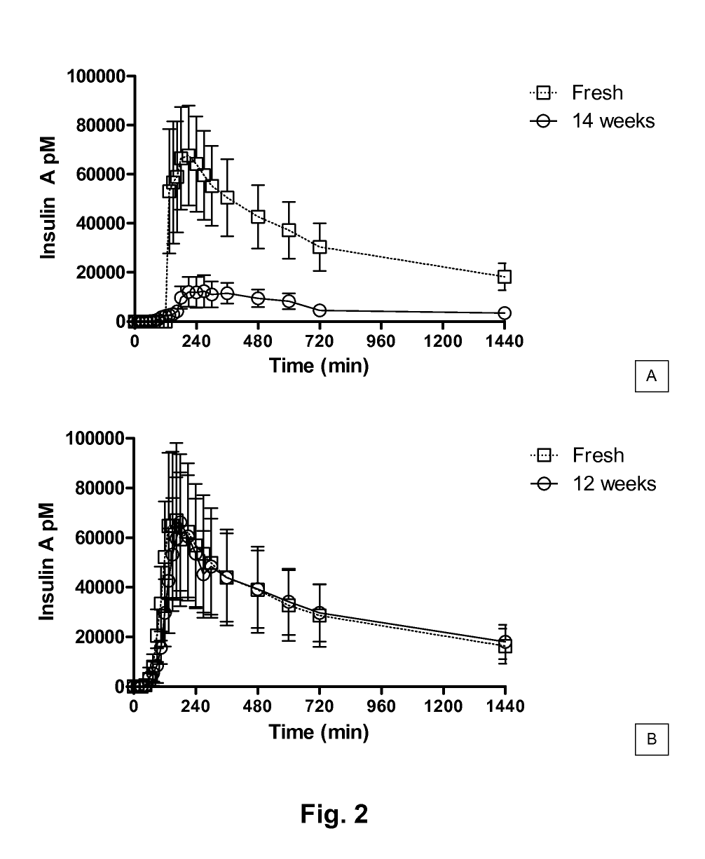

It was surprisingly found that a composition comprising a polyvinyl alcohol polymer coating (such as Opadry® II) used as separating layer between a tablet core and an anionic copolymer coating in a composition according to the present invention resulted in an unstable PK and bioavailability profile for the administered insulin in Beagle dogs (see FIG. 2A).

It was surprisingly found that compositions according to the present invention resulted in stable PK and bioavailability profiles for administered protease stabilised insulin in Beagle dogs (see FIG. 2B).

It was surprisingly found that omitting a polyvinyl alcohol polymer coating (such as Opadry® II) used as separating layer between tablet core and an anionic copolymer coating changed the dissolution profile of the anionic copolymer coating, which increased the bioavailability remarkably for the administered insulin. It was surprisingly found that omitting a polyvinyl alcohol polymer coating (such as Opadry®II) used as separating layer between tablet core and the anionic copolymer coating increased the dissolution profile of the anionic copolymer coating, which increased the bioavailability remarkably for the administered insulin.

It was surprisingly found that omitting a standard separating layer between tablet core and the anionic copolymer coating changed the dissolution profile of the anionic copolymer coating, which increased the bioavailability remarkably for the administered insulin. It was surprisingly found that omitting a standard separating layer between tablet core and the anionic copolymer coating increased the dissolution profile of the anionic copolymer coating, which increased the bioavailability remarkably for the administered insulin.

### Coating

One embodiment of the present invention regards a pharmaceutical composition wherein an anionic copolymer coating is based on an anionic copolymer. One embodiment of the present invention regards a pharmaceutical composition wherein an anionic copolymer coating comprises an anionic copolymer.

One embodiment of the present invention regards a pharmaceutical composition wherein an anionic copolymer coating based on anionic copolymer comprises at least 80% of said anionic copolymer.

One embodiment of the present invention regards a pharmaceutical composition wherein an anionic copolymer coating comprising anionic copolymer comprises at least 80% of said anionic copolymer. One embodiment of the present invention regards a pharmaceutical composition wherein an anionic copolymer coating based on anionic copolymer comprises 80% or more of said anionic copolymer. One embodiment of the present invention regards a pharmaceutical composition wherein an anionic copolymer coating comprising anionic copolymer comprises 80% or more of said anionic copolymer.

One embodiment of the present invention regards a pharmaceutical composition wherein an anionic copolymer coating is based on an anionic copolymer, wherein said copolymer is based on methyl acrylate, methyl methacrylate and methacrylic acid. One embodiment of the present invention regards a pharmaceutical composition wherein an anionic copolymer coating mainly comprises methyl acrylate, methyl methacrylate and methacrylic acid. One embodiment of the present invention regards a pharmaceutical composition wherein an anionic copolymer coating comprises 80% or more methyl acrylate, methyl methacrylate and methacrylic acid.

In one embodiment, an anionic copolymer as used in the invention is an anionic (meth)acrylate copolymer. In one embodiment an anionic copolymer as used in the invention is resistant against acidic juices of the stomach.

In one embodiment an anionic copolymer coating for use in the present invention is disclosed in WO 2008/049657.

One embodiment of the present invention regards a pharmaceutical composition comprising a coating, wherein said coating comprises between 25-35% such as 30% (meth)acrylate copolymer, wherein said (meth)acrylate copolymer consists of 10-30% (w/w) methyl methacrylate, 50-70% (w/w) methyl acrylate and 5-15% (w/w) methacrylic acid. In one embodiment, the (meth)acrylate copolymer consists of 25% (w/w) methyl methacrylate, 65% (w/w) methyl acrylate and 10% (w/w) methacrylic acid.

One embodiment of the present invention regards a pharmaceutical composition comprising a coating, wherein said coating comprises a EUDRAGIT® FS type coating e.g. as sold by Evonik Industries (in 2013). One embodiment of the present invention regards a pharmaceutical composition comprising a coating which is a EUDRAGIT FS30D® coating e.g. as sold by Evonik Industries (in 2013). One embodiment of the present invention regards a pharmaceutical composition wherein an anionic copolymer coating according to the present invention completely dissolves at a pH between about 6.5 and about 7.2. One embodiment of the present invention regards a pharmaceutical composition wherein an anionic copolymer coating according to the present invention completely dissolves at a pH between about 6.5 and about 7.2 and does not dissolve below the pH 5.5. One embodiment according to the present invention regards a pharmaceutical composition wherein an anionic copolymer coating is resistant to dissolution at pH below about 6.5. One embodiment according to the present invention regards a pharmaceutical composition wherein an anionic copolymer coating is resistant to dissolution at pH below about 5.5. In one embodiment the pH dissolution ranges of an anionic copolymer coating according to the present invention are determined by the method 6 provided in this application.

One embodiment of the present invention regards a pharmaceutical composition wherein an anionic copolymer coating completely dissolves at a pH above about 7.2.

One embodiment of the present invention regards a pharmaceutical composition wherein an anionic copolymer coating is resistant to dissolution at pH below about 5.5 and completely dissolves at pH above about 7.2.

One embodiment of the present invention regards a pharmaceutical composition wherein an anionic copolymer coating which is resistant to dissolution at pH below about 5.5 and completely dissolves at pH above about 7.2, wherein this pH range is determined by the method 6 provided in this application.

One embodiment of the present invention regards a pharmaceutical composition wherein an anionic copolymer coating completely dissolves at a pH above about 6.5.

One embodiment of the present invention regards a pharmaceutical composition wherein an anionic copolymer coating completely dissolves at a pH above about 6.5, wherein this pH value is determined by the method 6 provided in this application.

One embodiment of the present invention regards a pharmaceutical composition wherein an anionic copolymer coating is resistant to dissolution at a pH below about 5.5 and completely dissolves at a pH above about 6.5, wherein this pH value is determined by the method 6 provided in this application.

One embodiment of the present invention regards a pharmaceutical composition wherein an anionic copolymer coating completely dissolves at a pH above about 7.0.

One embodiment of the present invention regards a pharmaceutical composition wherein an anionic copolymer coating which completely dissolves at a pH above about 7.0, wherein this pH value is determined by the method 6 provided in this application. One embodiment of the present invention regards a pharmaceutical composition wherein an anionic copolymer coating is resistant to dissolution at a pH below about 6.5 and completely dissolves at a pH above about 7.0, wherein this pH value is determined by the method 6 provided in this application.

One embodiment of the present invention regards pharmaceutical compositions with dissolution profiles comparable to the profiles as presented in table 1 (for explanation of the table see table 2 in the Examples):

TABLE 1
Results presented as percent weight gain of enteric coated tablets.

| Functional | | | | | | | | |
|---|---|---|---|---|---|---|---|---|
| coat | | | | | | | | |
| (FS30D) | Weight gain (%) | | | | | | | |
| level | pH1.2 | pH1.2 | | | | | | |
| w/w % | (1 hr) | (2 hr) | pH4.5 | pH5.5 | pH6.0 | pH6.5 | pH7.0 | pH7.4 |
| 3.8 | 1.19 | 2.90 | 3.27 | 4.70 | 6.41 | 9.93 | −30.07 | −100.00 |
| 5.8 | 0.72 | 1.23 | 1.59 | 2.21 | 2.97 | 3.95 | 8.72 | −44.02 |
| 7.4 | 0.00 | 0.42 | 0.87 | 0.80 | 1.12 | 1.20 | 4.18 | 9.15 |

In one embodiment none of the ingredients in an anionic copolymer coating according to the present invention are mucoadhesive. In one embodiment none of the excepients in an anionic copolymer coating according to the present invention are mucoadhesive.

One embodiment of the present invention is a pharmaceutical composition consisting of a tablet core and an anionic copolymer coating, wherein said tablet core comprises a protease stabilised insulin and a salt of capric acid wherein said protease stabilised insulin comprises a linker and a fatty acid or fatty diacid chain having 14-22 carbon atoms.

One embodiment of the present invention is a pharmaceutical composition consisting of a tablet core and an anionic copolymer coating, wherein said tablet core comprises a protease stabilised insulinand a sodium salt of capric acid wherein said protease stabilised insulin comprises a linker and a fatty acid or fatty diacid chain having 14-22 carbon atoms.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises one or more additional disulfide bonds.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises a linker and a fatty acid or fatty diacid chain having 14-22 carbon atoms and optionally comprises one or more additional disulfide bonds.

One embodiment of the present invention is a pharmaceutical composition consisting of a tablet core and an anionic copolymer coating, wherein said tablet core comprises a protease stabilised insulin and a salt of capric acid wherein said protease stabilised insulin comprises a linker and a fatty acid or fatty diacid chain having 14-22 carbon atoms and wherein said pharmaceutical composition comprises an anionic copolymer coating which is resistant to dissolution at pH below about 5.5.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises one or more additional disulfide bonds and wherein said pharmaceutical composition comprises an anionic copolymer coating which is resistant to dissolution at pH below about 5.5.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises a linker and a fatty acid or fatty diacid chain having 14-22 carbon atoms and optionally comprises one or more additional disulfide bonds and wherein said pharmaceutical composition comprises an anionic copolymer coating which is resistant to dissolution at pH below about 5.5.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises one or more additional disulfide bonds and wherein said pharmaceutical composition comprises an anionic copolymer coating which is resistant to dissolution at pH below about 5.5.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises a linker and a fatty acid or fatty diacid chain having 14-22 carbon atoms and optionally comprises one or more additional disulfide bonds and wherein said pharmaceutical composition comprises an anionic copolymer coating which is resistant to dissolution at pH below about 5.5.

One embodiment of the present invention is a pharmaceutical composition consisting of a tablet core and an anionic copolymer coating, wherein said tablet core comprises a protease stabilised insulin and a salt of capric acid wherein said protease stabilised insulin comprises a linker and a fatty acid or fatty diacid chain having 14-22 carbon atoms and wherein said pharmaceutical composition comprises an anionic copolymer coating which is resistant to dissolution at pH below about 6.5.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises one or more additional disulfide bonds and wherein said pharmaceutical composition comprises an anionic copolymer coating which is resistant to dissolution at pH below about 6.5.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises a linker and a fatty acid or fatty diacid chain having 14-22 carbon atoms and optionally comprises one or more additional disulfide bonds and wherein said pharmaceutical composition comprises an anionic copolymer coating which is resistant to dissolution at pH below about 6.5.

One embodiment of the present invention is a pharmaceutical composition consisting of a tablet core and an anionic copolymer coating, wherein said tablet core comprises a protease stabilised insulin and a salt of capric acid wherein said protease stabilised insulin comprises a linker and a fatty acid or fatty diacid chain having 14-22 carbon atoms and wherein said pharmaceutical composition comprises an anionic copolymer coating which is resistant to dissolution at pH below about 5.5, wherein this pH value is determined by the method 6 provided in this application and illustrated in table 2.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises one or more additional disulfide bonds and wherein said pharmaceutical composition comprises an anionic copolymer coating which is resistant to dissolution at pH below about 5.5, wherein this pH value is determined by the method 6 provided in this application and illustrated in table 2.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises a linker and a fatty acid or fatty diacid chain having 14-22 carbon atoms and optionally comprises one or more additional disulfide bonds and wherein said pharmaceutical composition comprises an anionic copolymer coating which is resistant to dissolution at pH below about 5.5, wherein this pH value is determined by the method 6 provided in this application and illustrated in table 2.

One embodiment of the present invention is a pharmaceutical composition consisting of a tablet core and an anionic copolymer coating, wherein said tablet core comprises a protease stabilised insulin and a salt of capric acid wherein said protease stabilised insulin comprises a linker and a fatty acid or fatty diacid chain having 14-22 carbon atoms and wherein said pharmaceutical composition comprises an anionic copolymer coating which is resistant to dissolution at pH below about 6.5, wherein this pH value is determined by the method 6 provided in this application and illustrated in table 2.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises one or more additional disulfide bonds and wherein said pharmaceutical composition comprises an anionic copolymer coating which is resistant to dissolution at pH below about 6.5, wherein this pH value is determined by the method 6 provided in this application and illustrated in table 2.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises a linker and a fatty acid or fatty diacid chain having 14-22 carbon atoms and optionally comprises one or more additional disulfide bonds and wherein said pharmaceutical composition comprises an anionic copolymer coating which is resistant to dissolution at pH below about 6.5, wherein this pH value is determined by the method 6 provided in this application and illustrated in table 2.

One embodiment of the present invention is a pharmaceutical composition consisting of a tablet core and an anionic copolymer coating, wherein said tablet core comprises a protease stabilised insulin and a salt of capric acid wherein said protease stabilised insulin comprises a linker and a fatty acid or fatty diacid chain having 14-22 carbon atoms and wherein said pharmaceutical composition comprises an anionic copolymer coating which dissolves at pH above about 6.5.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises one or more additional disulfide bonds and wherein said pharmaceutical composition comprises an anionic copolymer coating which dissolves at pH above about 6.5.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises a linker and a fatty acid or fatty diacid chain having 14-22 carbon atoms and optionally comprises one or more additional disulfide bonds and wherein said pharmaceutical composition comprises an anionic copolymer coating which dissolves at pH above about 6.5.

One embodiment of the present invention is a pharmaceutical composition consisting of a tablet core and an anionic copolymer coating, wherein said tablet core comprises a protease stabilised insulin and a salt of capric acid wherein said protease stabilised insulin comprises a linker and a fatty acid or fatty diacid chain having 14-22 carbon atoms and wherein said pharmaceutical composition comprises an anionic copolymer coating which dissolves at pH above about 7.0.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises one or more additional disulfide bonds and wherein said pharmaceutical composition comprises an anionic copolymer coating which dissolves at pH above about 7.0.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises a linker and a fatty acid or fatty diacid chain having 14-22 carbon atoms and optionally comprises one or more additional disulfide bonds and wherein said pharmaceutical composition comprises an anionic copolymer coating which dissolves at pH above about 7.0.

One embodiment of the present invention is a pharmaceutical composition consisting of a tablet core and an anionic copolymer coating, wherein said tablet core comprises a protease stabilised insulin and a salt of capric acid wherein said protease stabilised insulin comprises a linker and a fatty acid or fatty diacid chain having 14-22 carbon atoms and wherein said pharmaceutical composition comprises an anionic copolymer coating is dissolved at pH above about 7.2.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises one or more additional disulfide bonds and wherein said pharmaceutical composition comprises an anionic copolymer coating is dissolved at pH above about 7.2.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises a linker and a fatty acid or fatty diacid chain having 14-22 carbon atoms and optionally comprises one or more additional disulfide bonds and wherein said pharmaceutical composition comprises an anionic copolymer coating is dissolved at pH above about 7.2.

One embodiment of the present invention is a pharmaceutical composition consisting of a tablet core and an anionic copolymer coating, wherein said tablet core comprises a protease stabilised insulin and a salt of capric acid wherein said protease stabilised insulin comprises a linker and a fatty acid or fatty diacid chain having 14-22 carbon atoms and wherein said pharmaceutical composition comprises an anionic copolymer coating which dissolves at pH above about 6.5, wherein this pH range is determined by the method 6 provided in this application and illustrated in table 2.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises one or more additional disulfide bonds and wherein said pharmaceutical composition comprises an anionic copolymer coating which dissolves at pH above about 6.5, wherein this pH range is determined by the method 6 provided in this application and illustrated in table 2.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises a linker and a fatty acid or fatty diacid chain having 14-22 carbon atoms and optionally comprises one or more additional disulfide bonds and wherein said pharmaceutical composition comprises an anionic copolymer coating which dissolves at pH above about 6.5, wherein this pH range is determined by the method 6 provided in this application and illustrated in table 2.

One embodiment of the present invention is a pharmaceutical composition consisting of a tablet core and an anionic copolymer coating, wherein said tablet core comprises a protease stabilised insulin and a salt of capric acid wherein said protease stabilised insulin comprises a linker and a fatty acid or fatty diacid chain having 14-22 carbon atoms and wherein said pharmaceutical composition comprises an anionic copolymer coating which dissolves at pH above about 7.0, wherein this pH range is determined by the method 6 provided in this application and illustrated in table 2.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises one or more additional disulfide bonds and wherein said pharmaceutical composition comprises an anionic copolymer coating which dissolves at pH above about 7.0, wherein this pH range is determined by the method 6 provided in this application and illustrated in table 2.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises a linker and a fatty acid or fatty diacid chain having 14-22 carbon atoms and optionally comprises one or more additional disulfide bonds and wherein said pharmaceutical composition comprises an anionic copolymer coating which dissolves at pH above about 7.0, wherein this pH range is determined by the method 6 provided in this application and illustrated in table 2.

One embodiment of the present invention is a pharmaceutical composition consisting of a tablet core and an anionic copolymer coating, wherein said tablet core comprises a protease stabilised insulin and a salt of capric acid wherein said protease stabilised insulin comprises a linker and a fatty acid or fatty diacid chain having 14-22 carbon atoms and wherein said pharmaceutical composition comprises an anionic copolymer coating which dissolves at pH above about 7.2, wherein this pH range is determined by the method 6 provided in this application and illustrated in table 2.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises one or more additional disulfide bonds and wherein said pharmaceutical composition comprises an anionic copolymer coating which dissolves at pH above about 7.2, wherein this pH range is determined by the method 6 provided in this application and illustrated in table 2. One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises a linker and a fatty acid or fatty diacid chain having 14-22 carbon atoms and optionally comprises one or more additional disulfide bonds and wherein said pharmaceutical composition comprises an anionic copolymer coating which dissolves at pH above about 7.2, wherein this pH range is determined by the method 6 provided in this application and illustrated in table 2.

One embodiment of the present invention is a pharmaceutical composition consisting of a tablet core and an anionic copolymer coating, wherein said tablet core comprises a protease stabilised insulin and a salt of capric acid wherein said protease stabilised insulin comprises a linker and a fatty acid or fatty diacid chain having 14-22 carbon atoms and wherein said pharmaceutical composition comprises an anionic copolymer coating which is resistant to dissolution at pH below about 5.5 and dissolves at pH above about 7.2.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises one or more additional disulfide bonds and wherein said pharmaceutical composition comprises an anionic copolymer coating which is resistant to dissolution at pH below about 5.5 and dissolves at pH above about 7.2.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises a linker and a fatty acid or fatty diacid chain having 14-22 carbon atoms and optionally comprises one or more additional disulfide bonds and wherein said pharmaceutical composition comprises an anionic copolymer coating which is resistant to dissolution at pH below about 5.5 and dissolves at pH above about 7.2.

One embodiment of the present invention is a pharmaceutical composition consisting of a tablet core and an anionic copolymer coating, wherein said tablet core comprises a protease stabilised insulin and a salt of capric acid wherein said protease stabilised insulin comprises a linker and a fatty acid or fatty diacid chain having 14-22 carbon atoms and wherein said pharmaceutical composition comprises an anionic copolymer coating which is resistant to dissolution at pH below about 5.5 and dissolves at pH above about 7.2, wherein this pH range is determined by the method 6 provided in this application and illustrated in table 2.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises one or more additional disulfide bonds and wherein said pharmaceutical composition comprises an anionic copolymer coating which is resistant to dissolution at pH below about 5.5 and dissolves at pH above about 7.2, wherein this pH range is determined by the method 6 provided in this application and illustrated in table 2.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises a linker and a fatty acid or fatty diacid chain having 14-22 carbon atoms and optionally comprises one or more additional disulfide bonds and wherein said pharmaceutical composition comprises an anionic copolymer coating which is resistant to dissolution at pH below about 5.5 and dissolves at pH above about 7.2, wherein this pH range is determined by the method 6 provided in this application and illustrated in table 2.

One embodiment of the present invention is a pharmaceutical composition consisting of a tablet core and an anionic copolymer coating, wherein said tablet core comprises a protease stabilised insulin and a salt of capric acid wherein said protease stabilised insulin comprises a linker and a fatty acid or fatty diacid chain having 14-22 carbon atoms and wherein said pharmaceutical composition comprises an anionic copolymer coating which is resistant to dissolution at pH below about 5.5 and dissolves at pH above about 6.5.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises one or more additional disulfide bonds and wherein said pharmaceutical composition comprises an anionic copolymer coating which is resistant to dissolution at pH below about 5.5 and dissolves at pH above about 6.5.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises a linker and a fatty acid or fatty diacid chain having 14-22 carbon atoms and optionally comprises one or more additional disulfide bonds and wherein said pharmaceutical composition comprises an anionic copolymer coating which is resistant to dissolution at pH below about 5.5 and dissolves at pH above about 6.5.

One embodiment of the present invention is a pharmaceutical composition consisting of a tablet core and an anionic copolymer coating, wherein said tablet core comprises a protease stabilised insulin and a salt of capric acid wherein said protease stabilised insulin comprises a linker and a fatty acid or fatty diacid chain having 14-22 carbon atoms and wherein said pharmaceutical composition comprises an anionic copolymer coating which is resistant to dissolution at pH below about 5.5 and dissolves at pH above about 6.5, wherein this pH range is determined by the method 6 provided in this application and illustrated in table 2.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises one or more additional disulfide bonds and wherein said pharmaceutical composition comprises an anionic copolymer coating which is resistant to dissolution at pH below about 5.5 and dissolves at pH above about 6.5, wherein this pH range is determined by the method 6 provided in this application and illustrated in table 2.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises a linker and a fatty acid or fatty diacid chain having 14-22 carbon atoms and optionally comprises one or more additional disulfide bonds and wherein said pharmaceutical composition comprises an anionic copolymer coating which is resistant to dissolution at pH below about 5.5 and dissolves at pH above about 6.5, wherein this pH range is determined by the method 6 provided in this application and illustrated in table 2.

One embodiment of the present invention is a pharmaceutical composition consisting of a tablet core and an anionic copolymer coating, wherein said tablet core comprises a protease stabilised insulin and a salt of capric acid wherein said protease stabilised insulin comprises a linker and a fatty acid or fatty diacid chain having 14-22 carbon atoms and wherein said pharmaceutical composition comprises an anionic copolymer coating which is resistant to dissolution at pH below about 6.5 and dissolves at pH above about 7.0.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises one or more additional disulfide bonds and wherein said pharmaceutical composition comprises an anionic copolymer coating which is resistant to dissolution at pH below about 6.5 and dissolves at pH above about 7.0.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises a linker and a fatty acid or fatty diacid chain having 14-22 carbon atoms and optionally comprises one or more additional disulfide bonds and wherein said pharmaceutical composition comprises an anionic copolymer coating which is resistant to dissolution at pH below about 6.5 and dissolves at pH above about 7.0.

One embodiment of the present invention is a pharmaceutical composition consisting of a tablet core and an anionic copolymer coating, wherein said tablet core comprises a protease stabilised insulin and a salt of capric acid wherein said protease stabilised insulin comprises a linker and a fatty acid or fatty diacid chain having 14-22 carbon atoms and wherein said pharmaceutical composition comprises an anionic copolymer coating which is resistant to dissolution at pH below about 6.5 and dissolves at pH above about 7.0, wherein this pH range is determined by the method 6 provided in this application and illustrated in table 2.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises one or more additional disulfide bonds and wherein said pharmaceutical composition comprises an anionic copolymer coating which is resistant to dissolution at pH below about 6.5 and dissolves at pH above about 7.0, wherein this pH range is determined by the method 6 provided in this application and illustrated in table 2.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises a linker and a fatty acid or fatty diacid chain having 14-22 carbon atoms and optionally comprises one or more additional disulfide bonds and wherein said pharmaceutical composition comprises an anionic copolymer coating which is resistant to dissolution at pH below about 6.5 and dissolves at pH above about 7.0, wherein this pH range is determined by the method 6 provided in this application and illustrated in table 2.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises one or more additional disulfide bonds and wherein said pharmaceutical composition comprises an anionic copolymer coating which is resistant to dissolution at pH below about 6.5 and dissolves at pH above about 7.0.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises one or more additional disulfide bonds and wherein said pharmaceutical composition comprises an anionic copolymer coating which is resistant to dissolution at pH below about 6.5 and dissolves at pH above about 7.0.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises a linker and a fatty acid or fatty diacid chain having 14-22 carbon atoms and optionally comprises one or more additional disulfide bonds and wherein said pharmaceutical composition comprises an anionic copolymer coating which is resistant to dissolution at pH below about 6.5 and dissolves at pH above about 7.0.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises a linker and a fatty acid or fatty diacid chain having 14-22 carbon atoms and optionally comprises one or more additional disulfide bonds and wherein said pharmaceutical composition comprises an anionic copolymer coating which is resistant to dissolution at pH below about 6.5 and dissolves at pH above about 7.0.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises one or more additional disulfide bonds and wherein said pharmaceutical composition comprises an anionic copolymer coating which is resistant to dissolution at pH below about 6.5 and dissolves at pH above about 7.0.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises a linker and a fatty acid or fatty diacid chain having 14-22 carbon atoms and optionally comprises one or more additional disulfide bonds and wherein said pharmaceutical composition comprises an anionic copolymer coating which is resistant to dissolution at pH below about 6.5 and dissolves at pH above about 7.0.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises one or more additional disulfide bonds and wherein said pharmaceutical composition comprises an anionic copolymer coating which is resistant to dissolution at pH below about 6.5 and dissolves at pH above about 7.0, wherein this pH range is determined by the method 6 provided in this application and illustrated in table 2.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises one or more additional disulfide bonds and wherein said pharmaceutical composition comprises an anionic copolymer coating which is resistant to dissolution at pH below about 6.5 and dissolves at pH above about 7.0, wherein this pH range is determined by the method 6 provided in this application and illustrated in table 2.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises a linker and a fatty acid or fatty diacid chain having 14-22 carbon atoms and optionally comprises one or more additional disulfide bonds and wherein said pharmaceutical composition comprises an anionic copolymer coating which is resistant to dissolution at pH below about 6.5 and dissolves at pH above about 7.0, wherein this pH range is determined by the method 6 provided in this application and illustrated in table 2.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises a linker and a fatty acid or fatty diacid chain having 14-22 carbon atoms and optionally comprises one or more additional disulfide bonds and wherein said pharmaceutical composition comprises an anionic copolymer coating which is resistant to dissolution at pH below about 6.5 and dissolves at pH above about 7.0, wherein this pH range is determined by the method 6 provided in this application and illustrated in table 2.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises one or more additional disulfide bonds and wherein said pharmaceutical composition comprises an anionic copolymer coating which is resistant to dissolution at pH below about 6.5 and dissolves at pH above about 7.0, wherein this pH range is determined by the method 6 provided in this application and illustrated in table 2.

One embodiment of the present invention concerns a pharmaceutical composition consisting of a tablet core and an anionic copolymer coating, wherein said tablet core comprises a salt of a medium-chain fatty acid and a protease stabilised insulin, wherein said protease stabilised insulin comprises a linker and a fatty acid or fatty diacid chain having 14-22 carbon atoms and optionally comprises one or more additional disulfide bonds and wherein said pharmaceutical composition comprises an anionic copolymer coating which is resistant to dissolution at pH below about 6.5 and dissolves at pH above about 7.0, wherein this pH range is determined by the method 6 provided in this application and illustrated in table 2.

### Contact Between Tablet Core and Coating

When referring to the contact between the anionic copolymer coating and the tablet core, if not indicated otherwise the contact is in the interface between the two interfaces and thus an inner surface of an anionic copolymer coating and an outer surface of a tablet core.

Thus one embodiment of the present invention regards a pharmaceutical composition wherein an inner surface of an anionic copolymer coating is at least partly in direct contact with an outer surface of a tablet core. Alternatively this could be described as; one embodiment of the present invention regards a pharmaceutical composition wherein an anionic copolymer coating is at least partly in direct contact with a tablet core. Another alternative way to describe the same contact could be; one embodiment of the present invention regards a pharmaceutical composition wherein an anionic copolymer coating is at least partly in direct contact with an outer surface of a tablet core.

One embodiment of the present invention regards a pharmaceutical composition wherein an anionic copolymer coating is in direct contact with 10% or more of an outer surface of a tablet core. One embodiment of the present invention regards a pharmaceutical composition wherein an anionic copolymer coating is in direct contact with 20% or more of an outer surface of a tablet core. One embodiment of the present invention regards a pharmaceutical composition wherein an anionic copolymer coating is in direct contact with 30% or more of an outer surface of a tablet core. One embodiment of the present invention regards a pharmaceutical composition wherein an anionic copolymer coating is in direct contact with 40% or more of an outer surface of a tablet core. One embodiment of the present invention regards a pharmaceutical composition wherein an anionic copolymer coating is in direct contact with 50% or more of an outer surface of a tablet core. One embodiment of the present invention regards a pharmaceutical composition wherein an anionic copolymer coating is in direct contact with 60% or more of an outer surface of a tablet core. One embodiment of the present invention regards a pharmaceutical composition wherein an anionic copolymer coating is in direct contact with 70% or more of an outer surface of a tablet core. One embodiment of the present invention regards a pharmaceutical composition wherein an anionic copolymer coating is in direct contact with 80% or more of an outer surface of a tablet core. One embodiment of the present invention regards a pharmaceutical composition wherein an anionic copolymer coating is in direct contact with 85% or more of an outer surface of a tablet core. One embodiment of the present invention regards a pharmaceutical composition wherein an anionic copolymer coating is in direct contact with 90% or more of an outer surface of a tablet core. One embodiment of the present invention regards a pharmaceutical composition wherein an anionic copolymer coating is in direct contact with 95% or more of an outer surface of a tablet core. One embodiment of the present invention regards a pharmaceutical composition wherein an anionic copolymer coating is in direct contact with 99% or more of an outer surface of a tablet core. One embodiment of the present invention regards a pharmaceutical composition wherein an anionic copolymer coating is in direct contact with 100% of an outer surface of a tablet core.

One embodiment of the present invention regards a pharmaceutical composition wherein an anionic copolymer coating is in direct contact with the majority of the surface of a tablet core. One embodiment of the present invention regards a pharmaceutical composition wherein an anionic copolymer coating is in direct contact with most of the surface of a tablet core. One embodiment of the present invention regards a pharmaceutical composition wherein an anionic copolymer coating is in direct contact with some of the surface of a tablet core.

One embodiment of the present invention regards a pharmaceutical composition wherein no separating layer is applied between an anionic copolymer coating and a tablet core. One embodiment of the present invention regards a pharmaceutical composition wherein no continuous separating layer is applied between an anionic copolymer coating and a tablet core.

One embodiment of the present invention regards a pharmaceutical composition wherein an anionic copolymer coating is in direct contact with the majority of the caprate, such as e.g. sodium caprate, exposed at an outer surface of a tablet core.

One embodiment of the present invention regards a pharmaceutical composition wherein an anionic copolymer coating is in direct contact with the majority of the caprate, such as e.g. sodium caprate, and protease stabilised insulin exposed at an outer surface of a tablet core.

One embodiment of the present invention regards a pharmaceutical composition wherein an anionic copolymer coating is in direct contact with the majority of the caprate, such as e.g. sodium caprate, and protease stabilised insulin exposed at an outer surface of a tablet core.

One embodiment of the present invention regards a pharmaceutical composition wherein an anionic copolymer coating is in direct contact with the majority of the caprate, e.g. sodium caprate, protease stabilised insulin and any additional excipients comprised in a tablet core which are exposed at an outer surface of a tablet core.

One embodiment of the present invention regards a pharmaceutical composition wherein an anionic copolymer coating is in direct contact with 10% or more of an outer surface of one or more particles of multiparticulate systems coated with said anionic copolymer coating. One embodiment of the present invention regards a pharmaceutical composition wherein an anionic copolymer coating is in direct contact with 20% or more of an outer surface of one or more particles of multiparticulate systems coated with said anionic copolymer coating. One embodiment of the present invention regards a pharmaceutical composition wherein an anionic copolymer coating is in direct contact with 30% or more of an outer surface of one or more particles of multiparticulate systems coated with said anionic copolymer coating. One embodiment of the present invention regards a pharmaceutical composition wherein an anionic copolymer coating is in direct contact with 40% or more of an outer surface of one or more particles of multiparticulate systems coated with said anionic copolymer coating. One embodiment of the present invention regards a pharmaceutical composition wherein an anionic copolymer coating is in direct contact with 50% or more of an outer surface of one or more particles of multiparticulate systems coated with said anionic copolymer coating. One embodiment of the present invention regards a pharmaceutical composition wherein an anionic copolymer coating is in direct contact with 60% or more of an outer surface of one or more particles of multiparticulate systems coated with said anionic copolymer coating. One embodiment of the present invention regards a pharmaceutical composition wherein an anionic copolymer coating is in direct contact with 70% or more of an outer surface of one or more particles of multiparticulate systems coated with said anionic copolymer coating. One embodiment of the present invention regards a pharmaceutical composition wherein an anionic copolymer coating is in direct contact with 80% or more of an outer surface of one or more particles of multiparticulate systems coated with said anionic copolymer coating. One embodiment of the present invention regards a pharmaceutical composition wherein an anionic copolymer coating is in direct contact with 85% or more of an outer surface of one or more particles of multiparticulate systems coated with said anionic copolymer coating. One embodiment of the present invention regards a pharmaceutical composition wherein an anionic copolymer coating is in direct contact with 90% or more of an outer surface of one or more particles of multiparticulate systems coated with said anionic copolymer coating. One embodiment of the present invention regards a pharmaceutical composition wherein an anionic copolymer coating is in direct contact with 95% or more of an outer surface of one or more particles of multiparticulate systems coated with said anionic copolymer coating. One embodiment of the present invention regards a pharmaceutical composition wherein an anionic copolymer coating is in direct contact with 99% or more of an outer surface of one or more particles of multiparticulate systems coated with said anionic copolymer coating. One embodiment of the present invention regards a pharmaceutical composition wherein an anionic copolymer coating is in direct contact with 100% or more of an outer surface of one or more particles of multiparticulate systems coated with said anionic copolymer coating.

One embodiment of the present invention regards a pharmaceutical composition wherein an anionic copolymer coating is in direct contact with the majority of the caprate, e.g. sodium caprate, protease stabilised insulin and any additional excipients comprised in said tablet core which are exposed at an outer surface of said tablet core.

One embodiment of the present invention regards a pharmaceutical composition wherein an anionic copolymer coating is in direct contact with the majority of the caprate, e.g. sodium caprate, protease stabilised insulin and any additional excipients comprised in said tablet core which are exposed at an outer surface of said tablet core.

One embodiment of the present invention regards a pharmaceutical composition wherein an anionic copolymer coating is in direct contact with the majority of the caprate, e.g. sodium caprate, protease stabilised insulin, sorbitol and stearic acid comprised in said tablet core which are exposed at an outer surface of said tablet core.

One embodiment of the present invention regards a pharmaceutical composition wherein an anionic copolymer coating is in direct contact with the majority of all ingredients comprised in said tablet core exposed at an outer surface of said tablet core.

### Tablet Core

One embodiment of the present invention is a pharmaceutical composition consisting of a tablet core and an anionic copolymer coating such as e.g. a (meth)acrylate copolymer coating, wherein said tablet core comprises a protease stabilised insulin and a salt of capric acid wherein said protease stabilised insulin comprises a linker and a fatty acid or fatty diacid chain having 14-22 carbon atoms.

One embodiment of the present invention is a pharmaceutical composition consisting of a tablet core and an anionic copolymer coating such as e.g. a (meth)acrylate copolymer coating, wherein said tablet core comprises a protease stabilised insulin and a sodium salt of capric acid wherein said protease stabilised insulin comprises a linker and a fatty acid or fatty diacid chain having 14-22 carbon atoms.

One embodiment of the present invention concerns a pharmaceutical composition consisting of a tablet core and an anionic copolymer coating such as e.g. a (meth)acrylate copolymer coating, wherein said tablet core comprises a salt of a medium-chain fatty acid and a protease stabilised, acylated insulin, wherein said a protease stabilised insulin comprises one or more additional disulfide bonds.

One embodiment of the present invention concerns a pharmaceutical composition consisting of a tablet core and an anionic copolymer coating such as e.g. a (meth)acrylate copolymer coating, wherein said tablet core comprises a salt of a medium-chain fatty acid and an acylated insulin, wherein said protease stabilised insulin comprises a linker and a fatty acid or fatty diacid chain having 14-22 carbon atoms and optionally comprises one or more additional disulfide bonds.

One embodiment of the present invention is a pharmaceutical composition consisting of a tablet core and an anionic copolymer coating such as e.g. a (meth)acrylate copolymer coating, wherein said tablet core comprises a protease stabilised insulin and a salt of capric acid.

One embodiment of the present invention is a pharmaceutical composition consisting of a tablet core and an anionic copolymer coating such as e.g. a (meth)acrylate copolymer coating, wherein said tablet core comprises a protease stabilised insulin and a sodium salt of capric acid.

In one embodiment of the present invention a tablet core coated with an anionic copolymer according to this invention contains a salt of capric acid. In one embodiment of the present invention a tablet core coated with an anionic copolymer according to this invention contains about 60-85% (w/w) or more salt of capric acid.

In one embodiment of the present invention a tablet core coated with an anionic copolymer according to this invention contains about 77% (w/w) or more salt of capric acid. In one embodiment of the present invention a tablet core coated with an anionic copolymer according to this invention contains a sodium salt of capric acid. In one embodiment of the present invention a tablet core coated with an anionic copolymer according to this invention contains about 60-85% (w/w) or more sodium salt of capric acid. In one embodiment of the present invention a tablet core coated with an anionic copolymer according to this invention contains about 77% (w/w) or more salt of capric acid.

In one embodiment the tablet core according to the present invention comprises 60-85% (w/w) salt of capric acid. In one embodiment the tablet core according to the present invention comprises 70%-85 (w/w) salt of capric acid. In one embodiment the tablet core according to the present invention comprises 75%-85 (w/w) salt of capric acid. In one embodiment the tablet core according to the present invention comprises 60% (w/w) salt of capric acid. In one embodiment the tablet core according to the present invention comprises about 70% (w/w) salt of capric acid. In one embodiment the tablet core according to the present invention comprises less than 75% (w/w) salt of capric acid. In one embodiment the tablet core according to the present invention comprises less than 80% (w/w) salt of capric acid. In one embodiment the tablet core according to the present invention comprises less than 85% (w/w) salt of capric acid.

In one embodiment excipients comprised in a tablet core according to the present invention have a molecular weight below 1000 g/mol. In one embodiment excipients comprised in a tablet core according to the present invention have a molecular weight below 900 g/mol. In one embodiment excipients comprised in a tablet core according to the present invention have a molecular weight below 800 g/mol. In one embodiment excipients comprised in a tablet core according to the present invention have a molecular weight below 700 g/mol. In one embodiment excipients comprised in a tablet core according to the present invention have a molecular weight below 600 g/mol. In one embodiment excipients comprised in a tablet core according to the present invention have a molecular weight below 500 g/mol. In one embodiment excipients comprised in a tablet core according to the present invention have a molecular weight below 400 g/mol. In one embodiment excipients comprised in a tablet core according to the present invention have a molecular weight below 300 g/mol.

In one embodiment all dry ingredients comprised in a tablet core according to the present invention have a molecular weight below 1000 g/mol. In one embodiment all dry ingredients comprised in a tablet core according to the present invention have a molecular weight below 900 g/mol. In one embodiment all dry ingredients comprised in a tablet core according to the present invention have a molecular weight below 800 g/mol. In one embodiment all dry ingredients comprised in a tablet core according to the present invention have a molecular weight below 700 g/mol. In one embodiment all dry ingredients comprised in a tablet core according to the present invention have a molecular weight below 600 g/mol. In one embodiment all dry ingredients comprised in a tablet core according to the present invention have a molecular weight below 500 g/mol. In one embodiment all dry ingredients comprised in a tablet core according to the present invention have a molecular weight below 400 g/mol. In one embodiment all dry ingredients comprised in a tablet core according to the present invention have a molecular weight below 300 g/mol.

In one embodiment a composition according to the present invention comprises a tablet core, wherein said tablet core comprises a salt of capric acid and one or more protease stabilised insulins. In one embodiment a composition according to the present invention comprises a tablet core, wherein said tablet core comprises a salt of capric acid and protease stabilised insulin and one or more excipients. In one embodiment a composition according to the present invention comprises a tablet core, wherein said tablet core comprises a salt of capric acid, insulin and one or more excipients, such as but not limited to sorbitol, magnesium stearate and stearic acid.

In one embodiment a composition according to the present invention comprises a tablet core, wherein said tablet core comprises one or more excipients, such as polyols and/or lubricants. In one embodiment a composition according to the present invention comprises polyols. In one embodiment a composition according to the present invention comprises a tablet core, wherein said tablet core comprises polyols, such as, but not limited to sorbitol and mannitol.

In one embodiment a composition according to the present invention comprises polyols, wherein said polyols are selected from the group consisting of sorbitol, mannitol or mixtures thereof.

In one embodiment a composition according to the present invention comprises a tablet core, wherein said tablet core comprises lubricants, such as, but not limited to stearic acid, magnesium stearate, stearate and colloidal silica. In one embodiment a composition according to the present invention comprises lubricants, wherein said lubricants are selected from the group consisting of stearic acid, magnesium stearate, stearate or mixtures thereof.

### Pharmaceutical Composition

In one embodiment a tablet core of a composition according to the present invention is a tablet. In one embodiment a tablet core of a composition according to the present invention is a capsule. In one embodiment a tablet core according to the present invention comprises one or more layers. The tablet may e a single or multilayer tablet having a compressed multiparticulate system in one, all or none of the layers. In one embodiment a multiparticulate system consists of granules compressed into a tablet.

In one embodiment a tablet core of a composition according to the present invention is a multiparticulate system. The multiparticulate system may be in the form of a tablet or contained in a capsule. In one embodiment a tablet core according to the present invention is a multiparticulate system comprising particles of the same dimensions. In one embodiment a tablet core according to the present invention is a multiparticulate system comprising particles of various dimensions.

In one embodiment the particles according to the present invention are coated with an anionic copolymer coating as herein defined, such as e.g. Eudragit FS30D as produced by Evonic Industries in 2013, in the same way as defined for tablet cores. In one embodiment a tablet core according to the present invention is a particle of a multiparticulate system according to the present invention and coated with an anionic copolymer coating as herein defined in the same way as defined for tablet cores.

In one embodiment one or more particles of multiparticulate systems according to the present invention are coated with an anionic copolymer coating as herein defined. In one embodiment one or more particles of multiparticulate systems according to the present invention are coated with an anionic copolymer coating as herein defined. In one embodiment one or more particles of multiparticulate systems according to the present invention are coated with an anionic copolymer coating as herein defined, wherein an anionic copolymer coating as herein defined is an EUDRAGIT® FS30D coating as sold by Evonik Industries (in 2013).

In one embodiment one or more particles of multiparticulate systems according to the present invention are individually coated with an anionic copolymer coating as herein defined. In one embodiment one or more particles of multiparticulate systems according to the present invention are individually coated with an anionic copolymer coating as herein defined, before pressed into a tablet.

In one embodiment individually coated one or more particles of a multiparticulate system according to the present invention are pressed into a tablet core. In one embodiment individually coated one or more particles of a multiparticulate system according to the present invention are pressed into a tablet core and the resulting tablet core is not coated with another layer of anionic copolymer coating. In one embodiment individually coated on or more particles of a multiparticulate system according to the present invention are pressed into a tablet core and said resulting tablet core is also coated with an anionic copolymer coating. In one embodiment on or more particles of multiparticulate systems according to the present invention are individually coated with anionic copolymer coating and pressed into a tablet and said resulting tablet is coated with an additional non-functional coating.

In one embodiment one or more particles of multiparticulate systems according to the present invention are collectively coated with an anionic copolymer coating as herein defined. In one embodiment one or more particles of multiparticulate systems according to the present invention are collectively coated with an anionic copolymer coating as herein defined, after being pressed into a tablet.

In one embodiment a composition of the present invention comprises a tablet core, wherein said tablet core comprises a salt of capric acid and one or more excipients.

In one embodiment none of the ingredients in a composition according to the present invention are mucoadhesive. In one embodiment none of the excepients in a composition according to the present invention are mucoadhesive. In one embodiment none of the ingredients in a tablet core according to the present invention are mucoadhesive. In one embodiment none of the excepients in a tablet according to the present invention are mucoadhesive.

In certain embodiments of the present invention, the pharmaceutical composition comprises a tablet core, wherein said tablet core may comprise additional excipients commonly found in a pharmaceutical composition, examples of such excipients include, but are not limited to enzyme inhibitors, stabilisers, preservatives, flavors, sweeteners and other components as described in ‘Handbook of Pharmaceutical Excipients’ Ainley Wade, Paul J. Weller, Arthur H. Kibbe, 3^{rd} edition, American Pharmacists Association (2000), which is hereby incorporated by reference or —‘Handbook of Pharmaceutical Excipients’, Rowe et al., Eds., 4th Edition, Pharmaceutical Press (2003), which is hereby incorporated by reference. In one embodiment none of the active ingredients, or the excipients in the tablet core according to the present invention exert any water uptake. In one embodiment the active ingredients and the excipients in the tablet core exert zero water uptake. In one embodiment the active ingredients and the excipients in the tablet core exert 0-9% water uptake. In one embodiment the active ingredients and the excipients in the tablet core exert below 10% water uptake. In one embodiment the active ingredients and the excipients in the tablet core exert below 9% water uptake. In one embodiment the active ingredients and the excipients in the tablet core exert below 8% water uptake.

### Use of the Composition

One embodiment of the present invention regards a method for manufacture of compositions according to the present invention.

In one embodiment, a composition according to the invention is used for the preparation of a medicament for the treatment or prevention of hyperglycemia, type 2 diabetes mellitus, impaired glucose tolerance and type 1 diabetes mellitus. The invention may also solve further problems that will be apparent from the disclosure of the exemplary embodiments.

In one embodiment a composition according to the present invention shows a Tmax between about 120-160 minutes after oral administration to a Beagle dog. In one embodiment a composition according to the present invention shows a Tmax at about 160 minutes after oral administration to a Beagle dog. In one embodiment a composition according to the present invention shows a Tmax at about 150. In one embodiment a composition according to the present invention shows a Tmax after about 140 minutes after oral administration to a Beagle dog. In one embodiment a composition according to the present invention shows a Tmax at about 130. In one embodiment a composition according to the present invention shows a Tmax after about 120 minutes after oral administration to a Beagle dog.

In one embodiment a composition according to the present invention shows a Tmax between about 120-160 minutes after oral administration to a Beagle dog with an empty stomach. In one embodiment a composition according to the present invention shows a Tmax at about 160 minutes after oral administration to a Beagle dog with an empty stomach. In one embodiment a composition according to the present invention shows a Tmax at about 150 with an empty stomach. In one embodiment a composition according to the present invention shows a Tmax after about 140 minutes after oral administration to a Beagle dog with an empty stomach. In one embodiment a composition according to the present invention shows a Tmax at about 130 with an empty stomach. In one embodiment a composition according to the present invention shows a Tmax after about 120 minutes after oral administration to a Beagle dog with an empty stomach. The term “empty stomach” as used herein means that the Beagle dog has no food contents in its stomach that can interfere with the absorption or disintergration/dissolution of a composition according to the present invention, such as demonstrated in example 7 at 360 minutes after feeding according to method 11.

In one embodiment a composition and/or an anionic copolymer coating according to the present invention comprises excipients known to the person skilled in the art. In one embodiment a composition and/or an anionic copolymer coating according to the present invention comprises anionic polymers that may be used in aqueous coating processes.

In one embodiment a composition according to the present invention comprises polymers that may be used in aqueous coating processes, wherein said polymers may be in the form of dispersions or solutions. In one embodiment polymers according to the present invention are cellulose derivatives or acrylate-methylacrylate-acrylic acid derivatives.

In one embodiment an anionic copolymer coating according to the present invention comprises polymers that may be used in aqueous coating processes, wherein said polymers may be in the form of dispersions or solutions. In one embodiment polymers according to the present invention are cellulose derivatives or acrylate-methylacrylate-acrylic acid derivatives.

In one embodiment a composition and/or an anionic copolymer coating according to the present invention comprise excipients as known to the person skilled in the art. Non-limiting examples of such known excipients are disclosed in “Direct compression and the role of filler-binders” (p 173-217): by B. A. C. Carlin, in “Disintegrants in tabletting” (p 217-251): by R. C. Moreton, and in “Lubricants, glidants and adherents” (p 251-269), by N. A. Armstrong, in Pharmaceutical dosage forms: Tablets“, Informa Healthcare, N.Y., vol 2, 2008, L. L. Augsburger and S. W. Hoag”, and incorporated herein by reference.

In one embodiment a composition according to the present invention is in the form of a solid oral formulation. In one embodiment a composition according to the present invention is manufactured into a tablet. In one embodiment a composition according to the present invention is manufactured into a tablet for oral administration.

In one embodiment a tablet core of a composition according to the present invention weights about 710 mg. In one embodiment a composition according to the present invention consisting of a tablet core and an anionic copolymer according to the present invention weighs about 760 mg.

In one embodiment a tablet core comprises about 77% (w/w) salt of capric acid. In one aspect a tablet core comprises about 0.5% (w/w) stearic acid.

In one embodiment a tablet core comprises about 22.5% (w/w) sorbitol. In one embodiment the sorbitol amount is adjusted relative to the amount of protease stabilised insulint. In one embodiment the sorbitol amount is adjusted relative to the amount of protease stabilised insulin. In one embodiment the sorbitol amount is adjusted relative to the amount of protease stabilised insulin after the principle of quantum satis (QS) meaning the amount which is needed to obtain a tablet with the desired weight. In one embodiment a tablet core comprises about 22.5% (w/w) sorbitol, when the amount of protease stabilised insulin is about 0% (w/w). In one embodiment a tablet core comprises about 22.5% (w/w) sorbitol, when the amount of protease stabilised insulin is 0% (w/w). In one embodiment the sorbitol amount is adjusted relative to the amount of protease stabilised insulin, wherein the amount of protease stabilised insulin is at least about 0.5% (w/w). In one embodiment the sorbitol amount is adjusted relative to the amount of protease stabilised insulin, wherein the amount of protease stabilised insulin is at least 0.5% (w/w). In one embodiment the sorbitol amount is adjusted relative to the amount of protease stabilised insulin, wherein the amount of protease stabilised insulin is about 0-22.5% (w/w).

In one embodiment a tablet core comprises about 21.0% (w/w) sorbitol, when the amount of protease stabilised insulin is 0.5% (w/w). In one embodiment a tablet core comprises about 20.5% (w/w) sorbitol, when the amount of protease stabilised insulin is 2% (w/w). In one embodiment a tablet core comprises about 19.5% (w/w) sorbitol, when the amount of protease stabilised insulin is 3% (w/w). In one embodiment a tablet core comprises about 22.5 minus X % (w/w) sorbitol, wherein X is the amount of protease stabilised insulin. In one embodiment a tablet core comprises about 22.5 minus X % (w/w) sorbitol, wherein X is the amount of protease stabilised insulin and X is from 0-22.5. In one embodiment a tablet core comprises about 22.5 minus X % (w/w) sorbitol, wherein X is the amount of protease stabilised insulin and X is about 0, 0.5, 1, 1.5, 2, 2.5, 3.0, 3.5, 4.0, 4.5 or 5.0. In one embodiment a tablet core comprises about 22.5 minus X % (w/w) sorbitol, wherein X is the amount of protease stabilised insulin and X is about 5.5, 6, 6.5, 7, 7.5, 8, 8.5, 9.0, 9.5 or 10.0. In one embodiment a tablet core comprises about 22.5 minus X % (w/w) sorbitol, wherein X is the amount of protease stabilised insulin and X is about 10.5, 11, 11.5, 12, 12.5, 13, 13.5, 14.0, 14.5 or 15.0. In one embodiment a tablet core comprises about 22.5 minus X % (w/w) sorbitol, wherein X is the amount of protease stabilised insulin and X is about 15.5, 16, 16.5, 17, 17.5, 18, 18.5, 19.0, 20.5, 21.0, 21.5, 22.0 or 22.5.

In one aspect an anionic copolymer coating of a composition according to the present invention is coated on to the surface of a tablet core according to the present invention in an amount of about 4-10% (w/w) relative to the tablet core. In one aspect an anionic copolymer coating of a composition according to the present invention is coated on to an outer surface of a tablet core according to the present invention in an amount of about 4% (w/w) relative to the tablet core. In one aspect an anionic copolymer coating of a composition according to the present invention is coated on to an outer surface of a tablet core according to the present invention in an amount of about 5% (w/w) relative to the tablet core. In one aspect an anionic copolymer coating of a composition according to the present invention is coated on to an outer surface of a tablet core according to the present invention in an amount of about 6% (w/w) relative to the tablet core. In one aspect an anionic copolymer coating of a composition according to the present invention is coated on to an outer surface of a tablet core according to the present invention in an amount of about 7% (w/w) relative to the tablet core. In one aspect an anionic copolymer coating of a composition according to the present invention is coated on to an outer surface of a tablet core according to the present invention in an amount of about 7.5% (w/w) relative to the tablet core. In one aspect an anionic copolymer coating of a composition according to the present invention is coated on to an outer surface of a tablet core according to the present invention in an amount of about 8% (w/w) relative to the tablet core. In one aspect an anionic copolymer coating of a composition according to the present invention is coated on to an outer surface of a tablet core according to the present invention in an amount of about 9% (w/w) relative to the tablet core.

In one aspect an anionic copolymer coating of a composition according to the present invention is coated on to an outer surface of a tablet core according to the present invention in an amount of about 10% (w/w) relative to the tablet core.

In one aspect an anionic copolymer coating of a composition according to the present invention is coated on to the surface of a tablet core according to the present invention in an amount of about 7% (w/w) relative to the tablet core. In one aspect an anionic copolymer coating of a composition according to the present invention is coated on to an outer surface of a tablet core according to the present invention in an amount of about 7% (w/w) relative to the tablet core.

In one embodiment the dried anionic copolymer coating coated on an outer surface of a tablet core according to the present invention is of a thickness of about 20-150 μm mm.

In one embodiment the dried anionic copolymer coating coated on an outer surface of a tablet core according to the present invention is of a thickness of about 20 μm or more and an anionic copolymer coating is intact, i.e. continuous.

In one embodiment the dried anionic copolymer coating coated on an outer surface of a tablet core according to the present invention is of a thickness enabling the coating to be intact, i.e. continuous.

In one embodiment one or more additional non-functional coatings are applied on top of an anionic copolymer coating. In one embodiment one or more additional continuous non-functional coatings are applied on top of an anionic copolymer coating. In one embodiment one or more additional discontinuous non-functional coatings are applied on top of an anionic copolymer coating. One embodiment of the present invention regards a pharmaceutical composition wherein a discontinuous additional non-functional coating is applied between an anionic copolymer coating and a tablet core. One embodiment of the present invention regards a pharmaceutical composition wherein an interrupted additional non-functional coating is applied between an anionic copolymer coating and a tablet core.

### Method of Production

In one embodiment the anionic copolymer coating of the present inventions is performed by any methods known to the person skilled in the art.

In one embodiment the coating of the present inventions is performed by any method disclosed in “Coating processes and equipment, by D. M. Jones in “Pharmaceutical dosage forms: Tablets”, Informa Healthcare, N.Y., vol 1, 2008 p 373-399, L. L. Augsburger and S. W. Hoag”, incorporated herein by reference. In one embodiment the tablet core is a tablet core manufactured by suitable methods for formulation solid oral compositions.

In one embodiment an insulin powder is sieved before formulation. In one embodiment a sorbitol (or any other equivalent excipient) powder is sieved before formulation. In one embodiment sorbitol and protease stabilised insulin powder are mixed together. In one embodiment equal amounts of sorbitol and protease stabilised insulin powder are mixed together. In one embodiment equal amounts of sorbitol and protease stabilised insulin powder are mixed by hand.

In one embodiment sorbitol and protease stabilised insulin powders are mixed by hand. In one embodiment sorbitol and protease stabilised insulin powders are initially mixed by hand. In one embodiment sorbitol and protease stabilised insulin powders are mixed by hand and by an automatized mixing process. In one embodiment sorbitol and protease stabilised insulin powders are mixed by hand and by an automatized mixing process, wherein said automatized mixing process is performed in a Tubular-mixer.

In one embodiment sorbitol and protease stabilised insulin powders are mixed by an automatized mixing process. In one embodiment sorbitol and protease stabilised insulin powders are mixed by an automatized mixing process, wherein said automatized mixing process is performed in a Tubular-mixer.

In one embodiment sorbitol and protease stabilised insulin powders are initially mixed by hand, followed by an automatized mixing process. In one embodiment sorbitol and protease stabilised insulin powders are initially mixed by hand until blended together well. In one embodiment sorbitol and protease stabilised insulin powders are initially mixed by hand until blended together well, followed by an automatized mixing process. In one embodiment sorbitol and protease stabilised insulin powders are initially mixed by hand, followed by an automatized mixing process, wherein said automatized mixing process is performed in a Tubular-mixer. In one embodiment sorbitol and protease stabilised insulin powders are initially mixed by hand until blended together well, wherein the degree of blending of said sorbitol and protease stabilised insulin powder is evaluated by eyeballing. In one embodiment sorbitol and protease stabilised insulin powders are initially mixed by hand until blended well, wherein the degree of blending of said sorbitol and protease stabilised insulin powder is evaluated by eyeballing, followed by an automatized mixing process.

In one embodiment equal amounts of sorbitol and protease stabilised insulin powder are mixed by hand and another portion of sorbitol is added in an amount twice as high as the first addition of sorbitol, which then is also stirred well by hand. When said last addition of sorbitol is admixed well, the powder is then subjected to mechanical mixing in a Turbula-mixer or any equivalent mixer to finalise the mixing process, resulting in a homogenous powder.

In one embodiment a salt of capric acid is added to said homogenous powder of sorbitol and protease stabilised insulin in amounts of 1:1. The addition may be performed in two steps and the mixing may initially performed by hand and finalised by mechanical mixing in a Turbula-mixer or any other automatized mixing device. The addition may be performed in two steps and the mixing is initially performed by hand and finalised by mechanical mixing in a Turbula-mixer or any equivalent mixer.

The powder may then be pressed in a tablet press as known to the person skilled in the art, resulting in a tablet core according to the present invention.

The powder may then be pressed in a rotary tablet press as known to the person skilled in the art, resulting in a tablet core according to the present invention. The powder may then be pressed in a single punch tablet press as known to the person skilled in the art, resulting in a tablet core according to the present invention. The powder may then be pressed in a excenter tablet press as known to the person skilled in the art, resulting in a tablet core according to the present invention.

In one embodiment an anionic copolymer coating as defined herein may be coated on top of a tablet core according to the present invention. In one embodiment anionic copolymer coating as defined herein may be coated on top of a tablet according to the present invention. In one embodiment an anionic copolymer coating as defined herein may be coated on top of an outer surface of a tablet core according to the present invention.

In one embodiment an anionic copolymer coating material as defined herein is dispersed in water resulting in “anionic copolymer dispersion”. In one embodiment a dispersion of water and an anionic copolymer coating material as defined herein is placed in a beaker on a suitable stirring apparatus.

In one embodiment an anionic copolymer dispersion or a dry polymer is coated on top of a tablet core according to this invention. In one embodiment an anionic copolymer dispersion or a dry polymer is coated on top of a tablet according to this invention.

In one embodiment the anionic copolymer dispersion is filtrated through a mesh filter prior to the actual coating prior to the actual coating procedure.

In one embodiment the anionic copolymer dispersion is stirred prior to a filtration through a mesh filter, prior to the actual coating procedure. In one embodiment the anionic copolymer dispersion is stirred prior to a filtration through an about 0.24 mm mesh filter, prior to the actual coating procedure.

In one embodiment excipients are added to an anionic copolymer dispersion. In one embodiment excipients are added to an anionic copolymer dispersion in the amount of about 10% (w/w) of the total dry coating material in an anionic copolymer dispersion. In one embodiment excipients are added to an anionic copolymer dispersion in the amount of about 10% (w/w) of the total dry coating material in an anionic copolymer dispersion, wherein said total dry coating material in an anionic copolymer dispersion comprises an anionic copolymer as defined in the present invention.

In one embodiment excipients are added to an anionic copolymer dispersion in the amount of about 10% (w/w) of the total dry coating material in an anionic copolymer dispersion, wherein said total dry coating material in an anionic copolymer dispersion comprises an anionic copolymer such as methyl acrylate, methyl methacrylate and methacrylic acid. In one embodiment excipients are added to an anionic copolymer dispersion in the amount of about 10% (w/w) of the total dry coating material in an anionic copolymer dispersion, wherein said total dry coating material in an anionic copolymer dispersion comprises an anionic copolymer such as EUDRAGIT FS30D® as sold by Evonik Industries (in 2013).

In one embodiment the anionic copolymer dispersion further comprising further excipients is filtrated through a mesh filter prior to the actual coating prior to the actual coating procedure.

In one embodiment the anionic copolymer dispersion comprising further excipients is stirred prior to a filtration through a mesh filter, prior to the actual coating procedure. In one embodiment the anionic copolymer dispersion further comprising further excipients is stirred prior to a filtration through an about 0.24 mm mesh filter, prior to the actual coating procedure.

In one aspect the actual coating procedure of tablet cores or tablets according to the present invention is performed in a pan coater or fluid bed coater. In one aspect the actual coating procedure of tablet cores or tablets according to the present invention is performed in a pan coater or fluid bed coater by spraying the anionic copolymer dispersion through a spray nozzle. In one aspect the actual coating procedure of tablet cores or tablets according to the present invention is performed in a pan coater or fluid bed coater by spraying the anionic copolymer dispersion further comprising further excipients through a spray nozzle.

In one embodiment an anionic copolymer coating processes and equipment may be used as disclosed by D. M. Jones in “Pharmaceutical dosage forms: Tablets”, Informa Healthcare, N.Y., vol. 1, 2008 p 373-399, L. L. Augsburger and S. W. Hoag”, which hereby in incorporated by reference.

### Insulin Peptide

In one embodiment a tablet core according to the present invention comprises an insulin.

In one embodiment a tablet core according to the present invention comprises an insulin analogue. In one embodiment a tablet core according to the present invention comprises a protease stabilised insulin. In one embodiment a tablet core according to the present invention comprises a protease stabilised insulin as defined in the following pages.

As used herein the term “protease stabilised insulin” shall mean an insulin analogue or derivative which is stabilised against proteolytic degradation, i.e. against rapid degradation in the gastro intestinal (GI) tract or elsewhere in the body and thus are protease stabilised insulins.

A protease stabilised insulin is herein to be understood as an insulin analogue or derivative, which is subjected to slower degradation by one or more proteases relativederivative according for use in a pharmaceutical composition according to the invention is subjected to slower degradation by one or more proteases relative to human insulin. In a further embodiment of the invention a protease stabilised insulin for use in the invention is stabilised against degradation by one or more enzymes selected from the group consisting of: pepsin (such as e.g. the isoforms pepsin A, pepsin B, pepsin C and/or pepsin F), chymotrypsin (such as e.g. the isoforms chymotrypsin A, chymotrypsin B and/or chymotrypsin C), trypsin, Insulin-Degrading Enzyme (IDE), elastase (such as e.g. the isoforms pancreatic elastase I and/or II), carboxypeptidase (e.g. the isoforms carboxypeptidase A, carboxypeptidase A2 and/or carboxypeptidase B), aminopeptidase, cathepsin D and other enzymes present in intestinal extracts derived from rat, pig or human.

In one embodiment a protease stabilised insulin for use in the invention is stabilised against degradation by one or more enzymes selected from the group consisting of: chymotrypsin, trypsin, Insulin-Degrading Enzyme (IDE), elastase, carboxypeptidases, aminopeptidases and cathepsin D. In a further embodiment a protease stabilised insulin for use in the invention is stabilised against degradation by one or more enzymes selected from the group consisting of: chymotrypsin, carboxypeptidases and IDE. In a yet further embodiment a protease stabilised insulin for use in the invention is stabilised against degradation by one or more enzymes selected from: chymotrypsin and IDE. In a yet further embodiment a protease stabilised insulin for use in the invention is stabilised against degradation by one or more enzymes selected from: chymotrypsin and carboxypeptidases. T½ may be determined as described in example 102 of WO2011/161125 as a measure of the proteolytical stability of a protease stabilised insulin for use in the invention towards protease enzymes such as chymotrypsin, pepsin and/or carboxypeptidase A or towards a mixture of enzymes such as tissue extracts (fromliver, kidney, duodenum, jejunum, ileum, colon, stomach, etc.). In one embodiment of the invention T½ is increased relative to human insulin. In a further embodiment T½ is increased relative to the protease stabilised insulin without one or more additional disulfide bonds. In a yet further embodiment T½ is increased at least 2-fold relative to human insulin. In a yet further embodiment T½ is increased at least 2-fold relative to the protease stabilised insulin without one or more additional disulfide bonds. In a yet further embodiment T½ is increased at least 3-fold relative to human insulin. In a yet further embodiment T½ is increased at least 3-fold relative to the protease stabilised insulin without one or more additional disulfide bonds. In a yet further embodiment T½ is increased at least 4-fold relative to human insulin. In a yet further embodiment T½ is increased at least 4-fold relative to the protease stabilised insulin without one or more additional disulfide bonds. In a yet further embodiment T½ is increased at least 5-fold relative to human insulin. In a yet further embodiment T½ is increased at least 5-fold relative to the protease stabilised insulin without one or more additional disulfide bonds. In a yet further embodiment T½ is increased at least 10-fold relative to human insulin. In a yet further embodiment T½ is increased at least 10-fold relative to the protease stabilised insulin without one or more additional disulfide bonds. T½ may also be expressed as the relative T½, relative to a proteolytically stabilised insulin analogue, A14E, B25H, desB30 human insulin as described in example 102 of WO2011/161125.

In one embodiment, a protease stabilised insulin may have increased solubility relative to human insulin. In a further embodiment, a protease stabilised insulin has increased solubility relative to human insulin at pH 3-9. In a yet further embodiment, a protease stabilised insulin has increased solubility relative to human insulin at pH 4-8.5. In a still further embodiment, a protease stabilised insulin has increased solubility relative to human insulin at pH 4-8. In a yet further embodiment, a protease stabilised insulin has increased solubility relative to human insulin at pH 4.5-8. In a further embodiment, a protease stabilised insulin has increased solubility relative to human insulin at pH 5-8. In a yet further embodiment, a protease stabilised insulin has increased solubility relative to human insulin at pH 5.5-8. In a further embodiment, a protease stabilised insulin has increased solubility relative to human insulin at pH 6-8.

In one embodiment, a protease stabilised insulin has increased solubility relative to human insulin at pH 2-4.

In one embodiment, a protease stabilised insulin may have increased solubility relative to the parent insulin. In a further embodiment, a protease stabilised insulin has increased solubility relative to the parent insulin at pH 3-9. In a yet further embodiment a protease stabilised insulin has increased solubility relative to parent insulin at pH 4-8.5. In a still further embodiment, a protease stabilised insulin has increased solubility relative to parent insulin at pH 4-8. In a yet further embodiment, a protease stabilised insulin has increased solubility relative to parent insulin at pH 4.5-8. In a still further embodiment, a protease stabilised insulin has increased solubility relative to parent insulin at pH 5-8. In a yet further embodiment, a protease stabilised insulin has increased solubility relative to parent insulin at pH 5.5-8. In a further embodiment, a protease stabilised insulin has increased solubility relative to parent insulin at pH 6-8.

In one embodiment, a protease stabilised insulin has increased solubility relative to parent insulin at pH 2-4.

By “increased solubility at a given pH” is meant that a larger concentration of a protease stabilised insulin dissolves in an aqueous or buffer solution at the pH of the solution relative to the parent insulin. Methods for determining whether the insulin contained in a solution is dissolved are known in the art.

In one embodiment, the solution may be subjected to centrifugation for 20 minutes at 30,000 g and then the insulin concentration in the supernatant may be determined by RP-HPLC. If this concentration is equal within experimental error to the insulin concentration originally used to make the composition, then the insulin is fully soluble in the composition of the invention. In one embodiment, the solubility of the insulin in a composition of the invention may simply be determined by examining by eye the container in which the composition is contained. The insulin is soluble if the solution is clear to the eye and no particulate matter is either suspended or precipitated on the sides/bottom of the container.

A protease stabilised insulin for use in the invention may have increased apparent potency and/or bioavalability relative to the parent insulin when compared upon measurement.

In a one embodiment of the invention, a fatty diacid of a side chain in a protease stabilised insulin for use in the present invention has from 6 to 40 carbon atoms. In a further embodiment of the invention, a fatty diacid of a side chain in a protease stabilised insulin for use in the present invention has from 8 to 26 carbon atoms. In a further embodiment of the invention a fatty diacid of a side chain in a protease stabilised insulin for use in the present invention has from 8 to 22 carbon atoms. In a further embodiment of the invention, a fatty diacid of a side chain in a protease stabilised insulin for use in the present invention has from 14 to 22 carbon atoms.

In a further embodiment of the invention, a fatty diacid of a side chain in a protease stabilised insulin for use in the present invention has from 16 to 22 carbon atoms.

In a further embodiment of the invention, a fatty diacid of a side chain in a protease stabilised insulin for use in the present invention has from 16 to 20 carbon atoms.

In a further embodiment of the invention, a fatty diacid of a side chain in a protease stabilised insulin for use in the present invention has from 16 to 18 carbon atoms.

In a further embodiment of the invention, a fatty diacid of a side chain in a protease stabilised insulin for use in the present invention has 16 carbon atoms. In a further embodiment of the invention, a fatty diacid of a side chain in a protease stabilised insulin for use in the present invention has 18 carbon atoms. In a further embodiment of the invention, a fatty diacid of a side chain in a protease stabilised insulin for use in the present invention has 20 carbon atoms. In a further embodiment of the invention, a fatty diacid of a side chain in a protease stabilised insulin for use in the present invention has 22 carbon atoms.

In one embodiment a tablet core according to the present invention comprises a protease stabilised insulin as disclosed and claimed in patent applications WO2009/115469 or WO2011/161125. Methods for preparation of such insulins as well as assays for characterizing such insulins, such as physical and chemical stability as well as potency and T½ are provided in patent applications WO2009/115469 or WO2011/161125. In one embodiment a tablet core according to the present invention comprises a protease stabilised insulin selected from the examples of patent applications WO2009/115469 or WO2011/161125.

In another embodiment, a protease stabilised insulin is an insulin analogue wherein
the amino acid in position A12 is Glu or Asp and/or the amino acid in position A13 is His, Asn, Glu or Asp and/or the amino acid in position A14 is Asn, Gln, Glu, Arg, Asp, Gly or His and/or the amino acid in position A15 is Glu or Asp; and
the amino acid in position B24 is His and/or the amino acid in position B25 is His and/or the amino acid in position B26 is His, Gly, Asp or Thr and/or the amino acid in position B27 is His, Glu, Gly or Arg and/or the amino acid in position B28 is His, Gly or Asp; and
   which optionally further comprises one or more additional mutations.

In another embodiment a protease stabilised insulin is an analogue or derivative comprising the A14E mutation.

In another embodiment a protease stabilised insulin is an analogue or derivative comprising the B25H mutation.

In another embodiment a protease stabilised insulin is an analogue or derivative comprising desB30 mutation.

In another embodiment a protease stabilised insulin is an analogue or derivative comprising desB27 mutation.

In another embodiment a protease stabilised insulin is an analogue or derivative comprising the B25H or B25N mutations in combination with mutations in B27, optionally in combination with other mutations.

In another embodiment a protease stabilised insulin is an analogue or derivative comprising the A14E, B25H or B25N alone or in combination.

In another embodiment a protease stabilised insulin is an analogue or derivative comprising the A14E, B25H or B25N mutations in combination with mutations in B27, optionally in combination with other mutations.

In another embodiment a protease stabilised insulin is an analogue or derivative comprising the A14E, B25H or B25N alone or in combination with the B27 mutations previously described or the desB30 or desB27 mutation.

In another embodiment a protease stabilised insulin is an analogue or derivative comprising the B25H in combination with mutations in desB27.

In another embodiment a protease stabilised insulin is an analogue or derivative comprising the B25H in combination with mutations in desB30.

In another embodiment a protease stabilised insulin is an analogue or derivative comprising the B25H or B25N mutations in combination with mutations in B27, optionally in combination with other mutations.

The mutations in position B27 can, for example, be Glu or Asp. These protease stabilised acyated insulin analogues or derivative comprising both the B25 and B27 mutations have advantageous properties.

In one embodiment a protease stabilised insulin is an acylated insulin analogue, wherein said protease stabilised insulin comprises an A-chain amino acid sequence of formula 1:

Formula (1)
(SEQ ID No: 1)
Xaa_{A(-2)}-Xaa_{A(-1)}-Xaa_{A0}-Gly-Ile-Val-Glu-Gln-Cys-
Cys-Xaa_{A8}-Ser-Ile-Cys-Xaa_{A12}-Xaa_{A13}-Xaa_{A14}-Xaa_{A15}-
Leu-Glu-Xaa_{A18}-Tyr-Cys-Xaa_{A21}

and a B-chain amino acid sequence of formula 2:

Formula (2)
(SEQ ID No: 2)
Xaa_{B(-2)}-Xaa_{B(-1)}-Xaa_{B0}-Xaa_{B1}-Xaa_{B2}-Xaa_{B3}-Xaa_{B4}-
His-Leu-Cys-Gly-Ser-Xaa_{B10}-Leu-Val-Glu-Ala-Leu-
Xaa_{B16}-Leu-Val-Cys-Gly-Glu-Arg-Gly-Xaa_{B24}-Xaa_{B25}-
Xaa_{B26}-Xaa_{B27}-Xaa_{B28}-Xaa_{B29}-Xaa_{B30}-Xaa_{B31}-Xaa_{B32}

wherein

Xaa_{A(-2)} is absent or Gly;

Xaa_{A(-1)} is absent or Pro;

Xaa_{A0} is absent or Pro;

Xaa_{A8} is independently selected from Thr and His;

Xaa_{A12} is independently selected from Ser, Asp and Glu;

Xaa_{A13} is independently selected from Leu, Thr, Asn, Asp, Gln, His, Lys, Gly, Arg, Pro, Ser and Glu;

Xaa_{A14} is independently selected from Tyr, Thr, Asn, Asp, Gln, His, Lys, Gly, Arg, Pro, Ser and Glu;

Xaa_{A15} is independently selected from Gln, Asp and Glu;

Xaa_{A18} is independently selected from Asn, Lys and Gln;

Xaa_{A21} is independently selected from Asn and Gln;

Xaa_{B(-2)} is absent or Gly;

Xaa_{B(-1)} is absent or Pro;

Xaa_{B0} is absent or Pro;

Xaa_{B1} is absent or independently selected from Phe and Glu;

Xaa_{B2} is absent or Val;

Xaa_{B3} is absent or independently selected from Asn and Gln;

Xaa_{B4} is independently selected from Gln and Glu;

Xaa_{B10} is independently selected from His, Asp, Pro and Glu;

Xaa_{B16} is independently selected from Tyr, Asp, Gln, His, Arg, and Glu;

Xaa_{B24} is independently selected from Phe and His;

Xaa_{B25} is independently selected from Asn, Phe and His;

Xaa_{B26} is absent or independently selected from Tyr, His, Thr, Gly and Asp;

Xaa_{B27} is absent or independently selected from Thr, Asn, Asp, Gln, His, Lys, Gly, Arg, Pro, Ser and Glu;

Xaa_{B28} is absent or independently selected from Pro, His, Gly and Asp;

Xaa_{B29} is absent or independently selected from Lys, Arg and Gln; and, preferably,

Xaa_{B29} is absent or independently selected from Lys and Gln;

Xaa_{B30} is absent or Thr;

Xaa_{B31} is absent or Leu;

Xaa_{B32} is absent or Glu;

wherein the A-chain amino acid sequence and the B-chain amino acid sequence are connected by disulfide bridges between the cysteines in position 7 of the A-chain and the cysteine in position 7 of the B-chain, and between the cysteine in position 20 of the A-chain and the cysteine in position 19 of the B-chain and wherein the cysteines in position 6 and 11 of the A-chain are connected by a disulfide bridge.

In one embodiment, a protease stabilised insulin is an acylated insulin analogue, wherein said protease stabilised insulin comprises an A-chain amino acid sequence of formula 3:

Formula (3)
(SEQ ID No: 3)
Gly-Ile-Val-Glu-Gln-Cys-Cys-Xaa_{A8}-Ser-Ile-Cys-
Xaa_{A12}-Xaa_{A13}-Xaa_{A14}-Xaa_{A15}-Leu-Glu-Xaa_{A18}-Tyr-
Cys-Xaa_{A21}

and a B-chain amino acid sequence of formula 4:

Formula (4)
(SEQ ID No: 4)
Xaa_{B1}-Val-Xaa_{B3}-Xaa_{B4}-His-Leu-Cys-Gly-Ser-Xaa_{B10}-
Leu-Val-Glu-Ala-Leu-Xaa_{B16}-Leu-Val-Cys-Gly-Glu-
Arg-Gly-Xaa_{B24}-His-Xaa_{B26}-Xaa_{B27}-Xaa_{B28}-Xaa_{B29}-
Xaa_{B30}

wherein

Xaa_{A8} is independently selected from Thr and His;

Xaa_{A12} is independently selected from Ser, Asp and Glu;

Xaa_{A13} is independently selected from Leu, Thr, Asn, Asp, Gln, His, Lys, Gly, Arg, Pro, Ser and Glu;

Xaa_{A14} is independently selected from Thr, Asn, Asp, Gln, His, Lys, Gly, Arg, Pro, Ser and Glu;

Xaa_{A15} is independently selected from Gln, Asp and Glu;

Xaa_{A18} is independently selected from Asn, Lys and Gln;

Xaa_{A21} is independently selected from Asn, and Gln;

Xaa_{B1} is independently selected from Phe and Glu;

Xaa_{B3} is independently selected from Asn and Gln;

Xaa_{B4} is independently selected from Gln and Glu;

Xaa_{B10} is independently selected from His, Asp, Pro and Glu;

Xaa_{B16} is independently selected from Tyr, Asp, Gln, His, Arg, and Glu;

Xaa_{B24} is independently selected from Phe and His;

Xaa_{B26} is absent or independently selected from Tyr, His, Thr, Gly and Asp;

Xaa_{B27} is absent or independently selected from Thr, Asn, Asp, Gln, His, Lys, Gly, Arg, Pro, Ser and Glu;

Xaa_{B28} is absent or independently selected from Pro, His, Gly and Asp;

Xaa_{B29} is absent or independently selected from Lys, Arg and Gln; and, preferably,

Xaa_{B29} is absent or independently selected from Lys and Gln;

Xaa_{B30} is absent or Thr;
wherein the A-chain amino acid sequence and the B-chain amino acid sequence are connected by disulfide bridges between the cysteines in position 7 of the A-chain and the cysteine in position 7 of the B-chain, and between the cysteine in position 20 of the A-chain and the cysteine in position 19 of the B-chain and wherein the cysteines in position 6 and 11 of the A-chain are connected by a disulfide bridge.

In one embodiment, a protease stabilised insulin is an acylated insulin analogue wherein

Xaa_{A8} is independently selected from Thr and His;

Xaa_{A12} is independently selected from Ser and Glu;

Xaa_{A13} is independently selected from Leu, Thr, Asn, Asp, Gln, His, Lys, Gly, Arg, Pro, Ser and Glu;

Xaa_{A14} is independently selected from Asp, His, and Glu;

Xaa_{A15} is independently selected from Gln and Glu;

Xaa_{A18} is independently selected from Asn, Lys and Gln;

Xaa_{A21} is independently selected from Asn, and Gln;

Xaa_{B1} is independently selected from Phe and Glu;

Xaa_{B3} is independently selected from Asn and Gln;

Xaa_{B4} is independently selected from Gln and Glu;

Xaa_{B10} is independently selected from His, Asp, Pro and Glu;

Xaa_{B16} is independently selected from Tyr, Asp, Gln, His, Arg, and Glu;

Xaa_{B24} is independently selected from Phe and His;

Xaa_{B25} is independently selected from Phe, Asn and His;

Xaa_{B26} is independently selected from Tyr, Thr, Gly and Asp;

Xaa_{B27} is independently selected from Thr, Asn, Asp, Gln, His, Lys, Gly, Arg, and Glu;

Xaa_{B28} is independently selected from Pro, Gly and Asp;

Xaa_{B29} is independently selected from Lys and Gln;

Xaa_{B30} is absent or Thr;

wherein the A-chain amino acid sequence and the B-chain amino acid sequence are connected by disulfide bridges between the cysteines in position 7 of the A-chain and the cysteine in position 7 of the B-chain, and between the cysteine in position 20 of the A-chain and the cysteine in position 19 of the B-chain and wherein the cysteines in position 6 and 11 of the A-chain are connected by a disulfide bridge.

For the sake of convenience, here follows the names of codable, natural amino acids with the usual three letter codes & one letter codes in parenthesis: Glycine (Gly & G), proline (Pro & P), alanine (Ala & A), valine (Val & V), leucine (Leu & L), isoleucine (Ile & I), methionine (Met & M), cysteine (Cys & C), phenylalanine (Phe & F), tyrosine (Tyr & Y), tryptophan (Trp & W), histidine (His & H), lysine (Lys & K), arginine (Arg & R), glutamine (Gln & Q), asparagine (Asn & N), glutamic acid (Glu & E), aspartic acid (Asp & D), serine (Ser & S) and threonine (Thr & T). If, due to typing errors, there are deviations from the commonly used codes, the commonly used codes apply. The amino acids present in the protease stabilised insulins for use in this invention are, preferably, amino acids which can be coded for by a nucleic acid. In one embodiment the protease stabilised insulin is substituted by Gly, Glu, Asp, His, Gln, Asn, Ser, Thr, Lys, Arg and/or Pro, and/or Gly, Glu, Asp, His, Gln, Asn, Ser, Thr, Lys, Arg and/or Pro is added to the protease stabilised insulin. In one embodiment the protease stabilised insulin is substituted by Glu, Asp, His, Gln, Asn, Lys and/or Arg and/or Glu, Asp, His, Gln, Asn, Lys and/or Arg is added to the protease stabilised insulin.

In one embodiment, an protease stabilised insulin for a pharmaceutical composition according to this invention is an acylated, protease stabilised insulin comprising a protease stabilised insulin before acylation and a side chain, wherein protease stabilised insulin is selected from the group consisting of: A14E, B25H, desB30 human insulin; A14H, B25H, desB30 human insulin; A14E, B1E, B25H, desB30 human insulin; A14E, B16E, B25H, desB30 human insulin; A14E, B25H, B28D, desB30 human insulin; A14E, B25H, B27E, desB30 human insulin; A14E, B1E, B25H, B27E, desB30 human insulin; A14E, B1E, B16E, B25H, B27E, desB30 human insulin; A8H, A14E, B25H, desB30 human insulin; A8H, A14E, B25H, B27E, desB30 human insulin; A8H, A14E, B1E, B25H, desB30 human insulin; A8H, A14E, B1E, B25H, B27E, desB30 human insulin; A8H, A14E, B1E, B16E, B25H, B27E, desB30 human insulin; A8H, A14E, B16E, B25H, desB30 human insulin; A14E, B25H, B26D, desB30 human insulin; A14E, B1E, B27E, desB30 human insulin; A14E, B27E, desB30 human insulin; A14E, B28D, desB30 human insulin; A14E, B28E, desB30 human insulin; A14E, B1E, B28E, desB30 human insulin; A14E, B1E, B27E, B28E, desB30 human insulin; A14E, B1E, B25H, B28E, desB30 human insulin; A14E, B1E, B25H, B27E, B28E, desB30 human insulin; A14D, B25H, desB30 human insulin; B25N, B27E, desB30 human insulin; A8H, B25N, B27E, desB30 human insulin; A14E, B27E, B28E, desB30 human insulin; A14E, B25H, B28E, desB30 human insulin; B25H, B27E, desB30 human insulin; B1E, B25H, B27E, desb30 human insulin; A8H, B1E, B25H, B27E, desB30 human insulin; A8H, B25H, B27E, desB30 human insulin; B25N, B27D, desB30 human insulin; A8H, B25N, B27D, desB30 human insulin; B25H, B27D, desB309 human insulin; A8H, B25H, B27D, desB30 human insulin; A(−1)P, A(0)P, A14E, B25H, desB30 human insulin; A14E, B(−1)P, B(0)P, B25H, desB30 human insulin; A(−1)P, A(0)P, A14E, B(−1)P, B(0)P, B25H, desB30 human insulin; A14E, B25H, 630T, B31L, B32E human insulin; A14E, B25H human insulin; A14E, B16H, B25H, desB30 human insulin; A14E, 610P, B25H, desB30 human insulin; A14E, B10E, B25H, desB30 human insulin; A14E, B4E, B25H, desB30 human insulin; A14H, B16H, B25H, desB30 human insulin; A14H, B10E, B25H, desB30 human insulin; A13H, A14E, B10E, B25H, desB30 human insulin; A13H, A14E, B25H, desB30 human insulin; A14E, A18Q, B3Q, B25H, desB30 human insulin; A14E, B24H, B25H, desB30 human insulin; A14E, B25H, B26G, B27G, B28G, desB30 human insulin; A14E, B25H, B26G, B27G, B28G, B29R, desB30 human insulin; A14E, A21G, B25H, B26G, B27G, B28G, desB30 human insulin; A14E, A21G, B25H, B26G, B27G, B28G, B29R, desB30 human insulin; A14E, A18Q, A21Q, B3Q, B25H, desB30 human insulin; A14E, A18Q, A21Q, B3Q, B25H, B27E, desB30 human insulin; A14E, A18Q, B3Q, B25H, desB30 human insulin; A13H, A14E, B1E, B25H, desB30 human insulin; A13N, A14E, B25H, desB30 human insulin; A13N, A14E, B1E, B25H, desB30 human insulin; A(−2)G, A(−1)P, A(0)P, A14E, B25H, desB30 human insulin; A14E, B(−2)G, B(−1)P, B(0)P, B25H, desB30 human insulin; A(−2)G, A(−1)P, A(0)P, A14E, B(−2)G, B(−1)P, B(0)P, B25H, desB30 human insulin; A14E, B27R, B28D, B29K, desB30 human insulin; A14E, B25H, B27R, B28D, B29K, desB30 human insulin; A14E, B25H, B26T, B27R, B28D, B29K, desB30 human insulin; A14E, B25H, B27R, desB30 human insulin; A14E, B25H, B27H, desB30 human insulin; A14E, A18Q, B3Q, B25H, desB30 human insulin; A13E, A14E, B25H, desB30 human insulin; A12E, A14E, B25H, desB30 human insulin; A15E, A14E, B25H, desB30 human insulin; A13E, B25H, desB30 human insulin; A12E, B25H, desB30 human insulin; A15E, B25H, desB30 human insulin; A14E, B25H, desB27, desB30 human insulin; A14E, B25H, B26D, B27E, desB30 human insulin; A14E, B25H, B27R, desB30 human insulin; A14E, B25H, B27N, desB30 human insulin; A14E, B25H, B27D, desB30 human insulin; A14E, B25H, B27Q, desB30 human insulin; A14E, B25H, B27E, desB30 human insulin; A14E, B25H, B27G, desB30 human insulin; A14E, B25H, B27H, desB30 human insulin; A14E, B25H, B27K, desB30 human insulin; A14E, B25H, B27P, desB30 human insulin; A14E, B25H, B27S, desB30 human insulin; A14E, B25H, B27T, desB30 human insulin; A13R, A14E, B25H, desB30 human insulin; A13N, A14E, B25H, desB30 human insulin; A13D, A14E, B25H, desB30 human insulin; A13Q, A14E, B25H, desB30 human insulin; A13E, A14E, B25H, desB30 human insulin; A13G, A14E, B25H, desB30 human insulin; A13H, A14E, B25H, desB30 human insulin; A13K, A14E, B25H, desB30 human insulin; A13P, A14E, B25H, desB30 human insulin; A13S, A14E, B25H, desB30 human insulin; A13T, A14E, B25H, desB30 human insulin; A14E, 616R, B25H, desB30 human insulin; A14E, 616D, B25H, desB30 human insulin; A14E, 616Q, B25H, desB30 human insulin; A14E, B16E, B25H, desB30 human insulin; A14E, B16H, B25H, desB30 human insulin; A14R, B25H, desB30 human insulin; A14N, B25H, desB30 human insulin; A14D, B25H, desB30 human insulin; A14Q, B25H, desB30 human insulin; A14E, B25H, desB30 human insulin; A14G, B25H, desB30 human insulin; A14H, B25H, desB30 human insulin; A8H, 610D, B25H human insulin; and A8H, A14E, B10E, B25H, desB30 human insulin and this embodiment may, optionally, comprise A14E, B25H, B29R, desB30 human insulin; B25H, desB30 human insulin; and B25N, desB30 human insulin.

In one embodiment, a protease stabilised insulin before acylation is selected from the group consisting of: A14E, B25H, desB30 human insulin, A14E, B16H, B25H, desB30 human insulin, A14E, B25H, desB27, desB30 human insulin and A14E, desB27, desB30 human insulin.

In one embodiment an protease stabilised insulin for use in the invention has a side chain. In one embodiment a side chain according to the present invention is an acyl moiety. In one embodiment the side chain is attached to the epsilon amino group of a lysine residue. In one embodiment the side chain is attached to the epsilon amino group of a lysine residue in the B-chain.

In one embodiment a protease stabilised insulin for use in the invention has two or more cysteine substitutions, the three disulfide bonds of human insulin retained and a side-chain which is attached to the epsilon amino group of a lysine residue such as in the B-chain.

Disulfide bonds are derived by the coupling of two thiol groups and are herein to be understood as the linkage between two sulfur atoms, i.e. a structure having the overall connectivity R—S—S—R. Disulfide bonds may also be called connecting disulfide bonds, SS-bonds or disulfide bridges. A disulfide bond is created by the introduction of two cysteine amino acid residues to a peptide with subsequent oxidation of the two thiol groups to a disulfide bond. Such oxidation may be performed chemically (as known by persons skilled in the art) or may happen during insulin expression in e.g. yeast.

In one embodiment a protease stabilised insulin for use in the invention is a modified insulin wherein two amino acid residues have been substituted by cysteine residues, a side chain has been introduced and optionally the amino acid in position B30 has been deleted relative to the amino acid sequence of human insulin.

In one embodiment a protease stabilised insulin for use in the invention comprises a side chain and between 2 and 9 mutations relative to human insulin wherein at least two substitutions are to cysteine residues, alternatively an protease stabilised insulin according to the invention comprises a side chain and between 2 and 8 mutations relative to human insulin wherein at least two substitutions are to cysteine residues, alternatively a side chain and between 2 and 7 mutations relative to human insulin wherein at least two substitutions are to cysteine residues, alternatively a side chain and between 2 and 6 mutations relative to human insulin wherein at least two substitutions are to cysteine residues, alternatively a side chain and between 2 and 5 mutations relative to human insulin wherein at least two substitutions are to cysteine residues, alternatively a side chain and between 2 and 4 mutations relative to human insulin wherein at least two substitutions are to cysteine residues, alternatively a side chain and between 2 and 3 mutations relative to human insulin wherein at least two substitutions are to cysteine residues, or alternatively a side chain and 2 cysteine substitutions relative to human insulin. When introducing cysteine residues into the protease stabilised insulin without one or more additional disulfide bonds, the cysteine residues are placed in the three dimensional structure of the folded insulin analogue to allow for the formation of one or more additional disulfide bonds. For example, if placing two new cysteine residues, the proximity of the new cysteine residues in the three dimensional structure is such that a disulfide bond may be formed between the two new cysteine residues.

The number of disulfide bonds in a protein (such as insulin) can be readily determined by accurate intact mass measurements as described, for example in the Examples. The disulfide bonds connectivity can be verified (determined) by standard techniques known in the art, such as peptide mapping. The general strategy for disulfide bond mapping in an insulin peptide includes the following steps: 1) Fragmentation of the non-reduced insulin into disulfide bonded peptides containing, if possible, only a single disulfide bond per peptide. The chosen conditions is also such that rearrangement of disulfide bonds is avoided, 2) Separation of disulfide bonded peptides from each other. 3) Identification of the cysteine residues involved in the individual disulfide bonds.

In one embodiment of the invention an protease stabilised insulin which has a side chain and at least two cysteine substitutions is provided, where the three disulfide bonds of human insulin are retained.

In one embodiment of the invention an protease stabilised insulin which has two or more cysteine substitutions is provided, where the three disulfide bonds of human insulin are retained, and wherein at least one amino acid residue in a position selected from the group consisting of A9, A10 and A12 of the A-chain is substituted with a cysteine, at least one amino acid residue in a position selected from the group consisting of B1, B2, B3, B4, B5 and B6 of the B-chain is substituted with a cysteine, a side chain is attached to the epsilon amino group of a lysine residue in the B-chain and optionally the amino acid in position B30 is deleted.

In one embodiment of the invention the amino acid residue in position A10 of the A-chain is substituted with a cysteine, at least one amino acid residue in a position selected from the group consisting of B1, B2, B3, and B4 of the B-chain is substituted with a cysteine, a side chain is attached to the epsilon amino group of a lysine residue in the B-chain and optionally the amino acid in position B30 is deleted.

In one embodiment of the invention at least one amino acid residue in a position selected from the group consisting of A9, A10 and A12 of the A-chain is substituted with a cysteine, at least one amino acid residue in a position selected from the group consisting of B1, B2, B3, B4, B5 and B6 of the B-chain is substituted with a cysteine, at least one amino acid residue in a position selected from the group consisting of A14, A21, B1, B3, B10, B16, B22, B25, B26, B27, B28, B29, B30, B31, B32 is substituted with an amino acid which is not a cysteine, a side chain is attached to the epsilon amino group of a lysine residue in the B-chain and optionally the amino acid in position B30 is deleted.

It is understood that when B1 or B3 is cysteine, the same amino acid cannot be an amino acid which is not cysteine, whereas if e.g. B1 is cysteine B3 may according to the embodiment of the invention be substituted with an amino acid which is not a cysteine and vice versa. In one embodiment of the invention, the amino acid residue in position A10 of the A-chain is substituted with a cysteine, at least one amino acid residue in a position selected from the group consisting of B1, B2, B3, and B4 of the B-chain is substituted with a cysteine, optionally at least one amino acid residue is substituted with an amino acid which is not a cysteine, a side chain is attached to the epsilon amino group of a lysine residue in the B-chain and optionally the amino acid in position B30 is deleted. In one embodiment of the invention, the amino acid residue in position A10 of the A-chain is substituted with a cysteine, at least one amino acid residue in a position selected from the group consisting of B3 and B4 of the B-chain is substituted with a cysteine, optionally at least one amino acid residue is substituted with an amino acid which is not a cysteine, a side chain is attached to the epsilon amino group of a lysine residue in the B-chain and optionally the amino acid in position B30 is deleted. In one embodiment of the invention, the amino acid residue in position A10 of the A-chain is substituted with a cysteine, the amino acid residue in position B3 of the B-chain is substituted with a cysteine, optionally at least one amino acid residue is substituted with an amino acid which is not a cysteine, a side chain is attached to the epsilon amino group of a lysine residue in the B-chain and optionally the amino acid in position B30 is deleted. In one embodiment of the invention, the amino acid residue in position A10 of the A-chain is substituted with a cysteine, the amino acid residue in B4 of the B-chain is substituted with a cysteine, optionally at least one amino acid residue is substituted with an amino acid which is not a cysteine, a side chain is attached to the epsilon amino group of a lysine residue in the B-chain and optionally the amino acid in position B30 is deleted.

An additional disulfide bond obtained by the invention may be connecting two cysteines of the same chain, i.e. two cysteines in the A-chain or two cysteines in the B-chain of the insulin, or connecting a cysteine in the A-chain with a cysteine in the B-chain of the insulin. In one embodiment, an protease stabilised insulin for use in the invention is obtained, wherein at least one additional disulfide bond is connecting two cysteines in the A-chain or connecting two cysteines in the B-chain.

In one embodiment, an protease stabilised insulin for use in invention is obtained, wherein at least one additional disulfide bond is connecting a cysteine in the A-chain with a cysteine in the B-chain.

In one embodiment of the invention, cysteines are substituted into two positions of the protease stabilised insulin, where the positions are selected from the group consisting of:

A10C, B1C;

A10C, B2C;

A10C, B3C;

A10C, B4C;

A10C, B5C; and

B1C, B4C.

In one embodiment of the invention, cysteines are substituted into two positions of the insulin analogue, where the positions are selected from the group consisting of:

A10C, B1C;

A10C, B2C;

A10C, B3C;

A10C, B4C; and

B1C, B4C.

In one embodiment of the invention, cysteines are substituted into two positions of the protease stabilised insulin, where the positions are selected from the group consisting of:

A10C, B1C;

A10C, B2C;

A10C, B3C; and

A10C, B4C.

In one embodiment of the invention, cysteines are substituted into two positions of the insulin analogue, where the positions are selected from the group consisting of:

A10C, B3C; and

A10C, B4C.

In one embodiment of the invention, cysteines are substituted into two positions of the insulin analogue, where the positions are A10C and B3C.

In one embodiment of the invention, cysteines are substituted into two positions of the insulin analogue, where the positions are A10C and B4C.

In one embodiment of the invention, protease stabilised insulins of the invention comprise in addition to the cysteine substitutions one or more amino acids selected from the group consisting of: A8H, A14E, A14H, A18L, A21G, B1G, B3Q, B3E, B3T, B3V, B3K, B3L, B16H, B16E, B22E, B24G, B25A, B25H, B25N, B27E, B27D, B27P, B28D, B28E, B28K, des61, desB24, desB25, desB27 and des630. In one embodiment of the invention, protease stabilised insulins of the invention comprise in addition to the cysteine substitutions one or more amino acids selected from the group consisting of: A8H, A14E, A21G, des61, B1G, B3Q, B3E, B10E, B16H, B16E, B24G, B25H, B25A, B25N, B25G, desB27, B27E, B28E, B28D, and des630.

In one embodiment of the invention, protease stabilised insulins of the invention comprise in addition to the cysteine substitutions one or more amino acids selected from the group consisting of: A21G, des61, B1G, B3Q, B3S, B3T and B3E.

In one embodiment of the invention, protease stabilised insulins of the invention comprise in addition to the cysteine substitutions one or more amino acids selected from the group consisting of: A8H, A14E, A14H, B16H, B10E, B16E, B25H, B25A, B25N, B27E, B27P, desB27, B28E and des630.

In one embodiment of the invention, protease stabilised insulins of the invention comprise in addition to the cysteine substitutions one or more amino acids selected from the group consisting of: B28E, B28D, desB27, desB30 and A14E.

In one embodiment of the invention, protease stabilised insulins of the invention comprise in addition to the cysteine substitutions one or more amino acids selected from the group consisting of: B3K, B29E, B27E, B27D, desB27, B28E, B28D, B28K and B29P

In one embodiment of the invention, protease stabilised insulins of the invention comprise in addition to the cysteine substitutions a C-peptide connecting the C-terminus of the B-chain with the N-terminus of the A-chain (to form a so called single-chain protease stabilised insulin). In one embodiment of the invention, the parent insulin is selected from the group consisting of single chain insulin analogues. In one embodiment of the invention, the parent insulin is selected from the group consisting of single chain insulin analogues listed in WO2007096332, WO2005054291 or WO2008043033, which patents are herein specifically incorporated by reference.

In one embodiment of the invention, a protease stabilised insulin is obtained which comprises two cysteine substitutions resulting in one additional disulfide bond relative to human insulin.

In one embodiment a protease stabilised insulin for use in the invention is an insulin analogue comprising at least two cysteine substitutions, wherein the insulin analogue is acylated in one or more amino acids of the insulin peptide.

Modifications in the insulin molecule are denoted stating the chain (A or B), the position, and the one or three letter code for the amino acid residue substituting the native amino acid residue.

Herein terms like “A1”, “A2” and “A3” etc. indicates the amino acid in position 1, 2 and 3 etc., respectively, in the A chain of insulin (counted from the N-terminal end). Similarly, terms like B1, B2 and B3 etc. indicates the amino acid in position 1, 2 and 3 etc., respectively, in the B chain of insulin (counted from the N-terminal end). Using the one letter codes for amino acids, a term like A10C designates that the amino acid in the A10 position is cysteine. Using the three letter codes for amino acids, the corresponding expression is A10Cys.

By “desB30”, “B(1-29)” or “desThrB30” is meant a natural insulin B chain or an analogue thereof lacking the B30 (threonine, Thr) amino acid and “A(1-21)” means the natural insulin A chain. Thus, e.g., A10C,B1C,desB30 human insulin or alternatively A10Cys,B1Cys,desB30 human insulin (or alternatively CysA10,CysB1,desThrB30 human insulin) is an analogue of human insulin where the amino acid in position 10 in the A chain is substituted with cysteine, the amino acid in position 1 in the B chain is substituted with cysteine, and the amino acid in position 30 (threonine, Thr) in the B chain is deleted.

Herein, the naming of the peptides or proteins is done according to the following principles: The names are given as mutations and modifications (such as acylations) relative to the parent peptide or protein such as human insulin. For the naming of the acyl moiety, the naming is done according to IUPAC nomenclature and in other cases as peptide nomenclature. For example, naming the acyl moiety:

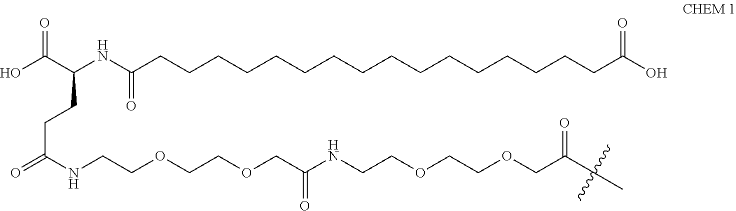 [SMILES: *C(=O)COCCOCCNC(=O)COCCOCCNC(=O)CC[C@H](NC(=O)CCCCCCCCCCCCCCCCC(=O)O)C(=O)O]

may e.g. be “octadecanedioyl-γGlu-OEG-OEG”, “octadecanedioyl-gGlu-OEG-OEG”, “octadecanedioyl-gGlu-2×OEG”, or “17-carboxyheptadecanoyl-γGlu-OEG-OEG”, wherein OEG is short hand notation for the amino acid residue, 8-amino-3,6-dioxaoctanoic acid, —NH(CH₂)₂O(CH₂)₂OCH₂CO—, and γGlu (or gGlu) is short hand notation for the amino acid gamma L-glutamic acid moiety.

One example is the insulin of example 1 in patent application WO2011/161125 (with the sequence/structure given below) is named “A10C, A14E, B4C, B25H, B29K(N^{ε}Octadecanedioyl-γGlu-OEG-OEG), desB30 human insulin” to indicate that the amino acid in position A10 in human insulin, has been mutated to C; A14, Y in human insulin, has been mutated to E; the amino acid in position B4, Q in human insulin, has been mutated to C; the amino acid in position B25, F in human insulin, has been mutated to H, the amino acid in position B29, K as in human insulin, has been modified by acylation on the epsilon nitrogen in the lysine residue of B29, denoted NE, by the residue octadecanedioyl-γGlu-OEG-OEG, and the amino acid in position B30, T in human insulin, has been deleted. Asterisks in the formula below indicate that the residue in question is different (i.e. mutated) as compared to human insulin. The disulfide bonds as found in human insulin are shown with sulphur atoms, and the additional disulfide bond of the invention is shown with a line.

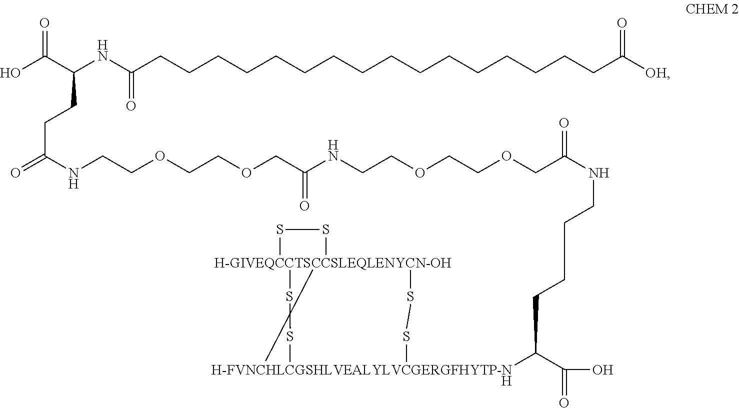

(SEQ ID NO: 5 and 6, 5 is the modified A chain and 6, the modified B chain of Chem 2)

In addition, the insulins of the invention may also be named according to IUPAC nomenclature (Open Eye, IUPAC style). According to this nomenclature, the above acylated insulin with an additional disulfide bridge is assigned the following name: N{Epsilon-B29}-[2-[2-[2-[[2-[2-[2-[[(4S)-4-carboxy-4-(17-carboxyheptadecanoylamino)butanoyl]amino]ethoxy]ethoxy]-acetyl]amino]ethoxy]ethoxy]acetyl]-[CysA10,GluA14,CysB4,HisB25],des-ThrB30-Insulin (human).

Herein, the term “amino acid residue” is an amino acid from which a hydroxy group has been removed from a carboxy group and/or from which a hydrogen atom has been removed from an amino group.

In one embodiment of the invention, the protease stabilised insulin for use in the invention comprises a side chain in the form of an acyl group on e.g. the ε-amino group of a Lys residue of the insulin amino acid sequence. In one embodiment the protease stabilised insulin comprises an “albumin binding residue”, i.e. a residue which under in vivo conditions binds to albumin when attached to a peptide or protein.

In a still further particular embodiment the albumin binding moiety comprises a portion in between the protracting moiety and the point of attachment to the peptide, which portion may be referred to as a “linker”, “linker moiety”, “spacer”, or the like. The linker may be optional, and hence in that case the albumin binding moiety may be identical to the protracting moiety.

In one embodiment, the albumin binding residue is a lipophilic residue. In a further embodiment, the lipophilic residue is attached to the insulin amino acid sequence via a linker.

In a further embodiment of the invention, the albumin binding residue is negatively charged at physiological pH. In another embodiment of the invention, the albumin binding residue comprises a group which may be negatively charged. One preferred group which may be negatively charged is a carboxylic acid group.

In one embodiment, the albumin binding residue is an α,ω-fatty diacid residue. In a further embodiment of the invention, the α,ω-fatty diacid residue of the lipophilic residue in the protease stabilised insulin has from 6 to 40 carbon atoms, from 8 to 26 carbon atoms or from 8 to 22 carbon atoms, or from 14 to 22 carbon atoms, or from 16 to 22 carbon atoms, or from 16 to 20 carbon atoms, or from 16 to 18 carbon atoms, or 16 carbon atoms, or 18 carbon atoms, or 20 carbon atoms, or 22 carbon atoms.

In one embodiment, the α,ω-fatty diacid residue of the lipophilic residue in the protease stabilised insulin has 18 carbon atoms. In one embodiment the tablet core of the present invention comprises an protease stabilised insulin, wherein the α,ω-fatty diacid residue of the lipophilic residue has 18 carbon atoms and provides higher values of protease stabilised insulin bioavailability relative to those comprising 20 carbon atoms. In one embodiment, the α,ω-fatty diacid residue in the protease stabilised insulin of the lipophilic residue has 20 carbon atoms. In one embodiment the tablet core of the present invention comprises an protease stabilised insulin, wherein the α,ω-fatty diacid residue of the lipophilic residue has 20 carbon atoms and provides lower values of protease stabilised insulin bioavailability relative to those comprising 18 carbon atoms. In one embodiment the tablet core of the present invention comprises an protease stabilised insulin, wherein the α,ω-fatty diacid residue of the lipophilic residue has 20 carbon atoms and provides lower values of protease stabilised insulin bioavailability, having a longer PK/PD profile relative to those comprising 18 carbon atoms.

In another embodiment of the invention, the albumin binding residue is an acyl group of a straight-chain or branched alkane α,ω-dicarboxylic acid. In a further embodiment the albumin binding residue is an acyl group of a straight-chain or branched alkane α,ω-dicarboxylic acid which includes an amino acid portion such as e.g. a gamma-Glu (γGlu) portion. In yet a further embodiment the albumin binding residue is an acyl group of a straight-chain or branched alkane α,ω-dicarboxylic acid which includes two amino acid portions such as e.g. a gamma-Glu portion and a 8-amino-3,6-dioxaoctanoic acid (OEG) portion. In yet a further embodiment the albumin binding residue is an acyl group of a straight-chain or branched alkane α,ω-dicarboxylic acid which includes more amino acid portions such as e.g. one gamma-Glu (yGlu) portion and consecutive 8-amino-3,6-dioxaoctanoic acid (OEG) portions. In one embodiment, the acyl moiety attached to the parent (e.g.protease stabilised) insulin analogue has the general formula:

Acy-AA1ₙ-AA2ₘ-AA3ₚ- CHEM 3

wherein n is 0 or an integer in the range from 1 to 3; m is 0 or an integer in the range from 1 to 10; p is 0 or an integer in the range from 1 to 10; Acy is a fatty acid or a fatty diacid comprising from about 8 to about 24 carbon atoms such as from about 14 to about 22 carbon atoms; AA1 is a neutral linear or cyclic amino acid residue; AA2 is an acidic amino acid residue; AA3 is a neutral, alkyleneglycol-containing amino acid residue; the order by which AA1, AA2 and AA3 appears in the formula may be interchanged independently; AA2 may occur several times along the formula (e.g., Acy-AA2-AA3₂-AA2-); AA2 may occur independently (=being different) several times along the formula (e.g., Acy-AA2-AA3₂-AA2-); the connections between Acy, AA1, AA2 and/or AA3 are amide (peptide) bonds which, formally, may be obtained by removal of a hydrogen atom or a hydroxyl group (water) from each of Acy, AA1, AA2 and AA3; and attachment to the insulin analogue may be from the C-terminal end of a AA1, AA2, or AA3 residue in the acyl moiety of CHEM 3 or from one of the side chain(s) of an AA2 residue present in the moiety of CHEM 3.

In another embodiment, the acyl moiety attached to the parent insulin analogue has the general formula Acy-AA1ₙ-AA2ₘ-AA3ₚ- (CHEM 3), wherein AA1 is selected from Gly, D- or L-Ala, βAla, 4-aminobutyric acid, 5-aminovaleric acid, 6-aminohexanoic acid, D- or L-Glu-α-amide, D- or L-Glu-γ-amide, D- or L-Asp-α-amide, D- or L-Asp-β-amide, or a group of one of the formula:

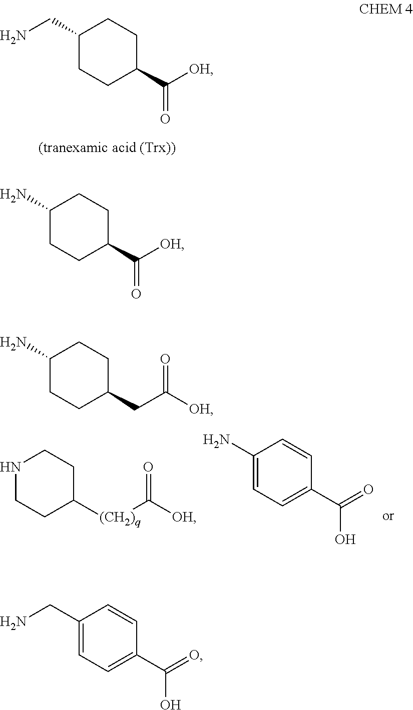

from which a hydrogen atom and/or a hydroxyl group has been removed and wherein q is 0, 1, 2, 3 or 4 and, in this embodiment, AA1 may, alternatively, be 7-aminoheptanoic acid or 8-aminooctanoic acid.

In another embodiment, the acyl moiety attached to the parent insulin analogue has the general formula Acy-AA1ₙ-AA2ₘ-AA3ₚ- (CHEM 3), wherein AA1 is as defined above and AA2 is selected from L- or D-Glu, L- or D-Asp, L- or D-homoGlu or any of the following:

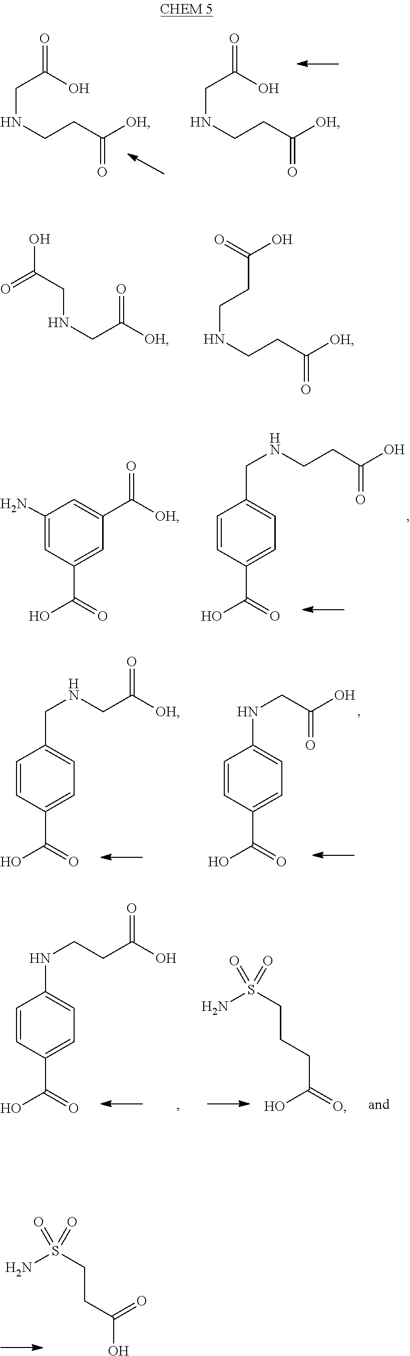

from which a hydrogen atom and/or a hydroxyl group has been removed and wherein the arrows indicate the attachment point to the amino group of AA1, AA2, AA3, or to the amino group of the insulin analogue.

In one embodiment, the neutral cyclic amino acid residue designated AA1 is an amino acid containing a saturated 6-membered carbocyclic ring, optionally containing a nitrogen hetero atom, and preferably the ring is a cyclohexane ring or a piperidine ring. Preferably, the molecular weight of this neutral cyclic amino acid is in the range from about 100 to about 200 Da.

The acidic amino acid residue designated AA2 is an amino acid with a molecular weight of up to about 200 Da comprising two carboxylic acid groups and one primary or secondary amino group. Alternatively, acidic amino acid residue designated AA2 is an amino acid with a molecular weight of up to about 250 Da comprising one carboxylic acid group and one primary or secondary sulphonamide group.

The neutral, alkyleneglycol-containing amino acid residue designated AA3 is an alkyleneglycol moiety, optionally an oligo- or polyalkyleneglycol moiety containing a carboxylic acid functionality at one end and a amino group functionality at the other end.

Herein, the term alkyleneglycol moiety covers mono-alkyleneglycol moieties as well as oligo-alkyleneglycol moieties. Mono- and oligoalkyleneglycols comprises mono- and oligoethyleneglycol based, mono- and oligopropyleneglycol based and mono- and oligobutyleneglycol based chains, i.e., chains that are based on the repeating unit —CH₂CH₂O—, —CH₂CH₂CH₂O— or —CH₂CH₂CH₂CH₂O—. The alkyleneglycol moiety is monodisperse (with well defined length/molecular weight). Monoalkyleneglycol moieties comprise —OCH₂CH₂O—, —OCH₂CH₂CH₂O— or —OCH₂CH₂CH₂CH₂O— containing different groups at each end.

As mentioned herein, the order by which AA1, AA2 and AA3 appears in the acyl moiety with CHEM 3 (Acy-AA1ₙ-AA2ₘ-AA3ₚ-) may be interchanged independently. Consequently, the formula Acy-AA1ₙ-AA2ₘ-AA3ₚ- also covers moieties like, e.g., the formula Acy-AA2ₘ-AA1ₙ-AA3ₚ-, the formula Acy-AA2-AA3ₙ-AA2-, and the formula Acy-AA3ₚ-AA2ₘ-AA1ₙ-, wherein Acy, AA1, AA2, AA3, n, m and p are as defined herein.

As mentioned herein, the connections between the moieties Acy, AA1, AA2 and/or AA3 are formally obtained by amide bond (peptide bond) formation (—CONH—) by removal of water from the parent compounds from which they formally are build. This means that in order to get the complete formula for the acyl moiety with the formula CHEM 3 (Acy-AA1ₙ-AA2ₘ-AA3ₚ-, wherein Acy, AA1, AA2, AA3, n, m and p are as defined herein), one has, formally, to take the compounds given for the terms Acy, AA1, AA2 and AA3 and remove a hydrogen and/or hydroxyl from them and, formally, to connect the building blocks so obtained at the free ends so obtained.

Non-limiting, specific examples of the acyl moieties of CHEM 3 Acy-AA1ₙ-AA2ₘ-AA3ₚ- which may be present in the acylated insulin analogues of this invention are listed in WO 2009/115469 A1, pp. 27-43:

Any of the above non-limiting specific examples of acyl moieties of the formula Acy-AA1ₙ-AA2ₘ-AA3ₚ- may be attached to an epsilon amino group of a lysine residue present in any of the above non-limiting specific examples of parent insulin analogues thereby giving further specific examples of acylated insulin analogues of this invention.

The parent insulin analogues may be converted into the acylated insulins containing additional disulfide bonds of this invention by introducing of the desired group of the formula Acy-AA1ₙ-AA2ₘ-AA3ₚ- in the lysine residue. The desired group of the formula Acy-AA1ₙ-AA2ₘ-AA3ₚ- may be introduced by any convenient method and many methods are disclosed in the prior art for such reactions. More details appear from the examples herein.

Non-limiting, specific examples of the acyl moieties of the formula Acy-AA1ₙ-AA2ₘ-AA3ₚ- which may be present in the acylated insulin analogues of this invention are the following:

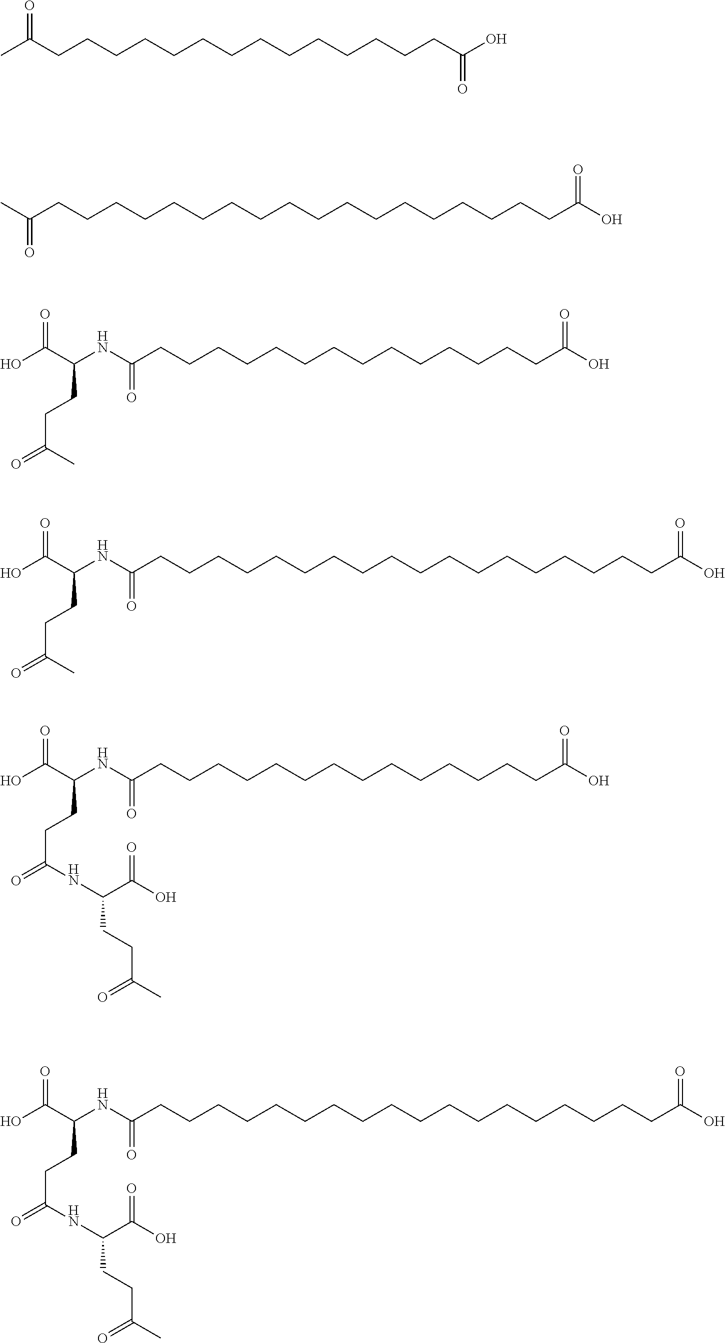 [SMILES: CC(=O)CCCCCCCCCCCCCCC(=O)O ; CC(=O)CCCCCCCCCCCCCCCCCCC(=O)O ; CC(=O)CC[C@H](NC(=O)CCCCCCCCCCCCCCC(=O)O)C(=O)O ; CC(=O)CC[C@H](NC(=O)CCCCCCCCCCCCCCCCCCC(=O)O)C(=O)O ; CC(=O)CC[C@H](NC(=O)CC[C@H](NC(=O)CCCCCCCCCCCCCCC(=O)O)C(=O)O)C(=O)O ; CC(=O)CC[C@H](NC(=O)CC[C@H](NC(=O)CCCCCCCCCCCCCCCCCCC(=O)O)C(=O)O)C(=O)O]

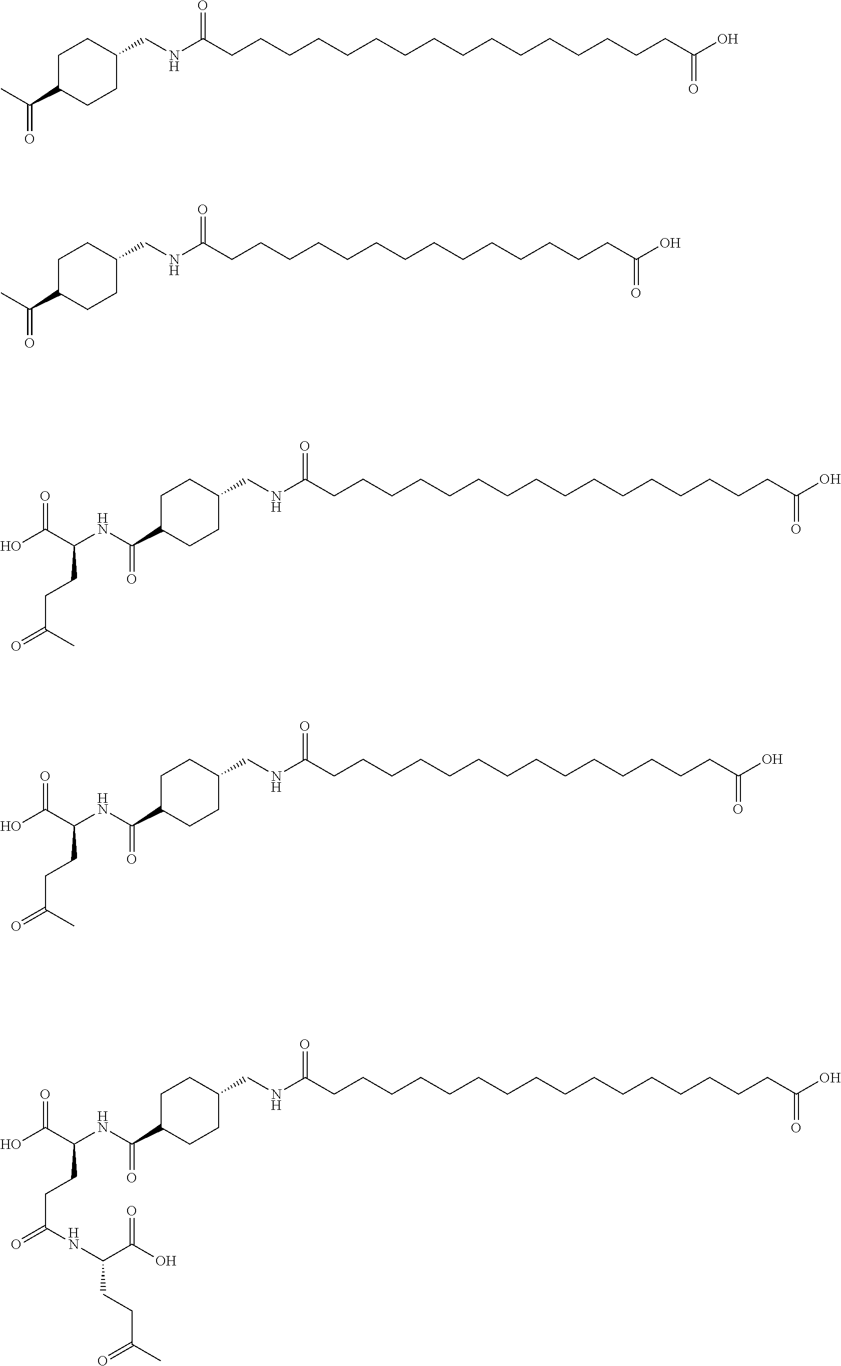

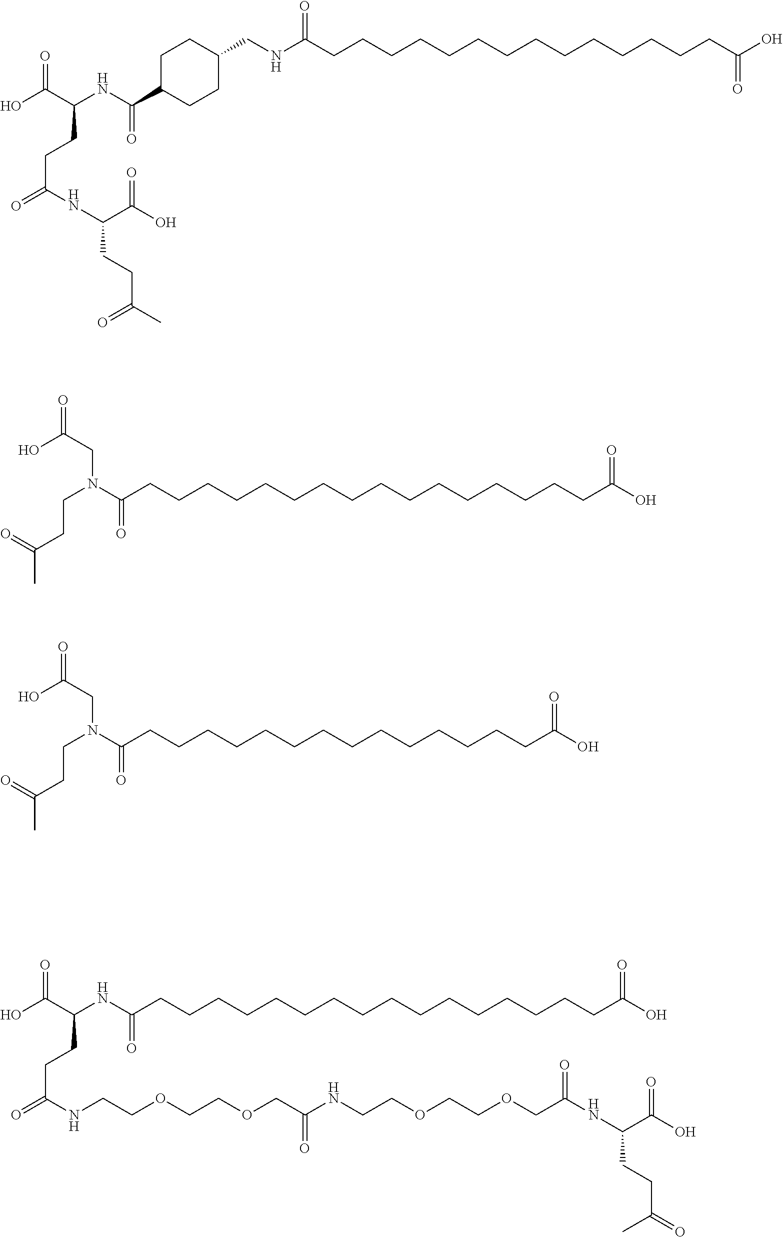

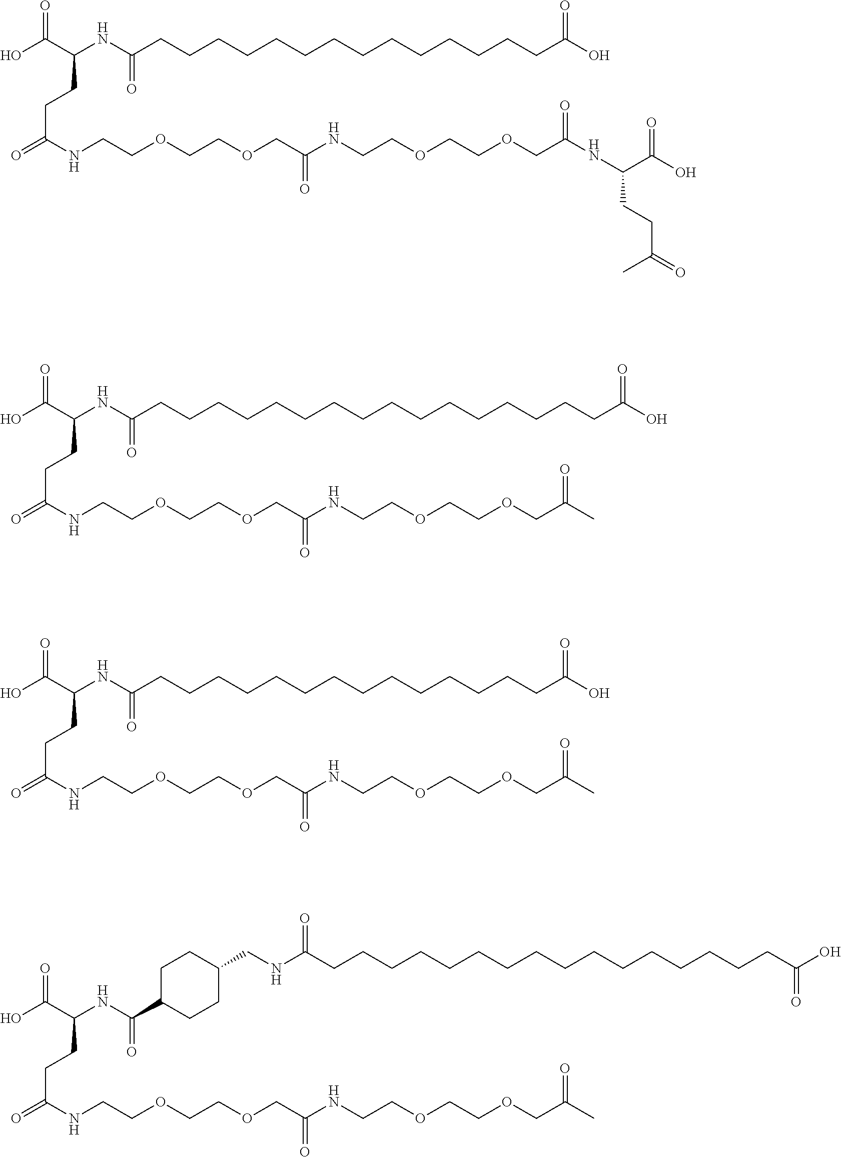

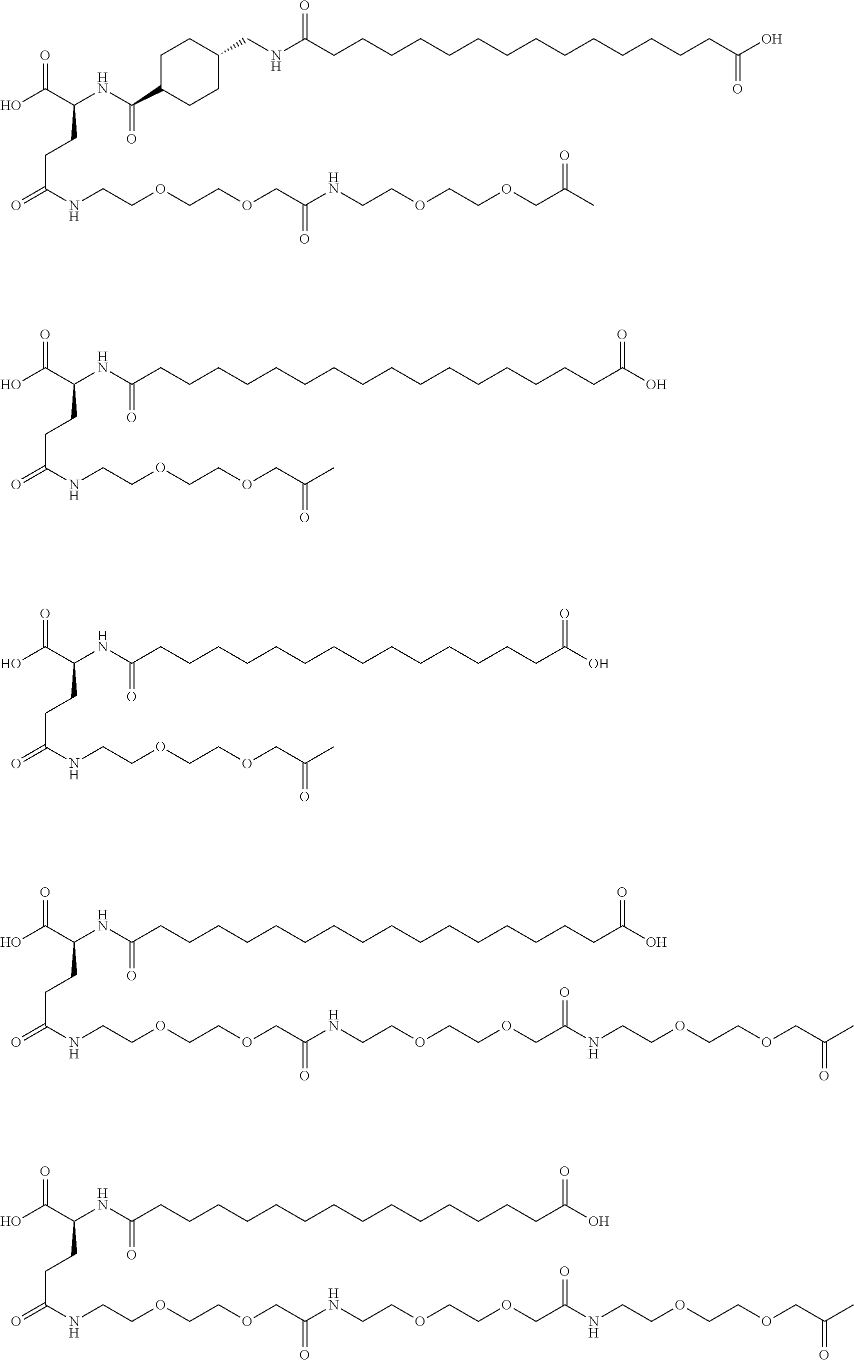

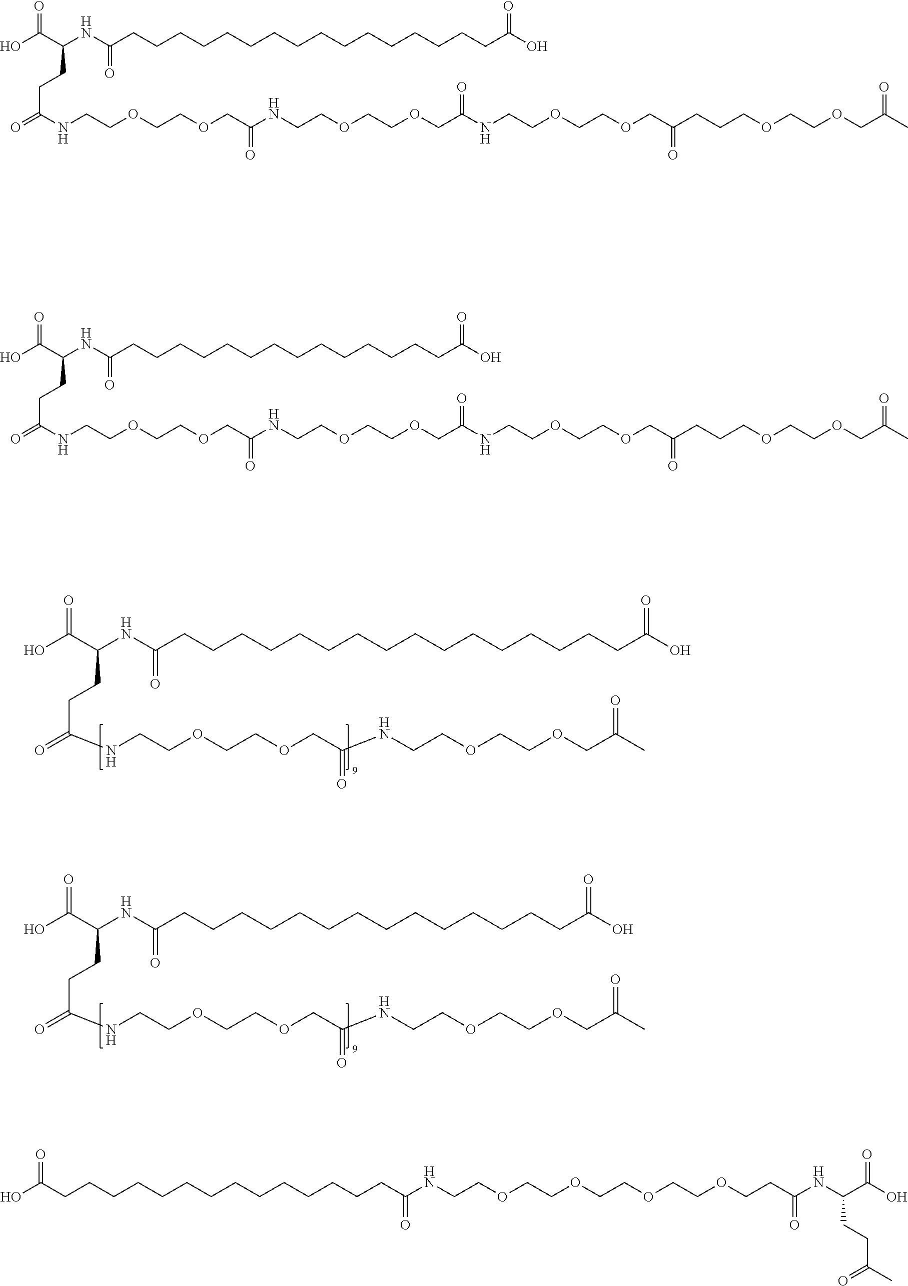

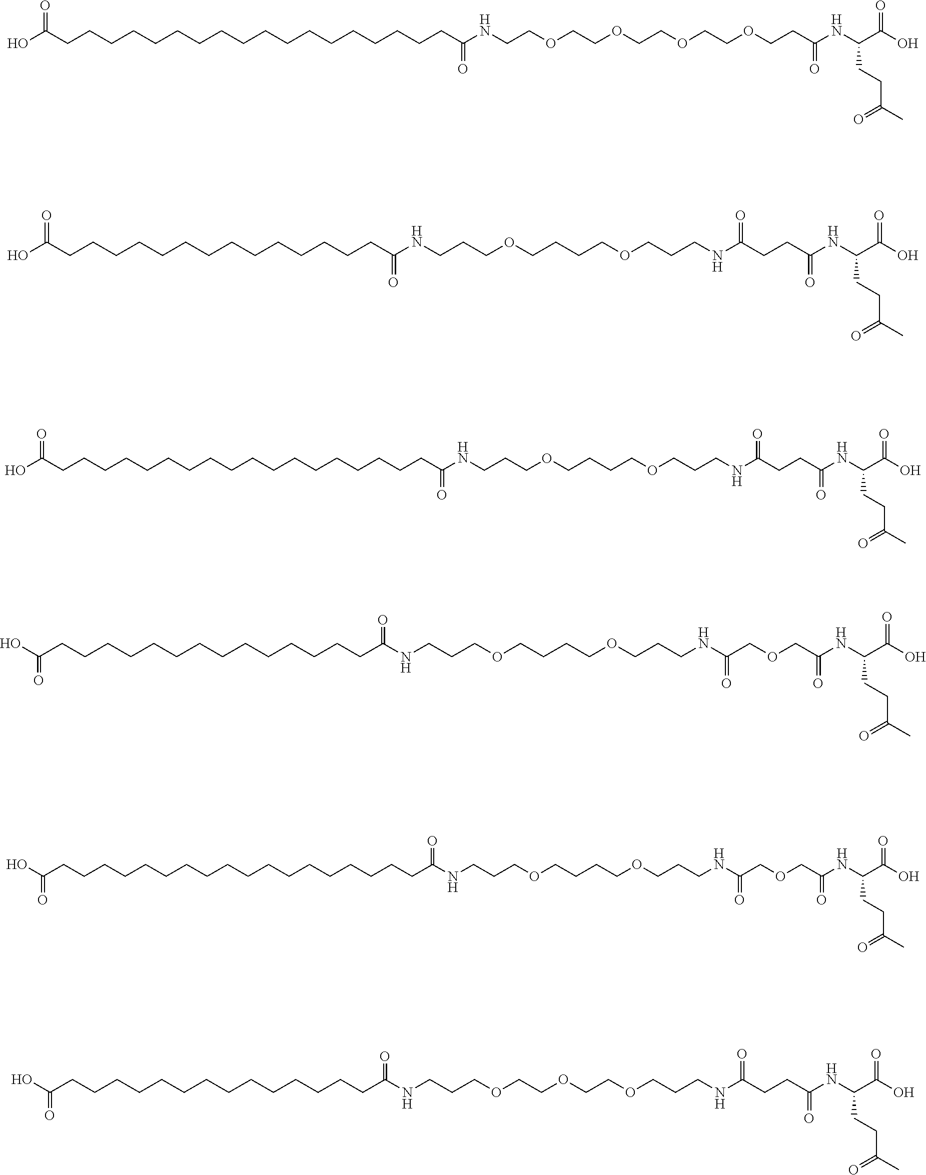 [SMILES: CC(=O)CC[C@H](NC(=O)CCOCCOCCOCCOCCNC(=O)CCCCCCCCCCCCCCCCCCC(=O)O)C(=O)O ; CC(=O)CC[C@H](NC(=O)CCC(=O)NCCCOCCCCOCCCNC(=O)CCCCCCCCCCCCCCC(=O)O)C(=O)O ; CC(=O)CC[C@H](NC(=O)CCC(=O)NCCCOCCCCOCCCNC(=O)CCCCCCCCCCCCCCCCCCC(=O)O)C(=O)O ; CC(=O)CC[C@H](NC(=O)COCC(=O)NCCCOCCCCOCCCNC(=O)CCCCCCCCCCCCCCC(=O)O)C(=O)O ; CC(=O)CC[C@H](NC(=O)COCC(=O)NCCCOCCCCOCCCNC(=O)CCCCCCCCCCCCCCCCCCC(=O)O)C(=O)O ; CC(=O)CC[C@H](NC(=O)CCC(=O)NCCCOCCOCCOCCCNC(=O)CCCCCCCCCCCCCCC(=O)O)C(=O)O]

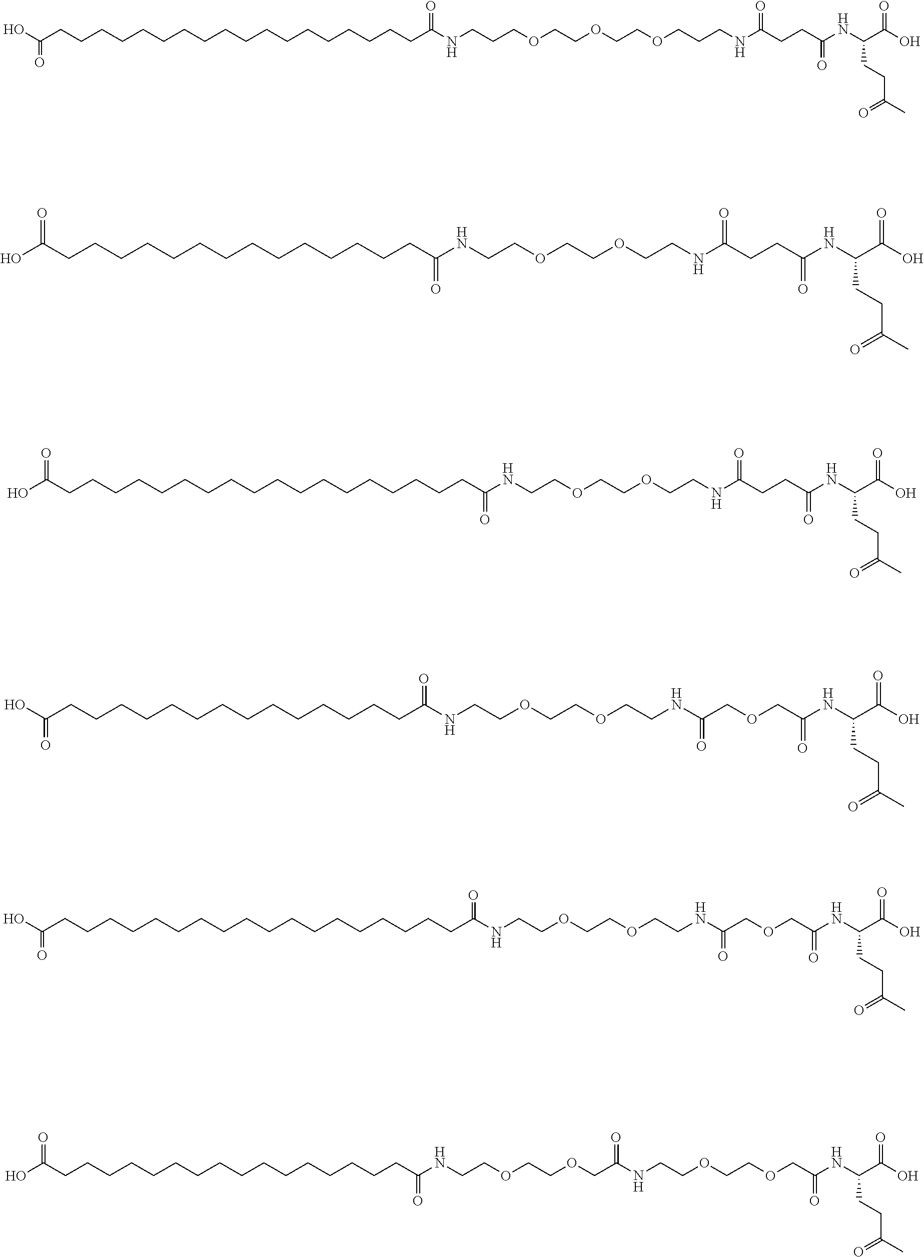 [SMILES: CC(=O)CC[C@H](NC(=O)CCC(=O)NCCCOCCOCCOCCCNC(=O)CCCCCCCCCCCCCCCCCCC(=O)O)C(=O)O ; CC(=O)CC[C@H](NC(=O)CCC(=O)NCCOCCOCCNC(=O)CCCCCCCCCCCCCCC(=O)O)C(=O)O ; CC(=O)CC[C@H](NC(=O)CCC(=O)NCCOCCOCCNC(=O)CCCCCCCCCCCCCCCCCCC(=O)O)C(=O)O ; CC(=O)CC[C@H](NC(=O)COCC(=O)NCCOCCOCCNC(=O)CCCCCCCCCCCCCCC(=O)O)C(=O)O ; CC(=O)CC[C@H](NC(=O)COCC(=O)NCCOCCOCCNC(=O)CCCCCCCCCCCCCCCCCCC(=O)O)C(=O)O ; CC(=O)CC[C@H](NC(=O)COCCOCCNC(=O)COCCOCCNC(=O)CCCCCCCCCCCCCCCCC(=O)O)C(=O)O]

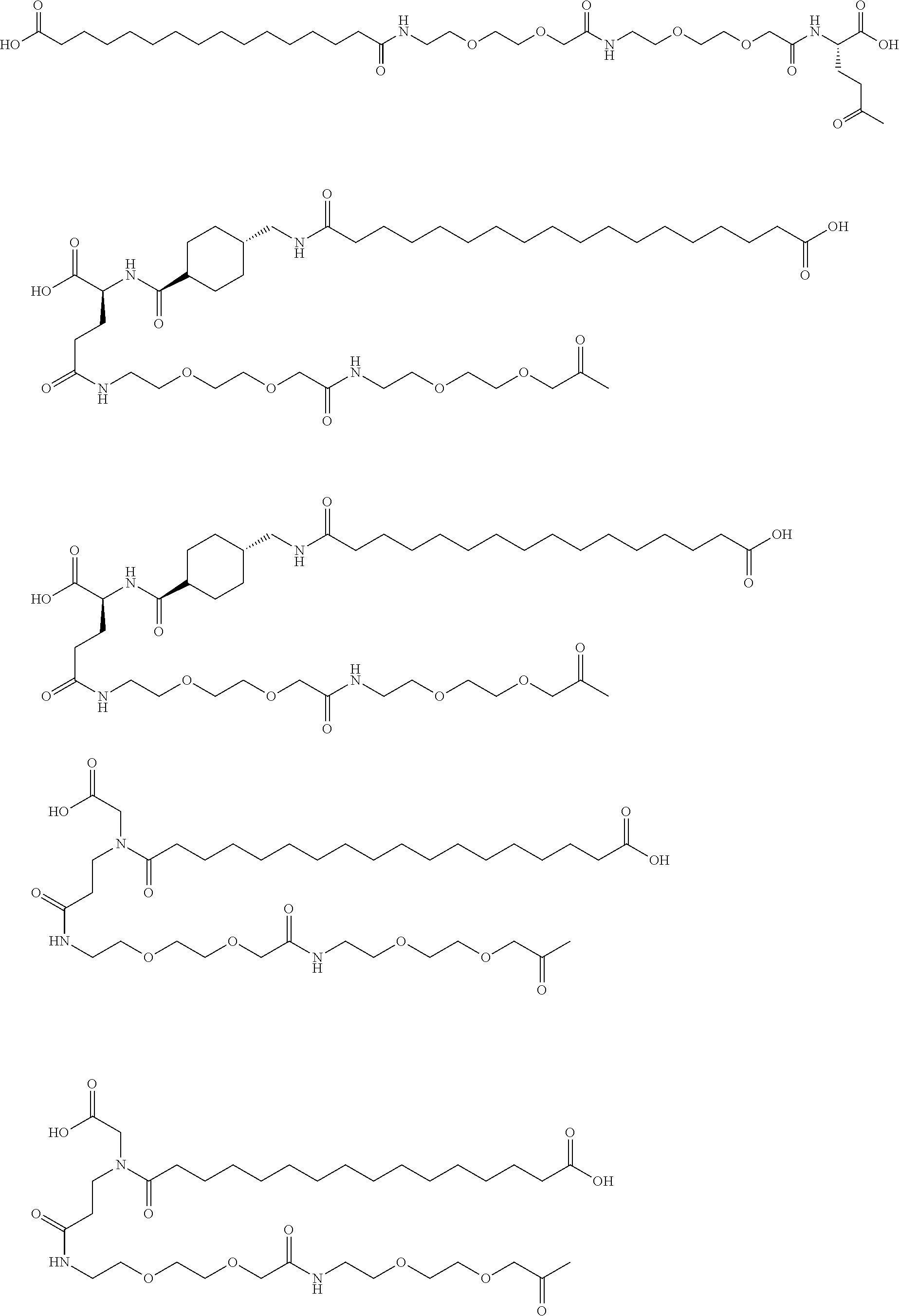

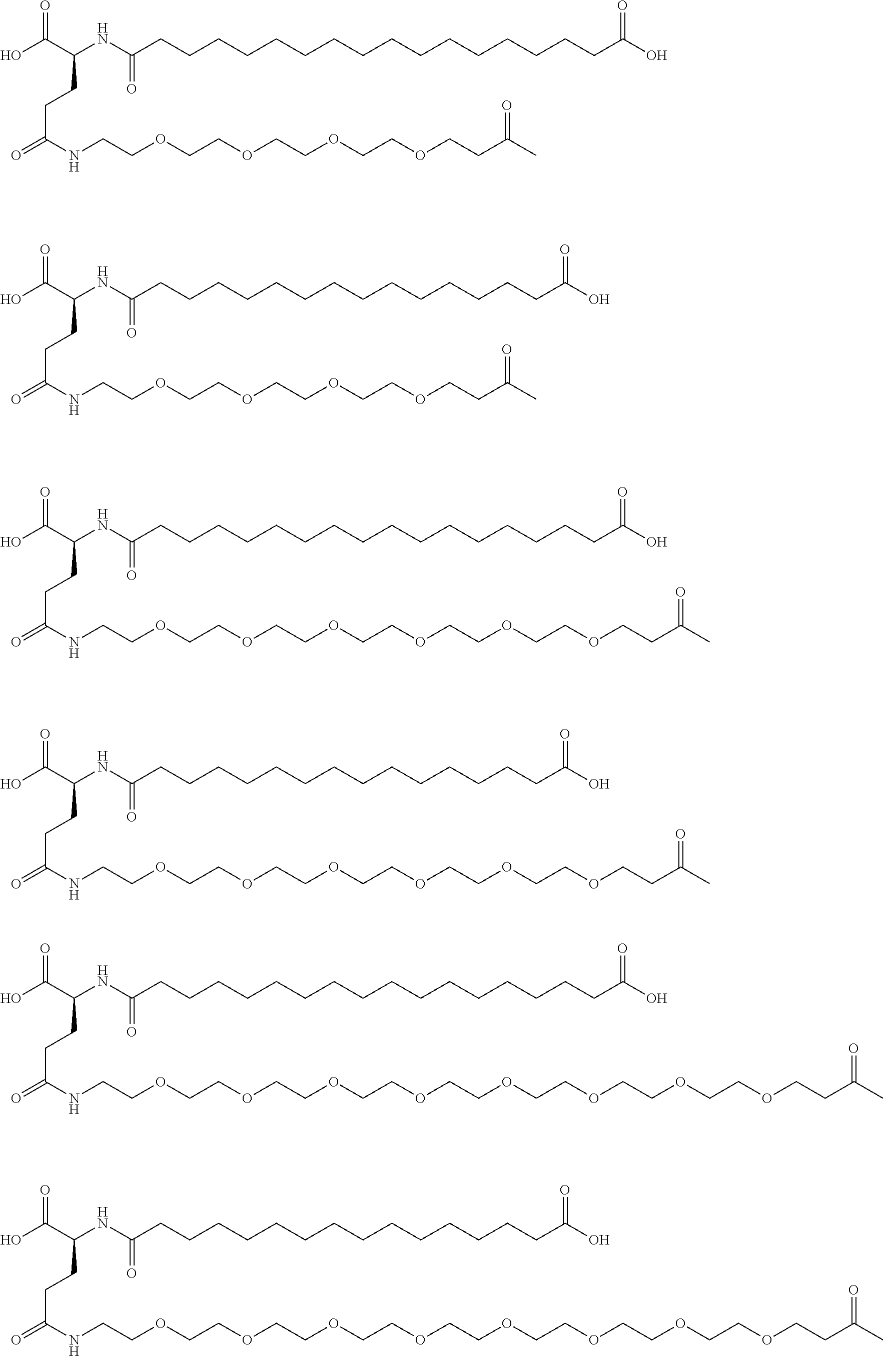 [SMILES: CC(=O)CCOCCOCCOCCOCCNC(=O)CC[C@H](NC(=O)CCCCCCCCCCCCCCCCC(=O)O)C(=O)O ; CC(=O)CCOCCOCCOCCOCCNC(=O)CC[C@H](NC(=O)CCCCCCCCCCCCCCC(=O)O)C(=O)O ; CC(=O)CCOCCOCCOCCOCCOCCOCCNC(=O)CC[C@H](NC(=O)CCCCCCCCCCCCCCCCC(=O)O)C(=O)O ; CC(=O)CCOCCOCCOCCOCCOCCOCCNC(=O)CC[C@H](NC(=O)CCCCCCCCCCCCCCC(=O)O)C(=O)O ; CC(=O)CCOCCOCCOCCOCCOCCOCCOCCOCCNC(=O)CC[C@H](NC(=O)CCCCCCCCCCCCCCCCC(=O)O)C(=O)O ; CC(=O)CCOCCOCCOCCOCCOCCOCCOCCOCCNC(=O)CC[C@H](NC(=O)CCCCCCCCCCCCCCC(=O)O)C(=O)O]

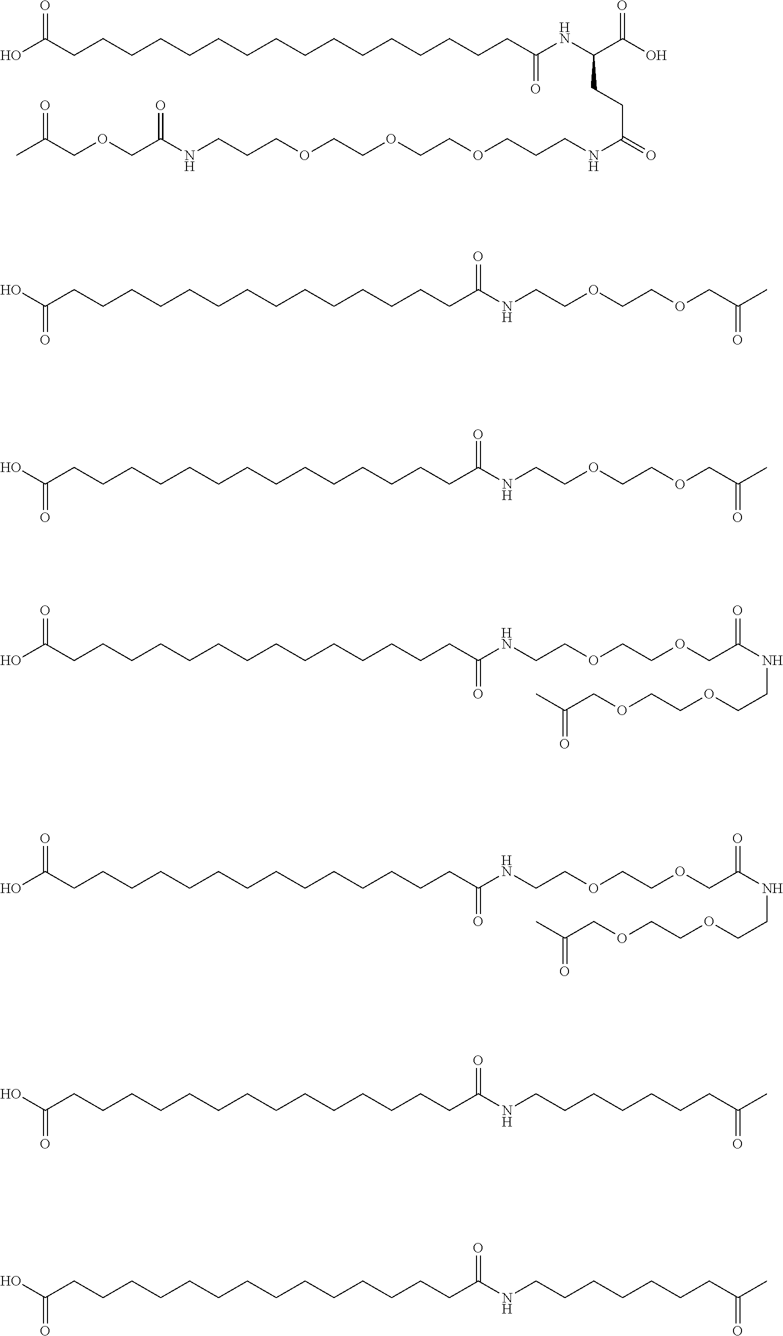

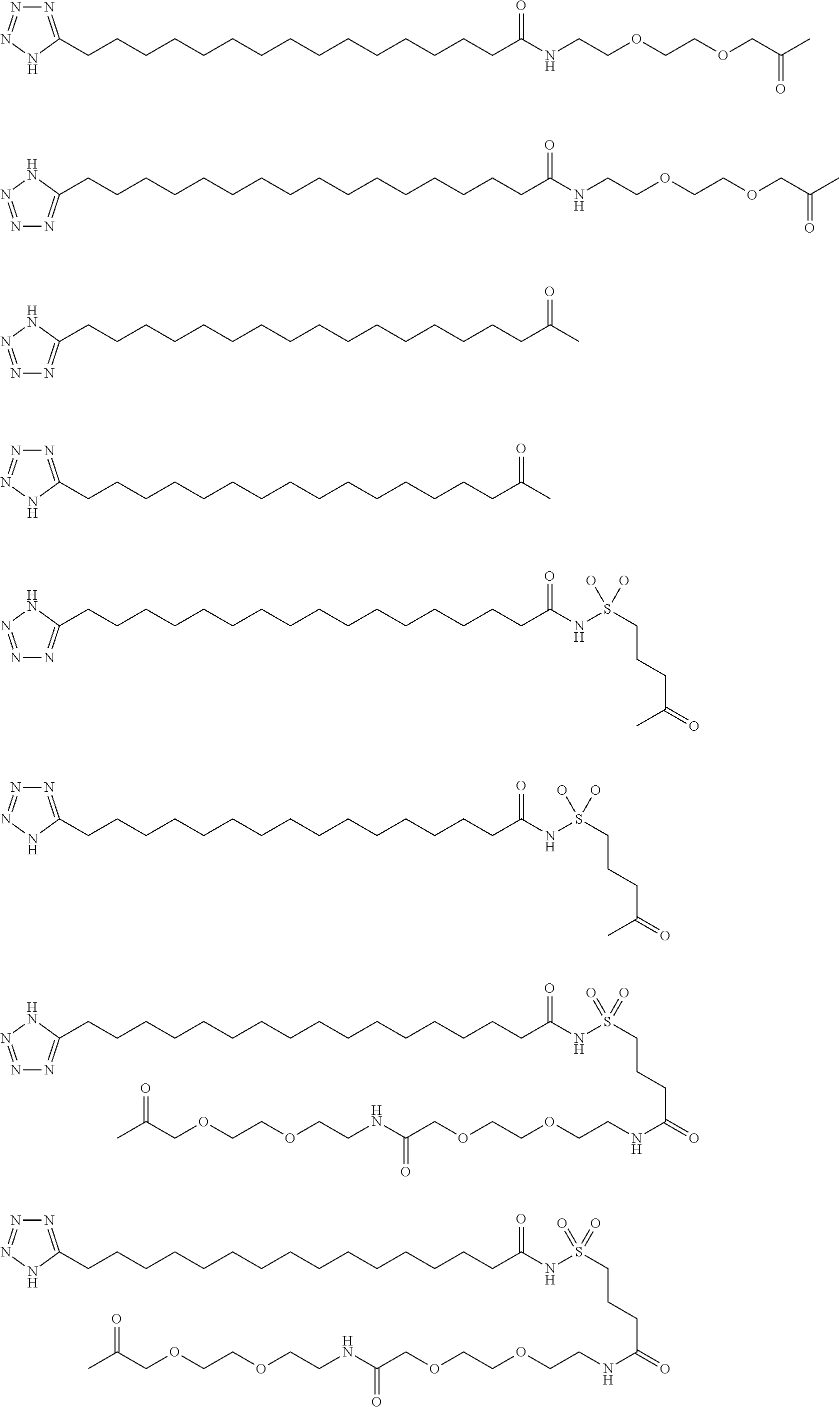

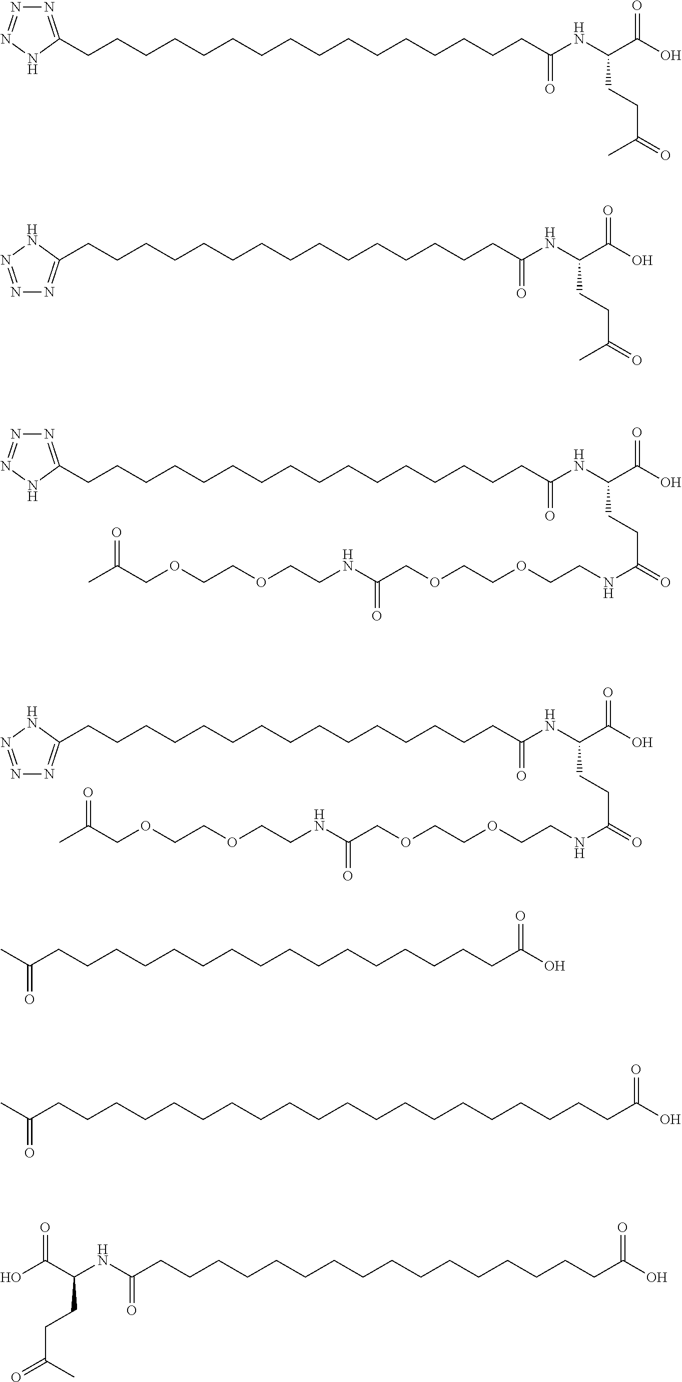

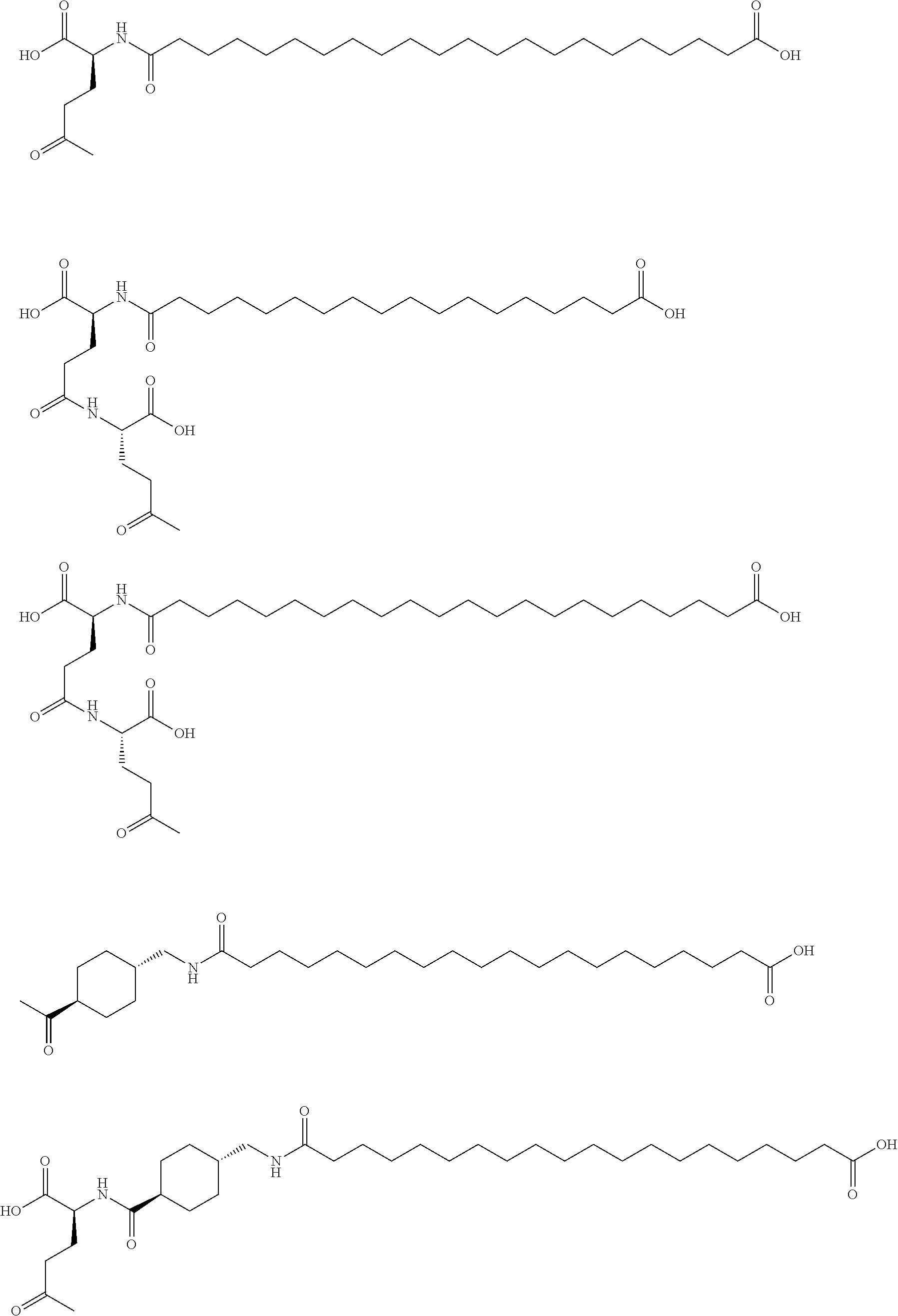 [SMILES: CC(=O)CC[C@H](NC(=O)CCCCCCCCCCCCCCCCCCCCC(=O)O)C(=O)O ; CC(=O)CC[C@H](NC(=O)CC[C@H](NC(=O)CCCCCCCCCCCCCCCCC(=O)O)C(=O)O)C(=O)O ; CC(=O)CC[C@H](NC(=O)CC[C@H](NC(=O)CCCCCCCCCCCCCCCCCCCCC(=O)O)C(=O)O)C(=O)O ; CC(=O)[C@H]1CC[C@H](CNC(=O)CCCCCCCCCCCCCCCCCCC(=O)O)CC1 ; CC(=O)CC[C@H](NC(=O)[C@H]1CC[C@H](CNC(=O)CCCCCCCCCCCCCCCCCCC(=O)O)CC1)C(=O)O]

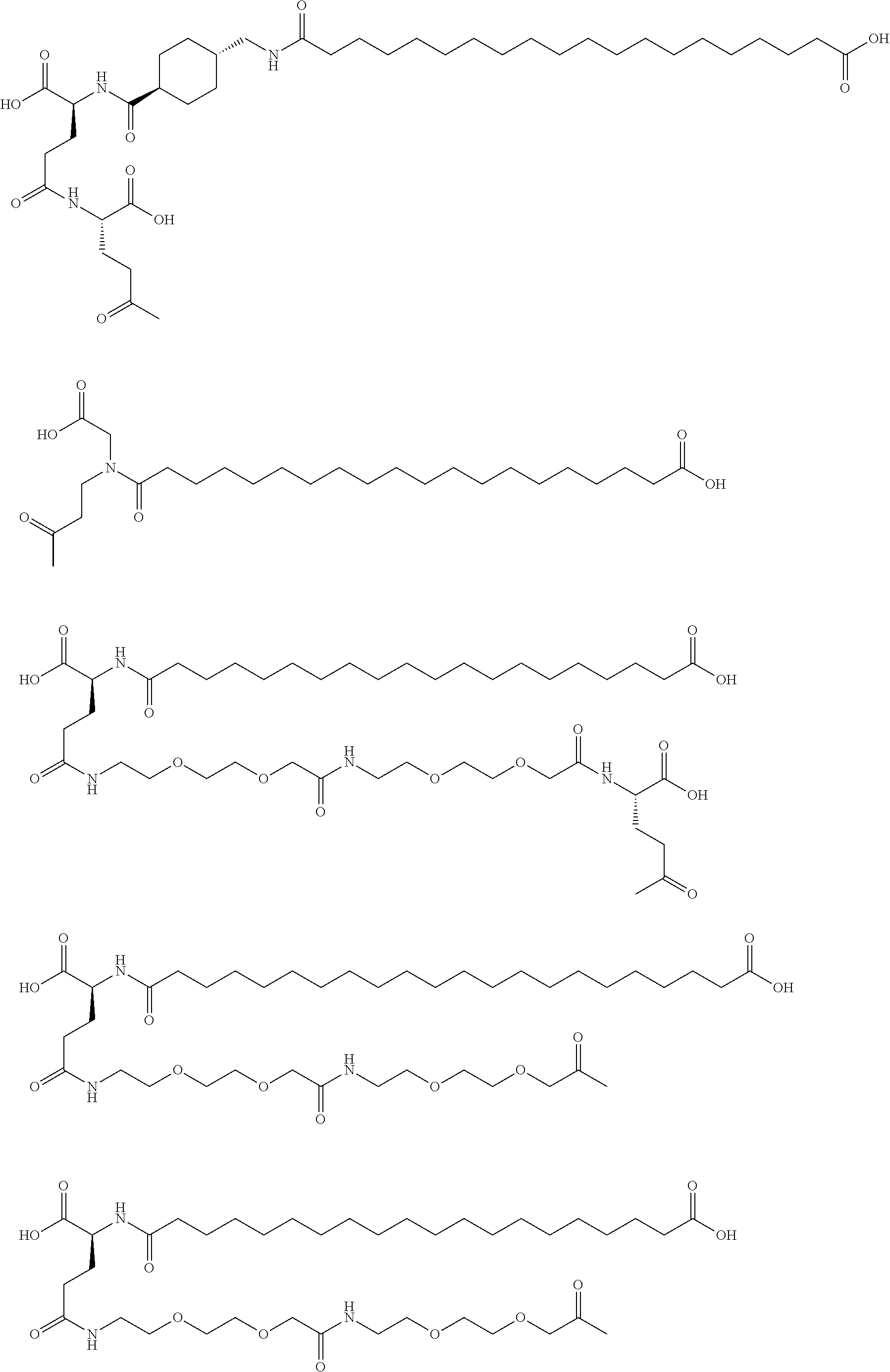 [SMILES: CC(=O)CC[C@H](NC(=O)CC[C@H](NC(=O)[C@H]1CC[C@H](CNC(=O)CCCCCCCCCCCCCCCCCCC(=O)O)CC1)C(=O)O)C(=O)O ; CC(=O)CCN(CC(=O)O)C(=O)CCCCCCCCCCCCCCCCCCC(=O)O ; CC(=O)CC[C@H](NC(=O)COCCOCCNC(=O)COCCOCCNC(=O)CC[C@H](NC(=O)CCCCCCCCCCCCCCCCCCC(=O)O)C(=O)O)C(=O)O ; CC(=O)COCCOCCNC(=O)COCCOCCNC(=O)CC[C@H](NC(=O)CCCCCCCCCCCCCCCCCCCCC(=O)O)C(=O)O ; CC(=O)COCCOCCNC(=O)COCCOCCNC(=O)CC[C@H](NC(=O)CCCCCCCCCCCCCCCCCCC(=O)O)C(=O)O]

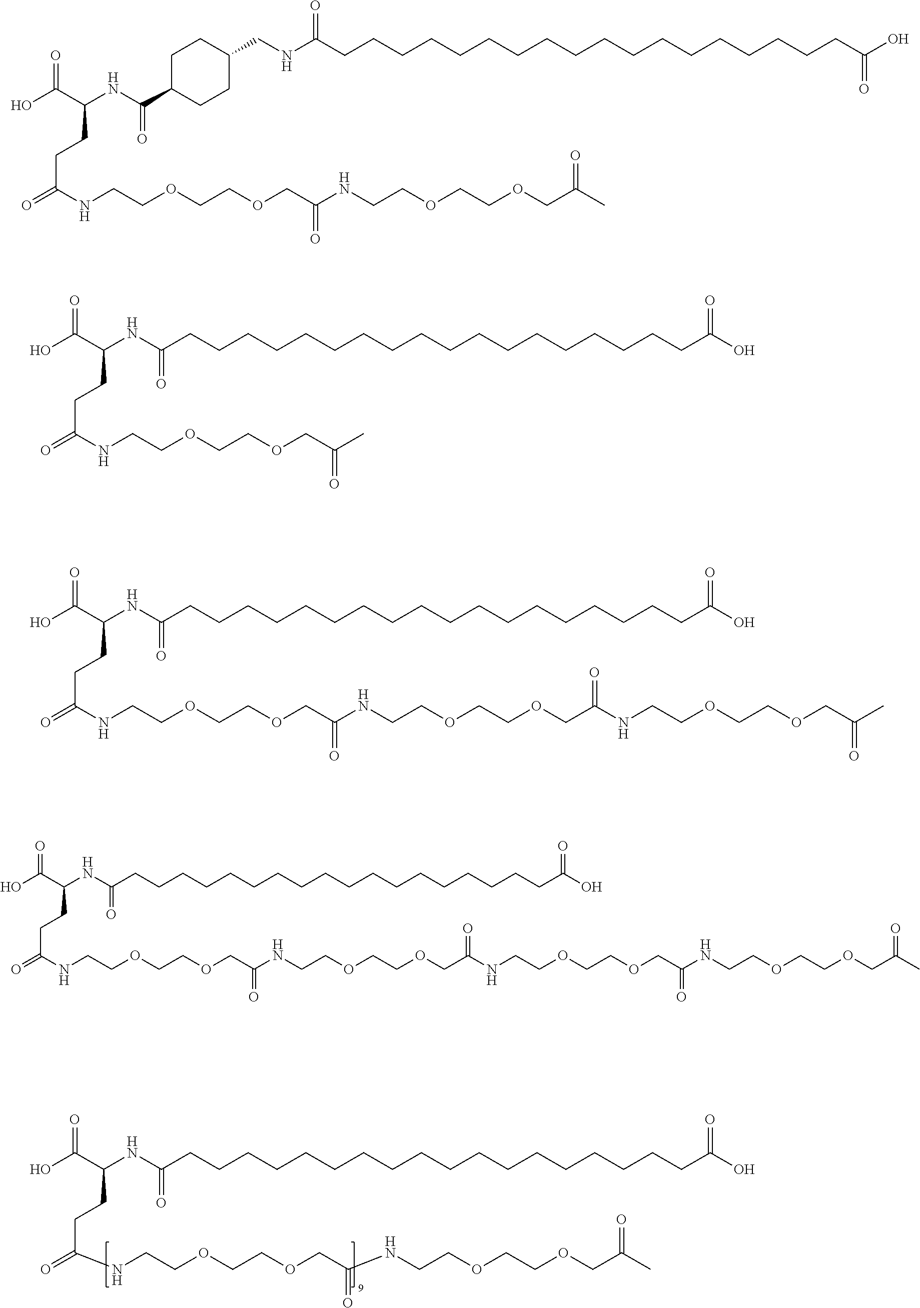

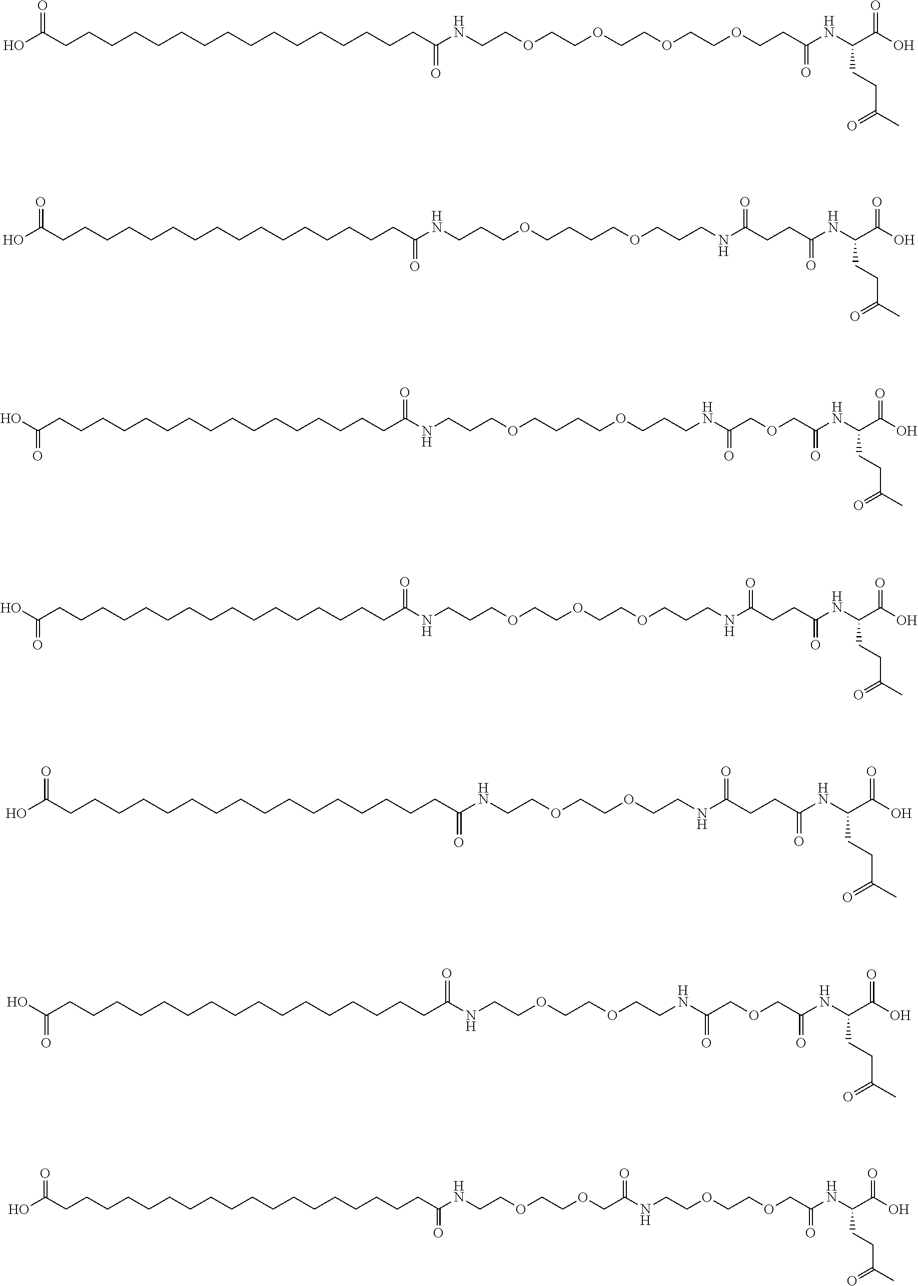

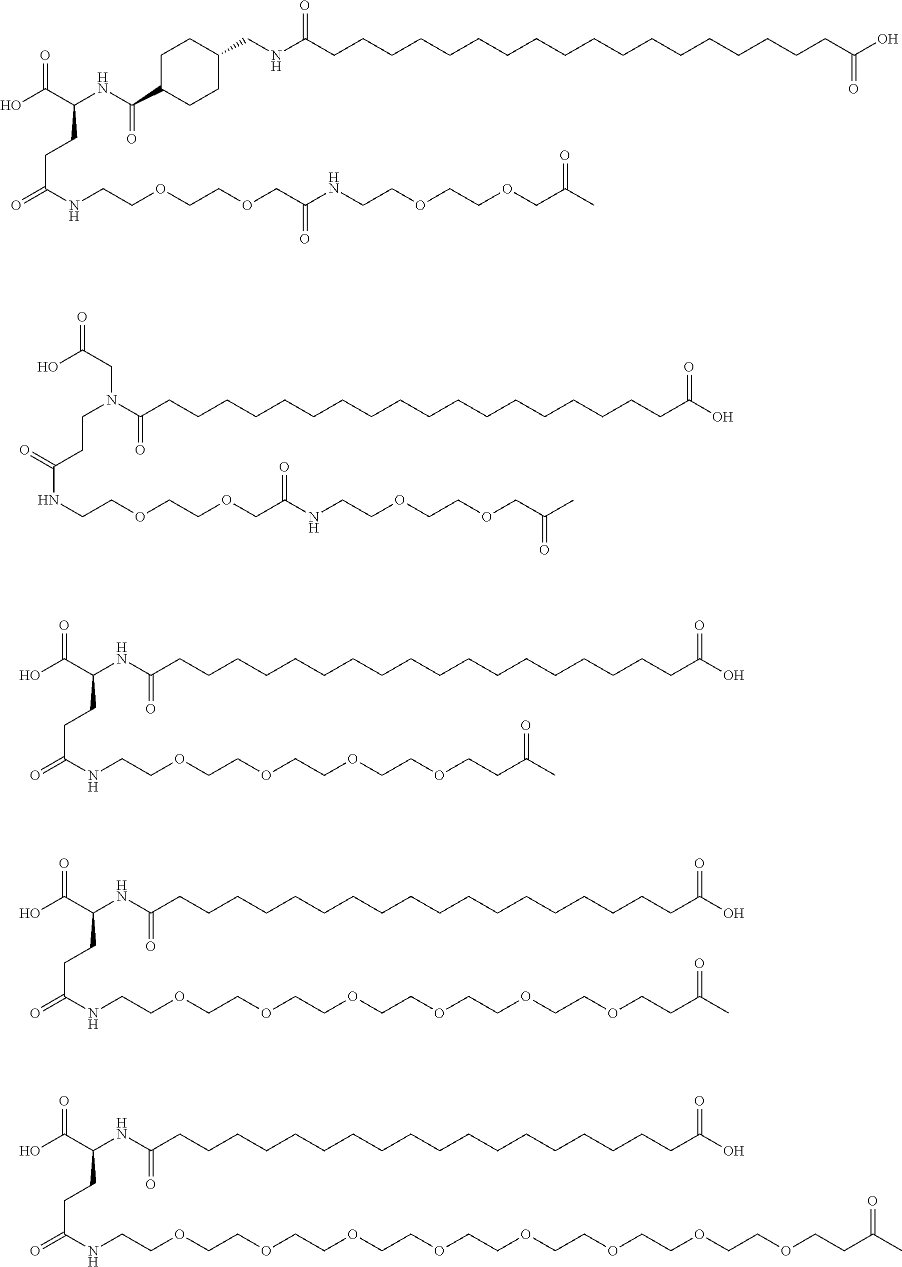

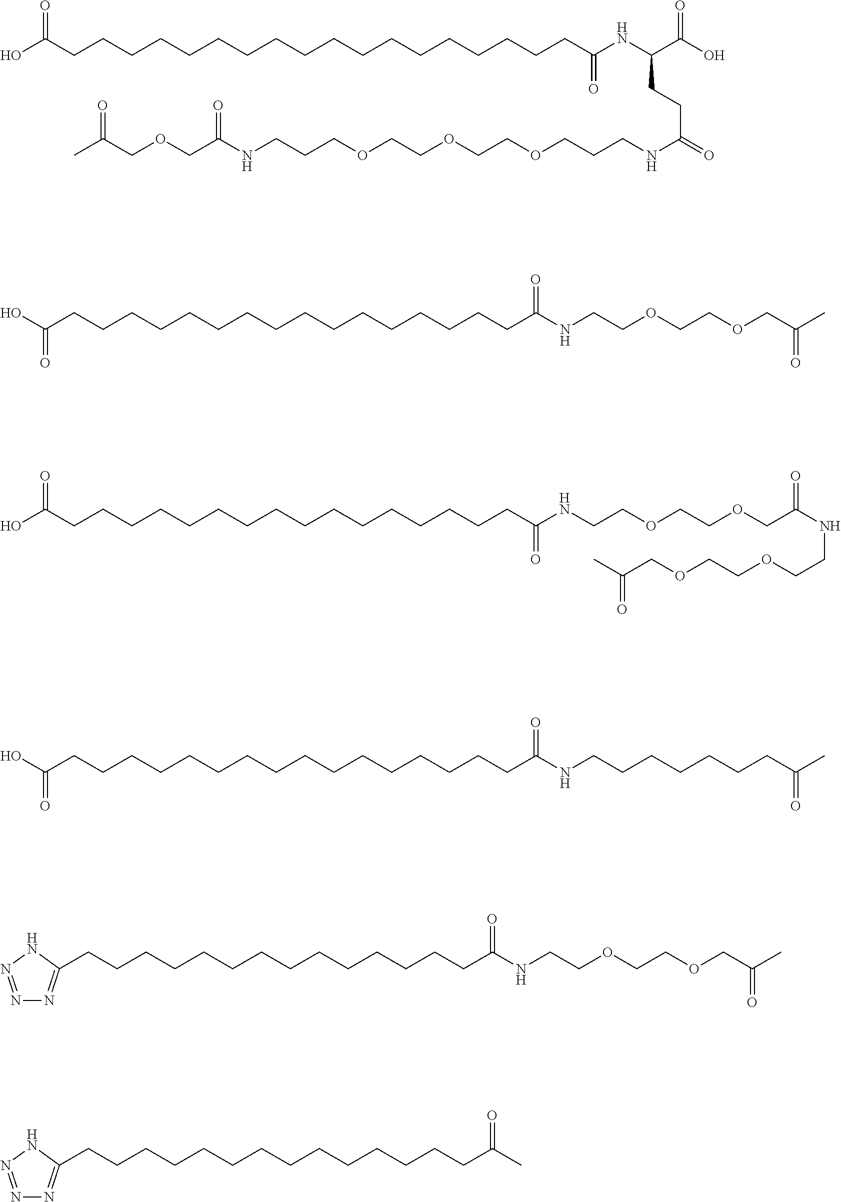 [SMILES: CC(=O)COCC(=O)NCCCOCCOCCOCCCNC(=O)CC[C@@H](NC(=O)CCCCCCCCCCCCCCCCCCC(=O)O)C(=O)O ; CC(=O)COCCOCCNC(=O)CCCCCCCCCCCCCCCCC(=O)O ; CC(=O)COCCOCCNC(=O)COCCOCCNC(=O)CCCCCCCCCCCCCCCCC(=O)O ; CC(=O)CCCCCCCNC(=O)CCCCCCCCCCCCCCCCC(=O)O ; CC(=O)COCCOCCNC(=O)CCCCCCCCCCCCCCc1nnn[nH]1 ; CC(=O)CCCCCCCCCCCCCCc1nnn[nH]1]

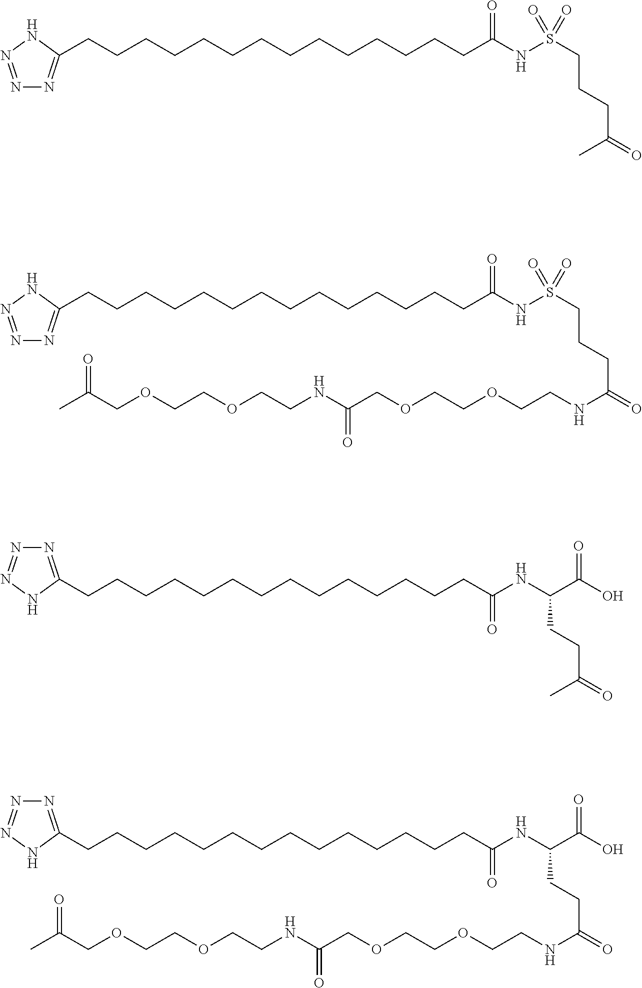 [SMILES: CC(=O)CCCS(=O)(=O)NC(=O)CCCCCCCCCCCCCCc1nnn[nH]1 ; CC(=O)COCCOCCNC(=O)COCCOCCNC(=O)CCCS(=O)(=O)NC(=O)CCCCCCCCCCCCCCc1nnn[nH]1 ; CC(=O)CC[C@H](NC(=O)CCCCCCCCCCCCCCc1nnn[nH]1)C(=O)O ; CC(=O)COCCOCCNC(=O)COCCOCCNC(=O)CC[C@H](NC(=O)CCCCCCCCCCCCCCc1nnn[nH]1)C(=O)O]

Any of the above non-limiting specific examples of side chains of the formula Acy-AA1ₙ-AA2ₘ-AA3ₚ- may be attached to an epsilon amino group of a lysine residue present in any of the above non-limiting specific examples of protease stabilised insulin analogues thereby giving further specific examples of acylated insulin analogues of this invention.

Any of the above non-limiting specific examples of side chains of the formula Acy-AA1ₙ-AA2ₘ-AA3ₚ- may be attached to an alpha amino group of an A1 residue present in any of the above non-limiting specific examples of protease stabilised insulin analogues thereby giving further specific examples of acylated insulin analogues of this invention.

In one embodiment a protease stabilised insulin according to for use in the invention has two or more cysteine substitutions in addition to the three disulfide bonds of human insulin which are retained.

In one embodiment, the sites of cysteine substitutions are chosen in such a way that the introduced cysteine residues are placed in the three dimensional structure of the folded protease stabilised insulin to allow for the formation of one or more additional disulfide bonds.

In one embodiment, protease stabilised insulins for use in the invention are more protracted than similar protease stabilised insulins without a side chain. With “more protracted” is herein meant that they have a longer elimination half-life or in other words an insulin effect for an extended period, i.e. a longer duration of action.

A non-limiting example of lipophilic substituents which may be used according to the invention may e.g. be found in the patent application WO 2009/115469, including as the lipophilic substituents of the acylated polypeptides as described in the passage beginning on page 25, line 3 of WO 2009/115469.

A non-limiting list of examples of protease stabilised insulins in the form of acylated protease stabilised insulin analogues which may be modified by cysteine substitutions according to the invention may e.g. be found in WO 2009/115469 A1.

In one embodiment a tablet core according to the present invention comprises a protease stabilised insulin, which is selected from the group consisting of:
1. A14E,B25H,B29K(N^{ε}-Hexadecandioyl),desB30 human insulin,
2. A14E,B25H,B29K(N^{ε}Octadecandioyl-γGlu),desB30 human insulin,
3. A14E,B25H,B29K(N^{ε}Eicosanedioyl-γGlu),desB30 human insulin,
4. A14E,B25H,B29K(N^{ε}3-Carboxy-5-octadecanedioylaminobenzoyl),desB30 human insulin,
5. A14E,B25H,B29K(N^{ε}-N-octadecandioyl-N-(2-carboxyethyl)glycyl),desB30 human insulin
6. A14E,B25H,B29K(N^{ε}(N-Octadecandioyl-N-carboxymethyl)-beta-alanyl),desB30 human insulin,
7. A14E,B25H,B29K(N^{ε}4-([4-({19-Carboxynonadecanoylamino}methyl)trans-cyclo-hexanecarbonyl]-γGlu),desB30 human insulin,
8. A14E,B25H,B29K(N^{ε}Heptadecanedioyl-γGlu),desB30 human insulin,
9. A14E,B25H,B29K(N^{ε}Octadecanedioyl-γGlu-OEG-OEG),desB30 human insulin,
10. A14E,B25H,B29K(N^{ε}Myristyl),desB30 human insulin,
11. A14E,B25H,B29K(N^{ε}Eicosanedioyl-γGlu-γGlu),desB30 human insulin,
12. A14E,B25H,B29K(N^{ε}4-([4-({19-Carboxynonadecanoylamino}methyl)trans-cyclohexanecarbonyl]-γGlu-γGlubdesB30 human insulin,
13. A14E,B25H,B29K(N^{ε}Octadecanedioyl-γGlu-γGlu),desB30 human insulin,
14. A14E,B25H,B29K(N^{ε}octadecandioyl-γGlu-PEG7),desB30 human insulin,
15. A14E,B25H,B29K(N^{ε}eicosanedioyl-γGlu-OEG-OEG), desB30 human insulin,
16. A14E,B25H,B29K(N^{ε}eicosanedioyl-γGlu-(3-(2-{2-[2-(2-aminoethoxy)ethoxy]ethoxy}ethoxy)propionyl-γGlubdesB30 human insulin,
17. A14E,B25H,B29K(N^{ε}Hexadecanedioyl-γGlu-OEG-OEG),desB30 human insulin,
18. A14E,B25H,B29K(N^{ε}Hexadecanedioyl-γGlu),desB30 human insulin,
19. A14E,B25H,B29K(N^{ε}heptadecanedioyl-γGlu-OEG-OEG),desB30 human insulin,
20. A14E,B25H,B29K(N^{ε}octadecanedioyl-γGlu-γGlu-γGlu-γGlu),desB30 human insulin,
21. A14E,B25H,B29K(N^{ε}Eicosanedioyl-γGlu-γGlu-γGlu),desB30 human insulin,
22. A14E,B25H,B27E,B29K(N^{ε}Octadecanedioyl-γGlu-OEG-OEG),desB30 human insulin,
23. A14E,B25H,B26G,B27G,B28G,B29K(N^{ε}Octadecanedioyl-γGlu-OEG-OEG),desB30 human insulin,
24. A14E,B16H,B25H,B29K(N^{ε}Octadecanedioyl-γGlu-OEG-OEG),desB30 human insulin,
25. A14E,B16E,B25H,B29K(N^{ε}Octadecanedioyl-γGlu-OEG-OEG),desB30 human insulin,
26. A14E,B16H,B25H,B29K(N^{ε}Hexadecanediol-γHGlu),desB30 human insulin,
27. A14E,B25H,B29K(N^{ε}Eicosanedioyl-γGlu-OEG-γGlu),desB30 human insulin,
28. A14E,B16E,B25H,B29K(N^{ε}Hexadecandioyl-γGlu),desB30 human insulin,
29. A14E,B16H,B25H,B29K(N^{ε}Octadecanedioyl-γGlu-γGlu-γGlu),desB30 human insulin,
30. A14E,B25H,B26G,B27G,B28G,B29K(N^{ε}Hexadecandioyl-γGlu),desB30 human insulin,
31. A14E,B16H,B25H,B29K(N^{ε}Octadecanedioyl-γGlu-γGlu),desB30 human insulin,
32. A14E,B16H,B25H,B29K(N^{ε}Eicosanedioyl-γGlu-OEG-OEG),desB30 human insulin,
33. A14E,B25H,B29K(N^{ε}Octadecanedioyl-OEG-γGlu-γGlu),desB30 human insulin,
34. A14E,A18L,B25H,B29K(N^{ε}Eicosanedioyl-γGlu-OEG-OEG),desB30 human insulin,
35. A14E,A18L,B25H,B29K(N^{ε}Octadecanedioyl-γGlu-OEG-OEG),desB30 human insulin,
36. A14E,B25H,B27E,B29K(N^{ε}Eicosanedioyl-γGlu-OEG-OEG),desB30 human insulin,
37. A1G(N^{α}Octadecandioyl-γGlu-OEG-OEG),A14E,B25H,B29R,desB30 human insulin,
38. A14E,B1F(N^{α}Octadecandioyl-γGlu-OEG-OEG),B25H,B29R,desB30 human insulin,
39. A1G(N^{α}Hexadecandioyl-γGlu),A14E,B25H,B29R,desB30 human insulin,
40. A14E,B25H, B29K(N^{ε}Octadecanedioyl-γGlu-Abu-Abu-Abu-Abu),desB30 human insulin,
41. A14E,B25H,B29K(N^{α}Eicosanedioyl),desB30 human insulin,
42. A14E,B25H,B29K(N^{α}4-[16-(1H-Tetrazol-5-yl)hexadecanoylsulfarnoyl]butanoyl), desB30 human insulin,
43. A1G(N^{α}Octadecandioyl-γGlu-OEG-OEG),A14E,A21G,B25H,desB30 human insulin,
44. A14E,B25H,B29K(N^{ε}Eicosanedioyl-OEG),desB30 human insulin,
45. A14E,B25H,B27K(N^{ε}Octadecanedioyl-γGlu-OEG-OEG),desB28,desB29,desB30 human insulin,
46. A14E,B25H,B29K(N^{ε}(5-Eicosanedioylaminoisophthalic acid)),desB30 human insulin,
47. A14E,B25H,B29K(N^{ε}Octadecanedioyl),desB30 human insulin,
48. A14E,B29K(N^{ε}Octadecanedioyl-γGlu-OEG-OEG),desB30 human insulin,
49. A14E,B25H,B26G,B27G,B28G,B29K(N^{ε}Eicosanedioyl-γGlu-OEG-OEG),desB30 human insulin,
50. A14E,B25H,B29K(N^{ε}Octadecanedioyl-γGlu-OEG),desB30 human insulin,
51. A14E,B25H,B29K(N^{ε}Eicosanedioyl-OEG-OEG),desB30 human insulin,
52. A14E,B25H,B29K(N^{ε}Eicosanedioyl-Aoc),desB30 human insulin,
53. A14E,B25H,B26G,B27G,B28G,B29K(N^{ε}Eicosanedioyl-γGlu-γGlu),desB30 human insulin,
54. A14E,B25H,B26G,B27G,B28G,B29K(N^{ε}Eicosanedioyl-γGlu-γGlu),desB30 human insulin,
55. A14E,B25H,B29K(N^{ε}Octadecanedioyl-OEG),desB30 human insulin,
56. A14E,B25H,desB27,B29K(N^{ε}Octadecanedioyl-γGlu-OEG-OEG),desB30 human insulin,
57. A14E,B25H,B16H,B29K(N^{ε}Octadecanedioyl-γGlu),desB30 human insulin,
58. A1G(N^{α}Octadecanedioyl),A14E,B25H,B29R,desB30 human insulin,
59. A14E,B16H,B25H,B29K(N^{ε}Eicosanedioyl-γGlu),desB30 human insulin,
60. A14E,B25H,B27K(N^{ε}Eicosanedioyl-γGlu),desB28,desB29,desB30 human insulin,
61. A14E,B25H,B29K(N^{ε}Octadecanedioyl-γGlu-γGlu-γGlu),desB30 human insulin,
62. A14E,B25H,B26G,B27G,B28G,B29K(N^{ε}Octadecandioyl-γGlu),desB30 human insulin,
63. A14E,B25H,B26G,B27G,B28G,B29K(N^{ε}Eicosanedioyl-γGlu),desB30 human insulin,
64. A14E,B25H,B26G,B27G,B28G,B29K(N^{ε}Octadecandioyl),desB30 human insulin,
65. A14E,B25H,B26G,B27G,B28G,B29K(N^{ε}Eicosanedioyl),desB30 human insulin,
66. A14E,B25H,B29K(N^{ε}Docosanedioyl-γGlu),desB30 human insulin,
67. A14E,B25H,B29K(N^{ε}Docosanedioyl-γGlu-γGlu),desB30 human insulin,
68. A14E,B25H,B29K(N^{ε}Icosanedioyl-γGlu-OEG-OEG-γGlu),desB30 human insulin,
69. A14E,B25H,B29K(N^{ε}Octadecanedioyl-γGlu-OEG-OEG-γGlu),desB30 human insulin,
70. A14E,B25H,B29K(N^{ε}(N-Icosanedioyl-N-carboxymethyl)-βAla),desB30 human insulin,
71. A14E,B25H,B29K(N^{ε}3-[2-(2-{2-[2-(17-Carboxyheptadecanoylamino)ethoxy]ethoxy}ethoxy)ethoxy]propionyl-γGlu),desB30 human insulin,
72. A14E,B25H,B29K(N^{ε}3-[2-(2-{2-[2-(19-Carboxynonadecanoylamino)ethoxy]ethoxy}ethoxy)ethoxy]propionyl-γGlu),desB30 human insulin,
73. A14E,B25H,B29K(N^{ε}Octadecandioyl-γGlu-(3-(2-{2-[2-(2-aminoethoxy)ethoxy]ethoxy}ethoxy)propionyl),desB30 human insulin,
74. A14E,B25H,B29K(N^{ε}Octadecandioyl-γGlu-(3-(2-{2-[2-(2-aminoethoxy)ethoxy]ethoxy}ethoxy)propionyl-γGlubdesB30 human insulin,
75. A14E,B25H,B29K(N^{ε}Icosanedioyl-γGlu-(3-(2-{2-[2-(2-aminoethoxy)ethoxy]ethoxy}ethoxy)propionyl),desB30 human insulin,
76. A14E,B25H,B29K(N^{ε}4-([4-({17-Carboxynonadecanoylamino}methyl)trans-cyclohexanecarbonyl]-γGlubdesB30 human insulin,
77. A14E,B25H,B29K(N^{ε}4-([4-({17-Carboxyheptadecanoylamino}methyl)trans-cyclohexanecarbonyl]-γGlu-γGlubdesB30 human insulin,
78. A14E,B1E,B25H,B28E,B29K(N^{ε}Hexadecandioyl-γGlu),desB30 human insulin,
79. A14E,B1E,B25H,B28E,B29K(N^{ε}Octadecandioyl-γGlu),desB30 human insulin,
80. A14E,B1E,B25H,B28E,B29K(N^{ε}Eicosanedioyl-γGlu),desB30 human insulin,
81. A14E,B1E,B25H,B28E,B29K(N^{ε}Hexadecandioyl-γGlu-OEG-OEG),desB30 human insulin,
82. A14E,B1E,B25H,B28E,B29K(N^{ε}Octadecandioyl-γGlu-OEG-OEG),desB30 human insulin,
83. A14E,B1E,B25H,B28E,B29K(N^{ε}Eicosanedioyl-γGlu-OEG-OEG),desB30 human insulin,
84. A14E,B1E,B25H,B27E,B28E,B29K(N^{ε}Hexadecandioyl-γGlu),desB30 human insulin,
85. A14E,B1E,B25H,B27E,B28E,B29K(N^{ε}Octadecandioyl-γGlu),desB30 human insulin,
86. A14E,B1E,B25H,B27E,B28E,B29K(N^{ε}Eicosanedioyl-γGlu),desB30 human insulin,
87. A14E,B1E,B25H,B27E,B28E,B29K(N^{ε}Hexadecandioyl-γGlu-OEG-OEG),desB30 human insulin,
88. A14E,B1E,B25H,B27E,B28E,B29K(N^{ε}Octadecandioyl-γGlu-OEG-OEG),desB30 human insulin,
89. A14E,B1E,B25H,B27E,B28E,B29K(N^{ε}Eicosanedioyl-γGlu-OEG-OEG),desB30 human insulin,
90. A14E,B25H,B29K(N^{ε}(N-Icosanedioyl-N-carboxymethyl)-βAla-OEG-OEG),desB30 human insulin,
91. A14E,B25H,B29K(N^{ε}(N-Octadecanedioyl-N-carboxymethyl)-βAla-OEG-OEG),desB30 human insulin,
92. A14E,B25H,B29K(N^{ε}(N-Hexadecanedioyl-N-carboxymethyl)-βAla-OEG-OEG),desB30 human insulin,
93. A14E,B25H,B29K(N^{ε}octadecanedioyl-γGlu-2-[(3-{2-[2-(3-aminopropoxy)ethoxy]ethoxy}propylcarbamoyl)methoxy]acetyl),desB30 human insulin, 94. A14E,B25H,B29K(N^{ε}eicosanedioyl-γGlu-2-[(3-{2-[2-(3-aminopropoxy)ethoxy]ethoxy}propylcarbamoyl)methoxy]acetyl),desB30 human insulin,
95. A14E,B16H,B25H,B29K(N^{ε}Octadecanedioyl-γGlu-2-[(3-{2-[2-(3-aminopropoxy)ethoxy]ethoxy}propylcarbamoyl)methoxy]acetyl),desB30 human insulin,
96. A14E, B16H, B25H, B29K(N^{ε}Eicosanedioyl-γGlu-2-[(3-{2-[2-(3-aminopropoxy)ethoxy]ethoxy}propylcarbamoyl)methoxy]acetyl),desB30 human insulin,
97. A14E,B25H,desB27,B29K(N^{ε}Octadecanedioyl),desB30 human insulin,
98. A14E,B25H,desB27,B29K(N^{ε}Eicosanedioyl),desB30 human insulin,
99. A14E,B25H,desB27,B29K(N^{ε}Octadecanedioyl-γGlu),desB30 human insulin,
100. A14E,B25H,desB27,B29K(N^{ε}Eicosanedioyl-γGlu),desB30 human insulin,
101. A14E,B25H,desB27,B29K(N^{ε}Eicosanedioyl-γGlu-OEG-OEG),desB30 human insulin,
102. A14E,A21G,B25H,desB27,B29K(N^{ε}Octadecanedioyl),desB30 human insulin,
103. A14E,A21G,B25H,desB27,B29K(N^{ε}Eicosanedioyl),desB30 human insulin,
104. A14E,A21G,B25H,desB27,B29K(N^{ε}Octadecanedioyl-γGlu),desB30 human insulin,
105. A14E,B25H,desB27,B29K(N^{ε}Eicosanedioyl-γGlu),desB30 human insulin,
106. A14E,A21G,B25H,desB27,B29K(N^{ε}Octadecanedioyl-γGlu-OEG-OEG),desB30 human insulin,
107. A14E,A21G,B25H,desB27,B29K(N^{ε}Eicosanedioyl-γGlu-OEG-OEG),desB30 human insulin,
108. A14E,A21G,B25H,B29K(N^{ε}Octadecanedioyl-γGlu-OEG-OEG),desB30 human insulin,
109. A14E,A21G,B25H,B29K(N^{ε}Eicosanedioyl-γGlu-OEG-OEG),desB30 human insulin,
110. A14E,A21G,B25H,B29K(N^{ε}Eicosanedioyl-γGlu),desB30 human insulin,
111. A14E,A21G,B25H,B29K(N^{ε}Eicosanedioyl),desB30 human insulin,
112. A14E,A21G,B25H,B29K(N^{ε}Octadecanedioyl-γGlu),desB30 human insulin,
113. A14E,A21G,B25H,B29K(N^{ε}Octadecanedioyl),desB30 human insulin,
114. A14E,B25H,B26G,B27G,B28G,B29K(N^{ε}Octadecanedioyl-γGlu),desB30 human insulin,
115. A14E,B25H,B26G,B27G,B28G,B29K(N^{ε}Octadecanedioyl),desB30 human insulin,
116. A14E,B25H,B26G,B27G,B28G,B29K(N^{ε}Eicosanedioyl-γGlu),desB30 human insulin,
117. A14E,B25H,B26G,B27G,B28G,B29K(N^{ε}Eicosanedioyl),desB30 human insulin,
118. A1G(N^{α}Octadecandioyl-γGlu),A14E,B25H,B26G,B27G,B28G,desB30 human insulin,
119. A1G(N^{α}Eicosanedioyl-γGlu),A14E,B25H,B26G,B27G,B28G,desB30 human insulin,
120. A1G(N^{α}Octadecandioyl-γGlu),A14E,B25H,B26G,B27G,B28G,B29R,desB30 human insulin,
121. A1G(N^{α}Eicosanedioyl-γGlu),A14E,B25H,B26G,B27G,B28G,B29R,desB30 human insulin,
122. A1G(N^{α}Octadecandioyl),A14E,B25H,B26G,B27G,B28G,desB30 human insulin,
123. A1G(N^{α}Eicosanedioyl),A14E,B25H,B26G,B27G,B28G,desB30 human insulin,
124. A1G(N^{α}Octadecandioyl),A14E,B25H,B26G,B27G,B28G,B29R,desB30 human insulin and
125. A1G(N^{α}Eicosanedioyl),A14E,B25H,B26G,B27G,B28G,B29R,desB30 human insulin.

In one embodiment a tablet core according to the present invention comprises a protease stabilised insulin, which is selected from the group consisting of:
1. A14E,B25H,B29K(N^{ε}-Hexadecandioyl),desB30 human insulin,
2. A14E,B25H,B29K(N^{ε}Octadecandioyl-γGlu),desB30 human insulin,
3. A14E,B25H,B29K(N^{ε}Eicosanedioyl-γGlu),desB30 human insulin,
4. A14E,B25H,B29K(N^{ε}3-Carboxy-5-octadecanedioylaminobenzoyl),desB30 human insulin,
5. A14E,B25H,B29K(N^{ε}-N-octadecandioyl-N-(2-carboxyethyl)glycyl),desB30 human insulin
6. A14E,B25H,B29K(N^{ε}(N-Octadecandioyl-N-carboxymethyl)-beta-alanyl),desB30 human insulin,
7. A14E,B25H,B29K(N^{ε}4-([4-({19-Carboxynonadecanoylamino}nnethyl)trans-cyclo-hexanecarbonyl]-γGlubdesB30 human insulin,
8. A14E,B25H,B29K(N^{ε}Heptadecanedioyl-γGlu),desB30 human insulin,
9. A14E,B25H,B29K(N^{ε}Octadecanedioyl-γGlu-OEG-OEG),desB30 human insulin,
10. A14E,B25H,B29K(N^{ε}Myristyl),desB30 human insulin,
11. A14E,B25H,B29K(N^{ε}Eicosanedioyl-γGlu-γGlu),desB30 human insulin,
12. A14E,B25H,B29K(N^{ε}4-([4-({19-Carboxynonadecanoylamino}methyl)trans-cyclohexanecarbonyl]-γGlu-γGlubdesB30 human insulin,
13. A14E,B25H,B29K(N^{ε}Octadecanedioyl-γGlu-γGlu),desB30 human insulin,
14. A14E,B28D,B29K(N^{ε}octadecandioyl-γGlu),desB30 human insulin,
15. A14E,B25H,B29K(N^{ε}octadecandioyl-γGlu-PEG7),desB30 human insulin,
16. A14E,B25H,B29K(N^{ε}eicosanedioyl-γGlu-OEG-OEG), desB30 human insulin,
17. A14E,B25H,B29K(N^{ε}eicosanedioyl-γGlu-(3-(2-{2-[2-(2-aminoethoxy)ethoxy]ethoxy}ethoxy)propionyl-γGlubdesB30 human insulin,
18. A14E,B25H,B29K(N^{ε}Hexadecanedioyl-γGlu-OEG-OEG),desB30 human insulin,
19. A14E,B25H,B29K(N^{ε}Hexadecanedioyl-γGlu),desB30 human insulin,
20. A14E,B25H,B29K(N^{ε}heptadecanedioyl-γGlu-OEG-OEG),desB30 human insulin,
21. A14E,B25H,B29K(N^{ε}octadecanedioyl-γGlu-γGlu-γGlu-γGlu),desB30 human insulin,
22. A14E,B25H,B29K(N^{ε}Eicosanedioyl-γGlu-γGlu-γGlu),desB30 human insulin,
23. A14E,B25H,B27E,B29K(N^{ε}Octadecanedioyl-γGlu-OEG-OEG),desB30 human insulin,
24. A14E,B25H,B26G,B27G,B28G,B29K(N^{ε}Octadecanedioyl-γGlu-OEG-OEG),desB30 human insulin,
25. A14E,B16H,B25H,B29K(N^{ε}Octadecanedioyl-γGlu-OEG-OEG),desB30 human insulin,
26. A14E,B16E,B25H,B29K(N^{ε}Octadecanedioyl-γGlu-OEG-OEG),desB30 human insulin,
27. A14E,B16H,B25H,B29K(N^{ε}Hexadecanedioyl-γGlu),desB30 human insulin,
28. A14E,B25H,B29K(N^{ε}Eicosanedioyl-γGlu-OEG-γGlu),desB30 human insulin,
29. A14E,B16E,B25H,B29K(N^{ε}Hexadecandioyl-γGlu),desB30 human insulin,
30. A14E,B16H,B25H,B29K(N^{ε}Octadecanedioyl-γGlu-γGlu-γGlu),desB30 human insulin,
31. A14E,B25H,B26G,B27G,B28G,B29K(N^{ε}Hexadecandioyl-γGlu),desB30 human insulin,
32. A14E,B16H,B25H,B29K(N^{ε}Octadecanedioyl-γGlu-γGlu),desB30 human insulin,
33. A14E,B16H,B25H,B29K(N^{ε}Eicosanedioyl-γGlu-OEG-OEG),desB30 human insulin,
34. A14E,B25H,B29K(N^{ε}Octadecanedioyl-OEG-γGlu-γGlu),desB30 human insulin,
35. A14E,A18L,B25H,B29K(N^{ε}Eicosanedioyl-γGlu-OEG-OEG),desB30 human insulin,
36. A14E,A18L,B25H,B29K(N^{ε}Octadecanedioyl-γGlu-OEG-OEG),desB30 human insulin,
37. A14E,B25H,B27E,B29K(N^{ε}Eicosanedioyl-γGlu-OEG-OEG),desB30 human insulin,
38. A1G(N^{α}OctadecandioyHGlu-OEG-OEG),A14E,B25H,B29R,desB30 human insulin,
39. A14E,B1F(N^{α}OctadecandioyHGlu-OEG-OEG),B25H,B29R,desB30 human insulin,
40. A1G(N^{α}Hexadecandioyl-γGlu),A14E,B25H,B29R,desB30 human insulin,
41. A14E,B25H, B29K(N^{ε}Octadecanedioyl-γGlu-Abu-Abu-Abu-Abu),desB30 human insulin,
42. A14E,B25H,B29K(N^{α}Eicosanedioyl),desB30 human insulin,
43. A14E,B25H,B29K(Na4-[16-(1H-Tetrazol-5-yl)hexadecanoylsulfarnoyl]butanoyl), desB30 human insulin,
44. A1G(N^{α}Octadecandioyl-γGlu-OEG-OEG),A14E,A21G,B25H,desB30 human insulin,
45. A14E,B25H,B29K(N^{ε}Eicosanedioyl-OEG),desB30 human insulin,
46. A14E,B25H,B27K(N^{ε}Octadecanedioyl-γGlu-OEG-OEG),desB28,desB29,desB30 human insulin,
47. A14E,B25H,B29K(N^{ε}(5-Eicosanedioylaminoisophthalic acid)),desB30 human insulin,
48. A14E,B25H,B29K(N^{ε}Octadecanedioyl),desB30 human insulin,
49. A14E,B29K(N^{ε}Octadecanedioyl-γGlu-OEG-OEG),desB30 human insulin,
50. A14E,B25H,B26G,B27G,B28G,B29K(N^{ε}Eicosanedioyl-γGlu-OEG-OEG),desB30 human insulin,
51. A14E,B25H,B29K(N^{ε}Octadecanedioyl-γGlu-OEG),desB30 human insulin,
52. A14E,B25H,B29K(N^{ε}Eicosanedioyl-OEG-OEG),desB30 human insulin,
53. A14E,B25H,B29K(N^{ε}Eicosanedioyl-Aoc),desB30 human insulin,
54. A14E,B25H,B26G,B27G,B28G,B29K(N^{ε}Eicosanedioyl-γGlu-γGlu),desB30 human insulin,
55. A14E,B25H,B26G,B27G,B28G,B29K(N^{ε}Eicosanedioyl-γGlu-γGlu),desB30 human insulin,
56. A14E,B25H,B29K(N^{ε}Octadecanedioyl-OEG),desB30 human insulin,
57. A14E,B25H,desB27,B29K(N^{ε}Octadecanedioyl-γGlu-OEG-OEG),desB30 human insulin,
58. A14E,B25H,B16H,B29K(N^{ε}Octadecanedioyl-γGlu),desB30 human insulin,
59. A1G(N^{α}Octadecanedioyl),A14E,B25H,B29R,desB30 human insulin,
60. A14E,B16H,B25H,B29K(N^{ε}Eicosanedioyl-γGlu),desB30 human insulin,
61. A14E,B25H,B27K(N^{ε}Eicosanedioyl-γGlu),desB28,desB29,desB30 human insulin,
62. A14E,B25H,B29K(N^{ε}Octadecanedioyl-γGlu-γGlu-γGlu),desB30 human insulin,
63. A14E,B25H,B26G,B27G,B28G,B29K(N^{ε}Octadecandioyl-γGlu),desB30 human insulin,
64. A14E,B25H,B26G,B27G,B28G,B29K(N^{ε}Eicosanedioyl-γGlu),desB30 human insulin,
65. A14E,B25H,B26G,B27G,B28G,B29K(N^{ε}Octadecandioyl),desB30 human insulin,
66. A14E,B25H,B26G,B27G,B28G,B29K(N^{ε}Eicosanedioyl),desB30 human insulin,
67. A14E,B25H,B29K(N^{ε}Docosanedioyl-γGlu),desB30 human insulin,
68. A14E,B25H,B29K(N^{ε}Docosanedioyl-γGlu-γGlu),desB30 human insulin, 69. A14E,B25H,B29K(N^{ε}Icosanedioyl-γGlu-OEG-OEG-γGlu),desB30 human insulin,
70. A14E,B25H,B29K(N^{ε}Octadecanedioyl-γGlu-OEG-OEG-γGlu),desB30 human insulin,
71. A14E,B25H,B29K(N^{ε}(N-Icosanedioyl-N-carboxymethyl)-βAla),desB30 human insulin,
72. A14E,B25H,B29K(N^{ε}3-[2-(2-{2-[2-(17-Carboxyheptadecanoylamino)ethoxy]ethoxy}ethoxy)ethoxy]propionyl-γGlu),desB30 human insulin,
73. A14E,B25H,B29K(N^{ε}3-[2-(2-{2-[2-(19-Carboxynonadecanoylamino)ethoxy]ethoxy}ethoxy)ethoxy]propionyl-γGlu),desB30 human insulin,
74. A14E,B25H,B29K(N^{ε}Octadecandioyl-γGlu-(3-(2-{2-[2-(2-aminoethoxy)ethoxy]ethoxy}ethoxy)propionyl),desB30 human insulin,
75. A14E,B25H,B29K(N^{ε}Octadecandioyl-γGlu-(3-(2-{2-[2-(2-aminoethoxy)ethoxy]ethoxy}ethoxy)propionyl-γGlubdesB30 human insulin,
76. A14E,B25H,B29K(N^{ε}Icosanedioyl-γGlu-(3-(2-{2-[2-(2-aminoethoxy)ethoxy]ethoxy}ethoxy)propionyl),desB30 human insulin,
77. A14E,B25H,B29K(N^{ε}4-([4-({17-Carboxynonadecanoylamino}methyl)trans-cyclohexanecarbonyl]-γGlubdesB30 human insulin,
78. A14E,B25H,B29K(N^{ε}4-([4-({17-Carboxyheptadecanoylamino}methyl)trans-cyclohexanecarbonyl]-γGlu-γGlubdesB30 human insulin,
79. A14E,B1E,B25H,B28E,B29K(N^{ε}Hexadecandioyl-γGlu),desB30 human insulin,
80. A14E,B1E,B25H,B28E,B29K(N^{ε}Octadecandioyl-γGlu),desB30 human insulin,
81. A14E,B1E,B25H,B28E,B29K(N^{ε}Eicosanedioyl-γGlu),desB30 human insulin,
82. A14E,B1E,B25H,B28E,B29K(N^{ε}Hexadecandioyl-γGlu-OEG-OEG),desB30 human insulin,
83. A14E,B1E,B25H,B28E,B29K(N^{ε}Octadecandioyl-γGlu-OEG-OEG),desB30 human insulin,
84. A14E,B1E,B25H,B28E,B29K(N^{ε}Eicosanedioyl-γGlu-OEG-OEG),desB30 human insulin,
85. A14E,B1E,B25H,B27E,B28E,B29K(N^{ε}Hexadecandioyl-γGlu),desB30 human insulin,
86. A14E,B1E,B25H,B27E,B28E,B29K(N^{ε}Octadecandioyl-γGlu),desB30 human insulin,
87. A14E,B1E,B25H,B27E,B28E,B29K(N^{ε}Eicosanedioyl-γGlu),desB30 human insulin,
88. A14E,B1E,B25H,B27E,B28E,B29K(N^{ε}Hexadecandioyl-γGlu-OEG-OEG),desB30 human insulin,
89. A14E,B1E,B25H,B27E,B28E,B29K(N^{ε}Octadecandioyl-γGlu-OEG-OEG),desB30 human insulin,
90. A14E,B1E,B25H,B27E,B28E,B29K(N^{ε}Eicosanedioyl-γGlu-OEG-OEG),desB30 human insulin,
91. A14E,B25H,B29K(N^{ε}(N-Icosanedioyl-N-carboxymethyl)-βAla-OEG-OEG),desB30 human insulin,
92. A14E,B25H,B29K(N^{ε}(N-Octadecanedioyl-N-carboxymethyl)-βAla-OEG-OEG),desB30 human insulin,
93. A14E,B25H,B29K(N^{ε}(N-Hexadecanedioyl-N-carboxymethyl)-βAla-OEG-OEG),desB30 human insulin,
94. A14E,B25H,B29K(N^{ε}octadecanedioyl-γGlu-2-[(3-{2-[2-(3-aminopropoxy)ethoxy]ethoxy}propylcarbamoyl)methoxy]acetyl),desB30 human insulin,
95. A14E,B25H,B29K(N^{ε}eicosanedioyl-γGlu-2-[(3-{2-[2-(3-aminopropoxy)ethoxy]ethoxy}propylcarbamoyl)methoxy]acetyl),desB30 human insulin,
96. A14E,B16H,B25H,B29K(N^{ε}Octadecanedioyl-γGlu-2-[(3-{2-[2-(3-aminopropoxy)-ethoxy]ethoxy}propylcarbamoyl)methoxy]acetyl),desB30 human insulin,
97. A14E, B16H, B25H, B29K(N^{ε}Eicosanedioyl-γGlu-2-[(3-{2-[2-(3-aminopropoxy)-ethoxy]ethoxy}propylcarbamoyl)methoxy]acetyl),desB30 human insulin,
98. A14E,B25H,desB27,B29K(N^{ε}Octadecanedioyl),desB30 human insulin,
99. A14E,B25H,desB27,B29K(N^{ε}Eicosanedioyl),desB30 human insulin,
100. A14E,B25H,desB27,B29K(N^{ε}Octadecanedioyl-γGlu),desB30 human insulin,
101. A14E,B25H,desB27,B29K(N^{ε}Eicosanedioyl-γGlu),desB30 human insulin,
102. A14E,B25H,desB27,B29K(N^{ε}Eicosanedioyl-γGlu-OEG-OEG),desB30 human insulin,
103. A14E,A21G,B25H,desB27,B29K(N^{ε}Octadecanedioyl),desB30 human insulin,
104. A14E,A21G,B25H,desB27,B29K(N^{ε}Eicosanedioyl),desB30 human insulin,
105. A14E,A21G,B25H,desB27,B29K(N^{ε}Octadecanedioyl-γGlu),desB30 human insulin,
106. A14E,B25H,desB27,B29K(N^{ε}Eicosanedioyl-γGlu),desB30 human insulin,
107. A14E,A21G,B25H,desB27,B29K(N^{ε}Octadecanedioyl-γGlu-OEG-OEG),desB30 human insulin,
108. A14E,A21G,B25H,desB27,B29K(N^{ε}Eicosanedioyl-γGlu-OEG-OEG),desB30 human insulin,
109. A14E,A21G,B25H,B29K(N^{ε}Octadecanedioyl-γGlu-OEG-OEG),desB30 human insulin,
110. A14E,A21G,B25H,B29K(N^{ε}Eicosanedioyl-γGlu-OEG-OEG),desB30 human insulin,
111. A14E,A21G,B25H,B29K(N^{ε}Eicosanedioyl-γGlu),desB30 human insulin,
112. A14E,A21G,B25H,B29K(N^{ε}Eicosanedioyl),desB30 human insulin,
113. A14E,A21G,B25H,B29K(N^{ε}Octadecanedioyl-γGlu),desB30 human insulin,
114. A14E,A21G,B25H,B29K(N^{ε}Octadecanedioyl),desB30 human insulin,
115. A14E,B25H,B26G,B27G,B28G,B29K(N^{ε}Octadecanedioyl-γGlu),desB30 human insulin,
116. A14E,B25H,B26G,B27G,B28G,B29K(N^{ε}Octadecanedioyl),desB30 human insulin,
117. A14E,B25H,B26G,B27G,B28G,B29K(N^{ε}Eicosanedioyl-γGlu),desB30 human insulin,
118. A14E,B25H,B26G,B27G,B28G,B29K(N^{ε}Eicosanedioyl),desB30 human insulin,
119. A1G(N^{α}Octadecandioyl-γGlu),A14E,B25H,B26G,B27G,B28G,desB30 human insulin,
120. A1G(N^{α}Eicosanedioyl-γGlu),A14E,B25H,B26G,B27G,B28G,desB30 human insulin,
121. A1G(N^{α}Octadecandioyl-γGlu),A14E,B25H,B26G,B27G,B28G,B29R,desB30 human insulin,
122. A1G(N^{α}Eicosanedioyl-γGlu),A14E,B25H,B26G,B27G,B28G,B29R,desB30 human insulin,
123. A1G(N^{α}Octadecandioyl),A14E,B25H,B26G,B27G,B28G,desB30 human insulin,
124. A1G(N^{α}Eicosanedioyl),A14E,B25H,B26G,B27G,B28G,desB30 human insulin,
125. A1G(N^{α}Octadecandioyl),A14E,B25H,B26G,B27G,B28G,B29R,desB30 human insulin and
126. A1G(N^{α}Eicosanedioyl),A14E,B25H,B26G,B27G,B28G,B29R,desB30 human insulin.

In one embodiment a tablet core according to the present invention comprises a protease stabilised insulin which is selected from the group consisting of:
1. A10C,A14E,B4C,B25H,B29K(N^{ε}Octadecanedioyl-γGlu-OEG-OEG),desB30 human insulin,
2. A10C,A14E,B3C,B25H,B29K(N^{ε}Octadecanedioyl-γGlu),desB30 human insulin,
3. A10C,A14E,B3C,B25H,B29K(N^{ε}Octadecanedioyl),desB30 human insulin,
4. A10C,A14E,B3C,B25H,B29K(N^{ε}Octadecanedioyl-γGlu),desB30 human insulin,
5. A10C,A14E,desB1,B4C,B25H,B29K(N^{ε}Octadecanedioyl-γGlu-OEG-OEG),desB30 human insulin,
6. A10C,A14H,B4C,B25H,B29K(N^{ε}Octadecanedioyl-γGlu-OEG-OEG),desB30 human insulin,
7. A10C,A14E,B3C,B25H,B29K(N^{ε}Eicosanedioyl-γGlu-OEG-OEG),desB30 human insulin,
8. A10C,A14E,B1C, B25H,B29K(N^{ε}Eicosanedioyl-γGlu-OEG-OEG),desB30 human insulin,
9. A10C,A14E,B4C B25H,B29K(N^{ε}Octadecanedioyl-γGlu),desB30 human insulin,
10. A10C,A14E, B3C,B25H,B29K(N^{ε}Octadecanedioyl-γGlu-OEG-OEG),desB30 human insulin,
11. A10C,A14E,B3C,B25H,B29K(N^{ε}Octadecanedioyl-γGlu-γGlu),desB30 human insulin,
12. A10C,A14E,B4C,B25H,desB27,B29K(N^{ε}Octadecanedioyl-γGlu),desB30 human insulin,
13. A10C,A14E,B4C,B25H,B29K(N^{ε}Octadecanedioyl),desB30 human insulin,
14. A10C,A14E,B4C,B25H,B29K(N^{ε}Octadecanedioyl-γGlu-γGlu),desB30 human insulin,
15. A10C,A14E,B2C,B25H,B29K(N^{ε}Octadecanedioyl-γGlu-OEG-OEG),desB30 human insulin,
16. A10C,A14E,61C, B25H, B29K(N^{ε}Octadecanedioyl-γGlu-OEG-OEG),desB30 human insulin,
17. A10C,A14E,B3C,B16H,B25H,B29K(N^{ε}Eicosanedioyl-γGlu-OEG-OEG),desB30 human insulin,
18. A10C,A14E, B4C,B25H,B29K(N^{ε}Myristyl),desB30 human insulin,
19. A10C,B4C,B29K(N^{ε}Myristyl),desB30 human insulin,
20. A10C,A14E,B3C,B25H,desB27,B29K(N^{ε}octadecanedioyl-γGlu),desB30 human insulin,
21. A10C,A14E,B3C,B25H,desB27,B29K(N^{ε}octadecanedioyl-γGlu-OEG-OEG),desB30 human insulin,
22. A10C,A14E,B3C,B25H,B29K(N^{ε}Eicosanedioyl-γGlu),desB30 human insulin,
23. A10C,A14E,B4C,B25H,B29K(N^{ε}eicosanedioyl-γGlu-OEG-OEG),desB30 human insulin,
24. A10C,A14E,B3C,B25H,desB27,B29K(N^{ε}eicosanedioyl-γGlu),desB30 human insulin,
25. A10C,A14E,B3C,B25H,desB27,B29K(N^{ε}eicosanedioyl-γGlu-OEG-OEG),desB30 human insulin,
26. A10C,A14E,4C,B25H,B29K(N^{ε}Hexadecanedioyl-γGlu),desB30 human insulin,
27. A10C,A14E,B4C,B25H,B29K(N^{ε}Hexadecanedioyl-γGlu-OEG-OEG),desB30 human insulin,
28. A10C,A14E,B4C,B25H,B29K(N^{ε}Hexadecanedioyl),desB30 human insulin,
29. A10C,A14E,B4C,B25H,B29K(N^{ε}Hexadecanedioyl-γGlu-γGlu),desB30 human insulin,
30. A10C,A14E,B4C,B25H,desB27,B29K(N^{ε}Octadecanedioyl-γGlu-OEG-OEG),desB30 human insulin,
31. A10C,A14E,B4C,B25H,desB27,B29K(N^{ε}Octadecanedioyl-γGlu-γGlu),desB30 human insulin,
32. A10C,A14E,B4C,B25H,desB27,B29K(N^{ε}Hexadecanedioyl-γGlu-OEG-OEG),desB30 human insulin,
33. A10C,A14E,B4C,B25H,desB27,B29K(N^{ε}Hexadecanedioyl-γGlu),desB30 human insulin,
34. A10C,A14E,B3C,B25H,B29K(N^{ε}Hexadecanedioyl-γGlu),desB30 human insulin,
35. A10C,A14E,B3C,B25H,B29K(N^{ε}Hexadecanedioyl-γGlu-OEG-OEG),desB30 human insulin,
36. A10C,A14E,B2C,B25H,B29K(N^{ε}Hexadecanedioyl-γGlu-OEG-OEG),desB30 human insulin,
37. A10C,A14E,B2C,B25H,B29K(N^{ε}Hexadecanedioyl-γGlu),desB30 human insulin,
38. A10C,A14E,B2C,B25H,B29K(N^{ε}Octadecanedioyl-γGlu),desB30 human insulin,
39. A10C,A14E,B1C,B25H,B29K(N^{ε}Octadecanedioyl-γGlu),desB30 human insulin,
40. A10C,A14E,B1C,B25H,B29K(N^{ε}Hexadecanedioyl-γGlu-OEG-OEG),desB30 human insulin,
41. A10C,A14E,B1C,B25H,B29K(N^{ε}Hexadecanedioyl-γGlu),desB30 human insulin,
42. A10C,B1C,B29K(N^{ε}Hexadecanedioyl-γGlu-OEG-OEG),desB30 human insulin,
43. A10C,B1C,B29K(N^{ε}Hexadecanedioyl-γGlu),desB30 human insulin,
44. A10C,B1C,B29K(N^{ε}Octadecanedioyl-γGlu-OEG-OEG),desB30 human insulin,
45. A10C,B1C,B29K(N^{ε}Octadecanedioyl-γGlu),desB30 human insulin,
46. A10C,B2C,B29K(N^{ε}Octadecanedioyl-γGlu-OEG-OEG),desB30 human insulin,
47. A10C,B2C,B29K(N^{ε}Octadecanedioyl-γGlu),desB30 human insulin,
48. A10C,B2C,B29K(N^{ε}Hexadecanedioyl-γGlu-OEG-OEG),desB30 human insulin,
49. A10C,B2C,B29K(N^{ε}Hexadecanedioyl-γGlu),desB30 human insulin,
50. A10C,B3C,B29K(N^{ε}Hexadecanedioyl-γGlu),desB30 human insulin,
51. 10C,B3B29K(N^{ε}Hexadecanedioyl-γGlu-OEG-OEG),desB30 human insulin,
52. A10C,B3C,B29K(N^{ε}Octadecanedioyl-γGlu-OEG-OEG),desB30 human insulin,
53. A10C,B3C,B29K(N^{ε}Octadecanedioyl-γGlu),desB30 human insulin,
54. A10C,B4C,B29K(N^{ε}Octadecanedioyl-γGlu),desB30 human insulin,
55. A10C,B4C,B29K(N^{ε}Octadecanedioyl-γGlu-OEG-OEG),desB30 human insulin,
56. A10C,B4C B29K(N^{ε}Hexadecanedioyl-γGlu-OEG-OEG),desB30 human insulin,
57. A10C,B4C,B29K(N^{ε}Hexadecanedioyl-γGlu),desB30 human insulin,
58. A10C,A14E,B1C,B16H,B25H,B29K(N^{ε}eicosanedioyl-γGlu-OEG-OEG),desB30 human insulin,
59. A10C,A14E,B1C,B16H,B25H,B29K(N^{ε}eicosanedioyl-γGlu),desB30 human insulin,
60. A10C,A14E,B1C,B16H,B25H,B29K(N^{ε}Octadecanedioyl-γGlu-OEG-OEG),desB30 human insulin,
61. A10C,A14E,B1C,B16H,B25H,B29K(N^{ε}Octadecanedioyl-γGlu),desB30 human insulin,
62. A10C A14E,B1C,B16H,B25H,B29K(N^{ε}Hexadecanedioyl-γGlu),desB30 human insulin,
63. A10C,A14E,B1C,B16H,B25H,B29K(N^{ε}Hexadecanedioyl-γGlu-OEG-OEG),desB30 human insulin,
64. A10C,A14E,B2C,B16H,B25H,B29K(N^{ε}Hexadecanedioyl-γGlu-OEG-OEG),desB30 human insulin,
65. A10C,A14E,B2C,B16H,B25H,B29K(N^{ε}Hexadecanedioyl-γGlu),desB30 human insulin,
66. A10C,A14E,B2C,B16H,B25H,B29K(N^{ε}Octadecanedioyl-γGlu),desB30 human insulin,
67. A10C,A14E,B2C,B16H,B25H,B29K(N^{ε}Octadecanedioyl-γGlu-OEG-OEG),desB30 human insulin,
68. A10C,A14E,B2C,B16H,B25H,B29K(N^{ε}Eicosanedioyl-γGlu-OEG-OEG),desB30 human insulin,
69. A10C,A14E,B2C,B16H,B25H,B29K(N^{ε}Eicosanedioyl-γGlu),desB30 human insulin,
70. A10C,A14E,B3C,B16H,B25H,B29K(N^{ε}Eicosanedioyl-γGlu),desB30 human insulin,
71. A10C,A14E,B3C,B16H,B25H,B29K(N^{ε}Octadecanedioyl-γGlu-OEG-OEG),desB30 human insulin,
72. A10C,A14E,B3C,B16H, B25H,B29K(N^{ε}Octadecanedioyl-γGlu),desB30 human insulin
73. A10C,A14E,B3C,B16H,B25H,B29K(N^{ε}Hexadecanedioyl-γGlu),desB30 human insulin,
74. A10C,A14E,B3C,B16H,B25H,B29K(N^{ε}Hexadecanedioyl-γGlu-OEG-OEG),desB30 human insulin,
75. A10C,A14E,B4C,B16H,B25H,B29K(N^{ε}Hexadecanedioyl-γGlu-OEG-OEG),desB30 human insulin,
76. A10C,A14E,B4C,B16H,B25H,B29K(N^{ε}Hexadecanedioyl-γGlu),desB30 human insulin,
77. A10C,A14E,B4C,B16H,B25H,B29K(N^{ε}Octadecanedioyl-γGlu),desB30 human insulin,
78. A10C,A14E,B4C,B16H,B25H B29K(N^{ε}Octadecanedioyl-γGlu-OEG-OEG),desB30 human insulin,
79. A10C,A14E,B4C,B16H B25H,B29K(N^{ε}Eicosanedioyl-γGlu-OEG-OEG),desB30 human insulin,
80. A10C,A14E,B4C,B16H,B25H,B29K(N^{ε}Eicosanedioyl-γGlu),desB30 human insulin,
81. A10C,A14E,B1C,B25H,B29K(N^{ε}eicosanedioyl-γGlu),desB30 human insulin,
82. A10C,A14E,B2C,B25H,B29K(N^{ε}eicosanedioyl-γGlu),desB30 human insulin,
83. A10C,A14E,B2C,B25H,B29K(N^{ε}eicosanedioyl-γGlu-OEG-OEG),desB30 human insulin,
84. A10C,A14E,B4C,B25H,desB27,B29K(N^{ε}eicosanedioyl-γGlu),desB30 human insulin,
85. A10C,A14E,B4C,B25H,desB27,B29K(N^{ε}eicosanedioyl-γGlu-OEG-OEG),desB30 human insulin,
86. A10C,A14E,B4C,B25H,B29K(N^{ε}eicosanedioyl-γGlu),desB30 human insulin,
87. A10C,A14E,B3C,B25H,desB27,B29K(N^{ε}hexadecanedioyl-γGlu),desB30 human insulin,
88. A10C,A14E,B3C,B25H,desB27,B29K(N^{ε}hexadecanedioyl-γGlu-OEG-OEG),desB30 human insulin,
89. A10C,A14E,B3C,desB27,B29K(N^{ε}hexadecanedioyl-γGlu),desB30 human insulin,
90. A10C,A14E,B3C,desB27,B29K(N^{ε}hexadecanedioyl-γGlu-OEG-OEG),desB30 human insulin,
91. A10C,A14E,B3C,desB27,B29K(N^{ε}octadecanedioyl-γGlu),desB30 human insulin,
92. A10C,A14E,B3C,desB27,B29K(N^{ε}octadecanedioyl-γGlu-OEG-OEG),desB30 human insulin,
93. A10C,A14E,B3C,desB27,B29K(N^{ε}eicosanedioyl-γGlu),desB30 human insulin,
94. A10C,A14E,B3C,desB27,B29K(N^{ε}eicosanedioyl-γGlu-OEG-OEG),desB30 human insulin,
95. A10C,A14E,B3C,B16H,B25H,B29K(N^{ε}eicosanedioyl-γGlu-γGlu),desB30 human insulin,
96. A10C,A14E,B3C,B16E,B25H,B29K(N^{ε}eicosanedioyl-γGlu-OEG-OEG),desB30 human insulin,
97. A10C,A14E,B4C,B16E,B25H,B29K(N^{ε}eicosanedioyl-γGlu-OEG-OEG),desB30 human insulin,
98. A10C,A14E,B3C,B16H,B25H,B29K(N^{ε}eicosanedioyl-γGlu-γGlu),desB30 human insulin and
99. A10C,A14E,B4C,B16E,B25H,B29K(N^{ε}eicosanedioyl-γGlu-γGlu),desB30 human insulin.

In one embodiment a tablet core according to the present invention comprises an protease stabilised insulin selected from the group consisting of:
A10C,A14E,B4C,B25H,B29K(N^{ε}Octadecanedioyl-γGlu-OEG-OEG),desB30 human insulin,
A10C,A14E,B3C,B25H,B29K(N^{ε}Octadecanedioyl-γGlu),desB30 human insulin,
A10C,A14E,B3C,B25H,B29K(N^{ε}Octadecanedioyl),desB30 human insulin, A10C,A14E,B3C,B25H,B29K(N^{ε}Octadecanedioyl-γGlu),desB30 human insulin,
A10C,A14H,B4C,B25H,B29K(N^{ε}Octadecanedioyl-γGlu-OEG-OEG),desB30 human insulin,
A10C,A14E,B3C,B25H,B29K(N^{ε}Eicosanedioyl-γGlu-OEG-OEG),desB30 human insulin,
A10C,A14E,B4C B25H,B29K(N^{ε}Octadecanedioyl-γGlu),desB30 human insulin, A10C,A14E, B3C,B25H,B29K(N^{ε}Octadecanedioyl-γGlu-OEG-OEG),desB30 human insulin,
A10C,A14E,B3C,B25H,B29K(N^{ε}Octadecanedioyl-γGlu-γGlu),desB30 human insulin,
A10C,A14E,B4C,B25H,desB27,B29K(N^{ε}Octadecanedioyl-γGlu),desB30 human insulin,
A10C,A14E,B4C,B25H,B29K(N^{ε}Octadecanedioyl),desB30 human insulin, A10C,A14E,B4C,B25H,B29K(N^{ε}Octadecanedioyl-γGlu-γGlu),desB30 human insulin,
A10C,A14E,B3C,B16H,B25H,B29K(N^{ε}Eicosanedioyl-γGlu-OEG-OEG),desB30 human insulin,
A10C,A14E, B4C,B25H,B29K(N^{ε}Myristyl),desB30 human insulin,
A10C,B4C, B29K(N^{ε}Myristyl),desB30 human insulin,
A10C,A14E,B3C,B25H,desB27,B29K(N^{ε}octadecanedioyl-γGlu),desB30 human insulin,
A10C,A14E,B3C,B25H,desB27,B29K(N^{ε}octadecanedioyl-γGlu-OEG-OEG),desB30 human insulin,
A10C,A14E,B3C,B25H,B29K(N^{ε}Eicosanedioyl-γGlu),desB30 human insulin,
A10C,A14E,B4C,B25H,B29K(N^{ε}eicosanedioyl-γGlu-OEG-OEG),desB30 human insulin,
A10C,A14E,B3C,B25H,desB27,B29K(N^{ε}eicosanedioyl-γGlu),desB30 human insulin,
A10C,A14E,B3C,B25H,desB27, 629K(N^{ε}eicosanedioyl-γGlu-OEG-OEG),desB30 human insulin,
A10C,A14E, 4C,B25H,B29K(N^{ε}Hexadecanedioyl-γGlu),desB30 human insulin,
A10C,A14E,B4C,B25H,B29K(N^{ε}Hexadecanedioyl-γGlu-OEG-OEG),desB30 human insulin,
A10C,A14E,B4C,B25H,B29K(N^{ε}Hexadecanedioyl),desB30 human insulin,
A10C,A14E,B4C,B25H,B29K(N^{ε}Hexadecanedioyl-γGlu-γGlu),desB30 human insulin,
A10C,A14E,B4C,B25H,desB27,B29K(N^{ε}Octadecanedioyl-γGlu-OEG-OEG),desB30 human insulin,
A10C,A14E,B4C,B25H,desB27,B29K(N^{ε}Octadecanedioyl-γGlu-γGlu),desB30 human insulin,
A10C,A14E,B4C,B25H,desB27,B29K(N^{ε}Hexadecanedioyl-γGlu-OEG-OEG),desB30 human insulin,
A10C,A14E,B4C,B25H,desB27,B29K(N^{ε}Hexadecanedioyl-γGlu),desB30 human insulin,
A10C,A14E,B3C,B25H,B29K(N^{ε}Hexadecanedioyl-γGlu),desB30 human insulin,
A10C,A14E,B3C,B25H,B29K(N^{ε}Hexadecanedioyl-γGlu-OEG-OEG),desB30 human insulin,
A10C,B3C,B29K(N^{ε}Hexadecanedioyl-γGlu),desB30 human insulin,
A10C,B3C B29K(N^{ε}Hexadecanedioyl-γGlu-OEG-OEG),desB30 human insulin,
A10C,B3C,B29K(N^{ε}Octadecanedioyl-γGlu-OEG-OEG),desB30 human insulin,
A10C,B3C,B29K(N^{ε}Octadecanedioyl-γGlu),desB30 human insulin,
A10C,B4C,B29K(N^{ε}Octadecanedioyl-γGlu),desB30 human insulin,
A10C,B4C,B29K(N^{ε}Octadecanedioyl-γGlu-OEG-OEG),desB30 human insulin,
A10C,B4C B29K(N^{ε}Hexadecanedioyl-γGlu-OEG-OEG),desB30 human insulin,
A10C,B4C,B29K(N^{ε}Hexadecanedioyl-γGlu),desB30 human insulin,
A10C,A14E,B3C,B16H,B25H,B29K(N^{ε}Eicosanedioyl-γGlu),desB30 human insulin,
A10C,A14E,B3C,B16H,B25H, B29K(N^{ε}Octadecanedioyl-γGlu-OEG-OEG),desB30 human insulin,
A10C,A14E,B3C,B16H, B25H,B29K(N^{ε}Octadecanedioyl-γGlu),desB30 human insulin
A10C,A14E,B3C,B16H,B25H,B29K(N^{ε}Hexadecanedioyl-γGlu),desB30 human insulin,
A10C,A14E,B3C,B16H,B25H, B29K(N^{ε}Hexadecanedioyl-γGlu-OEG-OEG),desB30 human insulin,
A10C,A14E,B4C,B16H,B25H, B29K(N^{ε}Hexadecanedioyl-γGlu-OEG-OEG),desB30 human insulin,
A10C,A14E,B4C,B16H,B25H,B29K(N^{ε}Hexadecanedioyl-γGlu),desB30 human insulin,
A10C,A14E,B4C,B16H,B25H,B29K(N^{ε}Octadecanedioyl-γGlu),desB30 human insulin,
A10C,A14E,B4C,B16H,B25H B29K(N^{ε}Octadecanedioyl-γGlu-OEG-OEG),desB30 human insulin,
A10C,A14E,B4C,B16H B25H,B29K(N^{ε}Eicosanedioyl-γGlu-OEG-OEG),desB30 human insulin,
A10C,A14E,B4C,B16H,B25H,B29K(N^{ε}Eicosanedioyl-γGlu),desB30 human insulin,
A10C,A14E,B4C,B25H,desB27,B29K(N^{ε}eicosanedioyl-γGlu),desB30 human insulin,
A10C,A14E,B4C,B25H,desB27,B29K(N^{ε}eicosanedioyl-γGlu-OEG-OEG),desB30 human insulin,
A10C,A14E,B4C,B25H,B29K(N^{ε}eicosanedioyl-γGlu),desB30 human insulin,
A10C,A14E,B3C,B25H,desB27,B29K(N^{ε}hexadecanedioyl-γGlu),desB30 human insulin,
A10C,A14E,B3C,B25H,desB27,B29K(N^{ε}hexadecanedioyl-γGlu-OEG-OEG),desB30 human insulin,
A10C,A14E,B3C,desB27,B29K(N^{ε}hexadecanedioyl-γGlu),desB30 human insulin,
A10C,A14E,B3C,desB27,B29K(N^{ε}hexadecanedioyl-γGlu-OEG-OEG),desB30 human insulin,
A10C,A14E,B3C,desB27,B29K(N^{ε}octadecanedioyl-γGlu),desB30 human insulin,
A10C,A14E,B3C,desB27,B29K(N^{ε}octadecanedioyl-γGlu-OEG-OEG),desB30 human insulin,
A10C,A14E,B3C,desB27,B29K(N^{ε}eicosanedioyl-γGlu),desB30 human insulin,
A10C,A14E,B3C,desB27,B29K(N^{ε}eicosanedioyl-γGlu-OEG-OEG),desB30 human insulin,
A10C,A14E,B3C,B16H,B25H,B29K(N^{ε}eicosanedioyl-γGlu-γGlu),desB30 human insulin,
A10C,A14E,B3C,B16E,B25H,B29K(N^{ε}eicosanedioyl-γGlu-OEG-OEG),desB30 human insulin,
A10C,A14E,B4C,B16E,B25H,B29K(N^{ε}eicosanedioyl-γGlu-OEG-OEG),desB30 human insulin,
A10C,A14E,B3C,B16H,B25H,B29K(N^{ε}eicosanedioyl-γGlu-γGlu),desB30 human insulin and
A10C,A14E,B4C,B16E,B25H,B29K(N^{ε}eicosanedioyl-γGlu-γGlu),desB30 human insulin,
A14E,B25H,B29K(N^{ε}Octadecanedioyl-γGlu-OEG-OEG),desB30 human insulin,
A14E,B16H,B25H,B29K(N^{ε}Octadecanedioyl-γGlu-OEG-OEG),desB30 human insulin,
A14E,B16H,B25H,B29K(N^{ε}Eicosanedioyl-γGlu-OEG-OEG),desB30 human insulin,
A14E,B25H,desB27,B29K(N^{ε}Octadecanedioyl-γGlu-OEG-OEG),desB30 human insulin,
A14E,B16H,B25H,B29K(N^{ε}Eicosanedioyl-γGlu),desB30 human insulin,
A14E,B25H,desB27,B29K(N^{ε}Octadecanedioyl-γGlu),desB30 human insulin,
A14E,B25H,desB27,B29K(N^{ε}Eicosanedioyl-γGlu),desB30 human insulin and
A14E,B25H,desB27,B29K(N^{ε}Eicosanedioyl-γGlu-OEG-OEG),desB30 human insulin,
A10C,A14E,B4C,B25H,B29K(N^{ε}Octadecanedioyl-γGlu-OEG-OEG),desB30 human insulin,
A10C,A14E,B3C,B25H,B29K(N^{ε}Octadecanedioyl-γGlu),desB30 human insulin,
A10C,A14E,B4C,B25H,desB27,B29K(N^{ε}Octadecanedioyl-γGlu),desB30 human insulin,
A10C,A14E,B3C,B16H,B25H,B29K(N^{ε}Eicosanedioyl-γGlu-OEG-OEG),desB30 human insulin,
A10C,A14E,B3C,B25H,desB27,B29K(N^{ε}octadecanedioyl-γGlu-OEG-OEG),desB30 human insulin,
A10C,A14E,B3C,B25H,desB27, B29K(N^{ε}eicosanedioyl-γGlu-OEG-OEG),desB30 human insulin,
A10C,A14E,B3C,B16H,B25H, B29K(N^{ε}Octadecanedioyl-γGlu-OEG-OEG),desB30 human insulin,
A10C,A14E,B4C,B16H,B25H B29K(N^{ε}Octadecanedioyl-γGlu-OEG-OEG),desB30 human insulin,
A10C,A14E,B4C,B16H B25H,B29K(N^{ε}Eicosanedioyl-γGlu-OEG-OEG),desB30 human insulin and
A10C,A14E,B4C,B25H,desB27,B29K(N^{ε}eicosanedioyl-γGlu-OEG-OEG),desB30 human insulin.

In one embodiment a tablet core according to the present invention comprises an protease stabilised insulin selected from the group consisting of:
A10C,A14E,B4C,B25H,B29K(N^{ε}Octadecanedioyl-γGlu-OEG-OEG),desB30 human insulin,
A10C,A14E,B3C,B25H,B29K(N^{ε}Octadecanedioyl-γGlu),desB30 human insulin,
A10C,A14E,B4C,B25H,desB27,B29K(N^{ε}Octadecanedioyl-γGlu),desB30 human insulin,
A10C,A14E,B3C,B16H,B25H,B29K(N^{ε}Eicosanedioyl-γGlu-OEG-OEG),desB30 human insulin,
A10C,A14E,B3C,B25H,desB27,B29K(N^{ε}octadecanedioyl-γGlu-OEG-OEG),desB30 human insulin,
A10C,A14E,B3C,B25H,desB27,B29K(N^{ε}eicosanedioyl-γGlu-OEG-OEG),desB30 human insulin,
A10C,A14E,B3C,B16H,B25H, B29K(N^{ε}Octadecanedioyl-γGlu-OEG-OEG),desB30 human insulin,
A10C,A14E,B4C,B16H,B25H B29K(N^{ε}Octadecanedioyl-γGlu-OEG-OEG),desB30 human insulin,
A10C,A14E,B4C,B16H B25H,B29K(N^{ε}Eicosanedioyl-γGlu-OEG-OEG),desB30 human insulin and
A10C,A14E,B4C,B25H,desB27,B29K(N^{ε}eicosanedioyl-γGlu-OEG-OEG),desB30 human insulin.

In one embodiment a tablet core according to the present invention comprises a protease stabilised insulin, which is selected from the group consisting of:
A14E,B25H,B29K(N^{ε}Octadecanedioyl-γGlu-OEG-OEG),desB30 human insulin,
A14E,B16H,B25H,B29K(N^{ε}Octadecanedioyl-γGlu-OEG-OEG),desB30 human insulin,
A14E,B16H,B25H,B29K(N^{ε}Eicosanedioyl-γGlu-OEG-OEG),desB30 human insulin,
A14E,B25H,desB27,B29K(N^{ε}Octadecanedioyl-γGlu-OEG-OEG),desB30 human insulin,
A14E,B16H,B25H,B29K(N^{ε}Eicosanedioyl-γGlu),desB30 human insulin,
A14E,B25H,desB27,B29K(N^{ε}Octadecanedioyl-γGlu),desB30 human insulin,
A14E,B25H,desB27,B29K(N^{ε}Eicosanedioyl-γGlu),desB30 human insulin and
A14E,B25H,desB27,B29K(N^{ε}Eicosanedioyl-γGlu-OEG-OEG),desB30 human insulin.

### TERMS AND DEFINITIONS

The term “parent insulin” as used herein is intended to mean an insulin optionally with one or more additional disulfide bonds relative to i.e. human insulin, desB30 human insulin or an insulin analogue with one or more additional disulfide bonds, before being derivatized with a side chain.

Herein, the term “acylated insulin” covers modification of human insulin or an insulin analogue by attachment of one or more side chains via a linker to the insulin. The term “acylated insulin” as used herein thus includes insulin derivatives. The terms “acylated insulin” and “insulin derivative” are used herein as synonyms.

The term “linker” is herein used for a portion in between the side chain and the point of attachment to the insulin peptide, which portion may also be referred to as “linker moiety”, “spacer”, or the like. The linker may be optional. In one embodiment, the linker comprises a neutral linear or cyclic amino acid residue, an acidic amino acid residue and/or a neutral, alkyleneglycol-containing amino acid residue, where the order by which these residues appear may be interchanged independently. The connections between the residues, the side chain and the insulin peptide are amide (peptide) bonds.

With “insulin”, “an insulin” or “the insulin” as used herein is meant human insulin, porcine insulin or bovine insulin with disulfide bridges between CysA7 and CysB7 and between CysA20 and CysB19 and an internal disulfide bridge between CysA6 and CysA11 or an insulin analogue or derivative thereof.

The term “human insulin” as used herein means the human insulin hormone in which the two dimensional and three dimensional structures and properties are well-known. The three dimensional structure of human insulin has been e.g. determined by NMR and X-ray crystallography under many different conditions and many of these structures are deposited in the Protein data bank (http://www.rcsb.org). Non-limiting examples of a human insulin structure is the T6 structure (http://www.rcsb.org/pdb/explore.do?structureId=1MSO) and the R6 structure (http://www.rcsb.org/pdb/explore.do?structureId=1EV3). Human insulin has two polypeptide chains, named the A-chain and the B-chain. The A-chain is a 21 amino acid peptide and the B-chain is a 30 amino acid peptide, the two chains being connected by disulfide bonds: a first bridge between the cysteine in position 7 of the A-chain and the cysteine in position 7 of the B-chain, and a second bridge between the cysteine in position 20 of the A-chain and the cysteine in position 19 of the B-chain. A third bridge is present between the cysteines in position 6 and 11 of the A-chain. Thus “an protease stabilised insulin where the three disulfide bonds of human insulin are retained” is herein understood as an protease stabilised insulin comprising the three disulfide bonds of human insulin, i.e. a disulfide bond between the cysteine in position 7 of the A-chain and the cysteine in position 7 of the B-chain, a disulfide bond between the cysteine in position 20 of the A-chain and the cysteine in position 19 of the B-chain and a disulfide bond between the cysteines in position 6 and 11 of the A-chain.

In the human body, the insulin hormone is synthesized as a single-chain precursor proinsulin (preproinsulin) consisting of a prepeptide of 24 amino acids followed by proinsulin containing 86 amino acids in the configuration: prepeptide-B-Arg Arg-C-Lys Arg-A, in which C is a connecting peptide of 31 amino acids. Arg-Arg and Lys-Arg are cleavage sites for cleavage of the connecting peptide from the A and B chains.

As used in this specification and appended embodiments, the singular forms “a”, “an” and “the” include plural referents unless the content clearly dictates otherwise. Thus, for example, reference to “an insulin” includes a protease stabilised insulins and a mixture of one or more protease stabilised insulins, and the like.

The term “insulin peptide” as used herein means a peptide which is either human insulin or an analogue or a derivative thereof with insulin activity.

The term “insulin analogue” as used herein means a modified insulin wherein one or more amino acid residues of the insulin have been substituted by other amino acid residues and/or wherein one or more amino acid residues have been deleted from the insulin and/or wherein one or more amino acid residues have been added and/or inserted to the insulin. An insulin analogue as used herein is a polypeptide which has a molecular structure which formally may be derived from the structure of a naturally occurring insulin, for example that of human insulin, by deleting and/or substituting at least one amino acid residue occurring in the natural insulin and/or by adding at least one amino acid residue.

In one embodiment an protease stabilised insulin according to the invention is an insulin analogue (as defined above) containing one or more additional disulfide bond(s) relative to human insulin and containing a side chain attached to the epsilon amino group of a lysine residue present in the B-chain of the molecule In one embodiment an insulin analogue according to the invention comprises less than 8 modifications (substitutions, deletions, additions) relative to human insulin. In one embodiment an insulin analogue comprises less than 7 modifications (substitutions, deletions, additions) relative to human insulin. In one embodiment an insulin analogue comprises less than 6 modifications (substitutions, deletions, additions) relative to human insulin. In one embodiment an insulin analogue comprises less than 5 modifications (substitutions, deletions, additions) relative to human insulin. In one embodiment an insulin analogue comprises less than 4 modifications (substitutions, deletions, additions) relative to human insulin. In one embodiment an insulin analogue comprises less than 3 modifications (substitutions, deletions, additions) relative to human insulin. In one embodiment an insulin analogue comprises less than 2 modifications (substitutions, deletions, additions) relative to human insulin.

A derivative of insulin or an “insulin derivative” according to the invention is a naturally occurring human insulin or an insulin analogue which has been chemically modified, e.g. by introducing a side chain in one or more positions of the insulin backbone or by oxidizing or reducing groups of the amino acid residues in the insulin or by converting a free carboxylic group to an ester group or to an amide group. Other derivatives are obtained by acylating a free amino group or a hydroxy group, such as in the B29 position of human insulin or desB30 human insulin. Non-limiting examples of such side chains may be found in the form of attachment of amides, carbohydrates, alkyl groups, acyl groups, esters, PEGylations, and the like. A derivative of insulin is thus human insulin or an insulin analogue which comprises at least one covalent modification such as a side-chain attached to one or more amino acids of the insulin peptide.

When used herein the term “additional disulfide bonds” or “additional disulfide bridge” are used as synonyms and mean one or more disulfide bonds which are not present in human insulin or insulin analogues comprising the same disulfide bonds (also known as bridges) as human insulin, i.e. meaning additional disulfide bonds/bridges relative to human insulin or analogues comprising the same disulfide bonds/bridges as human insulin.

The term “protease stabilised insulin without one or more additional disulfide bonds” as used herein is intended to mean an protease stabilised insulin having the three disulfide bonds naturally present in human insulin, i.e. a first bridge between the cysteine in position 7 of the A-chain and the cysteine in position 7 of the B-chain, a second bridge between the cysteine in position 20 of the A-chain and the cysteine in position 19 of the B-chain and a third bridge between the cysteines in position 6 and 11 of the A-chain, and a side chain attached to the insulin but no further disulfide bonds/bridges

The term “side chain” is used herein and is intended to mean a fatty acid or diacid (optionally via one or more linkers) coupled to the parent insulin of the invention, such as to the epsilon amino group of a lysine present in the B-chain of the parent insulin. The fatty acid or diacid part of the side chain is conferring affinity to serum albumin, and the linkers act either to modify (e.g. increase) the affinity for albumin, modify solubility of the insulin derivative, and/or modulate (increase/decrease) the affinity of the insulin derivative for the insulin receptor.

With the term “cysteine substitution” is herein meant replacing an amino acid which is present in human insulin with a cysteine. For example, isoleucine in position 10 in the A chain (IleA10) and glutamine in position 4 of the B chain of human insulin (GlnB4) may each be replaced by a cysteine residue. With the term “other amino acid residue substitution” is herein meant replacing an amino acid which is present in human insulin with an amino acid which is not cysteine.

A “lipophilic substituent” or “lipophilic residue” is herein understood as a side chain consisting of a fatty acid or a fatty diacid attached to the insulin, optionally via a linker, in an amino acid position such as LysB29, or equivalent. In one embodiment, the lipophilic substituent attached to the insulin has the general formula CHEM 3 as defined elsewhere herein.

With the term “oral bioavailability” is herein meant the fraction of the administered dose of drug that reaches the systemic circulation after having been administered orally. By definition, when a medication is administered intravenously, its bioavailability is 100%.

Generally, the term bioavailability refers to the fraction of an administered dose of the active pharmaceutical ingredient (API, i.e the protease stabilised insulin), such as a derivative of the invention that reaches the systemic circulation unchanged. By definition, when an API is administered intravenously, its bioavailability is 100%. However, when it is administered via other routes (such as orally), its bioavailability decreases (due to incomplete absorption and first-pass metabolism). Knowledge about bioavailability is essential when calculating dosages for non-intravenous routes of administration.

Absolute oral bioavailability compares the bioavailability (estimated as the area under the curve, or AUC) of the API in systemic circulation following oral administration, with the bioavailability of the same API following intravenous administration. It is the fraction of the API absorbed through non-intravenous administration compared with the corresponding intravenous administration of the same API. The comparison must be dose normalised if different doses are used; consequently, each AUC is corrected by dividing the corresponding dose administered.

A plasma API concentration vs. time plot is made after both oral and intravenous administration. The absolute bioavailability (F) is the dose-corrected AUC-oral divided by AUC-intravenous.

Standard assays for measuring insulin bioavailability are known to the person skilled in the art and include inter a/ia measurement of the relative areas under the curve (AUC) for the concentration of the insulin in question administered orally and intra venously (i.v.) in the same species. Quantitation of insulin concentrations in blood (plasma) samples may be done using for example antibody assays (ELISA) or by mass spectrometry.

However, when a drug is administered orally the bioavailability of the active ingredient (i.e. protease stabilised insulin) decreases due to incomplete absorption and first-pass metabolism. The biological activity of an insulin peptide may be measured in an assay as known by a person skilled in the art as e.g. described in WO 2005012347.

The term “preservative” as used herein refers to a chemical compound which is added to a pharmaceutical composition to prevent or delay microbial activity (growth and metabolism). Examples of pharmaceutically acceptable preservatives are phenol, m-cresol and a mixture of phenol and m-cresol.

The term “polypeptide” and “peptide” as used herein means a compound composed of at least two constituent amino acids connected by peptide bonds. The constituent amino acids may be from the group of the amino acids encoded by the genetic code and they may be natural amino acids which are not encoded by the genetic code, as well as synthetic amino acids. Commonly known natural amino acids which are not encoded by the genetic code are e.g., γ-carboxyglutannate, ornithine, phosphoserine, D-alanine and D-glutamine. Commonly known synthetic amino acids comprise amino acids manufactured by chemical synthesis, i.e. D-isomers of the amino acids encoded by the genetic code such as D-alanine and D-leucine, Aib (a-aminoisobutyric acid), Abu (a-aminobutyric acid), Tle (tert-butylglycine), β-alanine, β-aminomethyl benzoic acid, anthranilic acid.

The term “Protein” as used herein means a biochemical compound consisting of one or more polypeptides.

The term “drug”, “therapeutic”, “medicament” or “medicine” when used herein refer to an active ingredient such as e.g. a protease stabilised insulin used in a pharmaceutical composition.

The term “enteric coating” as used herein means a polymer coating that controls disintegration and release of the solid oral dosage form. The site of disintegration and release of the solid dosage form may be customized depending on the enteric coating ability to resist dissolution in a specific pH range.

The term “PK/PD profile” as used herein means pharmacokinetic/pharmacodynamic profile and is known to the person skilled in the art. The pharmacokinetic (PK) profile of an acylated insulin of a pharmecutical composition of the present invention may suitably be determined by in vivo PK studies. These studies are performed in order to evaluate how the acylated insulin is absorbed, distributed and eliminated from the body and how these processes affected the plasma concentration-time profile of the acylated insulin. In discovery and preclinical phase of drug development numerous methods and animal models may be utilized to understand the PK properties for the acylated insulin. For example, the beagle dog may be used to evaluate the PK properties of an acylated insulin in a pharmaceutical composition of the invention following oral administration.

Standard assays for measuring insulin pharmacokinetics are known to the person skilled in the art and include inter a/ia measurement of the concentration of the insulin in question administered orally and intra venously (i.v.) in the same species. Quantitation of insulin concentrations in blood (plasma) samples may be done using for example antibody assays (ELISA) or by mass spectrometry.

Similarly, the pharmacodynamic (PD) profile of an acylated insulin of a pharmecutical composition of the present invention may suitably be determined by the study of the biochemical and physiological effects of said acylated insulin on the body and the mechanisms of drug action and the relationship between drug concentration and effect.

The term “Tmax” as used herein means the time after administration of a drug when the maximum plasma concentration is reached (i.e. Cmax).

The term “Cmax” as used herein means the peak plasma concentration of a drug, i.e. insulin.

Herein, the term “fatty acid” covers a linear or branched, aliphatic carboxylic acids having at least two carbon atoms and being saturated or unsaturated. The term “fatty acid” as used herein does also include the term “fatty diacid” as defined below. Non limiting examples of fatty acids are myristic acid, palmitic acid, and stearic acid.

Herein, the term “fatty diacid” covers a linear or branched, aliphatic dicarboxylic acids having at least two carbon atoms and being saturated or unsaturated. Non limiting examples of fatty diacids are hexanedioic acid, octanedioic acid, decanedioic acid, dodecanedioic acid, tetradecanedioic acid, hexadecanedioic acid, heptadecanedioic acid, octadecanedioic acid, and eicosanedioic acid.

The term “medium-chain fatty acid” is herein used to mean a fatty acid having a medium length carbon chain such as e.g. carbon chains with between 6 to 12 carbon atoms. Non limiting examples of medium-chain fatty acids include hexanoic acid, octanoic acid, decanoic acid and dodecanoic acid.

Herein, the term “dispersion” means a dispersion, an emulsion or a system consisting of two non-miscible components.

The term “disintegration” or “disintegrated” as used herein and when referring to a coating, is to be understood as said coating being disintegrated into components, wherein some or all of the components are completely dissolved into the medium triggering said disintegration.

Herein, the term “dissolution” means the process of dissolving a solid substance into a solvent to make a solution.

The term “protease” or a “protease enzyme” as used herein refers to enzymes is a digestive enzyme which degrades proteins and peptides and which is found in various tissues of the human body such as e.g. the stomach (pepsin), the intestinal lumen (chymotrypsin, trypsin, elastase, carboxypeptidases, etc.) or mucosal surfaces of the GI tract (aminopeptidases, carboxypeptidases, enteropeptidases, dipeptidyl peptidases, endopeptidases, etc.), the liver (Insulin degrading enzyme, cathepsin D etc), and in other tissues.

Herein, the term “protease stabilised insulin” means the insulin analogue or derivative having an improved stability against degradation from proteases relative to human insulin. Some proetase stabilised insulins are disclosed in WO2009/115469, as are their protease stabilised properties. Thus these acylated protease stabilised insulins displays higher apparent potency and/or bioavailability than similar known acylated insulins that are not stabilised towards proteolytic degradation. More specifically, the protease stabilised insulin is an insulin molecule having two or more mutations of the A and/or B chain relative to the parent insulin. Surprisingly, it has been found that by substituting two or more hydrophobic amino acids within or in close proximity to two or more protease sites on an insulin with hydrophilic amino acids, an insulin analogue (i.e., a protease stabilised insulin) is obtained which is proteolytically more stable compared to the parent insulin. In a broad aspect, a protease stabilised insulin is an insulin analogue wherein at least two hydrophobic amino acids have been substituted with hydrophilic amino acids relative to the parent insulin, wherein the substitutions are within or in close proximity to two or more protease cleavage sites of the parent insulin and wherein such insulin analogue optionally further comprises one or more additional mutations.

Herein the term “immediate release coating” is used as the term is known to the person skilled in the art. Thus this term discloses coatings that are released immediately when contacted with any solution, being pH independent, including prime coating systems.

The term “about” as used herein means in reasonable vicinity of the stated numerical value, such as plus or minus 10%. The terms “mainly” and “majority” as used herein is a quantification to indicate; a part, area, size, and frequency that is greater than 50% including about 60%, 70%, 80%, 90% or more relative to the context that it refers to.

The term “stability” is herein used for a pharmaceutical composition comprising modified insulin to describe the shelf life of the composition. The term “stabilised” or “stable” when referring to a protease stabilised insulin thus refers to a composition with increased chemical stability or increased physical and chemical stability relative to a composition comprising a non-stabilised insulin.

The term “chemical stability” of an insulin as used herein refers to chemical covalent changes in the protein structure leading to formation of chemical degradation products with potential less biological potency and/or potential increased immunogenic properties compared to the native protein structure. Various chemical degradation products may be formed depending on the type and nature of the native protein and the environment to which the protein is exposed. Elimination of chemical degradation can most probably not be completely avoided and increasing amounts of chemical degradation products is often seen during storage and use of the pharmaceutical composition as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation can be mentioned as another variant of chemical degradation. The chemical stability of the protease stabilised insulin can be evaluated by measuring the amount of the chemical degradation products at various time-points after exposure to different environmental conditions (the formation of degradation products may often be accelerated by for instance increasing temperature). The amount of each individual degradation product is often determined by separation of the degradation products depending on molecule size, hydrophilicity, hydrophobicity, and/or charge using various chromatography techniques (e.g. SEC-HPLC and/or RP-HPLC).

Hence, as outlined above, “stabilised” or “stable” when referring to a protease stabilised insulin refers to a pharmaceutical composition comprising an insulin with increased chemical stability or increased physical and chemical stability relative to the corresponding non-modified parent protein. In general, a pharmaceutical composition must be stable during use and storage (in compliance with recommended use and storage conditions) until the expiration date is reached.

The term “direct contact” as used herein refers to the contact between the anionic copoymer coating of the present invention and the tablet core of the present invention. As used herein “direct contact” means that there is no physical barrier between the interface of outer surface of the tablet core and an inner surface of the anionic copolymer coating. Thus when the tablet core according to the present invention is “partly in direct contact” with the anionic copolymer coating according to the present invention, then at least some areas in the interface between the tablet core and the anionic copolymer are free of physical barriers in contrast to other areas of varying size which may comprise any kind of physical barrier. Thus in embodiment of the present invention regards a pharmaceutical composition wherein an anionic copolymer coating is in direct contact with 10% or more of an outer surface of a tablet core, i.e. this means that the anionic copolymer is partly in direct contact with the outer surface of the tablet core or vice versa. When “majority” as used herein is used in the context of “the anionic copolymer coating is at least partly in direct contact the majority of an outer surface of the tablet core” it is meant to indicate that the sum of area of direct contact between an outer surface of the tablet core and an inner surface of the anionic copolymer coating is greater than the sum of area where a physical barrier exists in the interface between these two surfaces. The term “physical barrier” as used herein covers any kind of physical barrier which diminishes or influences the physical contact between an outer surface of the tablet core and an inner surface of the anionic copolymer coating. Thus in a composition according to the present invention wherein an anionic copolymer coating is in direct contact with 50% or more of an outer surface of a tablet core, the anionic copolymer is in direct contact with the majority of outer surface of the tablet core or vice versa.

When used in formulations “mucoadhesive” properties may be introduced to a formulation by use of various polymeric compounds. Typically poly-anions e.g. poly-acrylic acids exert this property. The mucoadhesive property is inherently dependent on the interpenetration of the polymeric compounds both in the bio-mucosa and the formulation. In this way a physical bridge is made possible due to the large size of the polymer molecules. Low molecular weight compounds e.g. sodium caprate or sorbitol will therefore, not exert mucoadhesive properties. Molecules considered “non-mucoadhesive” are molecules with a molecular weight of below 1000 g/mol. We hereby include that molecules with a molecular weight below 900 g/mol, 8008/mol, 7008/mol, 6008/mol, 500 g/mol, 400 g/mol and 300 g/mol are included in this definition of molecules considered non-mucoadhesive in this patent application. The term “anionic copolymer” herein means a coplymer which comprises functional groups which are able to dissociate to attain a negative charge. A non limiting example of such functional group is e.g. a functional group having an acidic side chain. The anionic character of a copolymer is observed above specific pH values depending on the copolymer. In the context of this patent pH values from pH 4 to pH 7.4 are defining the pH value above which the copolymer has a negative charge. Thus, an anionic copolymer is herein a copolymer which has a net negative charge in the pH range from about pH 4.0 to pH 7.4.

The term “anionic copolymer coating” as used herein refers to a coating or film coating which comprises at least 80% (w/w) or more anionic copolymer in dry state. In one embodiment the term “anionic copolymer coating” includes a coating based on methyl acrylate, methyl methacrylate and methacrylic acid. In one embodiment the term “anionic copolymer coating” includes a EUDRAGITC®FS30D based coating as produced by Evonik Industries in 2013. In one embodiment the term “anionic copolymer coating” is based on methyl acrylate, methyl methacrylate and methacrylic acid. In one embodiment the term “anionic copolymer coating” includes a coating comprising methyl acrylate, methyl methacrylate and methacrylic acid. In one embodiment the term “anionic copolymer coating” includes an EUDRAGITC®FS30D coating as sold by Evonik Industries (in 2013). In one embodiment the term “anionic copolymer coating” includes an EUDRAGITC®FS30D comprising coating as sold by Evonik Industries (in 2013). The term “anionic copolymer coating” as used herein includes coating comprising at least 80%, at least 90% or about 100% (w/w) anionic copolymer. The term “coating based on anionic copolymer” as used herein refers to a coating which primarily comprises anionic copolymer, i.e. comprises about 80% (w/w) or more anionic copolymer and thus is covered by the term “anionic copolymer coating”.

In one embodiment, the anionic copolymer coating of the invention comprises a compound of CHEM 6:

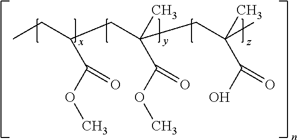

Wherein x=7, y=3, z=1 and n is about 1080. In one embodiment, the coating is Poly(methyl acrylate-co-methyl methacrylate-co-methacrylic acid) 7:3:1. In one embodiment, the coating of the invention has a weight average molar mass which is about 280,000 g/mol.

The term “copolymer coating material” as used herein refers to the material which is purchased or produced, often a dry powder and comprises all components of the anionic copolymer coating. This copolymer coating material is suspended for coating on top of a tablet or tablet core, where the copolymer material can form the anionic copolymer coating.

The term “functional” when referring to a coating is intended to indicate that said coating disintegrates dissolves in aqueous medium at specific pH intervals of said medium and/or time windows.

According to the above, tThe term “non-functional” when referring to a coating is intended to indicate that said coating disintegrates dissolves in aqueous medium regardless of the pH values of said medium. Functionality does herein not relate to changing of physical properties for the composition such as e.g. moisture barrier.

The term “additional separating layer” as used herein refers to any non-functional coating, such as another type of PVA coating or any other coating which is known by the skilled person as a non-functional coating and may also qualify as a sub coat for enteric coatings. A specific example of such a standard separating layer is OPADRY®II from Colocon® (as sold in 2013), which the skilled person in the art appreciates to be a commonly (i.e. standard) used sub coat for enteric coatings in oral formulations.

The term “additional non-functional coating” as used herein refers to any non-functional coating, such as another type of PVA coating or any other coating which is known by the skilled person as a non-functional coating and may also qualify as a sub coat for enteric coatings. A specific example of such a non-functional coating is OPADRY®II from Colocon® (as sold in 2013), which the skilled person in the art appreciates to be a commonly (i.e. standard) used sub coat for enteric coatings in oral formulations.

The term “insulin powder” as used herein refers to the active pharmaceutical ingredient (API, i.e. the protease stabilised insulin), which has been dried and is stored in the form of a powder, in this case the API is insulin, therefore the powder is a “insulin powder”.

The term “sorbitol powder” as used herein refers to any sorbitol or equivalent excipient, such as mannitol, which is dried and stored in the form of a powder.

The Following is a Non-Limiting List of Aspects Further Comprised within the Scope of the Invention:
1. A pharmaceutical composition comprising a tablet core and optionally an anionic copolymer coating, wherein said tablet core comprises a salt of a medium-chain fatty acid and an insulin derivative,
   wherein said insulin derivative comprises one or more an additional disulfide bridges or
   wherein said insulin derivative is an acylated insulin comprising a linker and a fatty acid or fatty diacid side chain having 14-22 carbon atoms and optionally further comprising one or more an additional disulfide bonds and
   wherein
   said anionic copolymer coating is resistant to disintegration at pH below 6.0 and disintegrates at pH above 7.0.
1A. A pharmaceutical composition comprising a tablet core and an anionic copolymer coating, wherein said tablet core comprises a salt of capric acid and a protease stabilised insulin,
   wherein said protease stabilised insulin comprises one or more additional disulfide bridges relative to human insulin or analogues comprising the same disulfide bridges as human insulin, or
   wherein said protease stabilised insulin comprises a linker and a fatty acid or fatty diacid side chain having 14-22 carbon atoms and optionally further comprises one or more additional disulfide bridges
   relative to human insulin or analogues comprising the same disulfide bridges as human insulin, and
   wherein said anionic copolymer coating is a dispersion comprising between 25-35% such as about 30% (meth)acrylate copolymer, wherein said (meth)acrylate copolymer consists of 10-30% (w/w) methyl methacrylate, 50-70% (w/w) methyl acrylate and 5-15% (w/w) methacrylic acid and is at least partly in direct contact with an outer surface of a tablet core.
2. The pharmaceutical composition according to aspect 1 or 1A, wherein said anionic copolymer coating comprises at least 80% anionic copolymer.
3. The pharmaceutical composition according any one of the preceding aspects, wherein said anionic copolymer coating is a coating based on methyl acrylate, methyl methacrylate and methacrylic acid.
3A. The pharmaceutical composition according any one of the preceding aspects, wherein said anionic copolymer coating is a coating comprises methyl acrylate, methyl methacrylate and methacrylic acid.
4. The pharmaceutical composition according to any one of the preceding aspects, wherein said anionic copolymer coating is an EUDRAGITC®FS30D coating as sold by Evonik Industries (in 2013).
5. The pharmaceutical composition according to any one of the preceding aspects, wherein said medium-chain fatty acid is capric acid.
6. The pharmaceutical composition according to any one of the preceding aspects, wherein said salt of a medium-chain fatty acid is sodium caprate.
6A. The pharmaceutical composition according aspect 1A, wherein said salt of capric acid is sodium caprate.
7. The pharmaceutical composition according to any of the preceding aspects, wherein said tablet core further comprises sorbitol, stearic acid and protease stabilised insulin.
8. The pharmaceutical composition according to any of the preceding aspects, wherein all ingredients of said tablet are of a molecular weight below about 300-1000 g/mol.
9. The pharmaceutical composition according to any of the preceding aspects, wherein all ingredients of said tablet are of a molecular weight below 1000 g/mol.
10. The pharmaceutical composition according to any of the preceding aspects, wherein all ingredients of said tablet are of a molecular weight below 800 g/mol.
11. The pharmaceutical composition according to any of the preceding aspects, wherein all ingredients of said tablet are of a molecular weight below 700 g/mol.
12. The pharmaceutical composition according to any of the preceding aspects, wherein all ingredients of said tablet are of a molecular weight below 600 g/mol.
13. The pharmaceutical composition according to any of the preceding aspects, wherein all ingredients of said tablet are of a molecular weight below 500 g/mol.
14. The pharmaceutical composition according to any of the preceding aspects, wherein all ingredients of said tablet are of a molecular weight below 4008/mol.
15. The pharmaceutical composition according to any of the preceding aspects, wherein all ingredients of said tablet are of a molecular weight below 300 g/mol.
16. The pharmaceutical composition according to any of the preceding aspects, wherein said tablet core is not mucoadhesive and/or does not comprise mucoadhesive ingredients.
17. The pharmaceutical composition according to any of the preceding aspects, wherein said tablet core does not adhere to the mucosa.
18. The pharmaceutical composition according to any of the preceding aspects, wherein said tablet core comprises ingredients and excipients with zero water uptake.
19. The pharmaceutical composition according to any of the preceding aspects, wherein said tablet core comprises ingredients and excipients exerting a total water uptake of about 0-9%.
20. The pharmaceutical composition according to any of the preceding aspects, wherein said tablet core comprises ingredients and excipients exerting a total water uptake of below about 10%.
21. The pharmaceutical composition according to any of the preceding aspects, wherein said tablet core comprises ingredients and excipients exerting a total water uptake of about 9%.
22. The pharmaceutical composition according to any of the preceding aspects, wherein said tablet core comprises ingredients and excipients exerting a total water uptake of below about 8%.
23. The pharmaceutical composition according to any one of the preceding aspects wherein said tablet core comprises about 60-85% (w/w) caprate, such as e.g. sodium caprate.
24. The pharmaceutical composition according to any one of the preceding aspects wherein said tablet core comprises about 60% (w/w) caprate, such as e.g. sodium caprate.
25. The pharmaceutical composition according to any one of the preceding aspects wherein said tablet core comprises about 70-80% (w/w) caprate, such as e.g. sodium caprate.
26. The pharmaceutical composition according to any one of the preceding aspects wherein said tablet core comprises about 75% (w/w) caprate, such as e.g. sodium caprate.
27. The pharmaceutical composition according to any one of the preceding aspects wherein said tablet core comprises about 75-80% (w/w) caprate, such as e.g. sodium caprate.
28. The pharmaceutical composition according to any one of the preceding aspects wherein said tablet core comprises about 77% (w/w) caprate, such as e.g. sodium caprate.
29. The pharmaceutical composition according to any one of the preceding aspects wherein said tablet core comprises about 80% (w/w) caprate, such as e.g. sodium caprate.
30. The pharmaceutical composition according to any one of the preceding aspects wherein said tablet core comprises about 85% (w/w) caprate, such as e.g. sodium caprate.
31. The pharmaceutical composition according to any one of the preceding aspects wherein said tablet core comprises about 77% (w/w) caprate, such as e.g. sodium caprate, about 22.5 minus X % (w/w) sorbitol, about X % (w/w) insulin and about 0.5% (w/w) stearic acid, wherein X is selected from the group consisting of: 0.1, 0.5, 1, 1.5, 2, 2.5, 3, 3.5, 4, 4.5 or 5.
32. The pharmaceutical composition according to any one of the preceding aspects wherein said tablet core comprises about 77% (w/w) caprate, such as e.g. sodium caprate, about 22.5 minus X % (w/w) sorbitol, about X % (w/w) insulin and about 0.5% (w/w) stearic acid, wherein X is selected from the group consisting of: 5.5, 6, 6.5, 7, 7.5, 8, 8.5, 9, 9.5 or 10.
33. The pharmaceutical composition according to any one of the preceding aspects wherein said tablet core comprises about 77% (w/w) caprate, such as e.g. sodium caprate, about 22.5 minus X % (w/w) sorbitol, about X % (w/w) insulin and about 0.5% (w/w) stearic acid, wherein X is selected from the group consisting of: 10.5, 11, 11.5, 12, 12.5, 13, 13.5, 14, 14.5 or 15.
34. The pharmaceutical composition according to any one of the preceding aspects wherein said tablet core comprises about 77% (w/w) caprate, such as e.g. sodium caprate, about 22.5 minus X % (w/w) sorbitol, about X % (w/w) insulin and about 0.5% (w/w) stearic acid, wherein X is selected from the group consisting of: 15.5, 16, 16.5, 17, 17.5, 18, 18.5, 19, 19.5, 20, 20.5, 21 or 21.5.
35. The pharmaceutical composition according to any one of the preceding aspects, wherein said anionic copolymer which is in direct contact with an outer surface of said tablet core is in direct contact with at about 100% of said outer surface of said tablet core.
35A. The pharmaceutical composition according to any one of the preceding aspects, wherein said anionic copolymer is in direct contact with at about 100% of said outer surface of said tablet core.
36. The pharmaceutical composition according to any one of the preceding aspects, wherein said anionic copolymer is in direct contact with at about 99% of said outer surface of said tablet core.
37. The pharmaceutical composition according to any one of the preceding aspects, wherein said anionic copolymer is in direct contact with at about 90% of said outer surface of said tablet core.
38. The pharmaceutical composition according to any one of the preceding aspects, wherein said anionic copolymer is in direct contact with at about 80% of said outer surface of said tablet core
39. The pharmaceutical composition according to any one of the preceding aspects, wherein said anionic copolymer is in direct contact with at about 70% of said outer surface of said tablet core.
40. The pharmaceutical composition according to any one of the preceding aspects, wherein said anionic copolymer is in direct contact with at about 60% of said outer surface of said tablet core.
41. The pharmaceutical composition according to any one of the preceding aspects, wherein said anionic copolymer is in direct contact with at about 50% of said outer surface of said tablet core.
42. The pharmaceutical composition according to any one of the preceding aspects, wherein said anionic copolymer is in direct contact with at about 40% of said outer surface of said tablet core.
43. The pharmaceutical composition according to any one of the preceding aspects, wherein said anionic copolymer is in direct contact with at about 30% of said outer surface of said tablet core.
44. The pharmaceutical composition according to any one of the preceding aspects, wherein said anionic copolymer is in direct contact with at about 20% of said outer surface of said tablet core.
45. The pharmaceutical composition according to any one of the preceding aspects, wherein said anionic copolymer is in direct contact with at about 10% of said outer surface of said tablet core.
46. The pharmaceutical composition according to any one of the preceding aspects, wherein said anionic copolymer coating is present in at amount of about 4-10% (w/w) relative to the tablet core.
47. The pharmaceutical composition according to any one of the preceding aspects, wherein said anionic copolymer coating is present in at amount of about 4% (w/w) relative to the tablet core.
48. The pharmaceutical composition according to any one of the preceding aspects, wherein said anionic copolymer coating is present in at amount of about 5% (w/w) relative to the tablet core.
49. The pharmaceutical composition according to any one of the preceding aspects, wherein said anionic copolymer coating is present in at amount of about 6% (w/w) relative to the tablet core.
50. The pharmaceutical composition according to any one of the preceding aspects, wherein said anionic copolymer coating is present in at amount of about 6.5% (w/w) relative to the tablet core.
51. The pharmaceutical composition according to any one of the preceding aspects, wherein said anionic copolymer coating is present in at amount of about 7% (w/w) relative to the tablet core.
52. The pharmaceutical composition according to any one of the preceding aspects, wherein said anionic copolymer coating is present in at amount of about 7.5% (w/w) relative to the tablet core.
53. The pharmaceutical composition according to any one of the preceding aspects, wherein said anionic copolymer coating is present in at amount of about 8% (w/w) relative to the tablet core.
54. The pharmaceutical composition according to any one of the preceding aspects, wherein said anionic copolymer coating is present in at amount of about 9% (w/w) relative to the tablet core.
55. The pharmaceutical composition according to any one of the preceding aspects, wherein said anionic copolymer coating is present in at amount of about 10% (w/w) relative to the tablet core.
56. The pharmaceutical composition according to any one of the preceding aspects, wherein an additional non-functional coating is applied on top of said anionic copolymer coating.
57. The pharmaceutical composition according to any one of the preceding aspects, wherein an additional continuous non-functional coating is applied on top of said anionic copolymer coating.
58. The pharmaceutical composition according to any one of the preceding aspects, wherein an additional discontinuous non-functional coating is applied on top of said anionic copolymer coating.
59. The pharmaceutical composition according to any one of the preceding aspects, wherein an additional dis continuous non-functional coating is applied between said tablet core and said anionic copolymer coating.
60. The pharmaceutical composition according to any one of the preceding aspects, wherein said composition does not comprise a continuous sub coat between said tablet core and said anionic copolymer.
61. The pharmaceutical composition according to any one of the preceding aspects, wherein said anioinc copolymer coating dissolves at a pH between about 6.5-7.2.
62. The pharmaceutical composition according to any one of the preceding aspects, wherein said anioinc copolymer coating dissolves at a pH between about 6.5-7.2 and does not dissolve below about pH 5.5.
63. The pharmaceutical composition according to any one of the preceding aspects, wherein said anioinc copolymer coating does not dissolve below about pH 5.5-6.5.
64. The pharmaceutical composition according to any one of the preceding aspects, wherein said anioinc copolymer coating does not dissolves below about pH 5.5.
65. The pharmaceutical composition according to any one of the preceding aspects, wherein said anioinc copolymer coating does not dissolves below about pH 6.0.
66. The pharmaceutical composition according to any one of the preceding aspects, wherein said anioinc copolymer coating does not dissolve below about pH 6.5.
67. The pharmaceutical composition according to the present invention wherein the composition shows a Tmax in a Beagle dog of between about 120-160 minutes, after oral administration.
68. The pharmaceutical composition according to the present invention wherein the composition shows a Tmax in a Beagle dog with an empty stomach of between about 120-160 minutes, after oral administration.
69. The pharmaceutical composition according to any of the preceding aspects, wherein said composition is administered orally.
70. The pharmaceutical composition according to any one of the preceding aspects in the form of a tablet.
71. The pharmaceutical composition according to any one of the preceding aspects in the form of a multi-particulate system
72. The pharmaceutical composition according to any one of the preceding aspects in the form of a multi-particulate system, wherein said particles in said system are individually or collectively coated with said anionic copolymer coating.
73. The pharmaceutical composition according to any one of the preceding aspects in the form of a pellet.
74. The pharmaceutical composition according to any one of the preceding aspects in the form of a uniform tablet, a single or multilayered tablet, a multiparticulate system, a capsule, a tablet contained in a capsule, multiple tablets contained in a capsule, multiple tablets contained in a tablet, a multiparticulate system in the form of a tablet contained in a capsule or in a form of multiparticulate system compressed in one, some or all layers of said tablet core.
75. The pharmaceutical composition according to any one of the preceding aspects, wherein said protease stabilised insulin comprises a linker and a fatty acid or fatty diacid chain having 14 carbon atoms.
76. The pharmaceutical composition according to any one of the preceding aspects, wherein said protease stabilised insulin comprises a linker and a fatty acid or fatty diacid chain having 16 carbon atoms.
77. The pharmaceutical composition according to any one of the preceding aspects, wherein said protease stabilised insulin comprises a linker and a fatty acid or fatty diacid chain having 18 carbon atoms.
78. The pharmaceutical composition according to any one of the preceding aspects, wherein said protease stabilised insulin comprises a linker and a fatty acid or fatty diacid chain having 20 carbon atoms.
79. The pharmaceutical composition according to any one of the preceding aspects, wherein said protease stabilised insulin comprises a linker and a fatty acid or fatty diacid chain having 22 carbon atoms.
80. The pharmaceutical composition according to any one of the preceding aspects wherein said protease stabilised insulin has two or more cysteine substitutions and a side chain attached to the insulin, where the three disulfide bonds of human insulin are retained, and the sites of cysteine substitutions are chosen in such a way that the introduced cysteine residues are placed in the three dimensional structure of the folded protease stabilised insulin to allow for the formation of one or more additional disulfide bonds not present in human insulin.
81. The pharmaceutical composition according to any one of the preceding aspects wherein said protease stabilised insulin has two or more cysteine substitutions and a side chain attached to the insulin, where the three disulfide bonds of human insulin are retained, and the sites of cysteine substitutions are chosen in such a way that the introduced cysteine residues are placed in the three dimensional structure of the folded protease stabilised insulin to allow for the formation of one or more additional disulfide bonds not present in human insulin, wherein said said chain comprises a linker and a fatty acid or fatty diacid chain having 14-22 carbon atoms.
82. The pharmaceutical composition according to any one of the preceding aspects wherein said protease stabilised insulin has two or more cysteine substitutions and a side chain attached to the insulin, where the three disulfide bonds of human insulin are retained, and the sites of cysteine substitutions are chosen in such a way that the introduced cysteine residues are placed in the three dimensional structure of the folded protease stabilised insulin to allow for the formation of one or more additional disulfide bonds not present in human insulin, wherein said said chain comprises a linker and a fatty acid or fatty diacid chain having 14 carbon atoms.
83. The pharmaceutical composition according to any one of the preceding aspects wherein said protease stabilised insulin has two or more cysteine substitutions and a side chain attached to the insulin, where the three disulfide bonds of human insulin are retained, and the sites of cysteine substitutions are chosen in such a way that the introduced cysteine residues are placed in the three dimensional structure of the folded protease stabilised insulin to allow for the formation of one or more additional disulfide bonds not present in human insulin, wherein said said chain comprises a linker and a fatty acid or fatty diacid chain having 16 carbon atoms.
84. The pharmaceutical composition according to any one of the preceding aspects wherein said protease stabilised insulin has two or more cysteine substitutions and a side chain attached to the insulin, where the three disulfide bonds of human insulin are retained, and the sites of cysteine substitutions are chosen in such a way that the introduced cysteine residues are placed in the three dimensional structure of the folded protease stabilised insulin to allow for the formation of one or more additional disulfide bonds not present in human insulin, wherein said said chain comprises a linker and a fatty acid or fatty diacid chain having 18 carbon atoms.
85. The pharmaceutical composition according to any one of the preceding aspects wherein said protease stabilised insulin has two or more cysteine substitutions and a side chain attached to the insulin, where the three disulfide bonds of human insulin are retained, and the sites of cysteine substitutions are chosen in such a way that the introduced cysteine residues are placed in the three dimensional structure of the folded protease stabilised insulin to allow for the formation of one or more additional disulfide bonds not present in human insulin, wherein said said chain comprises a linker and a fatty acid or fatty diacid chain having 20 carbon atoms.
86. The pharmaceutical composition according to any one of the preceding aspects wherein said protease stabilised insulin has two or more cysteine substitutions and a side chain attached to the insulin, where the three disulfide bonds of human insulin are retained, and the sites of cysteine substitutions are chosen in such a way that the introduced cysteine residues are placed in the three dimensional structure of the folded protease stabilised insulin to allow for the formation of one or more additional disulfide bonds not present in human insulin, wherein said said chain comprises a linker and a fatty acid or fatty diacid chain having 22 carbon atoms.
87. The pharmaceutical composition according to any one of the preceding aspects wherein the sites of cysteine substitutions are chosen in such a way that
(1) the introduced cysteine residues are placed in the three dimensional structure of the folded protease stabilised insulin to allow for the formation of one or more additional disulfide bonds not present in human insulin, and
(2) the human protease stabilised insulin retains the desired biological activities associated with human insulin.
88. The pharmaceutical composition according to any one of the preceding aspects wherein the sites of cysteine substitutions are chosen in such a way that
(1) the introduced cysteine residues are placed in the three dimensional structure of the folded protease stabilised insulin to allow for the formation of one or more additional disulfide bonds not present in human insulin,
(2) the human protease stabilised insulin retains the desired biological activities associated with human insulin, and
(3) the human protease stabilised insulin has increased physical stability relative to human insulin and/or parent insulin
89. The pharmaceutical composition according to any one of the preceding aspects wherein the sites of cysteine substitutions are chosen in such a way that
(1) the introduced cysteine residues are placed in the three dimensional structure of the folded protease stabilised insulin to allow for the formation of one or more additional disulfide bonds not present in human insulin,
(2) the human protease stabilised insulin retains the desired biological activities associated with human insulin, and
(3) the human protease stabilised insulin is stabilised against proteolytic degradation.
90. The pharmaceutical composition according to any one of the preceding aspects wherein the amino acid residue in position A10 of the A-chain is substituted with a cysteine, the amino acid residue in a position selected from the group consisting of B1, B2, B3 and B4 of the B-chain is substi-tuted with a cysteine, and optionally the amino acid in position B30 is deleted.
91. The pharmaceutical composition according to any one of the preceding aspects wherein one or more additional disulfide bonds are obtained between the A-chain and the B-chain
92. The pharmaceutical composition according to any one of the preceding aspects wherein said protease stabilised insulin comprises on or more additional disulfide bonds and has a more pro-tracted profile than an protease stabilised insulin without one or more additional disulfide bonds.
93. The pharmaceutical composition according to any one of the preceding aspects wherein said side chain is attached to the N-terminal of the insulin or the epsilon amino group of a lysine residue in the insulin.
94. The pharmaceutical composition according to any one of the preceding aspects, wherein said anionic copolymer is an anionic (meth)acrylate copolymer.
95. The pharmaceutical composition according to any one of the preceding aspects, wherein said anionic copolymer is a dispersion comprising between 25-35%, such as 30%, (meth)acrylate copolymer.
96. The pharmaceutical composition according to aspect 95, wherein said (meth)acrylate copolymer consists of 10-30% (w/w) methyl methacrylate, 50-70% (w/w) methyl acrylate and 5-15% (w/w) methacrylic acid.
97. The pharmaceutical composition according to any one of the preceding aspects, wherein said (meth)acrylate copolymer consists of about 25% (w/w) methyl methacrylate, about 65% (w/w) methyl acrylate and about 10% (w/w) methacrylic acid.
98. The pharmaceutical composition according to any one of the preceding aspects, wherein said anionic copolymer comprises a compound of formula CHEM 6:

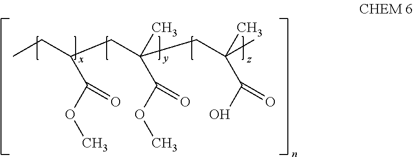

wherein x=7, y=3, z=1 and n is about 1080.
99. The pharmaceutical composition according to any one of the preceding aspects, wherein said anionic copolymer is poly(methyl acrylate-co-methyl methacrylate-co-methacrylic acid) 7:3:1.
100. The pharmaceutical composition according to any one of the preceding aspects, wherein said anionic copolymer has a weight average molar mass which is about 280,000 g/mol
101. The pharmaceutical composition according to any one of the preceding aspects, wherein said anionic copolymer is not bioadhesive.
102. The pharmaceutical composition according to any one of the preceding aspects, wherein said anionic copolymer is not mucoadhesive.
103. The pharmaceutical composition according to any one of the preceding aspects, wherein said protease stabilised insulin comprises a Glutamine in position A14, i.e. comprises the amino acid A14Glu.
104. The pharmaceutical composition according to any one of the preceding aspects, wherein said protease stabilised insulin comprises a Histidine in position B25, i.e. comprises the amino acid B25His.
105. The pharmaceutical composition according to any one of the preceding aspects, wherein said protease stabilised insulin comprises a Histidine in position B16, i.e. comprises the amino acid B16His.
106. The pharmaceutical composition according to any one of the preceding aspects, wherein the amino acid in position B27 of said protease stabilised insulin is deleted, i.e. said protease stabilised insulin comprises desB27.
107. The pharmaceutical composition according to any one of the preceding aspects, wherein the amino acid in position B30 of said protease stabilised insulin is deleted, i.e. said protease stabilised insulin comprises desB30.
108. The pharmaceutical composition according to any one of the preceding aspects, wherein said protease stabilised insulin is selected from the group consisting of:
A14E,B25H,B29K(N^{ε}-Hexadecandioyl),desB30 human insulin,
A14E,B25H,B29K(N^{ε}Octadecandioyl-γGlu),desB30 human insulin,
A14E,B25H,B29K(N^{ε}Eicosanedioyl-γGlu),desB30 human insulin,
A14E,B25H,B29K(N^{ε}3-Carboxy-5-octadecanedioylaminobenzoyl),desB30 human insulin,
A14E,B25H,B29K(N^{ε}-N-octadecandioyl-N-(2-carboxyethyl)glycyl),desB30 human insulin
A14E,B25H,B29K(N^{ε}(N-Octadecandioyl-N-carboxymethyl)-beta-alanyl),desB30 human insulin,
A14E,B25H,B29K(N^{ε}4-([4-({19-Carboxynonadecanoylamino}methyl)trans-cyclo-hexanecarbonyl]-γGlu),desB30 human insulin,
A14E,B25H,B29K(N^{ε}Heptadecanedioyl-γGlu),desB30 human insulin,
A14E,B25H,B29K(N^{ε}Octadecanedioyl-γGlu-OEG-OEG),desB30 human insulin,
A14E,B25H,B29K(N^{ε}Myristyl),desB30 human insulin,
A14E,B25H,B29K(N^{ε}Eicosanedioyl-γGlu-γGlu),desB30 human insulin,
A14E,B25H,B29K(N^{ε}4-([4-({19-Carboxynonadecanoylamino}methyl)trans-cyclo-hexanecarbonyl]-γGlu-γGlubdesB30 human insulin,
A14E,B25H,B29K(N^{ε}Octadecanedioyl-γGlu-γGlu),desB30 human insulin,
A14E,B28D,B29K(N^{ε}octadecandioyl-γGlu),desB30 human insulin,
A14E,B25H,B29K(N^{ε}octadecandioyl-γGlu-PEG7),desB30 human insulin,
A14E,B25H,B29K(N^{ε}eicosanedioyl-γGlu-OEG-OEG), desB30 human insulin,
A14E,B25H,B29K(N^{ε}eicosanedioyl-γGlu-(3-(2-{2-[2-(2-aminoethoxy)ethoxy]ethoxy}ethoxy)propionyl-γGlubdesB30 human insulin,
A14E,B25H,B29K(N^{ε}Hexadecanedioyl-γGlu-OEG-OEG),desB30 human insulin,
A14E,B25H,B29K(N^{ε}Hexadecanedioyl-γGlu),desB30 human insulin,
A14E,B25H,B29K(N^{ε}heptadecanedioyl-γGlu-OEG-OEG),desB30 human insulin,
A14E,B25H,B29K(N^{ε}octadecanedioyl-γGlu-γGlu-γGlu-γGlu),desB30 human insulin,
A14E,B25H,B29K(N^{ε}Eicosanedioyl-γGlu-γGlu-γGlu),desB30 human insulin,
A14E,B25H,B27E,B29K(N^{ε}Octadecanedioyl-γGlu-OEG-OEG),desB30 human insulin,
A14E,B25H,B26G,B27G,B28G,B29K(N^{ε}Octadecanedioyl-γGlu-OEG-OEG),desB30 human insulin,
A14E,B16H,B25H,B29K(N^{ε}Octadecanedioyl-Glu-OEG-OEG),desB30 human insulin,
A14E,B16E,B25H,B29K(N^{ε}Octadecanedioyl-Glu-OEG-OEG),desB30 human insulin,
A14E,B16H,B25H,B29K(N^{ε}Hexadecanedioyl-γGlu),desB30 human insulin,
A14E,B25H,B29K(N^{ε}Eicosanedioyl-γGlu-OEG-Glu),desB30 human insulin,
A14E,B16E,B25H,B29K(N^{ε}HexadecandioyHGlu),desB30 human insulin,
A14E,B16H,B25H,B29K(N^{ε}Octadecanedioyl-γGlu-γGlu-γGlu),desB30 human insulin,
A14E,B25H,B26G,B27G,B28G,B29K(N^{ε}Hexadecandioyl-γGlu),desB30 human insulin,
A14E,B16H,B25H,B29K(N^{ε}Octadecanedioyl-γGlu-γGlu),desB30 human insulin,
A14E,B16H,B25H,B29K(N^{ε}Eicosanedioyl-γGlu-OEG-OEG),desB30 human insulin,
A14E,B25H,B29K(N^{ε}Octadecanedioyl-OEG-Glu-Glu),desB30 human insulin,
A14E,A18L,B25H,B29K(N^{ε}Eicosanedioyl-γGlu-OEG-OEG),desB30 human insulin,
A14E,A18L,B25H,B29K(N^{ε}Octadecanedioyl-γGlu-OEG-OEG),desB30 human insulin,
A14E,B25H,B27E,B29K(N^{ε}Eicosanedioyl-γGlu-OEG-OEG),desB30 human insulin,
A1G(N^{α}Octadecandioyl-γGlu-OEG-OEG),A14E,B25H,B29R,desB30 human insulin,
A14E,B1F(N^{α}Octadecandioyl-γGlu-OEG-OEG),B25H,B29R,desB30 human insulin,
A1G(N^{α}HexadecandioyHGlu),A14E,B25H,B29R,desB30 human insulin,
A14E,B25H,B29K(N^{ε}Octadecanedioyl-Glu-Abu-Abu-Abu-Abu),desB30 human insulin,
A14E,B25H,B29K(N^{α}Eicosanedioyl),desB30 human insulin,
A14E,B25H,B29K(Na4-[16-(1H-Tetrazol-5-yl)hexadecanoylsulfarnoyl]butanoyl), desB30 human insulin,
A1G(N^{α}Octadecandioyl-γGlu-OEG-OEG),A14E,A21G,B25H,desB30 human insulin,
A14E,B25H,B29K(N^{ε}Eicosanedioyl-OEG),desB30 human insulin,
A14E,B25H,B27K(N^{ε}Octadecanedioyl-γGlu-OEG-OEG),desB28,desB29,desB30 human insulin,
A14E,B25H,B29K(N^{ε}(5-Eicosanedioylaminoisophthalic acid)),desB30 human insulin,
A14E,B25H,B29K(N^{ε}Octadecanedioyl),desB30 human insulin,
A14E,B29K(N^{ε}Octadecanedioyl-γGlu-OEG-OEG),desB30 human insulin,
A14E,B25H,B26G,B27G,B28G,B29K(N^{ε}Eicosanedioyl-γGlu-OEG-OEG),desB30 human insulin,
A14E,B25H,B29K(N^{ε}Octadecanedioyl-γGlu-OEG),desB30 human insulin,
A14E,B25H,B29K(N^{ε}Eicosanedioyl-OEG-OEG),desB30 human insulin,
A14E,B25H,B29K(N^{ε}Eicosanedioyl-Aoc),desB30 human insulin,
A14E,B25H,B26G,B27G,B28G,B29K(N^{ε}Eicosanedioyl-γGlu-γGlu),desB30 human insulin,
A14E,B25H,B26G,B27G,B28G,B29K(N^{ε}Eicosanedioyl-γGlu-γGlu),desB30 human insulin,
A14E,B25H,B29K(N^{ε}Octadecanedioyl-OEG),desB30 human insulin,
A14E,B25H,desB27,B29K(N^{ε}Octadecanedioyl-γGlu-OEG-OEG),desB30 human insulin,
A14E,B25H,B16H,B29K(N^{ε}Octadecanedioyl-γGlu),desB30 human insulin,
A1G(N^{α}Octadecanedioyl),A14E,B25H,B29R,desB30 human insulin,
A14E,B16H,B25H,B29K(N^{ε}Eicosanedioyl-γGlu),desB30 human insulin,
A14E,B25H,B27K(N^{ε}Eicosanedioyl-γGlu),desB28,desB29,desB30 human insulin,
A14E,B25H,B29K(N^{ε}Octadecanedioyl-γGlu-γGlu-γGlu),desB30 human insulin,
A14E,B25H,B26G,B27G,B28G,B29K(N^{ε}Octadecandioyl-γGlu),desB30 human insulin,
A14E,B25H,B26G,B27G,B28G,B29K(N^{ε}EicosanedioyHGlu),desB30 human insulin,
A14E,B25H,B26G,B27G,B28G,B29K(N^{ε}Octadecandioyl),desB30 human insulin,
A14E,B25H,B26G,B27G,B28G,B29K(N^{ε}Eicosanedioyl),desB30 human insulin,
A14E,B25H,B29K(N^{ε}Docosanedioyl-γGlu),desB30 human insulin,
A14E,B25H,B29K(N^{ε}Docosanedioyl-γGlu-γGlu),desB30 human insulin,
A14E,B25H,B29K(N^{ε}Icosanedioyl-γGlu-OEG-OEG-γGlu),desB30 human insulin,
A14E,B25H,B29K(N^{ε}Octadecanedioyl-γGlu-OEG-OEG-γGlu),desB30 human insulin,
A14E,B25H,B29K(N^{ε}(N-Icosanedioyl-N-carboxymethyl)-βAla),desB30 human insulin,
A14E,B25H,B29K(N^{ε}3-[2-(2-{2-[2-(17-Carboxyheptadecanoylamino)ethoxy]ethoxy}ethoxy)ethoxy]propionyl-γGlu),desB30 human insulin,
A14E,B25H,B29K(N^{ε}3-[2-(2-{2-[2-(19-Carboxynonadecanoylamino)ethoxy]ethoxy}ethoxy)ethoxy]propionyl-γGlu),desB30 human insulin,
A14E,B25H,B29K(N^{ε}Octadecandioyl-γGlu-(3-(2-{2-[2-(2-aminoethoxy)ethoxy]ethoxy}ethoxy)propionyl),desB30 human insulin,
A14E,B25H,B29K(N^{ε}Octadecandioyl-γGlu-(3-(2-{2-[2-(2-aminoethoxy)ethoxy]ethoxy}ethoxy)propionyl-γGlubdesB30 human insulin,
A14E,B25H,B29K(N^{ε}Icosanedioyl-γGlu-(3-(2-{2-[2-(2-aminoethoxy)ethoxy]ethoxy}ethoxy)propionyl),desB30 human insulin,
A14E,B25H,B29K(N^{ε}4-([4-({17-Carboxynonadecanoylamino}nnethyl)trans-cyclo-hexanecarbonyl]-γGlubdesB30 human insulin,
A14E,B25H,B29K(N^{ε}4-([4-({17-Carboxyheptadecanoylamino}nnethyl)trans-cyclo-hexanecarbonyl]-γGlu-γGlubdesB30 human insulin,
A14E,B28D,B29K(N^{ε}hexadecandioyl-γGlu),desB30 human insulin,
A14E,B28D,B29K(N^{ε}Eicosanedioyl-γGlu),desB30 human insulin,
A14E,B28D,B29K(N^{ε}Octadecandioyl-γGlu-OEG-OEG),desB30 human insulin,
A14E,B28D,B29K(N^{ε}Eicosanedioyl-γGlu-OEG-OEG),desB30 human insulin,
A14E,B28E,B29K(N^{ε}Hexadecandioyl-γGlu),desB30 human insulin,
A14E,B28E,B29K(N^{ε}Octadecandioyl-γGlu),desB30 human insulin,
A14E,B28E,B29K(N^{ε}Eicosanedioyl-γGlu),desB30 human insulin,
A14E,B28E,B29K(N^{ε}Octadecandioyl-γGlu-OEG-OEG),desB30 human insulin,
A14E,B28E,B29K(N^{ε}Eicosanedioyl-γGlu-OEG-OEG),desB30 human insulin,
A14E,B1E,B28E,B29K(N^{ε}Hexadecandioyl-γGlu),desB30 human insulin,
A14E,B1E,B28E,B29K(N^{ε}Octadecandioyl-γGlu),desB30 human insulin,
A14E,B1E,B28E,B29K(N^{ε}Eicosanedioyl-γGlu),desB30 human insulin,
A14E,B1E,B28E,B29K(N^{ε}Hexadecandioyl-γGlu-OEG-OEG),desB30 human insulin,
A14E,B1E,B28E,B29K(N^{ε}Octadecandioyl-γGlu-OEG-OEG),desB30 human insulin,
A14E,B1E,B28E,B29K(N^{ε}Eicosanedioyl-γGlu-OEG-OEG),desB30 human insulin,
A14E,B1E,B27E,B28E,B29K(N^{ε}Hexadecandioyl-γGlu),desB30 human insulin,
A14E,B1E,B27E,B28E,B29K(N^{ε}Octadecandioyl-γGlu),desB30 human insulin,
A14E,B1E,B27E,B28E,B29K(N^{ε}Eicosanedioyl-γGlu),desB30 human insulin,
A14E,B1E,B27E,B28E,B29K(N^{ε}Hexadecandioyl-γGlu-OEG-OEG),desB30 human insulin,
A14E,B1E,B27E,B28E,B29K(N^{ε}Octadecandioyl-γGlu-OEG-OEG),desB30 human insulin,
A14E,B1E,B27E,B28E,B29K(N^{ε}Eicosanedioyl-γGlu-OEG-OEG),desB30 human insulin,
A14E,B1E,B25H,B28E,B29K(N^{ε}Hexadecandioyl-γGlu),desB30 human insulin,
A14E,B1E,B25H,B28E,B29K(N^{ε}Octadecandioyl-γGlu),desB30 human insulin,
A14E,B1E,B25H,B28E,B29K(N^{ε}Eicosanedioyl-γGlu),desB30 human insulin,
A14E,B1E,B25H,B28E,B29K(N^{ε}Hexadecandioyl-γGlu-OEG-OEG),desB30 human insulin,
A14E,B1E,B25H,B28E,B29K(N^{ε}Octadecandioyl-γGlu-OEG-OEG),desB30 human insulin,
A14E,B1E,B25H,B28E,B29K(N^{ε}Eicosanedioyl-γGlu-OEG-OEG),desB30 human insulin,
A14E,B1E,B25H,B27E,B28E,B29K(N^{ε}Hexadecandioyl-γGlu),desB30 human insulin,
A14E,B1E,B25H,B27E,B28E,B29K(N^{ε}Octadecandioyl-γGlu),desB30 human insulin,
A14E,B1E,B25H,B27E,B28E,B29K(N^{ε}Eicosanedioyl-γGlu),desB30 human insulin,
A14E,B1E,B25H,B27E,B28E,B29K(N^{ε}Hexadecandioyl-γGlu-OEG-OEG),desB30 human insulin,
A14E,B1E,B25H,B27E,B28E,B29K(N^{ε}Octadecandioyl-γGlu-OEG-OEG),desB30 human insulin,
A14E,B1E,B25H,B27E,B28E,B29K(N^{ε}Eicosanedioyl-γGlu-OEG-OEG),desB30 human insulin,
A14E,B28D,B29K(N^{ε}Hexadecanedioyl-γGlu-OEG-OEG),desB30 human insulin,
A14E,B28E,B29K(N^{ε}Hexadecanedioyl-γGlu-OEG-OEG),desB30 human insulin,
B25N,B27E,B29K(N^{ε}Eicosanedioyl-γGlu-OEG-OEG),desB30 human insulin,
B25N,B27E,B29K(N^{ε}Octadecanedioyl-γGlu-OEG-OEG),desB30 human insulin,
B25N,B27E,B29K(N^{ε}Hexadecanedioyl-γGlu-OEG-OEG),desB30 human insulin,
B25N,B27E,B29K(N^{ε}Eicosanedioyl-γGlu),desB30 human insulin,
B25N,B27E,B29K(N^{ε}Octadecanedioyl-γGlu),desB30 human insulin,
B25N,B27E,B29K(N^{ε}Hexadecanedioyl-γGlu),desB30 human insulin,
A8H,B25N,B27E,B29K(N^{ε}Eicosanedioyl-γGlu-OEG-OEG),desB30 human insulin,
A8H,B25N,B27E,B29K(N^{ε}Octadecanedioyl-γGlu-OEG-OEG),desB30 human insulin,
A8H,B25N,B27E,B29K(N^{ε}Hexadecanedioyl-γGlu-OEG-OEG),desB30 human insulin,
A8H,B25N,B27E,B29K(N^{ε}Eicosanedioyl-γGlu),desB30 human insulin,
A8H,B25N,B27E,B29K(N^{ε}Octadecanedioyl-γGlu),desB30 human insulin,
A8H,B25N,B27E,B29K(N^{ε}Hexadecanedioyl-γGlu),desB30 human insulin,
14E,B25H,B29K(N^{ε}(N-Icosanedioyl-N-carboxymethyl)-βAla-OEG-OEG),desB30 human insulin,
A14E,B25H,B29K(N^{ε}(N-Octadecanedioyl-N-carboxymethyl)-βAla-OEG-OEG),desB30 human insulin,
A14E,B25H,B29K(N^{ε}(N-Hexadecanedioyl-N-carboxymethyl)-βAla-OEG-OEG),desB30 human insulin,
A14E,B25H,B29K(N^{ε}octadecanedioyl-γGlu-2-[(3-{2-[2-(3-aminopropoxy)ethoxy]ethoxy}propylcarbamoyl)methoxy]acetyl),desB30 human insulin,
A14E,B25H,B29K(N^{ε}eicosanedioyl-γGlu-2-[(3-{2-[2-(3-aminopropoxy)ethoxy]ethoxy}propylcarbamoyl)methoxy]acetyl),desB30 human insulin,
A14E,B16H,B25H,B29K(N^{ε}Octadecanedioyl-γGlu-2-[(3-{2-[2-(3-aminopropoxy)-ethoxy]ethoxy}propylcarbamoyl)methoxy]acetyl),desB30 human insulin,
A14E, B16H, B25H, B29K(N^{ε}Eicosanedioyl-γGlu-2-[(3-{2-[2-(3-aminopropoxy)-ethoxy]ethoxy}propylcarbamoyl)methoxy]acetyl),desB30 human insulin,
B25H,B29K(N^{ε}Octadecanedioyl-γGlu-OEG-OEG),desB30 human insulin,
B25H,B29K(N^{ε}Eicosanedioyl-γGlu-OEG-OEG),desB30 human insulin,
B25H,B29K(N^{ε}Octadecanedioyl-γGlu),desB30 human insulin,
B25H,B29K(N^{ε}Eicosanedioyl-γGlu),desB30 human insulin,
B25H,B29K(N^{ε}Octadecanedioyl),desB30 human insulin,
B25H,B29K(N^{ε}Eicosanedioyl),desB30 human insulin,
B25H,B29K(N^{ε}Octadecanedioyl-γGlu-OEG-OEG),desB30 human insulin,
B25H,B29K(N^{ε}Eicosanedioyl-γGlu-OEG-OEG),desB30 human insulin,
B25H,B29K(N^{ε}Octadecanedioyl-γGlu),desB30 human insulin,
B25H,B29K(N^{ε}Eicosanedioyl-γGlu),desB30 human insulin,
21G,B25H,B29K(N^{ε}Octadecanedioyl),desB30 human insulin,
A21G,B25H,B29K(N^{ε}Eicosanedioyl),desB30 human insulin,
A21G,B25H,B29K(N^{ε}Octadecanedioyl-γGlu-OEG-OEG),desB30 human insulin,
A21G,B25H,B29K(N^{ε}Eicosanedioyl-γGlu-OEG-OEG),desB30 human insulin,
A21G,B25H,B29K(N^{ε}Octadecanedioyl-γGlu),desB30 human insulin,
A21G,B25H,B29K(N^{ε}Eicosanedioyl-γGlu),desB30 human insulin,
A14E,B25H,desB27,B29K(N^{ε}Octadecanedioyl),desB30 human insulin,
A14E,B25H,desB27,B29K(N^{ε}Eicosanedioyl),desB30 human insulin,
A14E,B25H,desB27,B29K(N^{ε}Octadecanedioyl-γGlu),desB30 human insulin,
A14E,B25H,desB27,B29K(N^{ε}Eicosanedioyl-γGlu),desB30 human insulin,
A14E,B25H,desB27,B29K(N^{ε}Eicosanedioyl-γGlu-OEG-OEG),desB30 human insulin,
A14E,A21G,B25H,desB27,B29K(N^{ε}Octadecanedioyl),desB30 human insulin,
A14E,A21G,B25H,desB27,B29K(N^{ε}Eicosanedioyl),desB30 human insulin,
A14E,A21G,B25H,desB27,B29K(N^{ε}Octadecanedioyl-γGlu),desB30 human insulin,
A14E,B25H,desB27,B29K(N^{ε}Eicosanedioyl-γGlu),desB30 human insulin,
A14E,A21G,B25H,desB27,B29K(N^{ε}Octadecanedioyl-γGlu-OEG-OEG),desB30 human insulin,
A14E,A21G,B25H,desB27,B29K(N^{ε}Eicosanedioyl-γGlu-OEG-OEG),desB30 human insulin,
A14E,A21G,B25H,B29K(N^{ε}Octadecanedioyl-γGlu-OEG-OEG),desB30 human insulin,
A14E,A21G,B25H,B29K(N^{ε}Eicosanedioyl-γGlu-OEG-OEG),desB30 human insulin,
A14E,A21G,B25H,B29K(N^{ε}Eicosanedioyl-γGlu),desB30 human insulin,
A14E,A21G,B25H,B29K(N^{ε}Eicosanedioyl),desB30 human insulin,
A14E,A21G,B25H,B29K(N^{ε}Octadecanedioyl-γGlu),desB30 human insulin,
A14E,A21G,B25H,B29K(N^{ε}Octadecanedioyl),desB30 human insulin,
A14E,B25H,B26G,B27G,B28G,B29K(N^{ε}Octadecanedioyl-γGlu),desB30 human insulin,
A14E,B25H,B26G,B27G,B28G,B29K(N^{ε}Octadecanedioyl),desB30 human insulin,
A14E,B25H,B26G,B27G,B28G,B29K(N^{ε}Eicosanedioyl-γGlu),desB30 human insulin,
A14E,B25H,B26G,B27G,B28G,B29K(N^{ε}Eicosanedioyl),desB30 human insulin,
A1G(N^{α}Octadecandioyl-γGlu),A14E,B25H,B26G,B27G,B28G,desB30 human insulin,
A1G(N^{α}Eicosanedioyl-γGlu),A14E,B25H,B26G,B27G,B28G,desB30 human insulin,
A1G(N^{α}Octadecandioyl-γGlu),A14E,B25H,B26G,B27G,B28G,B29R,desB30 human insulin,
A1G(N^{α}Eicosanedioyl-γGlu),A14E,B25H,B26G,B27G,B28G,B29R,desB30 human insulin,
A1G(N^{α}Octadecandioyl),A14E,B25H,B26G,B27G,B28G,desB30 human insulin,
A1G(N^{α}Eicosanedioyl),A14E,B25H,B26G,B27G,B28G,desB30 human insulin,
A1G(N^{α}Octadecandioyl),A14E,B25H,B26G,B27G,B28G,B29R,desB30 human insulin and
A1G(N^{α}Eicosanedioyl),A14E,B25H,B26G,B27G,B28G,B29R,desB30 human insulin.
109. The pharmaceutical composition according to any one of the preceding aspects, wherein said protease stabilised insulin is selected from the group consisting of: A14E,B25H,B29K(N^{ε}-Hexadecandioyl),desB30 human insulin,
A14E,B25H,B29K(N^{ε}Octadecandioyl-γGlu),desB30 human insulin,
A14E,B25H,B29K(N^{ε}Eicosanedioyl-γGlu),desB30 human insulin,
A14E,B25H,B29K(N^{ε}3-Carboxy-5-octadecanedioylaminobenzoyl),desB30 human insulin,
A14E,B25H,B29K(N^{ε}-N-octadecandioyl-N-(2-carboxyethyl)glycyl),desB30 human insulin
A14E,B25H,B29K(N^{ε}(N-Octadecandioyl-N-carboxymethyl)-beta-alanyl),desB30 human insulin,
A14E,B25H,B29K(N^{ε}4-([4-({19-Carboxynonadecanoylamino}nnethyl)trans-cyclo-hexanecarbonyl]-γGlubdesB30 human insulin,
A14E,B25H,B29K(N^{ε}Heptadecanedioyl-γGlu),desB30 human insulin,
A14E,B25H,B29K(N^{ε}Octadecanedioyl-γGlu-OEG-OEG),desB30 human insulin,
A14E,B25H,B29K(N^{ε}Myristyl),desB30 human insulin,
A14E,B25H,B29K(N^{ε}Eicosanedioyl-γGlu-γGlu),desB30 human insulin,
A14E,B25H,B29K(N^{ε}4-([4-({19-Carboxynonadecanoylamino}nnethyptrans-cyclo-hexanecarbonyl]-γGlu-γGlubdesB30 human insulin,
A14E,B25H,B29K(N^{ε}Octadecanedioyl-γGlu-γGlu),desB30 human insulin,
A14E,B28D,B29K(N^{ε}octadecandioyl-γGlu),desB30 human insulin,
A14E,B25H,B29K(N^{ε}octadecandioyl-γGlu-PEG7),desB30 human insulin,
A14E,B25H,B29K(N^{ε}eicosanedioyl-γGlu-OEG-OEG), desB30 human insulin,
A14E,B25H,B29K(N^{ε}eicosanedioyl-γGlu-(3-(2-{2-[2-(2-aminoethoxy)ethoxy]ethoxy}ethoxy)propionyl-γGlubdesB30 human insulin,
A14E,B25H,B29K(N^{ε}Hexadecanedioyl-γGlu-OEG-OEG),desB30 human insulin,
A14E,B25H,B29K(N^{ε}Hexadecanedioyl-γGlu),desB30 human insulin,
A14E,B25H,B29K(N^{ε}heptadecanedioyl-γGlu-OEG-OEG),desB30 human insulin,
A14E,B25H,B29K(N^{ε}octadecanedioyl-γGlu-γGlu-γGlu-γGlu),desB30 human insulin,
A14E,B25H,B29K(N^{ε}Eicosanedioyl-γGlu-γGlu-γGlu),desB30 human insulin,
A14E,B25H,B27E,B29K(N^{ε}Octadecanedioyl-γGlu-OEG-OEG),desB30 human insulin,
A14E,B25H,B26G,B27G,B28G,B29K(N^{ε}Octadecanedioyl-γGlu-OEG-OEG),desB30 human insulin,
A14E,B16H,B25H,B29K(N^{ε}Octadecanedioyl-γGlu-OEG-OEG),desB30 human insulin,
A14E,B16E,B25H,B29K(N^{ε}Octadecanedioyl-γGlu-OEG-OEG),desB30 human insulin,
A14E,B16H,B25H,B29K(N^{ε}Hexadecanedioyl-xGlu),desB30 human insulin,
A14E,B25H,B29K(N^{ε}Eicosanedioyl-γGlu-OEG-xGlu),desB30 human insulin,
A14E,B16E,B25H,B29K(N^{ε}Hexadecandioyl-xGlu),desB30 human insulin,
A14E,B16H,B25H,B29K(N^{ε}Octadecanedioyl-γGlu-xGlu-xGlu),desB30 human insulin,
A14E,B25H,B26G,B27G,B28G,B29K(N^{ε}Hexadecandioyl-xGlu),desB30 human insulin,
A14E,B16H,B25H,B29K(N^{ε}Octadecanedioyl-γGlu-xGlu),desB30 human insulin,
A14E,B16H,B25H,B29K(N^{ε}Eicosanedioyl-γGlu-OEG-OEG),desB30 human insulin,
A14E,B25H,B29K(N^{ε}Octadecanedioyl-OEG-γGlu-γGlu),desB30 human insulin,
A14E,A18L,B25H,B29K(N^{ε}Eicosanedioyl-γGlu-OEG-OEG),desB30 human insulin,
A14E,A18L,B25H,B29K(N^{ε}Octadecanedioyl-γGlu-OEG-OEG),desB30 human insulin,
A14E,B25H,B27E,B29K(N^{ε}Eicosanedioyl-γGlu-OEG-OEG),desB30 human insulin,
A1G(N^{α}Octadecandioyl-γGlu-OEG-OEG),A14E,B25H,B29R,desB30 human insulin,
A14E,B1F(N^{α}Octadecandioyl-γGlu-OEG-OEG),B25H,B29R,desB30 human insulin,
A1G(N^{α}Hexadecandioyl-γGlu),A14E,B25H,B29R,desB30 human insulin,

A14E,B25H,B29K(N^{ε}Octadecanedioyl-γGlu-Abu-Abu-Abu-Abu),desB30 human insulin,
A14E,B25H,B29K(N^{α}Eicosanedioyl),desB30 human insulin,
A14E,B25H,B29K(N^{α}4-[16-(1H-Tetrazol-5-yl)hexadecanoylsulfarnoyl]butanoyl), desB30 human insulin,

A1G(N^{α}Octadecandioyl-γGlu-OEG-OEG),A14E,A21G,B25H,desB30 human insulin,
A14E,B25H,B29K(N^{ε}Eicosanedioyl-OEG),desB30 human insulin,
A14E,B25H,B27K(N^{ε}Octadecanedioyl-γGlu-OEG-OEG),desB28,desB29,desB30 human insulin,
A14E,B25H,B29K(N^{ε}(5-Eicosanedioylaminoisophthalic acid)),desB30 human insulin,
A14E,B25H,B29K(N^{ε}Octadecanedioyl),desB30 human insulin,
A14E,B29K(N^{ε}Octadecanedioyl-γGlu-OEG-OEG),desB30 human insulin,
A14E,B25H,B26G,B27G,B28G,B29K(N^{ε}Eicosanedioyl-γGlu-OEG-OEG),desB30 human insulin,
A14E,B25H,B29K(N^{ε}Octadecanedioyl-γGlu-OEG),desB30 human insulin,
A14E,B25H,B29K(N^{ε}Eicosanedioyl-OEG-OEG),desB30 human insulin,
A14E,B25H,B29K(N^{ε}Eicosanedioyl-Aoc),desB30 human insulin,
A14E,B25H,B26G,B27G,B28G,B29K(N^{ε}Eicosanedioyl-γGlu-γGlu),desB30 human insulin,
A14E,B25H,B26G,B27G,B28G,B29K(N^{ε}Eicosanedioyl-γGlu-γGlu),desB30 human insulin,
A14E,B25H,B29K(N^{ε}Octadecanedioyl-OEG),desB30 human insulin,
A14E,B25H,desB27,B29K(N^{ε}Octadecanedioyl-γGlu-OEG-OEG),desB30 human insulin,
A14E,B25H,B16H,B29K(N^{ε}Octadecanedioyl-γGlu),desB30 human insulin,
A1G(N^{α}Octadecanedioyl),A14E,B25H,B29R,desB30 human insulin,
A14E,B16H,B25H,B29K(N^{ε}Eicosanedioyl-γGlu),desB30 human insulin,
A14E,B25H,B27K(N^{ε}Eicosanedioyl-γGlu),desB28,desB29,desB30 human insulin,
A14E,B25H,B29K(N^{ε}Octadecanedioyl-γGlu-γGlu-γGlu),desB30 human insulin,
A14E,B25H,B26G,B27G,B28G,B29K(N^{ε}Octadecandioyl-γGlu),desB30 human insulin,
A14E,B25H,B26G,B27G,B28G,B29K(N^{ε}EicosanedioyHGlu),desB30 human insulin,
A14E,B25H,B26G,B27G,B28G,B29K(N^{ε}Octadecandioyl),desB30 human insulin,
A14E,B25H,B26G,B27G,B28G,B29K(N^{ε}Eicosanedioyl),desB30 human insulin,
A14E,B25H,B29K(N^{ε}Docosanedioyl-γGlu),desB30 human insulin,
A14E,B25H,B29K(N^{ε}Docosanedioyl-γGlu-γGlu),desB30 human insulin,
A14E,B25H,B29K(N^{ε}Icosanedioyl-γGlu-OEG-OEG-γGlu),desB30 human insulin,
A14E,B25H,B29K(N^{ε}Octadecanedioyl-γGlu-OEG-OEG-γGlu),desB30 human insulin,
A14E,B25H,B29K(N^{ε}(N-Icosanedioyl-N-carboxymethyl)-βAla),desB30 human insulin,
A14E,B25H,B29K(N^{ε}3-[2-(2-{2-[2-(17-Carboxyheptadecanoylamino)ethoxy]ethoxy}ethoxy)ethoxy]propionyl-γGlu),desB30 human insulin,
A14E,B25H,B29K(N^{ε}3-[2-(2-{2-[2-(19-Carboxynonadecanoylamino)ethoxy]ethoxy}ethoxy)ethoxy]propionyl-γGlu),desB30 human insulin,
A14E,B25H,B29K(N^{ε}Octadecandioyl-γGlu-(3-(2-{2-[2-(2-aminoethoxy)ethoxy]ethoxy}ethoxy)propionyl),desB30 human insulin,
A14E,B25H,B29K(N^{ε}Octadecandioyl-γGlu-(3-(2-{2-[2-(2-aminoethoxy)ethoxy]ethoxy}ethoxy)propionyl-γGlubdesB30 human insulin,
A14E,B25H,B29K(N^{ε}Icosanedioyl-γGlu-(3-(2-{2-[2-(2-aminoethoxy)ethoxy]ethoxy}ethoxy)propionyl),desB30 human insulin,
A14E,B25H,B29K(N^{ε}4-([4-({17-Carboxynonadecanoylamino}nnethyl)trans-cyclo-hexanecarbonyl]-γGlubdesB30 human insulin,
A14E,B25H,B29K(N^{ε}4-([4-({17-Carboxyheptadecanoylamino}methyl)trans-cyclo-hexanecarbonyl]-γGlu-γGlubdesB30 human insulin,
A14E,B28D,B29K(N^{ε}hexadecandioyl-γGlu),desB30 human insulin,
A14E,B28D,B29K(N^{ε}Eicosanedioyl-γGlu),desB30 human insulin,
A14E,B28D,B29K(N^{ε}Octadecandioyl-γGlu-OEG-OEG),desB30 human insulin,
A14E,B28D,B29K(N^{ε}Eicosanedioyl-γGlu-OEG-OEG),desB30 human insulin,
A14E,B28E,B29K(N^{ε}Hexadecandioyl-γGlu),desB30 human insulin,
A14E,B28E,B29K(N^{ε}Octadecandioyl-γGlu),desB30 human insulin,
A14E,B28E,B29K(N^{ε}Eicosanedioyl-γGlu),desB30 human insulin,
A14E,B28E,B29K(N^{ε}Octadecandioyl-γGlu-OEG-OEG),desB30 human insulin,
A14E,B28E,B29K(N^{ε}Eicosanedioyl-γGlu-OEG-OEG),desB30 human insulin,
A14E,B1E,B28E,B29K(N^{ε}Hexadecandioyl-γGlu),desB30 human insulin,
A14E,B1E,B28E,B29K(N^{ε}Octadecandioyl-γGlu),desB30 human insulin,
A14E,B1E,B28E,B29K(N^{ε}Eicosanedioyl-γGlu),desB30 human insulin,
A14E,B1E,B28E,B29K(N^{ε}Hexadecandioyl-γGlu-OEG-OEG),desB30 human insulin,
A14E,B1E,B28E,B29K(N^{ε}Octadecandioyl-γGlu-OEG-OEG),desB30 human insulin,
A14E,B1E,B28E,B29K(N^{ε}Eicosanedioyl-γGlu-OEG-OEG),desB30 human insulin,
A14E,B1E,B27E,B28E,B29K(N^{ε}Hexadecandioyl-γGlu),desB30 human insulin,
A14E,B1E,B27E,B28E,B29K(N^{ε}Octadecandioyl-γGlu),desB30 human insulin,
A14E,B1E,B27E,B28E,B29K(N^{ε}Eicosanedioyl-γGlu),desB30 human insulin,
A14E,B1E,B27E,B28E,B29K(N^{ε}Hexadecandioyl-γGlu-OEG-OEG),desB30 human insulin,
A14E,B1E,B27E,B28E,B29K(N^{ε}Octadecandioyl-γGlu-OEG-OEG),desB30 human insulin,
A14E,B1E,B27E,B28E,B29K(N^{ε}Eicosanedioyl-γGlu-OEG-OEG),desB30 human insulin,
A14E,B1E,B25H,B28E,B29K(N^{ε}Hexadecandioyl-γGlu),desB30 human insulin,
A14E,B1E,B25H,B28E,B29K(N^{ε}Octadecandioyl-γGlu),desB30 human insulin,
A14E,B1E,B25H,B28E,B29K(N^{ε}Eicosanedioyl-γGlu),desB30 human insulin,
A14E,B1E,B25H,B28E,B29K(N^{ε}Hexadecandioyl-γGlu-OEG-OEG),desB30 human insulin,
A14E,B1E,B25H,B28E,B29K(N^{ε}Octadecandioyl-γGlu-OEG-OEG),desB30 human insulin,
A14E,B1E,B25H,B28E,B29K(N^{ε}Eicosanedioyl-γGlu-OEG-OEG),desB30 human insulin,
A14E,B1E,B25H,B27E,B28E,B29K(N^{ε}Hexadecandioyl-γGlu),desB30 human insulin,
A14E,B1E,B25H,B27E,B28E,B29K(N^{ε}Octadecandioyl-γGlu),desB30 human insulin,
A14E,B1E,B25H,B27E,B28E,B29K(N^{ε}Eicosanedioyl-γGlu),desB30 human insulin,
A14E,B1E,B25H,B27E,B28E,B29K(N^{ε}Hexadecandioyl-γGlu-OEG-OEG),desB30 human insulin,
A14E,B1E,B25H,B27E,B28E,B29K(N^{ε}Octadecandioyl-γGlu-OEG-OEG),desB30 human insulin,
A14E,B1E,B25H,B27E,B28E,B29K(N^{ε}Eicosanedioyl-γGlu-OEG-OEG),desB30 human insulin,
A14E,B28D,B29K(N^{ε}Hexadecanedioyl-γGlu-OEG-OEG),desB30 human insulin,
A14E,B28E,B29K(N^{ε}Hexadecanedioyl-γGlu-OEG-OEG),desB30 human insulin,
B25N,B27E,B29K(N^{ε}Eicosanedioyl-γGlu-OEG-OEG),desB30 human insulin,
B25N,B27E,B29K(N^{ε}Octadecanedioyl-γGlu-OEG-OEG),desB30 human insulin,
B25N,B27E,629K(N^{ε}Hexadecanedioyl-γGlu-OEG-OEG),desB30 human insulin,
B25N,B27E,629K(N^{ε}Eicosanedioyl-γGlu),desB30 human insulin,
B25N,B27E,629K(N^{ε}Octadecanedioyl-γGlu),desB30 human insulin,
B25N,B27E,629K(N^{ε}Hexadecanedioyl-γGlu),desB30 human insulin,
A8H,B25N,B27E,629K(N^{ε}Eicosanedioyl-γGlu-OEG-OEG),desB30 human insulin,
A8H,B25N,B27E,629K(N^{ε}Octadecanedioyl-γGlu-OEG-OEG),desB30 human insulin,
A8H,B25N,B27E,629K(N^{ε}Hexadecanedioyl-γGlu-OEG-OEG),desB30 human insulin,
A8H,B25N,B27E,629K(N^{ε}Eicosanedioyl-γGlu),desB30 human insulin,
A8H,B25N,B27E,629K(N^{ε}Octadecanedioyl-γGlu),desB30 human insulin,
A8H,B25N,B27E,629K(N^{ε}Hexadecanedioyl-γGlu),desB30 human insulin,
14E,B25H,B29K(N^{ε}(N-Icosanedioyl-N-carboxymethyl)-βAla-OEG-OEG),desB30 human insulin,
A14E,B25H,B29K(N^{ε}(N-Octadecanedioyl-N-carboxymethyl)-βAla-OEG-OEG),desB30 human insulin,
A14E,B25H,B29K(N^{ε}(N-Hexadecanedioyl-N-carboxymethyl)-βAla-OEG-OEG),desB30 human insulin,
A14E,B25H,B29K(N^{ε}octadecanedioyl-γGlu-2-[(3-{2-[2-(3-aminopropoxy)ethoxy]ethoxy}propylcarbamoyl)methoxy]acetyl),desB30 human insulin,
A14E,B25H,B29K(N^{ε}eicosanedioyl-γGlu-2-[(3-{2-[2-(3-aminopropoxy)ethoxy]ethoxy}propylcarbamoyl)methoxy]acetyl),desB30 human insulin,
A14E,B16H,B25H,B29K(N^{ε}Octadecanedioyl-γGlu-2-[(3-{2-[2-(3-aminopropoxy)-ethoxy]ethoxy}propylcarbamoyl)methoxy]acetyl),desB30 human insulin,
A14E, B16H, B25H, B29K(N^{ε}Eicosanedioyl-γGlu-2-[(3-{2-[2-(3-aminopropoxy)-ethoxy]ethoxy}propylcarbamoyl)methoxy]acetyl),desB30 human insulin,
B25H,B29K(N^{ε}Octadecanedioyl-γGlu-OEG-OEG),desB30 human insulin,
B25H,B29K(N^{ε}Eicosanedioyl-γGlu-OEG-OEG),desB30 human insulin,
B25H,B29K(N^{ε}Octadecanedioyl-γGlu),desB30 human insulin,
B25H,B29K(N^{ε}Eicosanedioyl-γGlu),desB30 human insulin,
B25H,B29K(N^{ε}Octadecanedioyl),desB30 human insulin,
B25H,B29K(N^{ε}Eicosanedioyl),desB30 human insulin,
B25H,B29K(N^{ε}Octadecanedioyl-γGlu-OEG-OEG),desB30 human insulin,
B25H,B29K(N^{ε}Eicosanedioyl-γGlu-OEG-OEG),desB30 human insulin,
B25H,B29K(N^{ε}Octadecanedioyl-γGlu),desB30 human insulin,
B25H,B29K(N^{ε}Eicosanedioyl-γGlu),desB30 human insulin,
21G,B25H,B29K(N^{ε}Octadecanedioyl),desB30 human insulin,
A21G,B25H,B29K(N^{ε}Eicosanedioyl),desB30 human insulin,
A21G,B25H,B29K(N^{ε}Octadecanedioyl-γGlu-OEG-OEG),desB30 human insulin,
A21G,B25H,B29K(N^{ε}Eicosanedioyl-γGlu-OEG-OEG),desB30 human insulin,
A21G,B25H,B29K(N^{ε}Octadecanedioyl-γGlu),desB30 human insulin,
A21G,B25H,B29K(N^{ε}Eicosanedioyl-γGlu),desB30 human insulin,
A14E,B25H,desB27,B29K(N^{ε}Octadecanedioyl),desB30 human insulin,
A14E,B25H,desB27,B29K(N^{ε}Eicosanedioyl),desB30 human insulin,
A14E,B25H,desB27,B29K(N^{ε}Octadecanedioyl-γGlu),desB30 human insulin,
A14E,B25H,desB27,B29K(N^{ε}Eicosanedioyl-γGlu),desB30 human insulin,
A14E,B25H,desB27,B29K(N^{ε}Eicosanedioyl-γGlu-OEG-OEG),desB30 human insulin,
A14E,A21G,B25H,desB27,B29K(N^{ε}Octadecanedioyl),desB30 human insulin,
A14E,A21G,B25H,desB27,B29K(N^{ε}Eicosanedioyl),desB30 human insulin,
A14E,A21G,B25H,desB27,B29K(N^{ε}Octadecanedioyl-γGlu),desB30 human insulin,
A14E,B25H,desB27,B29K(N^{ε}Eicosanedioyl-γGlu),desB30 human insulin,
A14E,A21G,B25H,desB27,B29K(N^{ε}Octadecanedioyl-γGlu-OEG-OEG),desB30 human insulin,
A14E,A21G,B25H,desB27,B29K(N^{ε}Eicosanedioyl-γGlu-OEG-OEG),desB30 human insulin,
A14E,A21G,B25H,B29K(N^{ε}Octadecanedioyl-γGlu-OEG-OEG),desB30 human insulin,
A14E,A21G,B25H,B29K(N^{ε}Eicosanedioyl-γGlu-OEG-OEG),desB30 human insulin,
A14E,A21G,B25H,B29K(N^{ε}Eicosanedioyl-γGlu),desB30 human insulin,
A14E,A21G,B25H,B29K(N^{ε}Eicosanedioyl),desB30 human insulin,
A14E,A21G,B25H,B29K(N^{ε}Octadecanedioyl-γGlu),desB30 human insulin,
A14E,A21G,B25H,B29K(N^{ε}Octadecanedioyl),desB30 human insulin,
A14E,B25H,B26G,B27G,B28G,B29K(N^{ε}Octadecanedioyl-γGlu),desB30 human insulin,
A14E,B25H,B26G,B27G,B28G,B29K(N^{ε}Octadecanedioyl),desB30 human insulin,
A14E,B25H,B26G,B27G,B28G,B29K(N^{ε}Eicosanedioyl-γGlu),desB30 human insulin,
A14E,B25H,B26G,B27G,B28G,B29K(N^{ε}Eicosanedioyl),desB30 human insulin,
A1G(N^{α}Octadecandioyl-γGlu),A14E,B25H,B26G,B27G,B28G,desB30 human insulin,
A1G(N^{α}Eicosanedioyl-γGlu),A14E,B25H,B26G,B27G,B28G,desB30 human insulin,
A1G(N^{α}Octadecandioyl-γGlu),A14E,B25H,B26G,B27G,B28G,B29R,desB30 human insulin,
A1G(N^{α}Eicosanedioyl-γGlu),A14E,B25H,B26G,B27G,B28G,B29R,desB30 human insulin,
A1G(N^{α}Octadecandioyl),A14E,B25H,B26G,B27G,B28G,desB30 human insulin,
A1G(N^{α}Eicosanedioyl),A14E,B25H,B26G,B27G,B28G,desB30 human insulin,
A1G(N^{α}Octadecandioyl),A14E,B25H,B26G,B27G,B28G,B29R,desB30 human insulin and
A1G(N^{α}Eicosanedioyl),A14E,B25H,B26G,B27G,B28G,B29R,desB30 human insulin.
110. In one embodiment a tablet core according to the present invention comprises an protease stabilised insulin selected from the group consisting of: A10C,A14E,B4C,B25H,B29K(N^{ε}Octadecanedioyl-γGlu-OEG-OEG),desB30 human insulin,
A10C,A14E,B3C,B25H,B29K(N^{ε}Octadecanedioyl-γGlu),desB30 human insulin,
A10C,A14E,B3C,B25H,B29K(N^{ε}Octadecanedioyl),desB30 human insulin, A10C,A14E,B3C,B25H,B29K(N^{ε}Octadecanedioyl-γGlu),desB30 human insulin,
A10C,A14E,desB1,B4C,B25H,B29K(N^{ε}Octadecanedioyl-γGlu-OEG-OEG),desB30 human insulin,
A10C,A14H,B4C,B25H,B29K(N^{ε}Octadecanedioyl-γGlu-OEG-OEG),desB30 human insulin,
A10C,A14E,B3C,B25H,B29K(N^{ε}Eicosanedioyl-γGlu-OEG-OEG),desB30 human insulin,
A10C,A14E,B1C, B25H,B29K(N^{ε}Eicosanedioyl-γGlu-OEG-OEG),desB30 human insulin,
A10C,A14E,B4C B25H,B29K(N^{ε}Octadecanedioyl-γGlu),desB30 human insulin,
A10C,A14E, B3C,B25H,B29K(N^{ε}Octadecanedioyl-γGlu-OEG-OEG),desB30 human insulin,
A10C,A14E,B3C,B25H,B29K(N^{ε}Octadecanedioyl-γGlu-γGlu),desB30 human insulin,
A10C,A14E,B4C,B25H,desB27,B29K(N^{ε}Octadecanedioyl-γGlu),desB30 human insulin,
A10C,A14E,B4C,B25H,B29K(N^{ε}Octadecanedioyl),desB30 human insulin,
A10C,A14E,B4C,B25H,B29K(N^{ε}Octadecanedioyl-γGlu-γGlu),desB30 human insulin,
A10C,A14E,B2C,B25H,B29K(N^{ε}Octadecanedioyl-γGlu-OEG-OEG),desB30 human insulin,
A10C,A14E,B1C,B25H,B29K(N^{ε}Octadecanedioyl-γGlu-OEG-OEG),desB30 human insulin, A10C,A14E,B3C,B16H,B25H,B29K(N^{ε}Eicosanedioyl-γGlu-OEG-OEG),desB30 human insulin,
A10C,A14E, B4C,B25H,B29K(N^{ε}Myristyl),desB30 human insulin,
A10C,B4C, B29K(N^{ε}Myristyl),desB30 human insulin,
A10C,A14E,B3C,B25H,desB27,B29K(N^{ε}octadecanedioyl-γGlu),desB30 human insulin, A10C,A14E,B3C,B25H,desB27,B29K(N^{ε}octadecanedioyl-γGlu-OEG-OEG),desB30 human insulin,
A10C,A14E,B3C,B25H,B29K(N^{ε}Eicosanedioyl-γGlu),desB30 human insulin,
A10C,A14E,B4C,B25H,B29K(N^{ε}eicosanedioyl-γGlu-OEG-OEG),desB30 human insulin,
A10C,A14E,B3C,B25H,desB27,B29K(N^{ε}eicosanedioyl-γGlu),desB30 human insulin, A10C,A14E,B3C,B25H,desB27, 629K(N^{ε}eicosanedioyl-γGlu-OEG-OEG),desB30 human insulin,
A10C,A14E, 4C,B25H,B29K(N^{ε}Hexadecanedioyl-γGlu),desB30 human insulin,
A10C,A14E,B4C,B25H,B29K(N^{ε}Hexadecanedioyl-γGlu-OEG-OEG),desB30 human insulin,
A10C,A14E,B4C,B25H,B29K(N^{ε}Hexadecanedioyl),desB30 human insulin,
A10C,A14E,B4C,B25H,B29K(N^{ε}Hexadecanedioyl-γGlu-γGlu),desB30 human insulin,
A10C,A14E,B4C,B25H,desB27,B29K(N^{ε}Octadecanedioyl-γGlu-OEG-OEG),desB30 human insulin,
A10C,A14E,B4C,B25H,desB27,B29K(N^{ε}Octadecanedioyl-γGlu-γGlu),desB30 human insulin,
A10C,A14E,B4C,B25H,desB27,B29K(N^{ε}Hexadecanedioyl-γGlu-OEG-OEG),desB30 human insulin,
A10C,A14E,B4C,B25H,desB27,B29K(N^{ε}Hexadecanedioyl-γGlu),desB30 human insulin,
A10C,A14E,B3C,B25H,B29K(N^{ε}Hexadecanedioyl-γGlu),desB30 human insulin,
A10C,A14E,B3C,B25H,B29K(N^{ε}Hexadecanedioyl-γGlu-OEG-OEG),desB30 human insulin,
A10C,A14E,B2C,B25H,B29K(N^{ε}Hexadecanedioyl-γGlu-OEG-OEG),desB30 human insulin,
A10C,A14E,B2C,B25H,B29K(N^{ε}Hexadecanedioyl-γGlu),desB30 human insulin,
A10C,A14E,B2C,B25H,B29K(N^{ε}Octadecanedioyl-γGlu),desB30 human insulin,
A10C,A14E,B1C,B25H,B29K(N^{ε}Octadecanedioyl-γGlu),desB30 human insulin,
A10C,A14E,B1C,B25H,B29K(N^{ε}Hexadecanedioyl-γGlu-OEG-OEG),desB30 human insulin,
A10C,A14E,B1C,B25H,B29K(N^{ε}Hexadecanedioyl-γGlu),desB30 human insulin,
A10C,B1C,B29K(N^{ε}Hexadecanedioyl-γGlu-OEG-OEG),desB30 human insulin,
A10C,B1C,B29K(N^{ε}Hexadecanedioyl-γGlu),desB30 human insulin,
A10C,B1C,B29K(N^{ε}Octadecanedioyl-γGlu-OEG-OEG),desB30 human insulin,
A10C,B1C,B29K(N^{ε}Octadecanedioyl-γGlu),desB30 human insulin,
A10C,B2C,B29K(N^{ε}Octadecanedioyl-γGlu-OEG-OEG),desB30 human insulin,
A10C,B2C,B29K(N^{ε}Octadecanedioyl-γGlu),desB30 human insulin,
A10C,B2C,B29K(N^{ε}Hexadecanedioyl-γGlu-OEG-OEG),desB30 human insulin,
A10C,B2C,B29K(N^{ε}Hexadecanedioyl-γGlu),desB30 human insulin,
A10C,B3C,B29K(N^{ε}Hexadecanedioyl-γGlu),desB30 human insulin,
10C,B3C B29K(N^{ε}Hexadecanedioyl-γGlu-OEG-OEG),desB30 human insulin,
A10C,B3C,B29K(N^{ε}Octadecanedioyl-γGlu-OEG-OEG),desB30 human insulin,
A10C,B3C,B29K(N^{ε}Octadecanedioyl-γGlu),desB30 human insulin,
A10C,B4C,B29K(N^{ε}Octadecanedioyl-γGlu),desB30 human insulin,
A10C,B4C,B29K(N^{ε}Octadecanedioyl-γGlu-OEG-OEG),desB30 human insulin,
A10C,B4C B29K(N^{ε}Hexadecanedioyl-γGlu-OEG-OEG),desB30 human insulin,
A10C,B4C,B29K(N^{ε}Hexadecanedioyl-γGlu),desB30 human insulin,
A10C,A14E,B1C,B16H,B25H,B29K(Ngeicosanedioyl-γGlu-OEG-OEG),desB30 human insulin,
A10C,A14E,B1C,B16H,B25H,B29K(Ngeicosanedioyl-γGlu),desB30 human insulin,
A10C,A14E,B1C,B16H,B25H, B29K(N^{ε}Octadecanedioyl-γGlu-OEG-OEG),desB30 human insulin,
A10C,A14E,B1C,B16H,B25H,B29K(N^{ε}Octadecanedioyl-γGlu),desB30 human insulin,
A10C A14E,B1C,B16H,B25H,B29K(N^{ε}Hexadecanedioyl-γGlu),desB30 human insulin,
A10C,A14E,B1C,B16H,B25H,B29K(N^{ε}Hexadecanedioyl-γGlu-OEG-OEG),desB30 human insulin,
A10C,A14E,B2C,B16H,B25H,B29K(N^{ε}Hexadecanedioyl-γGlu-OEG-OEG),desB30 human insulin,
A10C,A14E,B2C,B16H,B25H,B29K(N^{ε}Hexadecanedioyl-γGlu),desB30 human insulin,
A10C,A14E,B2C,B16H,B25H,B29K(N^{ε}Octadecanedioyl-γGlu),desB30 human insulin,
A10C,A14E,B2C,B16H,B25H,B29K(N^{ε}Octadecanedioyl-γGlu-OEG-OEG),desB30 human insulin,
A10C,A14E,B2C,B16H,B25H,B29K(N^{ε}Eicosanedioyl-γGlu-OEG-OEG),desB30 human insulin,
A10C,A14E,B2C,B16H,B25H,B29K(N^{ε}Eicosanedioyl-γGlu),desB30 human insulin,
A10C,A14E,B3C,B16H,B25H,B29K(N^{ε}Eicosanedioyl-γGlu),desB30 human insulin, A10C,A14E,B3C,B16H,B25H,B29K(N^{ε}Octadecanedioyl-γGlu-OEG-OEG),desB30 human insulin,
A10C,A14E,B3C,B16H,B25H,B29K(N^{ε}Octadecanedioyl-γGlu),desB30 human insulin
A10C,A14E,B3C,B16H,B25H,B29K(N^{ε}Hexadecanedioyl-γGlu),desB30 human insulin,
A10C,A14E,B3C,B16H,B25H,B29K(N^{ε}Hexadecanedioyl-γGlu-OEG-OEG),desB30 human insulin,
A10C,A14E,B4C,B16H,B25H, B29K(N^{ε}Hexadecanedioyl-γGlu-OEG-OEG),desB30 human insulin,
A10C,A14E,B4C,B16H,B25H,B29K(N^{ε}Hexadecanedioyl-γGlu),desB30 human insulin,
A10C,A14E,B4C,B16H,B25H,B29K(N^{ε}Octadecanedioyl-γGlu),desB30 human insulin,
A10C,A14E,B4C,B16H,B25H B29K(N^{ε}Octadecanedioyl-γGlu-OEG-OEG),desB30 human insulin,
A10C,A14E,B4C,B16H B25H,B29K(N^{ε}Eicosanedioyl-γGlu-OEG-OEG),desB30 human insulin,
A10C,A14E,B4C,B16H,B25H,B29K(N^{ε}Eicosanedioyl-γGlu),desB30 human insulin,
A10C,A14E,B1C,B25H,B29K(N^{ε}eicosanedioyl-γGlu),desB30 human insulin,
A10C,A14E,B2C,B25H,B29K(N^{ε}eicosanedioyl-γGlu),desB30 human insulin,
A10C,A14E,B2C,B25H,B29K(N^{ε}eicosanedioyl-γGlu-OEG-OEG),desB30 human insulin,
A10C,A14E,B4C,B25H,desB27,B29K(N^{ε}eicosanedioyl-γGlu),desB30 human insulin,
A10C,A14E,B4C,B25H,desB27,B29K(N^{ε}eicosanedioyl-γGlu-OEG-OEG),desB30 human insulin,
A10C,A14E,B4C,B25H,B29K(N^{ε}eicosanedioyl-γGlu),desB30 human insulin,
A10C,A14E,B3C,B25H,desB27,B29K(N^{ε}hexadecanedioyl-γGlu),desB30 human insulin,
A10C,A14E,B3C,B25H,desB27,B29K(N^{ε}hexadecanedioyl-γGlu-OEG-OEG),desB30 human insulin,
A10C,A14E,B3C,desB27,B29K(N^{ε}hexadecanedioyl-γGlu),desB30 human insulin,
A10C,A14E,B3C,desB27,B29K(N^{ε}hexadecanedioyl-γGlu-OEG-OEG),desB30 human insulin,
A10C,A14E,B3C,desB27,B29K(N^{ε}octadecanedioyl-γGlu),desB30 human insulin, A10C,A14E,B3C,desB27,B29K(N^{ε}octadecanedioyl-γGlu-OEG-OEG),desB30 human insulin,
A10C,A14E,B3C,desB27,B29K(N^{ε}eicosanedioyl-γGlu),desB30 human insulin,
A10C,A14E,B3C,desB27,B29K(N^{ε}eicosanedioyl-γGlu-OEG-OEG),desB30 human insulin,
A10C,A14E,B3C,B16H,B25H,B29K(N^{ε}eicosanedioyl-γGlu-γGlu),desB30 human insulin,
A10C,A14E,B3C,B16E,B25H,B29K(N^{ε}eicosanedioyl-γGlu-OEG-OEG),desB30 human insulin,
A10C,A14E,B4C,B16E,B25H,B29K(N^{ε}eicosanedioyl-γGlu-OEG-OEG),desB30 human insulin,
A10C,A14E,B3C,B16H,B25H,B29K(N^{ε}eicosanedioyl-γGlu-γGlu),desB30 human insulin and A10C,A14E,B4C,B16E,B25H,B29K(N^{ε}eicosanedioyl-γGlu-γGlu),desB30 human insulin.
111. The pharmaceutical composition according to any one of the preceding aspects, wherein said protease stabilised insulin is selected from the group consisting of:
A10C,A14E,B4C,B25H,B29K(N^{ε}Octadecanedioyl-γGlu-OEG-OEG),desB30 human insulin,
A10C,A14E,B3C,B25H,B29K(N^{ε}Octadecanedioyl-γGlu),desB30 human insulin,
A10C,A14E,B3C,B25H,B29K(N^{ε}Octadecanedioyl),desB30 human insulin,
A10C,A14E,B3C,B25H,B29K(N^{ε}Octadecanedioyl-γGlu),desB30 human insulin,
A10C,A14H,B4C,B25H,B29K(N^{ε}Octadecanedioyl-γGlu-OEG-OEG),desB30 human insulin,
A10C,A14E,B3C,B25H,B29K(N^{ε}Eicosanedioyl-γGlu-OEG-OEG),desB30 human insulin,
A10C,A14E,B4C B25H,B29K(N^{ε}Octadecanedioyl-γGlu),desB30 human insulin,
A10C,A14E, B3C,B25H,B29K(N^{ε}Octadecanedioyl-γGlu-OEG-OEG),desB30 human insulin,
A10C,A14E,B3C,B25H,B29K(N^{ε}Octadecanedioyl-γGlu-γGlu),desB30 human insulin,
A10C,A14E,B4C,B25H,desB27,B29K(N^{ε}Octadecanedioyl-γGlu),desB30 human insulin,
A10C,A14E,B4C,B25H,B29K(N^{ε}Octadecanedioyl),desB30 human insulin,
A10C,A14E,B4C,B25H,B29K(N^{ε}Octadecanedioyl-γGlu-γGlu),desB30 human insulin,
A10C,A14E,B3C,B16H,B25H,B29K(N^{ε}Eicosanedioyl-γGlu-OEG-OEG),desB30 human insulin,
A10C,A14E, B4C,B25H,B29K(N^{ε}Myristyl),desB30 human insulin,
A10C,B4C, B29K(N^{ε}Myristyl),desB30 human insulin,
A10C,A14E,B3C,B25H,desB27,B29K(N^{ε}octadecanedioyl-γGlu),desB30 human insulin,
A10C,A14E,B3C,B25H,desB27,B29K(N^{ε}octadecanedioyl-γGlu-OEG-OEG),desB30 human insulin,
A10C,A14E,B3C,B25H,B29K(N^{ε}Eicosanedioyl-γGlu),desB30 human insulin,
A10C,A14E,B4C,B25H,B29K(N^{ε}eicosanedioyl-γGlu-OEG-OEG),desB30 human insulin,
A10C,A14E,B3C,B25H,desB27,B29K(N^{ε}eicosanedioyl-γGlu),desB30 human insulin,
A10C,A14E,B3C,B25H,desB27,B29K(N^{ε}eicosanedioyl-γGlu-OEG-OEG),desB30 human insulin,
A10C,A14E, 4C,B25H,B29K(N^{ε}Hexadecanedioyl-γGlu),desB30 human insulin,
A10C,A14E,B4C,B25H,B29K(N^{ε}Hexadecanedioyl-γGlu-OEG-OEG),desB30 human insulin,
A10C,A14E,B4C,B25H,B29K(N^{ε}Hexadecanedioyl),desB30 human insulin,
A10C,A14E,B4C,B25H,B29K(N^{ε}Hexadecanedioyl-γGlu-γGlu),desB30 human insulin,
A10C,A14E,B4C,B25H,desB27,B29K(N^{ε}Octadecanedioyl-γGlu-OEG-OEG),desB30 human insulin,
A10C,A14E,B4C,B25H,desB27,B29K(N^{ε}Octadecanedioyl-γGlu-γGlu),desB30 human insulin,
A10C,A14E,B4C,B25H,desB27,B29K(N^{ε}Hexadecanedioyl-γGlu-OEG-OEG),desB30 human insulin,
A10C,A14E,B4C,B25H,desB27,B29K(N^{ε}Hexadecanedioyl-γGlu),desB30 human insulin,
A10C,A14E,B3C,B25H,B29K(N^{ε}Hexadecanedioyl-γGlu),desB30 human insulin,
A10C,A14E,B3C,B25H,B29K(N^{ε}Hexadecanedioyl-γGlu-OEG-OEG),desB30 human insulin,
A10C,B3C,B29K(N^{ε}Hexadecanedioyl-γGlu),desB30 human insulin,
10C,B3C B29K(N^{ε}Hexadecanedioyl-γGlu-OEG-OEG),desB30 human insulin,
A10C,B3C,B29K(N^{ε}Octadecanedioyl-γGlu-OEG-OEG),desB30 human insulin,
A10C,B3C,B29K(N^{ε}Octadecanedioyl-γGlu),desB30 human insulin,
A10C,B4C,B29K(N^{ε}Octadecanedioyl-γGlu),desB30 human insulin,
A10C,B4C,B29K(N^{ε}Octadecanedioyl-γGlu-OEG-OEG),desB30 human insulin,
A10C,B4C B29K(N^{ε}Hexadecanedioyl-γGlu-OEG-OEG),desB30 human insulin,
A10C,B4C,B29K(N^{ε}Hexadecanedioyl-γGlu),desB30 human insulin,
A10C,A14E,B3C,B16H,B25H,B29K(N^{ε}Eicosanedioyl-γGlu),desB30 human insulin,
A10C,A14E,B3C,B16H,B25H, B29K(N^{ε}Octadecanedioyl-γGlu-OEG-OEG),desB30 human insulin,
A10C,A14E,B3C,B16H, B25H,B29K(N^{ε}Octadecanedioyl-γGlu),desB30 human insulin
A10C,A14E,B3C,B16H,B25H,B29K(N^{ε}Hexadecanedioyl-γGlu),desB30 human insulin,
A10C,A14E,B3C,B16H,B25H, B29K(N^{ε}Hexadecanedioyl-γGlu-OEG-OEG),desB30 human insulin,
A10C,A14E,B4C,B16H,B25H,B29K(N^{ε}Hexadecanedioyl-γGlu-OEG-OEG),desB30 human insulin,
A10C,A14E,B4C,B16H,B25H,B29K(N^{ε}Hexadecanedioyl-γGlu),desB30 human insulin,
A10C,A14E,B4C,B16H,B25H,B29K(N^{ε}Octadecanedioyl-γGlu),desB30 human insulin,
A10C,A14E,B4C,B16H,B25H B29K(N^{ε}Octadecanedioyl-γGlu-OEG-OEG),desB30 human insulin,
A10C,A14E,B4C,B16H B25H,B29K(N^{ε}Eicosanedioyl-γGlu-OEG-OEG),desB30 human insulin,
A10C,A14E,B4C,B16H,B25H,B29K(N^{ε}Eicosanedioyl-γGlu),desB30 human insulin,
A10C,A14E,B4C,B25H,desB27,B29K(N^{ε}eicosanedioyl-γGlu),desB30 human insulin, A10C,A14E,B4C,B25H,desB27,B29K(N^{ε}eicosanedioyl-γGlu-OEG-OEG),desB30 human insulin,
A10C,A14E,B4C,B25H,B29K(N^{ε}eicosanedioyl-γGlu),desB30 human insulin,
A10C,A14E,B3C,B25H,desB27,B29K(N^{ε}hexadecanedioyl-γGlu),desB30 human insulin,
A10C,A14E,B3C,B25H,desB27,B29K(N^{ε}hexadecanedioyl-γGlu-OEG-OEG),desB30 human insulin,
A10C,A14E,B3C,desB27,B29K(N^{ε}hexadecanedioyl-γGlu),desB30 human insulin,
A10C,A14E,B3C,desB27,B29K(N^{ε}hexadecanedioyl-γGlu-OEG-OEG),desB30 human insulin,
A10C,A14E,B3C,desB27,B29K(N^{ε}octadecanedioyl-γGlu),desB30 human insulin,
A10C,A14E,B3C,desB27,B29K(N^{ε}octadecanedioyl-γGlu-OEG-OEG),desB30 human insulin,
A10C,A14E,B3C,desB27,B29K(N^{ε}eicosanedioyl-γGlu),desB30 human insulin,
A10C,A14E,B3C,desB27,B29K(N^{ε}eicosanedioyl-γGlu-OEG-OEG),desB30 human insulin,
A10C,A14E,B3C,B16H,B25H,B29K(N^{ε}eicosanedioyl-γGlu-γGlu),desB30 human insulin,
A10C,A14E,B3C,B16E,B25H,B29K(N^{ε}eicosanedioyl-γGlu-OEG-OEG),desB30 human insulin,
A10C,A14E,B4C,B16E,B25H,B29K(N^{ε}eicosanedioyl-γGlu-OEG-OEG),desB30 human insulin, A10C,A14E,B3C,B16H,B25H,B29K(N^{ε}eicosanedioyl-γGlu-γGlu),desB30 human insulin and
A10C,A14E,B4C,B16E,B25H,B29K(N^{ε}eicosanedioyl-γGlu-γGlu),desB30 human insulin.
112. The pharmaceutical composition according to any one of the preceding aspects, wherein said protease stabilised insulin is selected from the group consisting of:
A14E,B25H,B29K(N^{ε}Octadecanedioyl-γGlu-OEG-OEG),desB30 human insulin,
A14E,B16H,B25H,B29K(N^{ε}Octadecanedioyl-γGlu-OEG-OEG),desB30 human insulin,
A14E,B16H,B25H,B29K(N^{ε}Eicosanedioyl-γGlu-OEG-OEG),desB30 human insulin,
A14E,B25H,desB27,B29K(N^{ε}Octadecanedioyl-γGlu-OEG-OEG),desB30 human insulin, A14E,B16H,B25H,B29K(N^{ε}Eicosanedioyl-γGlu),desB30 human insulin,
A14E,B25H,desB27,B29K(N^{ε}Octadecanedioyl-γGlu),desB30 human insulin,
A14E,B25H,desB27,B29K(N^{ε}Eicosanedioyl-γGlu),desB30 human insulin and
A14E,B25H,desB27,B29K(N^{ε}Eicosanedioyl-γGlu-OEG-OEG),desB30 human insulin,
A10C,A14E,B4C,B25H,B29K(N^{ε}Octadecanedioyl-γGlu-OEG-OEG),desB30 human insulin,
A10C,A14E,B3C,B25H,B29K(N^{ε}Octadecanedioyl-γGlu),desB30 human insulin,
A10C,A14E,B4C,B25H,desB27,B29K(N^{ε}Octadecanedioyl-γGlu),desB30 human insulin,
A10C,A14E,B3C,B16H,B25H,B29K(N^{ε}Eicosanedioyl-γGlu-OEG-OEG),desB30 human insulin,
A10C,A14E,B3C,B25H,desB27,B29K(N^{ε}octadecanedioyl-γGlu-OEG-OEG),desB30 human insulin,
A10C,A14E,B3C,B25H,desB27, 629K(N^{ε}eicosanedioyl-γGlu-OEG-OEG),desB30 human insulin,
A10C,A14E,B3C,B16H,B25H, B29K(N^{ε}Octadecanedioyl-γGlu-OEG-OEG),desB30 human insulin,
A10C,A14E,B4C,B16H,B25H B29K(N^{ε}Octadecanedioyl-γGlu-OEG-OEG),desB30 human insulin,
A10C,A14E,B4C,B16H B25H,B29K(N^{ε}Eicosanedioyl-γGlu-OEG-OEG),desB30 human insulin and
A10C,A14E,B4C,B25H,desB27,B29K(N^{ε}eicosanedioyl-γGlu-OEG-OEG),desB30 human insulin.
113. The pharmaceutical composition according to any one of the preceding aspects, wherein said protease stabilised insulin is selected from the group consisting of:
A10C,A14E,B4C,B25H,B29K(N^{ε}Octadecanedioyl-γGlu-OEG-OEG),desB30 human insulin,
A10C,A14E,B3C,B25H,B29K(N^{ε}Octadecanedioyl-γGlu),desB30 human insulin,
A10C,A14E,B4C,B25H,desB27,B29K(N^{ε}Octadecanedioyl-γGlu),desB30 human insulin, A10C,A14E,B3C,B16H,B25H,B29K(N^{ε}Eicosanedioyl-γGlu-OEG-OEG),desB30 human insulin,
A10C,A14E,B3C,B25H,desB27,B29K(N^{ε}octadecanedioyl-γGlu-OEG-OEG),desB30 human insulin,
A10C,A14E,B3C,B25H,desB27, 629K(N^{ε}eicosanedioyl-γGlu-OEG-OEG),desB30 human insulin,
A10C,A14E,B3C,B16H,B25H, B29K(N^{ε}Octadecanedioyl-γGlu-OEG-OEG),desB30 human insulin,
A10C,A14E,B4C,B16H,B25H B29K(N^{ε}Octadecanedioyl-γGlu-OEG-OEG),desB30 human insulin,
A10C,A14E,B4C,B16H B25H,B29K(N^{ε}Eicosanedioyl-γGlu-OEG-OEG),desB30 human insulin and
A10C,A14E,B4C,B25H,desB27,B29K(N^{ε}eicosanedioyl-γGlu-OEG-OEG),desB30 human insulin.
114. The pharmaceutical composition according to any one of the preceding aspects, wherein said protease stabilised insulin is selected from the group consisting of:
A14E,B25H,B29K(N^{ε}Octadecanedioyl-γGlu-OEG-OEG),desB30 human insulin,
A14E,B16H,B25H,B29K(N^{ε}Octadecanedioyl-γGlu-OEG-OEG),desB30 human insulin,
A14E,B16H,B25H,B29K(N^{ε}Eicosanedioyl-γGlu-OEG-OEG),desB30 human insulin,
A14E,B25H,desB27,B29K(N^{ε}Octadecanedioyl-γGlu-OEG-OEG),desB30 human insulin,
A14E,B16H,B25H,B29K(N^{ε}Eicosanedioyl-γGlu),desB30 human insulin,
A14E,B25H,desB27,B29K(N^{ε}Octadecanedioyl-γGlu),desB30 human insulin,
A14E,B25H,desB27,B29K(N^{ε}Eicosanedioyl-γGlu),desB30 human insulin and
A14E,B25H,desB27,B29K(N^{ε}Eicosanedioyl-γGlu-OEG-OEG),desB30 human insulin.
115. The pharmaceutical composition according to any one of the preceding aspects for use as a medicament.
116. The pharmaceutical composition according to any one of aspects 1-114 for use in treating diabetes mellitus.
117. The pharmaceutical composition according to any one of aspects 1-114 for use in treating type 1 and/or type 2 diabetes mellitus.
118. A method for producing a pharmaceutical composition according to any one of the aspects 1-114, comprising the steps of preparing a tablet core and directly coating said anionic copolymer on said outer surface of said tablet core.
119. The method according to aspect 118, wherein said tablet core is in the form of a uniform tablet, a single or multilayered tablet, a multiparticulate system, a capsule, a tablet contained in a capsule, multiple tablets contained in a capsule multiple tablets contained in a tablet, a multiparticulate system in the form of a tablet contained in a capsule or in a form of multiparticulate system compressed in one, some or all layers of said tablet core.
120. A method for producing a single layered tablet according to aspect 118, comprising the steps of preparing a tablet core pressed into a tablet form and directly coating said anionic copolymer coating on said outer surface of said tablet core.

### Materials and Methods

### LIST OF ABBREVIATIONS

βAla is beta-alanyl,

Aoc is 8-aminooctanoic acid,

tBu is tert-butyl,

CV is column volumes,

DCM is dichloromethane,

DIC is diisopropylcarbodiimide,

DIPEA=DIEA is N,N-disopropylethylamine,

DMF is N,N-dimethylformamide,

DMSO is dimethyl sulphoxide,

EtOAc is ethyl acetate,

Fmoc is 9-fluorenylmethyloxycarbonyl,

γGlu is gamma L-glutamyl,

HCl is hydrochloric acid,

HOBt is 1-hydroxybenzotriazole,

NMP is N-methylpyrrolidone,

MeCN is acetonitrile,

OEG is [2-(2-aminoethoxy)ethoxy]ethylcarbonyl,

Su is succinimidyl-1-yl=2,5-dioxo-pyrrolidin-1-yl,

OSu is succinimidyl-1-yloxy=2,5-dioxo-pyrrolidin-1-yloxy,

RPC is reverse phase chromatography,

RT is room temperature,

TFA is trifluoroacetic acid,

THF is tetrahydrofuran,

TNBS is 2,4,6-trinitrobenzenesulfonic acid,

TRIS is tris(hydroxymethyl)aminomethane

TSTU is O—(N-succinimidyI)-1,1,3,3-tetramethyluronium tetrafluoroborate.

### Method 1: General Methods of Preparation of Protease Stabilised Insulins

The production of polypeptides and peptides such as insulin is well known in the art. Polypeptides or peptides may for instance be produced by classical peptide synthesis, e.g. solid phase peptide synthesis using t-Boc or Fmoc chemistry or other well established techniques, see e.g. Greene and Wuts, “Protective Groups in Organic Synthesis”, John Wiley & Sons, 1999. The polypeptides or peptides may also be produced by a method which comprises culturing a host cell containing a DNA sequence encoding the (poly)peptide and capable of expressing the (poly)peptide in a suitable nutrient medium under conditions permitting the expression of the peptide. For (poly)peptides comprising non-natural amino acid residues, the recombinant cell should be modified such that the non-natural amino acids are incorporated into the (poly)peptide, for instance by use of tRNA mutants. To effect covalent attachment of the polymer molecule(s) to the polypeptide, the hydroxyl end groups of the polymer molecule are provided in activated form, i.e. with reactive functional groups. Suitable activated polymer molecules are commercially available, e.g. from Shearwater Corp., Huntsville, Ala., USA, or from PoIyMASC Pharmaceuticals plc, UK. Alternatively, the polymer molecules may be activated by conventional methods known in the art, e.g. as disclosed in WO 90/13540. Specific examples of activated linear or branched polymer molecules for use in the present invention are described in the Shearwater Corp. 1997 and 2000 Catalogs (Functionalized Biocompatible Polymers for Research and pharmaceuticals, Polyethylene Glycol and Derivatives, incorporated herein by reference). Specific examples of activated PEG polymers include the following linear PEGs: NHS-PEG (e.g. SPA-PEG, SSPA-PEG, SBA-PEG, SS-PEG, SSA-PEG, SC-PEG, SG-PEG, and SCM-PEG), and NOR-PEG), BTC-PEG, EPDX-PEG, NCO-PEG, NPC-PEG, CDI-PEG, ALD-PEG, TRES-PEG, VS-PEG, IODO-PEG, and MAL-PEG, and branched PEGs such as PEG2-NHS and those disclosed in U.S. Pat. No. 5,932,462 and U.S. Pat. No. 5,643,575.

The conjugation of the polypeptide and the activated polymer molecules is conducted by use of any conventional method, e.g. as described in the following references (which also describe suitable methods for activation of polymer molecules): R. F. Taylor, (1991), “Protein immobilisation. Fundamental and applications”, Marcel Dekker, N.Y.; S. S. Wong, (1992), “Chemistry of Protein Conjugation and Crosslinking”, CRC Press, Boca Raton; G. T. Hermanson et al., (1993), “Immobilized Affinity Ligand Techniques”, Academic Press, N.Y.). The skilled person will be aware that the activation method and/or conjugation chemistry to be used depends on the attachment group(s) of the polypeptide (examples of which are given further above), as well as the functional groups of the polymer (e.g. being amine, hydroxyl, carboxyl, aldehyde, sulfydryl, succinimidyl, maleimide, vinysulfone or haloacetate).

The following examples are offered by way of illustration, not by limitation. The preparation of the insulin analogues or derivatives used in the composition of the present invention are described by the chemical reactions described in their general applicability to the preparation. Occasionally, the reaction may not be applicable as described to each compound included within the disclosed scope of the invention. The insulin analogues or derivatives for which this occurs will be readily recognised by those skilled in the art. In these cases the reactions may be successfully performed by conventional modifications known to those skilled in the art, which is, by appropriate protection of interfering groups, by changing to other conventional reagents, or by routine modification of reaction conditions. Alternatively, other reactions disclosed herein or otherwise conventional will be applicable to the preparation of the corresponding insulin analogue or derivatives of the invention. In all preparative methods, all starting materials are known or may easily be prepared from known starting materials. All temperatures are set forth in degrees Celsius and unless otherwise indicated, all parts and percentages are by weight when referring to yields and all parts are by volume when referring to solvents and eluents.

The insulin analogues or derivatives used in the invention may be purified by employing one or more of the following procedures which are typical within the art. These procedures may—if needed—be modified with regard to gradients, pH, salts, concentrations, flow, columns and so forth. Depending on factors such as impurity profile, solubility of the insulins in question etcetera, these modifications may readily be recognised and made by a person skilled in the art.

After acidic HPLC or desalting, the insulin analogue or derivative is isolated by lyophilisation of the pure fractions.

After neutral HPLC or anion exchange chromatography, the compounds are desalted, precipitated at isoelectrical pH, or purified by acidic HPLC.

### Method 2: Typical Insulin Purification Procedures

The HPLC system is a Gilson system consisting of the following: Model 215 Liquid handler, Model 322-H2 Pump and a Model 155 UV Dector. Detection is typically at 210 nm and 280 nm.

The Äkta Purifier FPLC system (GE Health Care) consists of the following: Model P-900 Pump, Model UV-900 UV detector, Model pH/C-900 pH and conductivity detector, Model Frac-950 Fraction collector. UV detection is typically at 214 nm, 254 nm and 276 nm. The Äkta Explorer Air FPLC system (Amersham BioGE Health Caresciences) consists of the following: Model P-900 Pump, Model UV-900 UV detector, Model pH/C-900 pH and conductivity detector, Model Frac-950 Fraction collector. UV detection is typically at 214 nm, 254 nm and 276 nm

Acidic HPLC:

Column: Phenomenex, Gemini, 5μ, C18, 110 Å, 250×30 cm

Flow: 20 ml/min′

Eluent: A: 0,1% TFA in water B: 0,1% TFA in CH₃CN

Gradient:

| | |
|---|---|
| 0-7.5 min: | 10% B |
| 7.5-87.5 min: | 10% B to 60% B |
| 87.5-92.5 min: | 60% B |
| 92.5-97.5 min: | 60% B to 100% B |

Neutral HPLC:
Column: Phenomenex, Gemini, C18, 5 μm 250×30.00 mm, 110 Å
Flow: 20 ml/min
Eluent: A: 20% CH₃CN in aqueous 10 mM TRIS+15 mM (NH₄)SO₄ pH=7.3 B: 80% CH₃CN, 20% water
   Gradient:

| | |
|---|---|
| 0-7.5 min: | 0% B |
| 7.5-52.5 min: | 0% B to 60% B |
| 52.5-57.5 min: | 60% B |
| 57.5-58 min: | 60% B to 100% B |
| 58-60 min: | 100% B |
| 60-63 min: | 10% B |

Anion Exchange Chromatography:

Column: RessourceQ, 6 ml,

Flow: 6 ml/min

Buffer A: 0.09% NH₄HCO₃, 0.25% NH₄OAc, 42.5% ethanol pH 8.4

Buffer B: 0.09% NH₄HCO₃, 2.5% NH₄OAc, 42.5% ethanol pH 8.4

Gradient: 100% A to 100% B during 30 CV
Column: Source 30Q, 30×250 mm
Flow: 80 ml/min
Buffer A: 15 mM TRIS, 30 mM Ammoniumacetat i 50% Ethanol, pH 7.5 (1.25 mS/cm)
Buffer B: 15 mM TRIS, 300 mM Ammoniumacetat i 50% Ethanol pH 7.5 (7.7 mS/cm)
Gradient: 15% B to 70% B over 40 CV

Desalting:

Column: Daiso 200 Å 15 um FeFgel 304, 30×250 mm

Buffer A: 20 v/v % Ethanol, 0.2% acetic acid

Buffer B: 80% v/v % Ethanol, 0.2% acetic acid

Gradient: 0-80% B over 1.5 CV

Flow: 80 ml/min

Column: HiPrep 26/10

Flow: 10 ml/min,

Gradient: 6 CV

Buffer: 10 mM NH₄HCO₃

General Procedure for the Solid Phase Synthesis of Acylation Reagents of the General Formula CHEM 3:

Acy-AA1n-AA2m-AA3p-Act, CHEM 3:

wherein Acy, AA1, AA2, AA3, n, m, and p are as defined above and Act is the leaving group of an active ester, such as N-hydroxysuccinimide (OSu), or 1-hydroxybenzotriazole, and

wherein carboxylic acids within the Acy and AA2 moieties of the acyl moiety are protected as tert-butyl esters.

Insulin analogue or derivatives of general formula CHEM 3 used according to the invention may be synthesised on solid support using procedures well known to skilled persons in the art of solid phase peptide synthesis. This procedure comprises attachment of a Fmoc protected amino acid to a polystyrene 2-chlorotritylchloride resin. The attachment can, e.g., be accomplished using the free N-protected amino acid in the presence of a tertiary amine, like triethyl amine or N,N-di-isopropylethylamine (see references below). The C-terminal end (which is attached to the resin) of this amino acid is at the end of the synthetic sequence being coupled to the parent insulins of the invention. After attachment of the Fmoc amino acid to the resin, the Fmoc group is deprotected using, e.g., secondary amines, like piperidine or diethyl amine, followed by coupling of another (or the same) Fmoc protected amino acid and deprotection. The synthetic sequence is terminated by coupling of mono-tert-butyl protected fatty (α, ω) diacids, like hexadecanedioic, heptadecanedioic, octadecanedioic or eicosanedioic acid mono-tert-butyl esters. Cleavage of the compounds from the resin is accomplished using diluted acid like 0.5-5% TFA/DCM (trifluoroacetic acid in dichloromethane), acetic acid (e.g., 10% in DCM, or HOAc/triflouroethanol/DCM 1:1:8), or hecafluoroisopropanol in DCM (See, e.g., “Organic Synthesis on Solid Phase”, F. Z. Dörwald, Wiley-VCH, 2000. ISBN 3-527-29950-5, “Peptides: Chemistry and Biology”, N. Sewald & H.-D. Jakubke, Wiley-VCH, 2002, ISBN 3-527-30405-3 or “The Combinatorial Cheemistry Catalog” 1999, Novabiochem AG, and references cited therein). This ensures that tert-butyl esters present in the compounds as carboxylic acid protecting groups are not deprotected. Finally, the C-terminal carboxy group (liberated from the resin) is activated, e.g., as the N-hydroxysuccinimide ester (OSu) and used either directly or after purification as coupling reagent in attachment to parent insulins of the invention. This procedure is described in example 9 in, WO09115469.

Alternatively, the acylation reagents of the general formula CHEM 3 above may be prepared by solution phase synthesis as described below.

Mono-tert-butyl protected fatty diacids, such as hexadecanedioic, heptadecanedioic, octadecanedioic or eicosanedioic acid mono-tert-butyl esters are activated, e.g., as OSu-esters as described below or as any other activated ester known to those skilled in the art, such as HOBt- or HOAt-esters. This active ester is coupled with one of the amino acids AA1, mono-tert-butyl protected AA2, or AA3 in a suitable solvent such as THF, DMF, NMP (or a solvent mixture) in the presence of a suitable base, such as DIPEA or triethylamine. The intermediate is isolated,e.g., by extractive procedures or by chromatographic procedures. The resulting intermediate is again subjected to activation (as described above) and to coupling with one of the amino acids AA1, mono-tert-butyl protected AA2, or AA3 as described above. This procedure is repeated until the desired protected intermediate Acy-AA1n-AA2m-AA3p-OH is obtained. This is in turn activated to afford the acylation reagents of the general formula CHEM 3 Acy-AA1n-AA2m-AA3p-Act. This procedure is described in example 11 in WO09115469.

The acylation reagents prepared by any of the above methods may be (tert-butyl) de-protected after activation as OSu esters. This may be done by TFA treatment of the OSu-activated tert-butyl protected acylation reagent. After acylation of any insulin, the resulting unprotected acylated protease stabilied insulin of the invention is obtained. This procedure is described in example 16 in WO09115469. If the reagents prepared by any of the above methods are not (tert-butyl) de-protected after activation as OSu esters, acylation of any insulin affords the corresponding tert-butyl protected acylated insulin of the invention. In order to obtain the unprotected acylated insulin of the invention, the protected insulin is to be de-protected. This may be done by TFA treatment to afford the unprotected acylated insulin of the invention. This procedure is described in example 1 in WO05012347.

Methods for preparation of acylated insulins may be found in WO09115469. In one embodiment of the invention, acylated insulin used in a composition according to the present invention, wherein the insulin is an acylated, protease stabilised insulin.

Method 3: Preparing a Tablet Core According to this Invention

The tablets according to this invention are prepared so that a person skilled in the art of pharmaceutical tablet production easily can make the tablets. The formulation of a tablet core material according to the present invention was performed as outlined here, this example concerns formulations of the present invention comprising:

| | |
|---|---|
| protease stabilised insulin | 1.17% (w/w) |
| Sodium decanoate | 77.00% (w/w) |
| (i.e. sodium salt of capric acid) | |
| Sorbitol | 21.33% (w/w) |
| Stearic acid | 0.50% (w/w) |

When 100 g of tablet core material comprising protease stabilised insulin, sodium caprate (i.e. sodium salt of capric acid), sorbitol and stearic acid was manufactured acording to the above listed ingredients and in the corresponding ratios, the following steps were used:

The procedure was performed as follows:

Insulin powder was put through a sieve with a mesh size of 0.25 mm. After sieving the correct amount of protease stabilised insulin was weighed. Sorbitol powder was put through a sieve with a mesh size of 0.5 mm. After sieving the correct amount was weighed.

In a small container insulin and sorbitol was mixed. An amount of sorbitol equivalent to the amount of protease stabilised insulin was added to said container and stirred by hand. Then the double amount of sorbitol relative to the previous addition was added and stirred by hand until insulin and all sorbitol were mixed well. This step was followed by a mechanical mixing in a Turbula-mixer to finalize the mixing to obtain a homogeneous powder.

Sodium salt of capric acid (in the form of granulate) was then added to the insulin-sorbitol powder according to equal volumes principle. This was done in two steps and finalized with a mechanical mixing step in a Turbula-mixer.

Finally stearic acid was put through a sieve with a mesh size of 0.25 mm. Stearic acid was weighed and added to the powder and mixed mechanically.

The final granulate may then be subjected to a standard tabletting proces, such as a in a Fette 102I tabletting press. Tablets are produces to a technical level allowing for further processing such as e.g. coating.

METHOD 4: Preparing a Tablet Core with a Sub Coat

The powder prepared according to method 3 was then compressed in or a tablet press to form tablets of a mass of 710 mg. A tablet core prepared by this method was then coated with immediate release coating, comprising polyvinyl alcohol. The coating solution was prepared by dispersing the 20 g immediate release coating material, comprising polyvinyl alcohol in 80 g pure water. The concentration of immediate release coating comprising polyvinyl alcohol in the coating solution was 20%-volume. Under intense mixing using a standard magnetic stirrer the polymer powder was added to the water. After addition of polymer the mixture was stirred at low intensity for 30 minutes. The resulting coating solution was sieved to remove lumps. The coating of tablet cores was performed in a pan coater or fluid bed coater. In a pan coater with the pan size of 8.5″, with a conventional patterned air Schlick spray nozzle with an orifice of 1.0 mm, an atomizing and pattern air pressure of 0.5 bar, inlet air temperature of 38° C. and air flow of 130 kg/hour, the coating was performed by pumping the polymer solution in through the nozzle. After addition of 4.5% (w/w) polymer distributed evenly on the tablet cores the spraying is stopped and the tablets are allowed to dry for up to 30 minutes inside the pan.

Method 5: Preparing an Anionic Copolymer Coated Tablet Core

A tablet core is prepared according to method 3 (for producing a tablet comprising no sub coat) or method 4 (for producing a tablet comprising a sub coat) and coated with an anionic copolymer as described below:

A tablet core according to method 3 or a tablet core with a sub coating according to method 4 was coated with an outer coating.

For this purpose polymers of the copolymer family denominated “methyl acrylate-co-methyl methacrylate-co-methacrylic acid” (Brand name EUDRAGIT FS30D® as sold by Evonik Industries (in 2013)) were used.

121.2 g of an aqueous dispersion of methyl acrylate-co-methyl methacrylate-co-methacrylic acid (Brand name EUDRAGIT FS30D® as sold by Evonik Industries (in 2013)) is placed in a beaker on a suitable stirring apparatus. Glycerol monostearate, plasticizing agent triethyl citrate and polyoxyethylene (20) sorbitan monooleate in the form of 18.2 g PlasAcryl T20® and 60.6 pure water were added to the amount of 10% of the total dry polymer. The ingredients were added to said aqueous emulsion of methyl acrylate-co-methyl methacrylate-co-methacrylic acid (Brand name EUDRAGIT FS30D® as sold by Evonik Industries (in 2013)). The mixture was allowed to mix for 10 minutes prior to a filtration through a 0.24 mm mesh filter to remove lumps. The coating of tablet cores with an inner coat as well as tablets without an inner coat was performed in a pan coater or fluid bed coater. In a pan coater with the pan size of 8.5″, with a conventional patterned air Schlick spray nozzle with an orifice of 1.0 mm, an atomizing and pattern air pressure of 0.5-0.6 bar, inlet air temperature of 35 C, air flow of 130 kg/hours, the coating was performed by pumping the polymer solution in through the nozzle. After addition of 5-7% w/w polymer distributed evenly on the tablet cores including and excluding an inner coating as prepared in method 3 and 4, the spraying was stopped.

Method 6: Determining the Solubility pH of the Composition

Solubility of coated tablets according to the present invention, wherein the tablet core was coated with EUDRAGIT®FS30D coating as sold by Evonik Industries (in 2013) were tested at various pH values, results are shown in table 2. Tablets containing a tablet core, an Opadry-II sub coat (4.5% w/w) and a EUDRAGIT®FS30D coating as sold by Evonik Industries (in 2013) were also tested for comparison.

Tablets were placed in beakers under the pH conditions specified in table 2. After treatment the individual tablets were weighed. Weight was recorded as positive if the tablet increased in weight or negative if the tablet lost weight relative to the initial weight. Initially tablets were subjected to 0.1N HCl adjusted to pH1.2 for two periods of 1 hr each. The pH was increased to the below indicated set points with mixtures of 1M NaH₂PO₄ and 0.5M NaH₂PO₄ and the tablets were kept at this set point pH for 30 minutes.

TABLE 2
Results presented as percent weight gain of enteric coated tablets.

| | Functional | | | | | | | | |
|---|---|---|---|---|---|---|---|---|---|
| Sub- | coat | Percent weight gain | | | | | | | |
| coat | (FS30D) | (%) | | | | | | | |
| level | level | pH1.2 | pH1.2 | | | | | | |
| w/w % | w/w % | (1 hr) | (2 hr) | pH4.5 | pH5.5 | pH6.0 | pH6.5 | pH7.0 | pH7.4 |
| 0 | 3.8 | 1.19 | 2.90 | 3.27 | 4.70 | 6.41 | 9.93 | −30.07 | −100.00 |
| 0 | 5.8 | 0.72 | 1.23 | 1.59 | 2.21 | 2.97* | 3.95 | 8.72 | −44.02 |
| 0 | 7.4 | 0.00 | 0.42 | 0.87 | 0.80 | 1.12 | 1.20 | 4.18 | 9.15 |
| 4.5 | 4.4 | 1.13 | 2.08 | 3.10 | 3.84 | 5.80 | 8.91 | 27.85 | −100.00 |
| 4.5 | 6.9 | 0.48 | 1.16 | 1.37 | 1.55 | 1.79 | 2.07 | 7.73 | 15.25 |
| 4.5 | 7.8 | 0.20 | 0.65 | 0.78 | 0.92 | 1.24 | 1.49 | 5.84 | 16.64 |

The results in table 2 are presented as percent weight gain of enteric coated tablets exposed for different pH conditions. Weight gain indicates the hydration of the given enteric polymer coating as a function of pH. The results show that, as pH increases weight gain is seen in all cases. However, as the amount of coating is increased from about 3% to about 8%, pH for steep weight gain is moving towards higher pH value. Once the coating reaches its maximum limit pH for enteric protection, the tablets starts to disintigrate. This is observed as a negative weight gain and is thus indicating a weight loss due to loss of protective coating.

Method 7: Measuring Disolution Rate In Vitro

In an appropriate dissolution apparatus e.g. USP dissolution apparatus 2 a standard dissolution test according to the pharmacopoeia may be performed to measure dissolution in-vitro. In this test the tablets were exposed to a dissolution medium with a pH of 6.8. Under stirring the tablet dissolution was followed by sampling at pre-defined time intervals and analysed by HPLC chromatography.

Method 8: Collecting Samples for Measuring Bioavailabilty, Tmax for a Composition from Beagle Dogs

The dogs were fasted overnight before the test, (no food—only tap water). The day before the experiment the dogs were weighed and dogs were taken out for a couple of hours.

On the day of the experiment the dogs were placed on test couch and a Venflon 20 G is placed in v. cephalica. Blood samples were taken from the catheter. The venflon were removed 6 hours post dosing and the dogs were returned to their box, and offered exercise in the outside run. Hereafter the dogs were lead into a test room for blood sampling from v.jugularis (or v.cephalica).

Per Os Administration.

Blood samples for glucose and insulin were taken at: 0, 15, 30, 45, 60, 75, 90, 105, 120, 135, 150, 165, 180, 210, 240, 270, 300, 360, 480, 600, 720, 1440, 1800, 2880 and 4320 minutes.

The tablet was administrated right after the t=0 min sample was drawn. The tablet was placed in the back of the mouth so the dog would swallow the tablet without chewing it. After the dog had swallowed the tablet, 10 ml water was administrated into the mouth by a syringe.

Blood Sampling:

Before sampling the first drops of blood was collected on a tissue.

Approx. 800 μl blood was collected in 1.5 ml EDTA Eppendorf tubes for plasma and a 10 μL capillary tube was filled with full blood for glucose analysis.

The EDTA blood samples were centrifuged at 4000×g (4° C.) for 4 min.

All samples were kept on wet ice until analysis or stored at −80° C. until analysis.

After each sampling were the Venflon flushed with 0.5 ml heparin (10 IU).

Male Beagle dogs used weigh approximately from 12 to 18 kg approximately.

Plasma samples were analysed by either sandwich immunoassay or Liquid chromatography-mass spectrometry. Plasma concentration-time profiles were analysed by non-compartmental pharmacokinetics analysis using WinNonlin Professional 5.2 (Pharsight Inc., Mountain View, Calif., USA).

Method 9: Bioavailability and Pharmacokinetics Profile

Generally, the term bioavailability refers to the fraction of an administered dose of the active pharmaceutical ingredient (API), such as a derivative of the invention that reaches the systemic circulation unchanged. By definition, when an API is administered intravenously, its bioavailability is 100%. However, when it is administered via other routes (such as orally), its bioavailability decreases (due to degradation and/or incomplete absorption and first-pass metabolism). Knowledge about bioavailability is important when calculating dosages for non-intravenous routes of administration.

A plasma concentration versus time plot is made after both oral and intravenous administration. The absolute bioavailability (F) is the (AUC-oral divided by dose), divided by (AUC-intravenous divided by dose).

Increasing terminal half-life and/or decreasing of the clearance means that the compound in question is eliminated slower from the body. For the derivatives of the invention this entails an extended duration of pharmacological effect.

Increased oral bioavailability means that a larger fraction of the dose administered orally reach the systemic circulation from where it may distribute to exhibit pharmacological effect.

The pharmacokinetic properties of the derivatives of the invention may suitably be determined in-vivo in pharmacokinetic (PK) studies. Such studies are conducted to evaluate how pharmaceutical compounds are absorbed, distributed, and eliminated in the body, and how these processes affect the concentration of the compound in the body, over the course of time.

In the discovery and preclinical phase of pharmaceutical drug development, animal models such as the mouse, rat, monkey, dog, or pig, may be used to perform this characterisation. Any of these models may be used to test the pharmacokinetic properties of the derivatives of the invention.

In such studies, animals are typically administered with a single dose of the drug, either intravenously, subcutaneously (s.c.), or orally (p.o.) in a relevant formulation. Blood samples are drawn at predefined time points after dosing, and samples are analysed for concentration of drug with a relevant quantitative assay. Based on these measurements, time-plasma concentration profiles for the compound of study are plotted and a so-called non-compartmental pharmacokinetic analysis of the data is performed.

For most compounds, the terminal part of the plasma-concentration profiles will be linear when drawn in a semi-logarithmic plot, reflecting that after the initial absorption and distribution, drug is removed from the body at a constant fractional rate. The rate (lambda Z or λ_{z}) is equal to minus the slope of the terminal part of the plot. From this rate, also a terminal half-life may be calculated, as t½=ln(2)/λ_{z} (see, e.g., Johan Gabrielsson and Daniel Weiner: Pharmacokinetics and Pharmacodynamic Data Analysis. Concepts & Applications, 3rd Ed., Swedish Pharmaceutical Press, Stockholm (2000)).

Clearance may be determined after i.v. administration and is defined as the dose (D) divided by area under the curve (AUC) on the plasma concentration versus time profile (Rowland, M and Tozer T N: Clinical Pharmacokinetics: Concepts and Applications, 3^{rd} edition, 1995 Williams Wilkins).

The estimate of terminal half-life and/or clearance is relevant for evaluation of dosing regimens and an important parameter in drug development, in the evaluation of new drug compounds.

Method 10: Identifying “Absorbers” for Dog Studies

The oral exposure of protease stabilised insulin, detectable in blood/plasma samples of Beagle dogs is known to vary from dog to dog. If a dog is not showing exposure, i.e. if no insulin is detectable in the blood/plasma samples after administration of oral insulin, then the dog is a “non-absorber” and not used in the studies. When a dog however shows exposure, i.e. detectable values of protease stabilised insulin in the blood/plasma samples are recognised, then this dog is an “absorber” and may be used in studies of oral absorption.

Method 11: Testing Food Interferance

The testing of food interaction was investigated by sequential oral administration of pharmaceutical tablet and food. The set-up was as this: A tablet was given orally according to the method described. After pre-defined intervals food was given to the dogs.

### EXAMPLES

### Example 1

### Dissolution Rate of Compositions According to the Present Invention with/without Sub Coat

Tablet cores according to this invention where prepared by mixing the following ingredients according to method 3 and coated either according to method 4 and 5 or solely according to tablet 5 resulting in tablets comprising a tablet core, an Opadry®II sub coat and a EUDRAGIT®FS30D coating as sold by Evonik Industries (in 2013) or a tablet core, no sub coat and a EUDRAGIT®FS30D coating as sold by Evonik Industries (in 2013) in direct contact with the tablet core. The dose of protease stabilised insulin in dogs is in studies for the present patent application was set to 120 nmol/kg. Thus the absolute amount of protease stabilised insulin in said tablet core was adjusted according to the weight of the dog which was to receive said tablet for oral administration. In the present example the dog weighed 18 kg and the insulin thus amounted to 14.8 mg (120 nmol/kg).

Table 3 shows a composition according to the present invention comprising 14.8 mg A14E, B25H, B29K(N^{ε}Octadecanedioyl-γGlu-OEG-OEG), desB30 human insulin in a tablet core comprising sodium caprate and is coated with EUDRAGIT®FS30D coating as sold by Evonik Industries (in 2013). The tablet core weight is 710.1 mg, the enteric coated tablet without sub coat weight is 759.8 mg

TABLE 3

| | | | Final coated |
|---|---|---|---|
| | | | tablet (% |
| | mg/ | Core | w/w) |
| Tablet Excipient | tablet | (% w/w) | FS30D |
| A14E, B25H, | 14.8 | 2.1 | 1.9 |
| B29K(N^{ε}Octadecanedioyl-γGlu-OEG- | | | |
| OEG), desB30 human insulin | | | |
| Sodium caprate | 546.7 | 77 | 72 |
| Sorbitol | 145 | 20.4 | 19.1 |
| Stearic Acid | 3.6 | 0.5 | 0.5 |
| EUDRAGIT ® FS30D | 49.7 | N/A | 6.5 |

Dissolution is performed in USP2 (Paddle) at 50 rpm 37° C.±0.5° C. (Ph Eur 2.9.3). It is carried out as a solvent addition method. Initially dissolution is performed in 500 ml, 0.1N HCl, pH 1 pH for 120 minutes. Then 400 ml 0.12M phosphate solution is added to neutralize the acid and bring pH to 6.8 or 7.4. Hereafter, dissolution is further followed for 120 min. Samples of 2 ml are collected at given time points and quantified in for A14E, B25H, B29K(N^{ε}Octadecanedioyl-γGlu-OEG-OEG), desB30 human insulin as well as Sodium caprate.

### Example 2

### PK profiles of A14E, B25H, B29K(N^{ε}Octadecanedioyl-γGlu-OEG-OEG), desB30 Human Insulin in a Tablet Core Coated with/without Sub Coat Below an EUDRAGIT® FS30D Coating as Sold by Evonik Industries (in 2013)

Tablet cores were prepared according to method 3 comprising A14E, B25H, B29K(N^{ε}Octadecanedioyl-γGlu-OEG-OEG), desB30 human insulin and coated either according to method 4 with Opadry®II (when sub coat was applied) and method 5 with EUDRAGIT®FS30D as sold by Evonik Industries (in 2013) in combination or method 5 alone (when no sub coat was applied under said EUDRAGIT®FS30D coating as sold by Evonik Industries (in 2013)).

For the coading according to method 5 polymers of the copolymer family denominated “methyl acrylate-co-methyl methacrylate-co-methacrylic acid” (in this example EUDRAGIT®FS30D as sold by Evonik Industries (in 2013)) were used. 121.2 g of an aqueous dispersion of methyl acrylate-co-methyl methacrylate-co-methacrylic acid (Brand name EUDRAGIT®FS30D as sold by Evonik Industries (in 2013)) is placed in a beaker on a suitable stirring apparatus. Glycerol monostearate, plasticizing agent triethyl citrate and polyoxyethylene (20) sorbitan monooleate in the form of 18.2 g PlasAcryl T20® and 60.6 pure water was added to the amount of 10% of the total dry polymer. The ingredients were added to said aqueous emulsion of methyl acrylate-co-methyl methacrylate-co-methacrylic acid (Brand name EUDRAGIT®FS30D as sold by Evonik Industries (in 2013)). The mixture was allowed to mix for 10 minutes prior to a filtration through a 0.24 mm mesh filter to remove lumps. The coating of tablet cores with an inner coat as well as tablets without an inner coat was performed in a pan coater or fluid bed coater. In a pan coater with the pan size of 8.5″, with a conventional patterned air Schlick spray nozzle with an orifice of 1.0 mm, an atomizing and pattern air pressure of 0.5-0.6 bar, inlet air temperature of 35° C., air flow of 130 kg/hours, the coating was performed by pumping the polymer solution in through the nozzle. After addition of 5-7% w/w polymer distributed evenly on the tablet cores including and excluding an inner coating as prepared in method 3 and 4, the spraying was stopped. FIG. 2A shows the PK profiles for this insulin in tablet cores with Opadry®II sub coat below an EUDRAGIT FS30D coating as sold by Evonik Industries (in 2013), squares show the PK profile for tablets tested at time 0 and circles show the PK profile for tablets tested after 14 weeks storage at 5° C. Mean±SEM; n=8 FIG. 2B shows the PK profiles for this insulin in tablet coatcores without sub coat below an EUDRAGIT®FS30D coating as sold by Evonik Industries (in 2013), squares show the PK profile for tablets tested at time 0 and circles show the PK profile for tablets tested after 12 weeks storage at 5° C. Mean±SEM; n=8

Comparing the two FIGS. 2A and 2B is is clear that the tablet PK profile for A14E, B25H, B29K(N^{ε}Octadecanedioyl-γGlu-OEG-OEG), desB30 human insulin tablets without sub coat is stable, whereas the PK profile for the same insulin is not stable with an Opadry-II sub coat.

### Example 3

### Bioavailability of Freshly Coated Tablet Cores Comprising A14E, B25H, B29K(N^{ε}ctadecanedioyl-γGlu-OEG-OEG), desB30 Human Insulin Vs. Stored Compositions According to the Present Invention

Tablet cores were prepared according to method 3 comprising A14E, B25H, B29K(N^{ε}Octadecanedioyl-γGlu-OEG-OEG), desB30 human insulin and coated either according to method 4 with Opadry®II (when sub coat was applied) and method 5 with EUDRAGIT®FS30D as sold by Evonik Industries (in 2013) in combination or method 5 alone (when no sub coat was applied under said EUDRAGIT®FS30D coating as sold by Evonik Industries (in 2013)).

The bioavailability was tested at time 0 (i.e. shortly after the tablet preparation was completed) and after storage at 5° C. for 12 to 14 weeks after preparation was completed.

The results are given in the table 4, indicating that the highest bioavailability: The bioavailability was assessed according to the method description of in-vivo experiments in method 6.

The dogs were fasted overnight before the test, (no food—only tap water). The day before the experiment the dogs were weighed and dogs were taken out for a couple of hours.

On the day of the experiment the dogs were placed on test couch and a Venflon 20 G was placed in v. cephalica. Blood samples will be taken from the catheter. The venflon will be removed 6 hours post dosing and the dogs will be returned to their box, and offered exercise in the outside run. Hereafter the dogs will be lead into a test room for blood sampling from v.jugularis (or v.cephalica).

Per Os Administration.

Blood samples for glucose and insulin were taken at: 0, 15, 30, 45, 60, 75, 90, 105, 120, 135, 150, 165, 180, 210, 240, 270, 300, 360, 480, 600, 720, 1440, 1800, 2880 and 4320 minutes.

The tablet was administered right after the t=0 min sample was drawn. The tablet was placed in the back of the mouth so the dog will swallow the tablet without chewing it. After the dog has swallowed the tablet, 10 ml water was administered into the mouth by a syringe. Blood sampling: Before sampling the first drops of blood was collected on a tissue. Approx. 800 μl blood was collected in 1.5 ml EDTA Eppendorf tubes for plasma and a 10 μL capillary tube was filled with full blood for glucose analysis. The EDTA blood samples were centrifuged at 4000×g (4° C.) for 4 min. All samples were kept on wet ice until analysis or stored at −80° C. until analysis. After each sampling, the Venflon was flushed with 0.5 ml heparin (10 IU). Male Beagle dogs weighed approximately from 12 to 18 kg. Plasma samples were analysed by either sandwich immunoassay or liquid chromatography-mass spectrometry (LC-MS). Plasma concentration-time profiles were analysed by non-compartmental pharmacokinetics analysis using WinNonlin Professional 5.2 (Pharsight Inc., Mountain View, Calif., USA).

TABLE 4

| | | |
|---|---|---|
| Coating | Time 0 | Time 12-14 weeks |

With sub coat

| | | |
|---|---|---|
| Opadry-II (%) + | 2.3 ± 2.7% (n = 8) | N/A |
| Acryl-EZE ® 930 (%) | | |
| Opadry-II (%) + | 1.5 ± 1.9% (n = 16) | N/A |
| Acryl-EZE ® 93A (%) | | |
| Opadry-II + EUDRAGIT ® | 7.4 ± 6.5% (n = 8) | 1.4 ± 1.5% (n = 8) |
| FS30D (%) | | |

Without sub coat

| | | |
|---|---|---|
| EUDRAGIT ® FS30D | 6.7 ± 8.6% (n = 8) | 6.6 ± 7.6% (n = 8) |

### Example 4

### Tmax of Freshly Coated Tablet Cores Comprising A14E, B25H, B29K(WOctadecanedioyl-γGlu-OEG-OEG), desB30 Human Insulin Vs. Stored Compositions According to the Present Invention

Tablets were prepared according to method 3: Tmax was determined according to method 9 and the results are shown in table 5:

TABLE 5

| | | |
|---|---|---|
| | | Time |
| Coating | Time 0 | 12-14 weeks |

With sub coat

| | | |
|---|---|---|
| Opadry-II (%) + | 68 minutes (n = 8) | N/A |
| Acryl-EZE ® 930 (%) | | |
| Opadry-II (%) + | 75 minutes (n = 16) | N/A |
| Acryl-EZE ® 93A (%) | | |
| Opadry-II + EUDRAGIT ® | 165 minutes (n = 8) | 270 (n = 8) |
| FS30D (%) | | |

Without sub coat

| | | |
|---|---|---|
| EUDRAGIT ® FS30D | 150 minutes (n = 8) | 150 (n = 8) |

### Example 5

### Bioavailability of Eyprotease Stabilised Insulins in Compositions According to the Present Invention Compared to Compositions with an Opadry-II Sub Coat

Bioavailability was determined according to method 9. Tablet cores where prepared with different protease stabilised insulins according to method 3 in either un-coated or coated tablet cores according to method 5 with EUDRAGIT®FS30D as sold by Evonik Industries (in 2013).

The results are given in table 6 and show that the tablet core alone does not have the same positive effect on bioavailability as the tablet core and the EUDRAGIT®FS30D coat as sold by Evonik Industries (in 2013):

TABLE 6
The bioavailabilities (mean ± SD) derived from ¹ones daily multiple dose
studies, ²multiple single dose studies or ⁴one single study.
EUDRAGIT ® FS30D as sold by Evonik Industries (in 2013);
n = 48 (***p = 0.002) against tablet core in ³single dose studies
(F = 2.3 ± 2.7%)

| | | Tablet |
|---|---|---|
| | | core + |
| | Tablet | FS30D |
| Insulin | core alone | coat |
| A14E, B25H, | 2.8 ± 1.7¹% | 6.9 ± 7.1***²% |
| B29K(N^{ε}Octadecanedioyl- | | |
| γGlu-OEG-OEG), desB30 human | | |
| insulin | | |
| A14E, B16H, B25H, | 1.5 ± 1.4¹% | 5.0 ± 5.1⁴% |
| B29K(N^{ε}Eicosanedioyl-γGlu-OEG- | | |
| OEG), desB30 human insulin | | |
| A14E, B25H, desB27, | | 4.1 ± 3.4% (n = 16) |
| B29K(N^{ε}-(octadecandioyl-γGlu), | | |
| desB30 human insulin | | |
| A10C, A14E, B3C, B16H, B25H, | | 7.1 ± 5.1% (n = 16) |
| B29K(N^{ε}Eicosanedioyl-γGlu- | | |
| OEG-OEG), desB30 human insulin | | |

### Example 6

### Real Time Stability Studies 0-12 Weeks, of A14E, B25H, B29K(WOctadecanedioyl-γGlu-OEG-OEG), desB30 Human Insulin in a Tablet Core Coated without Sub Coat Below FS30D Coating (n=8)

Tablets according to the present invention were prepared according to table 1 (example 1) and method 3 and coated according to method 5 with an EUDRAGIT®FS30D coating as sold by Evonik Industries (in 2013) on top of said tablet core without a sub coat Tablets were produced and coated, packaged in duma-containers with a dessicant, stored at 5° C. and administered to Beagle dogs. Samples were collected as described in method 6.

Time points for this testing are specified in the table 3 to be 0, 3, 6, 9 and 12 weeks.

TABLE 7

| | | A14E, B25H, B29K(N^{ε}Octadecanedioyl-γGlu-OEG- | | | | | |
|---|---|---|---|---|---|---|---|
| | | OEG), desB30 human insulin F(%) in Beagle dogs | | | | | |
| | | Week | | | | | |
| | PH | 0 | 3 | 6 | 9 | 12 | mean |
| EUDRAGIT ® FS30D | 7.2 | 6.0 ± 5.5 | 8.0 ± 7.5 | 7.2 ± 6.9 | 7.9 ± 9.0 | 6.6 ± 7.6 | 7.0 ± 7.0 |

The results shown in Table 7 confirm that the composition according to this invention is stable upon storage, which is confirmed by the PK profile of the same insulin in FIG. 2A (example 2).

### Example 7

### Food Interaction on A14E, B25H, B29K(N^{ε}Octadecanedioyl-γGlu-OEG-OEG), desB30 Human Insulin Bioavailability in Composition According to the Present Invention

Tablets according to the present invention were prepared according to method 3 and coated according to method 4. The tablets were administered to Beagle dogs and samples were collected as described in method 6. Food interaction was tested according to method 11. The results are shown in Table 8.

TABLE 8

| | Feeding post | Number of | | |
|---|---|---|---|---|
| | dosing | absorbers | Mean F | Median |
| Coating | (minutes) | (%) | (%) ± SD | Tₘₐₓ ± SD |
| EUDRAGIT | 360 | 8 (100%) | 6.7 ± 8.6 | 150 ± 45 |
| FS30D | 60 | 7 (87.5%) | 3.6 ± 4.0 | 90 ± 19 |
| | 30 | 6 (75%) | 5.2 ± 12.0 | 75 ± 47 |
| | 15 | 4 (50%) | 2.0 ± 5.1 | 120 ± 178 |

1. A pharmaceutical composition comprising a tablet core and an anionic copolymer coating,
wherein said tablet core comprises a salt of capric acid and a protease stabilised insulin,
wherein said protease stabilised insulin comprises one or more additional disulfide bridges relative to human insulin or analogues comprising the same disulfide bridges as human insulin, and/or
wherein said protease stabilised insulin comprises a linker and a fatty acid or fatty diacid side chain having 14-22 carbon atoms and optionally further comprises one or more additional disulfide bridges relative to human insulin or analogues comprising the same disulfide bridges as human insulin, and
wherein said anionic copolymer coating is a dispersion comprising between 25-35% such as 30% (meth)acrylate copolymer, wherein said (meth)acrylate copolymer consists of 10-30% (w/w) methyl methacrylate, 50-70% (w/w) methyl acrylate and 5-15% (w/w) methacrylic acid and is at least partly in direct contact with an outer surface of a tablet core.

2. The pharmaceutical composition according to claim 1, wherein said anionic copolymer coating is in direct contact with at least 10% of said tablet core.

3. The pharmaceutical composition according to claim 1, wherein said anionic copolymer coating is in direct contact with at least 50% of said tablet core.

4. The pharmaceutical composition according to claim 1, wherein said anionic copolymer coating is a coating comprising methyl acrylate, methyl methacrylate and methacrylic acid.

5. The pharmaceutical composition according to claim 1, wherein said anionic copolymer coating is an EUDRAGIT®FS30D as sold by Evonik Industries (in 2013) comprising coating.

6. The pharmaceutical composition according to claim 1, wherein said salt of capric acid is sodium caprate.

7. The pharmaceutical composition according to claim 1, wherein all ingredients of said tablet core are of a molecular weight below about 300-1000 g/mol.

8. The pharmaceutical composition according to claim 1 wherein said tablet core comprises about 60-85% (w/w) caprate.

9. The pharmaceutical composition according to claim 1, wherein said tablet core comprises about 77% (w/w) caprate, such as e.g. sodium caprate, about 22.5 minus X % (w/w) sorbitol, about X % (w/w) protease stabilised insulin and about 0.5% (w/w) stearic acid, wherein Xis selected from the group consisting of: 0.1, 0.5, 1, 1.5, 2, 2.5, 3, 3.5, 4, 4.5 or 5.

10. The pharmaceutical composition according to claim 1, wherein said anionic copolymer is present in an amount of about 4-10% (w/w) relative to the tablet core.

11. The pharmaceutical composition according to claim 1, wherein an additional continuous or discontinuous non-functional coating is applied on top of said anionic copolymer coating or an additional discontinuous non-functional coating is applied between said tablet core and said anionic copolymer coating and wherein said composition does not comprise a continuous sub coat between said tablet core and said anionic copolymer.

12. The pharmaceutical composition according to claim 1 in the form of a tablet.

13. (canceled)

14. A method for treating type 1 and/or type 2 diabetes mellitus, comprising administering a pharmaceutical composition according to claim 1 to a subject in need thereof.

15. A method for producing a pharmaceutical composition according to claim 1, comprising the steps of preparing a tablet core and directly coating said anionic copolymer on said outer surface of said tablet core.

16. The pharmaceutical composition according to claim 1, wherein said tablet core comprises about 60-85% (w/w) sodium caprate.


SEQUENCE LISTING (6 sequences)

SEQ ID NO: 1 - protein, 24 aa, Artificial
  Modified Insulin A chain
GPPGIVEQCCTSICSLYDLEDYCN

SEQ ID NO: 2 - protein, 35 aa, Artificial
  Modified Insulin B chain
GPPFVDQHLCGSHLVEALYLVCGERGFFYTPYTLE

SEQ ID NO: 3 - protein, 21 aa, Artificial
  Modified insulin A chain
GIVEQCCTSICSLTDLEQYCN

SEQ ID NO: 4 - protein, 30 aa, Artificial
  Modified insulin B chain
FVNEHLCGSHLVEALYLVCGERGFHYTPYT

SEQ ID NO: 5 - protein, 21 aa, Artificial
  Modified A chain
GIVEQCCTSCCSLEQLENYCN

SEQ ID NO: 6 - protein, 29 aa, Artificial
  Modified B chain
FVNCHLCGSHLVEALYLVCGERGFHYTPK